(19) United States
(12) Patent Application Publication
(10) Pub. No.: US 2019/0247357 A1
(43) Pub. Date: Aug. 15, 2019

(54) NANOPARTICLE FORMULATIONS AND METHODS OF MAKING AND USING THEREOF

(71) Applicant: Abraxis BioScience, LLC, Summit, NJ (US)

(72) Inventors: Willard FOSS, San Diego, CA (US); Viktor PEYKOV, San Diego, CA (US)

(21) Appl. No.: 16/340,676

(22) PCT Filed: Oct. 10, 2017
(86) PCT No.: PCT/US2017/055902

§ 371 Date: Apr. 9, 2019

Related U.S. Application Data
(60) Provisional application No. 62/406,367, filed on Oct. 10, 2016.

(51) Int. Cl. A61K 31/337; A61K 47/69; A61K 9/51; A61K 47/64; A61P 35/00; A61K 31/436; A61K 47/68; C07K 16/24; C07K 16/22; A61K 9/19; A61K 9/00

(52) U.S. Cl. CPC A61K 31/337; A61K 47/6929; A61K 9/5169; A61K 47/643; A61P 35/00; A61K 9/0019; A61K 47/6845; C07K 16/24; C07K 16/22; A61K 9/19; A61K 31/436

(57) ABSTRACT

The present invention provides compositions comprising nanoparticles comprising (a) a hydrophobic drug, (b) an albumin, and (c) a bioactive polypeptide. The present invention also provides method of making compositions comprising nanoparticles comprising (a) a hydrophobic drug, (b) an albumin, and (c) a bioactive polypeptide. Further provided are methods of use, pharmaceutical compositions, medicines, and kits thereof.

### CROSS-REFERENCE TO RELATED APPLICATION

This application claims priority from U.S. Provisional Patent Application No. 62/406,367, filed Oct. 10, 2016, the contents of which are incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name 638772021740SEQLIST.txt, date recorded: Oct. 9, 2017, size: 6 KB).

### FIELD OF THE INVENTION

The present disclosure relates to compositions comprising nanoparticles comprising (a) a hydrophobic drug, (b) an albumin, and (c) a bioactive polypeptide.

### BACKGROUND

Albumin-based nanoparticle compositions have been developed as a drug delivery system for delivering hydrophobic drugs such as a taxane. See, for example, U.S. Pat. Nos. 5,916,596; 6,506,405; 6,749,868; 6,537,579; 7,820,788; and 7,923,536. Abraxane®, an albumin stabilized nanoparticle formulation of paclitaxel, was approved in the United States in 2005 and subsequently in various other countries for treating metastatic breast cancer. It was recently also approved for treating locally advanced or metastatic non-small cell lung cancer and metastatic pancreatic cancer in the United States, Europe and other global markets.

Bevacizumab, sold under the trade name Avastin®, is an antiangiogenic antibody that targets vascular endothelial growth factor A (VEGF-A) and is effective for the treatment of several cancers. Attempts to combine the therapeutic potential of albumin-based nanoparticle compositions with antibody therapy have previously been made (see, for example, U.S. Pat. Nos. 9,427,477; 9,446,148; and PCT App. No. WO 2014/055415). But there remains a need for efficient nanoparticle production and higher quality albumin-based nanoparticles containing both a hydrophobic drug and an immunotherapeutic.

The disclosures of all publications, patents, patent applications, and published patent applications referred to herein are hereby incorporated herein by reference in their entirety.

### BRIEF SUMMARY OF THE INVENTION

Described herein are nanoparticles comprising (a) a hydrophobic drug, (b) an albumin, and (c) a bioactive polypeptide conjugated to the albumin. In some embodiments, the bioactive polypeptide is an antibody or a fragment thereof. In some embodiments, the bioactive polypeptide is conjugated (either covalently or non-covalently) to the albumin of the nanoparticle. In some embodiments, the bioactive polypeptide is embedded in the nanoparticle. Further described herein are methods of manufacturing such nanoparticle compositions.

In one aspect, there is provided a composition comprising nanoparticles comprising (a) a hydrophobic drug, (b) an albumin, and (c) a bioactive polypeptide conjugated to the albumin. In some embodiments, the bioactive polypeptide is covalently crosslinked to the albumin. In some embodiments, the bioactive polypeptide is covalently crosslinked to the albumin through a chemical crosslinker. In some embodiments, the bioactive polypeptide is covalently crosslinked to the albumin through a disulfide bond. In some embodiments, the bioactive polypeptide is conjugated to the albumin through a non-covalent crosslinker. In some embodiments, the bioactive polypeptide comprises a first component of the non-covalent crosslinker and the albumin comprises a second component of the non-covalent crosslinker, and wherein the first component specifically binds to the second component. In some embodiments, the non-covalent crosslinker comprises nucleic acid molecules, wherein at least a portion of the nucleic acid molecules are complementary.

In another aspect, there is provided a composition comprising nanoparticles comprising (a) a solid core comprising a hydrophobic drug, (b) an albumin associated with a surface of the nanoparticle, and (c) a bioactive polypeptide embedded in the surface of the nanoparticle or the solid core. In some embodiments, the bioactive polypeptide is embedded in the surface of the nanoparticles. In some embodiments, the bioactive polypeptide is embedded in the solid core.

In some embodiments of the nanoparticles described above, at least 75% of the bioactive polypeptide in the composition is associated with the nanoparticles. In some embodiments, the nanoparticles comprise at least about 100 bioactive polypeptides.

In some embodiments of the nanoparticles described above, the bioactive polypeptide is an antibody or fragment thereof. In some embodiments, the bioactive polypeptide is bevacizumab, trastuzumab, BGB-A317, or tocilizumab.

In some embodiments of the nanoparticles described above, the weight ratio of the hydrophobic drug to the bioactive polypeptide in the nanoparticles in the composition is about 1:1 to about 100:1. In some embodiments, the weight ratio of the albumin to the bioactive polypeptide in the nanoparticles in the composition is about 1:1 to about 1000:1. In some embodiments, the weight ratio of the albumin to the hydrophobic drug in the nanoparticles in the composition is about 1:1 to about 20:1. In some embodiments, the weight of the hydrophobic drug is determined by reverse-phase high performance liquid chromatography (HPLC), and the weight of the bioactive polypeptide and the albumin is determined by size exclusion chromatography (SEC); or the weight of the hydrophobic drug is determined by reverse-phase high performance liquid chromatography (HPLC), the weight of the albumin is determined by size exclusion chromatography (SEC), and the weight of bioactive polypeptide is determined by an enzyme-linked immunosorbent assay (ELISA).

In some embodiments of the nanoparticles described above, the composition further comprises bioactive polypeptide not associated with the nanoparticles.

In some embodiments, at least about 40% of the albumin in the nanoparticle portion of the composition is crosslinked by disulfide bonds.

In some embodiments, the average diameter of the nanoparticles as measured by dynamic light scattering is no greater than about 200 nm.

In some embodiments, the composition further comprises albumin not associated with the nanoparticles.

In some embodiments, the hydrophobic drug is a taxane or a limus drug. In some embodiments, the hydrophobic drug is paclitaxel. In some embodiments, the hydrophobic drug is rapamycin.

In another aspect, there is provided a method of making a composition comprising nanoparticles comprising a hydrophobic drug, an albumin and a bioactive polypeptide, the method comprising: (i) subjecting a mixture of an organic solution and an aqueous solution to high pressure homogenization, thereby forming an emulsion, wherein the organic solution comprises the hydrophobic drug dissolved in one or more organic solvents, and wherein the aqueous solution comprises the albumin and the bioactive polypeptide; and (ii) removing at least a portion of the one or more organic solvents from the emulsion, thereby forming the composition. In some embodiments, the bioactive polypeptide is conjugated to the albumin in the aqueous solution.

In another aspect, there is provided a method of making a composition comprising nanoparticles comprising a hydrophobic drug, an albumin and a bioactive polypeptide, the method comprising: (i) subjecting a mixture of an organic solution and an aqueous solution to high pressure homogenization, thereby forming an emulsion, wherein the organic solution comprises a hydrophobic drug dissolved in one or more organic solvents, and wherein the aqueous solution comprises the albumin; (ii) adding the bioactive polypeptide to the emulsion; and (iii) removing at least a portion of the one or more organic solvents from the emulsion, thereby forming the composition.

In another aspect, there is provided a method of making a composition comprising nanoparticles comprising a hydrophobic drug, an albumin and a bioactive polypeptide, the method comprising: (i) subjecting a mixture of an organic solution and an aqueous solution to high pressure homogenization, thereby forming an emulsion, wherein the organic solution comprises a hydrophobic drug dissolved in one or more organic solvents, and wherein the aqueous solution comprises the albumin; (ii) removing at least a portion of the one or more organic solvents from the emulsion to obtain a post-evaporated suspension, and (iii) adding the bioactive polypeptide to the post-evaporated suspension, thereby forming the composition.

In another aspect, there is provided a method of making a composition comprising nanoparticles comprising a hydrophobic drug, an albumin and a bioactive polypeptide, the method comprising: (i) subjecting a mixture of an organic solution and an aqueous solution to high pressure homogenization, thereby forming an emulsion, wherein the organic solution comprises a hydrophobic drug dissolved in one or more organic solvents, and wherein the aqueous solution comprises the albumin; (ii) removing at least a portion of but not all of the one or more organic solvents from the emulsion to obtain an emulsion-suspension intermediate; (iii) adding the bioactive polypeptide to the emulsion-suspension intermediate; and (iv) removing an additional portion of the one or more organic solvents from the emulsion-suspension intermediate comprising the bioactive polypeptide, thereby forming the composition.

In another aspect, there is provided a method of making a composition comprising nanoparticles comprising a hydrophobic drug, an albumin and a bioactive polypeptide, the method comprising: (i) subjecting a mixture of an organic solution and an aqueous solution to high pressure homogenization, thereby forming an emulsion, wherein the organic solution comprises a hydrophobic drug dissolved in one or more organic solvents, and wherein the aqueous solution comprises the albumin, wherein the albumin is derivatized with a crosslinker moiety; (ii) removing at least a portion of the one or more organic solvents from the emulsion to obtain a post-evaporated suspension, and (iii) adding the bioactive polypeptide to the post-evaporated suspension, wherein the bioactive polypeptide is derivatized with a crosslinker moiety, thereby forming the composition. In some embodiments, the method further comprises replacing the derivatized albumin not associated with the nanoparticles with non-derivatized albumin

In another aspect, there is provided a method of making a composition comprising nanoparticles comprising a hydrophobic drug, an albumin and a bioactive polypeptide, the method comprising: (i) subjecting a mixture of an organic solution and an aqueous solution to high pressure homogenization, thereby forming an emulsion, wherein the organic solution comprises a hydrophobic drug dissolved in one or more organic solvents, and wherein the aqueous solution comprises the albumin, wherein at least a portion of the albumin is conjugated to the bioactive polypeptide; and (ii) removing at least a portion of the one or more organic solvents from the emulsion, thereby forming the composition. In some embodiments, the method further comprises replacing the bioactive polypeptide-conjugated albumin not associated with the nanoparticles with unconjugated albumin.

In another aspect, there is provided a method of making a composition comprising nanoparticles comprising a hydrophobic drug, an albumin, and a bioactive polypeptide conjugated to the albumin, comprising conjugating the bioactive polypeptide to nanoparticles comprising the hydrophobic drug and albumin.

In some embodiments of the methods described above, the method further comprises sterile filtering the composition.

In some embodiments of the methods described above, the bioactive polypeptide is an antibody or fragment thereof. In some embodiments, the bioactive polypeptide is bevacizumab, trastuzumab, BGB-A317, or tocilizumab.

Further provided herein is a composition obtained by the method of any one of methods described above

Also provided herein, there is a pharmaceutical composition comprising any one of the compositions described above, and a pharmaceutically acceptable excipient.

In another aspect, there is provided a method of treating a disease in an individual, comprising administering to the individual an effective amount of the any of the compositions described above. In some embodiments, the disease is a cancer. In some embodiments, the individual is human.

These and other aspects and advantages of the present invention will become apparent from the subsequent detailed description and the appended claims. It is to be understood that one, some, or all of the properties of the various embodiments described herein may be combined to form other embodiments of the present invention.

### BRIEF DESCRIPTION OF THE DRAWINGS

FIG. 1 shows a schematic illustrating one embodiment of a method for making a composition comprising nanoparticles described herein by including a bioactive polypeptide and albumin in an aqueous solution.

FIG. 2 shows a schematic illustrating one embodiment of a method for making a composition comprising nanoparticles described herein by adding a bioactive polypeptide to a crude mixture comprising an aqueous solution (comprising albumin and water) and an organic solution (comprising one or more organic solvents and a hydrophobic drug).

FIG. 3 shows a schematic illustrating one embodiment of a method for making a composition comprising nanoparticles described herein by adding a bioactive polypeptide to an emulsion comprising an aqueous solution (comprising albumin and water) and an organic solution (comprising one or more organic solvents and a hydrophobic drug).

FIG. 4 shows a one embodiment of a method for making a composition comprising nanoparticles described herein by adding a bioactive polypeptide to a post-evaporation nanoparticle suspension, wherein the nanoparticles comprise albumin and a hydrophobic drug.

FIG. 5 shows a schematic illustrating one embodiment of a method for making a composition comprising nanoparticles described herein by adding a bioactive polypeptide to pre-manufactured nanoparticles comprising albumin and a hydrophobic drug.

FIG. 6 shows a schematic illustrating one embodiment of a method for making a composition comprising nanoparticles described herein by adding a bioactive polypeptide to a nanoparticle suspension, wherein the nanoparticles comprise derivatized albumin and a hydrophobic drug. The bioactive polypeptide conjugates to the derivatized albumin associated with the nanoparticles.

FIG. 7A is an image taken by optical microscopy of Abraxane® reconstituted to 10 mg/mL with 100% of Avastin® buffer (containing sodium phosphate buffer and α,α-trehalose, but excluding polysorbate 20), and without adjusting the pH (the pH was measured as 6.4) after incubating for 24 hours at room temperature.

FIG. 7B is an image taken by optical microscopy of Abraxane® reconstituted to 10 mg/mL with 20% of Avastin® buffer (containing sodium phosphate buffer and α,α-trehalose, but excluding polysorbate 20) and 80% normal saline, and the pH was adjusted to 5 after incubating for 24 hours at 58° C.

FIG. 7C is an image taken by optical microscopy of Abraxane® reconstituted to 10 mg/mL with 100% of Avastin® buffer (containing each of sodium phosphate buffer and α,α-trehalose, and polysorbate 20), and without adjusting the pH (the pH was measured as 6.8), after incubating for 24 hours at room temperature.

FIG. 8A shows results from size exclusion chromatography measurements of bevacizumab without albumin at various points during the nanoparticle manufacturing process.

FIG. 8B shows the fraction of bevacizumab (without albumin) recovered after each step of the manufacturing process relative to the initial concentration added to the beginning of the manufacturing process.

FIG. 9A shows results from size exclusion chromatography measurements of bevacizumab with 10% human albumin at various points during the nanoparticle manufacturing process.

FIG. 9B shows the fraction of bevacizumab (with 0%, 1%, 2.5%, 5%, or 10% human albumin) recovered after each step of the manufacturing process relative to the initial concentration added to the beginning of the manufacturing process, when the bevacizumab is provided in the initial aqueous solution.

FIG. 9C shows the fraction of bevacizumab monomer (with 0%, 1%, 2.5%, 5%, or 10% human albumin) remaining after each step of the manufacturing process relative to the initial concentration added to the beginning of the manufacturing process, when the bevacizumab is provided in the initial aqueous solution.

FIG. 10A shows the fraction of bevacizumab recovered after each step of the manufacturing process relative to the amount of bevacizumab provided, when the bevacizumab is provided in the initial aqueous solution with 5% human albumin or provided to the emulsion after the high-pressure homogenization of the albumin aqueous solution and organic solution.

FIG. 10B shows the fraction of bevacizumab monomer remaining after each step of the manufacturing process relative to the amount of bevacizumab provided, when the bevacizumab is provided in the initial aqueous solution with 5% human albumin or provided to the emulsion after the high-pressure homogenization of the albumin aqueous solution and organic solution.

FIG. 11 shows nanoparticle size (determined by dynamic light scattering) for nab-paclitaxel (“Abx”), admixtures of bevacizumab and nab-paclitaxel at different ratios (“Bev:Abx (8:10)” and “Bev:Abx (8:10)”), admixtures of trastuzumab and nab-paclitaxel at different ratios (“Tras:Abx (8:10)” and “Tras:Abx (8:10)”), and bevacizumab alone (“Bev”) at different saline concentrations.

FIG. 12 shows a percent change of tumor volume seven days after administering an admixture of nab-paclitaxel and bevacizumab (“AB160”), bevacizumab and nab-paclitaxel administered on the same day (“BEV12+ABX30—Same Day”) or one day apart (“BEV12+ABX30—1 Day Apart”), bevacizumab alone (“BEV12”), nab-paclitaxel alone (“ABX30”) or a vehicle control (“Vehicle”).

FIG. 13 shows a schematic for conjugation of an antibody and free human serum albumin (HSA).

FIGS. 14A-14C show deconvoluted mass spectra of trastuzumab and SM(PEG)₆ activated trastuzumab species. FIG. 14A shows a mass spectrum of trastuzumab. FIG. 14B shows a mass spectrum of SM(PEG)₆ activated trastuzumab using a trastuzumab:linker ratio of 1:5. FIG. 14C shows a mass spectrum of SM(PEG)₆ activated trastuzumab using a trastuzumab:linker ratio of 1:10.

FIG. 15 shows a SEC chromatogram of a 1:1 trastuzumab-HSA conjugate, trastuzumab, and HSA.

FIG. 16 shows a SDS-PAGE gel (4-12%) of trastuzumab-HSA conjugation products.

FIG. 17 shows a native gel of trastuzumab-HSA conjugation and trastuzumab conjugation products.

FIG. 18 shows a schematic for conjugation of an activated antibody and an isolated nab-paclitaxel particle.

FIG. 19 shows a schematic for conjugation of an activated antibody and a thiolated, isolated nab-paclitaxel particle.

FIG. 20 shows a 3-8% Tris-acetate SDS PAGE gel of samples from the conjugation reaction of isolated nab-paclitaxel nanoparticles or a nab-paclitaxel formulation (ABX) and trastuzumab.

FIG. 21 shows a schematic for conjugation of an antibody and an isolated nab-paclitaxel particle using copper-free click chemistry.

FIG. 22 shows a schematic for boronic acid modification of an isolated nab-paclitaxel particle.

FIG. 23 shows a schematic for conjugation of an activated antibody and an activated isolated nab-paclitaxel particle using a DNA crosslinker.

FIG. 24 shows deconvoluted mass spectra from LC-MS analyses of nivolumab and species of activated nivolumab.

FIGS. 25A-25D show percentage tumor volume change for a smaller tumor study at time points following treatment administration. FIGS. 25A and 25B show percentage tumor volume change for the smaller tumor study on day 7 following treatment administration on day 0. FIGS. 23C-D show percentage tumor volume change for the smaller tumor study on day 14 following treatment administration on days 0 and 7.

FIGS. 26A-26B show percentage tumor volume change for the larger tumor study at time points following treatment administration. FIG. 26A shows percentage tumor volume change for the larger tumor study on day 7 following treatment administration on day 0. FIG. 26B shows percentage tumor volume change for the larger tumor study on day 14 following treatment administration on days 0 and 7.

FIGS. 27A-27B show percentage tumor volume change for BT-474 xenograft tumors at time points following treatment administration. FIG. 27A shows percentage tumor volume change for BT-474 xenograft tumors on day 7 following treatment administration on day 0. FIG. 27B shows percentage tumor volume change for BT-474 xenograft tumors on day 14 following treatment administration on days 0 and 7.

FIGS. 28A-28D show percentage tumor volume change for BT-474 xenograft tumors at time points following treatment administration. FIGS. 28A and 28B show percentage tumor volume change for BT-474 xenograft tumors on day 7 following treatment administration on day 0. FIGS. 28C and 28D show percentage tumor volume change for BT-474 xenograft tumors on day 14 following treatment administration on days 0 and 7.

### DETAILED DESCRIPTION OF THE INVENTION

The present application provides compositions comprising nanoparticles comprising (a) a hydrophobic drug, (b) an albumin, and (c) a bioactive polypeptide (such as an antibody or fragment thereof). For example, in some embodiments, there is provided a composition comprising nanoparticles comprising (a) a hydrophobic drug (such as a taxane, for example paclitaxel), (b) an albumin (such as human albumin or a derivatized albumin), and (c) a bioactive polypeptide (such as an antibody, such as a therapeutic antibody). In some embodiments, the composition further comprises another therapeutic agent. In some embodiments, the composition further comprises an albumin and/or a bioactive polypeptide not associated with the nanoparticle portion of the composition.

In certain embodiments, the bioactive polypeptide is embedded into the nanoparticle. The embedded bioactive polypeptide may be embedded into the surface albumin of the nanoparticle, or may be embedded into the hydrophobic core of the nanoparticle. The bioactive polypeptide can be embedded into the nanoparticles by including the bioactive polypeptide in one or more manufacturing stages of the nanoparticle, as opposed to mixing the bioactive polypeptide with a pre-formed, lyophilized nanoparticle composition (i.e., an admixture). As described in further detail herein, in certain embodiments, manufacture of a nanoparticle suspension, wherein the nanoparticles contain albumin and a hydrophobic drug, includes i) forming an emulsion (e.g., by high-pressure homogenization) of an organic solvent containing the hydrophobic drug and an aqueous solution containing the albumin, and ii) removing at least a portion of the organic solvents from the emulsion (for example, by evaporation) to form a nanoparticle suspension. In certain embodiments, the manufacturing process can further include formulating the nanoparticle suspension (e.g., by adding albumin or water) and/or lyophilizing the suspension to form the nanoparticle composition. In some embodiments, the bioactive polypeptide is added to one or more nanoparticle composition precursors, such as the aqueous solution containing the albumin prior to forming the emulsion, the formed emulsion (either prior to or during removal of the organic solvent), or to the suspension after removal of the organic solvent (i.e. the “post-evaporation suspension”).

In certain embodiments, the bioactive polypeptide is conjugated to a nanoparticle, for example through a crosslinker, which may be a covalent crosslinker or a noncovalent crosslinker. The crosslink can link a segment of the bioactive polypeptide to a segment of the albumin, thereby associating the bioactive polypeptide to the albumin (and thus, the nanoparticle). In some embodiments, the crosslinker provides a covalent linkage between the bioactive polypeptide and the albumin That is, the crosslinker is covalently linked to the bioactive polypeptide and covalently linked to the albumin, thereby providing a covalent bridge between the two entities. In certain aspects, the crosslinker provides a non-covalent linkage between the bioactive polypeptide and the albumin. For example, the albumin can be covalently conjugated to a first crosslinker component and the bioactive polypeptide can be covalently conjugated to a second crosslinker component, wherein the first crosslinker component and the second crosslinker component bind together (for example, complementary nucleic acid molecules, such as complementary DNA).

The present application further provides methods of making compositions comprising nanoparticles comprising (a) a hydrophobic drug, (b) an albumin, and (c) a bioactive polypeptide. In some embodiments, the method comprises i) subjecting a mixture of an organic solution and an aqueous solution to high-pressure homogenization, thereby forming an emulsion, wherein the organic solution comprises the hydrophobic drug dissolved in one or more organic solvents, and wherein the aqueous solution comprises the albumin and the bioactive polypeptide; and ii) removing at least a portion of the one or more organic solvents from the emulsion, thereby forming the composition. In some embodiments, the method comprises i) subjecting a mixture of an organic solution and an aqueous solution to high-pressure homogenization, thereby forming an emulsion, wherein the organic solution comprises the hydrophobic drug dissolved in one or more organic solvents, and wherein the aqueous solution comprises the albumin; ii) adding the bioactive polypeptide to the emulsion; and iii) removing at least a portion of the one or more organic solvents from the emulsion, thereby forming the composition. In some embodiments, the method comprises i) subjecting a mixture of an organic solution and an aqueous solution to high pressure homogenization, thereby forming an emulsion, wherein the organic solution comprises the hydrophobic drug dissolved in one or more organic solvents, and wherein the aqueous solution comprises the albumin; ii) removing at least a portion of the one or more organic solvents from the emulsion to obtain a post-evaporated suspension, and iii) adding the bioactive polypeptide to the post-evaporated suspension, thereby forming the composition.

In certain embodiments, at least a portion of the albumin included in the aqueous solution during the nanoparticle manufacturing process is conjugated to a bioactive polypeptide. For example, in some embodiments, the method comprises i) subjecting a mixture of an organic solution and an aqueous solution to high pressure homogenization, thereby forming an emulsion, wherein the organic solution comprises the hydrophobic drug dissolved in one or more organic solvents, and wherein the aqueous solution comprises the albumin, wherein at least a portion of the albumin is conjugated to the bioactive polypeptide; and ii) removing at least a portion of the one or more organic solvents from the emulsion, thereby forming the composition.

In certain embodiments, at least a portion of the albumin included in the aqueous solution during the nanoparticle manufacturing process is derivatized (for example by thiolation of the albumin or covalently attaching a first segment of a noncovalent crosslinker to the albumin). In some embodiments, the method comprises i) subjecting a mixture of an organic solution and an aqueous solution to high pressure homogenization, thereby forming an emulsion, wherein the organic solution comprises the hydrophobic drug dissolved in one or more organic solvents, and wherein the aqueous solution comprises the albumin, wherein at least a portion of the albumin is derivatized; ii) removing at least a portion of the one or more organic solvents from the emulsion to obtain a post-evaporated suspension; and iii) adding a bioactive polypeptide to the post-evaporated suspension. In some embodiments, the bioactive polypeptide is derivatized. For example, in some embodiments, the method comprises i) subjecting a mixture of an organic solution and an aqueous solution to high pressure homogenization, thereby forming an emulsion, wherein the organic solution comprises the hydrophobic drug dissolved in one or more organic solvents, and wherein the aqueous solution comprises the albumin, wherein at least a portion of the albumin is derivatized; ii) removing at least a portion of the one or more organic solvents from the emulsion to obtain a post-evaporated suspension; and iii) adding a derivatized bioactive polypeptide to the post-evaporated suspension.

In certain embodiments, the nanoparticles are manufactured by conjugating the bioactive polypeptide to pre-formed nanoparticles, which may be lyophilized or in a suspension (for example, a reconstituted suspension wherein the nanoparticles were previously lyophilized, or in a post-evaporation nanoparticle suspension).

The compositions (such as pharmaceutical compositions) disclosed herein are useful for treating various diseases, such as cancer. The present application thus provides compositions (such as pharmaceutical compositions) comprising nanoparticles comprising (a) a hydrophobic drug, (b) an albumin, and (c) a bioactive polypeptide, as well as methods of using such compositions for the treatment of diseases, including cancer. Further provided herein are combination treatments comprising administering an effective amount of a composition described herein and an effective amount of another therapeutic agent (such as a chemotherapeutic agent). Also provided are kits, medicines, medicaments, compositions for use, unit dosage forms, pharmaceutical compositions (such as lyophilized compositions), comprising the compositions described herein.

Sectional headings provided below are for organizational purposes only, and are not intended to limit the scope of the invention.

### Definitions

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from a disease, diminishing the extent of a disease, stabilizing a disease (e.g., preventing or delaying the worsening of the disease), preventing or delaying the spread (e.g., metastasis) of a disease, preventing or delaying the recurrence of a disease, delaying or slowing the progression of a disease, ameliorating a disease state, providing remission (partial or total) of a disease, decreasing the dose of one or more other medications required to treat a disease, delaying the progression of a disease, increasing the quality of life, and/or prolonging survival. Also encompassed by “treatment” is a reduction of a pathological consequence of a disease (such as cancer). The methods of the invention contemplate any one or more of these aspects of treatment.

The term “individual” refers to a mammal and includes, but is not limited to, human, bovine, horse, feline, canine, rodent, or primate. In some embodiments, the individual is a human. In some embodiments, the human is male. In some embodiments, the human is female.

As used herein, the term “antibody” includes, but is not limited to, a monoclonal antibody, polyclonal, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding fragment thereof, bifunctional hybrid antibodies, a single chain antibody, and a Fc-containing polypeptide, such as an immunoadhesion. In some embodiments, the antibody may be of any heavy chain isotype (e.g., IgG, IgA, IgM, IgE, or IgD). In some embodiments, the antibody may be of any light chain isotype (e.g., kappa or gamma). The antibody may be non-human (e.g., from mouse, goat, or any other animal), fully human, humanized, or chimeric. In some embodiments, the antibody is a derivatized antibody.

As used herein, an “at risk” individual is an individual who is at risk of developing a disease (such as cancer). An individual “at risk” may or may not have detectable disease, and may or may not have displayed detectable disease prior to the treatment methods described herein. “At risk” denotes that an individual has one or more so-called risk factors, which are measurable parameters that correlate with development of a disease, which are described herein. An individual having one or more of these risk factors has a higher probability of developing a disease than an individual without these risk factor(s).

“Adjuvant setting” refers to a clinical setting in which an individual has had a history of a disease, and generally (but not necessarily) been responsive to therapy, which includes, but is not limited to, surgery (e.g., surgical resection), radiotherapy, and/or chemotherapy. However, because of their history of a disease, these individuals are considered at risk of development of the disease. Treatment or administration in the “adjuvant setting” refers to a subsequent mode of treatment. The degree of risk (e.g., when an individual in the adjuvant setting is considered as “high risk” or “low risk”) depends upon several factors, most usually the extent of a disease when first treated.

As used herein, “bioactive polypeptide” refers to a molecule comprising two or more amino acids linked by peptide (amide) bonds comprising a portion thereof with activity associated with binding of a ligand or receptor or activity associated with inhibiting another agent binding a ligand or receptor. Bioactive polypeptides include, but are not limited to, oligopeptides, peptides, polypeptide aptamers, proteins, multimeric proteins, fusion proteins, antibodies, or fragments thereof. In some embodiments, the bioactive polypeptide comprises a portion for associating with an albumin-hydrophobic drug nanoparticle.

“Neoadjuvant setting” refers to a clinical setting in which the method is carried out before the primary/definitive therapy.

As used herein, “delaying” the development of a disease means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of a disease is a method that reduces probability of disease development in a given time frame and/or reduces the extent of the disease in a given time frame, when compared to not using the method. Such comparisons are typically based on clinical studies, using a statistically significant number of subjects. Disease development can be detectable using standard methods, including, but not limited to, computerized axial tomography (CAT Scan), Magnetic Resonance Imaging (MRI), abdominal ultrasound, clotting tests, arteriography, or biopsy. Development may also refer to disease progression that may be initially undetectable and includes occurrence, recurrence, and onset.

The term “effective amount” used herein refers to an amount of a compound or composition sufficient to treat a specified disorder, condition, or disease, such as ameliorate, palliate, lessen, and/or delay one or more of its symptoms. In reference to a disease such as a cancer, an effective amount comprises an amount sufficient to cause a tumor to shrink and/or to decrease the growth rate of the tumor (such as to suppress tumor growth) or to prevent or delay other unwanted cell proliferation in the cancer. In some embodiments, the effective amount is an amount sufficient to delay development of a cancer. In some embodiments, the effective amount is an amount sufficient to prevent or delay recurrence. An effective amount can be administered in one or more administrations. In the case of a cancer, the effective amount of the drug or composition may: (i) reduce the number of epithelioid cells; (ii) reduce tumor size; (iii) inhibit, retard, slow to some extent and preferably stop the cancer cells infiltration into peripheral organs; (iv) inhibit (e.g., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

As used herein, by “combination therapy” is meant that a first agent be administered in conjunction with another therapeutic agent. “In conjunction with” refers to administration of one treatment modality in addition to another treatment modality, such as administration of a nanoparticle composition described herein in addition to administration of another therapeutic agent to the same individual. As such, “in conjunction with” refers to administration of one treatment modality before, during, or after delivery of the other treatment modality to an individual.

The term “simultaneous administration,” as used herein, means that a first therapy and second therapy in a combination treatment are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20 or more minutes, 30 or more minutes, 40 or more minutes, 50 or more minutes, 60 or more minutes, 2 or more hours, 4 or more hours, 6 or more hours, 12 or more hours, or 24 or more hours. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, the term “nab” stands for nanoparticle albumin-bound. For example, nab-paclitaxel is a nanoparticle albumin-bound formulation of paclitaxel.

The term “emulsion” as used herein refers to a liquid with a dispersed organic phase comprising droplets having an average diameter of about 1 micrometer or less in a continuous aqueous phase.

The term “hydrophobic drug” refers to a drug with a solubility of about 1 mg/mL or less in water at pH 7 at about 25° C.

It is understood that aspects and embodiments of the invention described herein include “consisting” and/or “consisting essentially of” aspects and embodiments.

Reference to “about” a value or parameter herein includes (and describes) variations that are directed to that value or parameter per se. For example, description referring to “about X” includes description of “X.” Additionally, use of “about” preceding any series of numbers includes “about” each of the recited numbers in that series. For example, description referring to “about X, Y, or Z” is intended to describe “about X, about Y, or about Z.”

As used herein and in the appended claims, the singular forms “a,” “or,” and “the” include plural referents unless the context clearly dictates otherwise.

Compositions Comprising Nanoparticles

The compositions described herein comprise nanoparticles comprising (a) a hydrophobic drug, (b) an albumin, and (c) a bioactive polypeptide. In some embodiments, the compositions described herein further comprise albumin and/or bioactive polypeptide not associated with the nanoparticles. In some embodiments, the compositions described herein further comprise another therapeutic agent. In some embodiments, the compositions described herein further comprise a pharmaceutically acceptable carrier.

The nanoparticles described herein comprise a hydrophobic drug. In some embodiments, the nanoparticles comprise a solid core comprising a hydrophobic drug. As used herein, “core” refers to an inner portion of a nanoparticle wherein substantial all of a hydrophobic drug associated with the nanoparticle is located. In some embodiments, the nanoparticle comprises a solid core comprising a hydrophobic drug. In some embodiments, the nanoparticle comprises a solid core of a hydrophobic drug.

In some embodiments, the hydrophobic drug in a nanoparticle constitutes more than about 50%, 60%, 70%, 80%, 90%, 95%, or 99% of the nanoparticle by weight. In some embodiments, the nanoparticle has a non-polymeric matrix. In some embodiments, the nanoparticle comprises a solid core of hydrophobic drug that is substantially free of polymeric materials (such as a polymeric matrix).

The solid core of a hydrophobic drug in a nanoparticle, in some embodiments, further comprises a portion of a bioactive polypeptide. In some embodiments, the nanoparticle comprises a solid core comprising a hydrophobic drug and a portion of a bioactive polypeptide.

As described herein, the nanoparticles comprise an albumin. Contemplated within the invention are albumins including, but not limited to, human albumin, human serum albumin, recombinant albumin, and derivatives thereof. In some embodiments, the albumin is human albumin. In some embodiments, the albumin is human serum albumin. In some embodiments, the human albumin is human serum albumin. In some embodiments, the albumin is recombinant albumin. In some embodiments, the albumin is human recombinant albumin. In some embodiments, the recombinant albumin is human recombinant albumin.

Albumins, such as human serum albumin (HSA), are highly soluble globular proteins. For example, human serum albumin consists of 585 amino acids and has a molecular weight of about 66 kDa. HSA is the most abundant protein in human plasma and accounts for 70-80% of the colloid osmotic pressure of human plasma. The amino acid sequence of HSA contains a total of 17 disulphide bridges, one free thiol (Cys 34), and a single tryptophan (Trp 214). Intravenous use of HSA solution has been indicated for the prevention and treatment of hypovolumic shock (see, e.g., Tullis, JAMA, 237, 355-360, 460-463 (1977); Houser et al., Surgery, Gynecology and Obstetrics, 150, 811-816 (1980)) and in conjunction with exchange transfusion in the treatment of neonatal hyperbilirubinemia (see, e.g., Finlayson, Seminars in Thrombosis and Hemostasis, 6, 85-120, (1980)).

Albumins, such as human serum albumin (HSA), have multiple hydrophobic binding sites. HSA has eight binding sites for fatty acids and binds a diverse set of hydrophobic drugs, for example taxanes, including neutral and negatively charged hydrophobic compounds (Goodman et al., The Pharmacological Basis of Therapeutics, 9^{th} ed, McGraw-Hill New York (1996)). Two high affinity binding sites have been proposed in subdomains HA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vorum, Dan. Med. Bull., 46, 379-99 (1999), Kragh-Hansen, Dan. Med. Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (199b), and Carter et al., Adv. Protein. Chem., 45, 153-203 (1994)). Paclitaxel and propofol have been shown to bind HSA (see, e.g., Paal et al., Eur. J. Biochem., 268(7), 2187-91 (200a), Purcell et al., Biochim. Biophys. Acta, 1478(a), 61-8 (2000), Altmayer et al., Arzneimittelforschung, 45, 1053-6 (1995), and Garrido et al., Rev. Esp. Anestestiol. Reanim., 41, 308-12 (1994)). In addition, docetaxel has been shown to bind to human plasma proteins (see, e.g., Urien et al., Invest. New Drugs, 14(b), 147-51 (1996)).

Other albumins are contemplated, such as bovine serum albumin Use of such non-human albumins may be appropriate, for example, in the context of use of these compositions in non-human mammals, such as the veterinary (including domestic pets and agricultural context).

Also contemplated within the scope of the disclosure are derivatized albumins. As used herein, a “derivatized albumin” is an albumin that has been modified after expression of the albumin. In some embodiments, the derivatized albumin is an albumin conjugated with a chemical crosslinker. In some embodiments, the derivatized albumin is an albumin conjugated with a chemical crosslinker moiety reactive (such as specifically reactive) to another chemical crosslinker moiety conjugated to a bioactive polypeptide. For example, in some embodiments, the derivatized albumin is conjugated with a chemical crosslinker comprising an alkyne moiety and the derivatized bioactive polypeptide is conjugated with a chemical crosslinker comprising an azide moiety. In some embodiments, the derivatized albumin is conjugated with a chemical crosslinker comprising an azide moiety and the derivatized bioactive polypeptide is conjugated with a chemical crosslinker comprising an alkyne moiety. Other pairs of crosslinking moieties useful for associating an albumin and a bioactive polypeptide include, but are not limited to, a strained alkyne and an azide, a strained alkyne and a nitrone (such as a 1,3-nitrone), a strained alkene and an azide, a strained alkene and a tetrazine, and a strained alkene and a tetrazole. In some embodiments, the derivatized albumin is thiolated, for example by reacting one or more amines of the albumin to form a sulfhydryl group (e.g., but reacting the amine with 2-iminothiolane (Traut's reagent) or other thiolating reagent). In some embodiments, the derivatized albumin is covalently attached to a first component of a noncovalent crosslinker (such as a nucleic acid molecule, such as DNA), wherein the bioactive polypeptide can be crosslinked to a second component of the noncovalent crosslinker that can specifically bind to the first component of the crosslinker (e.g., a nucleic acid strand complementary to the nucleic acid strand of the first component of the crosslinker). In some embodiments, the derivatized albumin is derivatized human albumin. In some embodiments, the derivatized albumin is derivatized human serum albumin. In some embodiments, the derivatized human albumin is derivatized human serum albumin. In some embodiments, the derivatized albumin is derivatized recombinant albumin. In some embodiments, the derivatized albumin is derivatized human recombinant albumin. In some embodiments, the derivatized recombinant albumin is derivatized human recombinant albumin.

In some embodiments, the albumin has sulfhydral groups that can form disulfide bonds. In some embodiments, the albumin forms an intermolecular disulfide bond with another albumin. In some embodiments, the albumin forms an intermolecular disulfide bond with a bioactive polypeptide. In some embodiments, at least about 5% (including for example at least about 10%, 15%, 20%, 25%, 30%, 40%, 50%, 60%, 70%, 80%, or 90%) of the albumin in a nanoparticle portion of a composition are crosslinked (for example crosslinked through one or more disulfide bonds). In some embodiments, the composition comprises a nanoparticle portion, wherein at least about 40%, 50%, 60%, 70%, or 80% of an albumin in a nanoparticle portion of the composition is crosslinked by disulfide bonds. In some embodiments, about 40%, 50%, 60%, 70%, or 80% of the albumin in a nanoparticle portion of a composition is crosslinked by disulfide bonds. In some embodiments, about 40% to about 80%, about 40% to about 70%, or about 50% to about 80% of the albumin in a nanoparticle portion of a composition is crosslinked by disulfide bonds.

In certain embodiments, the albumin in the nanoparticles is conjugated to a bioactive polypeptide by attaching the bioactive polypeptide to a thiol group on the albumin (e.g., Cys34). In some embodiments, nanoparticles comprising a hydrophobic drug, an albumin, and a bioactive polypeptide conjugated to the albumin have fewer free thiols than the nanoparticle comprising the hydrophobic drug and the albumin without a bioactive polypeptide conjugated to the albumin. In some embodiments, free thiols on the nanoparticle comprising a hydrophobic drug, an albumin, and a bioactive polypeptide conjugated to the albumin are decreased by about 5% or more (such as about 10%, 15%, 20%, 25%, 30%, 40%, 45%, or 50% or more) compared to the nanoparticle comprising the hydrophobic drug and the albumin without a bioactive polypeptide conjugated to the albumin. In some embodiments, nanoparticles comprising a hydrophobic drug, an albumin, and a bioactive polypeptide conjugated to the albumin have fewer free surface thiols than the nanoparticle comprising the hydrophobic drug and the albumin without a bioactive polypeptide conjugated to the albumin. In some embodiments, free thiols on a surface of the nanoparticle comprising a hydrophobic drug, an albumin, and a bioactive polypeptide conjugated to the albumin are decreased by about 5% or more (such as about 10%, 15%, 20%, 25%, 30%, 40%, 45%, or 50% or more) compared to the nanoparticle comprising the hydrophobic drug and the albumin without a bioactive polypeptide conjugated to the albumin. In some embodiments, nanoparticles comprising a hydrophobic drug, an albumin, and a bioactive polypeptide conjugated to the albumin have fewer albumin monomers (i.e., albumin not conjugated to another albumin, bioactive polypeptide, or any other polypeptide) than the nanoparticle comprising the hydrophobic drug and the albumin without a bioactive polypeptide conjugated to the albumin. In some embodiments, the amount of albumin monomers associated with nanoparticles comprising a hydrophobic drug, an albumin, and a bioactive polypeptide conjugated to the albumin is decreased by about 5% or more (such as about 10%, 15%, 20%, 25%, 30%, 40%, 45%, or 50% or more) compared to the amount of albumin monomers associated with nanoparticles comprising the hydrophobic drug and the albumin without a bioactive polypeptide conjugated to the albumin.

In some embodiments, the nanoparticle comprises (a) a hydrophobic drug, (b) an albumin, and (c) a bioactive polypeptide, wherein a biologically active portion of the bioactive polypeptide on the nanoparticle remains exposed. For example, in some embodiments, the bioactive polypeptide is positioned on the nanoparticle to allow for a portion of the bioactive polypeptide to bind a ligand or receptor. In some embodiments, the nanoparticle comprises a bioactive polypeptide with therapeutic activity.

In some embodiments, at least about 15%, 20%, 25%, 30%, 35%, 40%, 45%, 50%, 55%, 60%, 65%, 70%, 75%, 80%, 85%, 90%, or 95% of the bioactive polypeptide in a composition is associated with nanoparticles. In some embodiments, about 15%, 20%, 25%, 30%, 35%, 40%, 45%, 50%, 55%, 60%, 65%, 70%, 75%, 80%, 85%, 90%, or 95% of the bioactive polypeptide in a composition is associated with nanoparticles. In some embodiments, about 15% to about 90%, about 20% to about 85%, about 30% to about 80%, about 40% to about 80%, about 50% to about 75%, about 60% to about 85%, about 65% to about 80%, or about 70% to about 80% of the bioactive polypeptide in a composition is associated with nanoparticles.

In some embodiments, the nanoparticle comprises at least about 50, 100, 150, 250, 300, 350, 400, 450, 500, 550, 600, 650, 700, 750, 800, 850, 900, 950, 1000, 1050, 1100, 1150, or 1200 bioactive polypeptides. In some embodiments, the nanoparticle comprises about 50, 100, 150, 250, 300, 350, 400, 450, 500, 550, 600, 650, 700, 750, 800, 850, 900, 950, 1000, 1050, 1100, 1150, or 1200 bioactive polypeptides. In some embodiments, the nanoparticle comprises about 100 to about 900, about 100 to about 500, about 150 to about 700, about 100 to about 800, about 300 to about 600, about 200 to about 900, about 500 to about 1000, about 500 to about 800, about 600 to about 800 bioactive polypeptides. In some embodiments, the composition comprises nanoparticles, wherein the average number of bioactive polypeptides per nanoparticle is at least about 50, 100, 150, 250, 300, 350, 400, 450, 500, 550, 600, 650, 700, 750, 800, 850, 900, 950, 1000, 1050, 1100, 1150, or 1200 bioactive polypeptides. In some embodiments, the composition comprises nanoparticles, wherein the average number of bioactive polypeptides per nanoparticle is about 50, 100, 150, 250, 300, 350, 400, 450, 500, 550, 600, 650, 700, 750, 800, 850, 900, 950, 1000, 1050, 1100, 1150, or 1200 bioactive polypeptides.

The weight ratio of the hydrophobic drug to the bioactive polypeptide in nanoparticles in compositions may be optimized for the intended therapeutic use. In some embodiments, the weight ratio of the hydrophobic drug to the bioactive polypeptide in nanoparticles in a composition is between about 1:1 and 200:1 (such as between about 1:1 and about 100:1, about 1:1 and about 80:1, about 1:1 and about 60:1, about 1:1 and about 50:1, about 2:1 and about 40:1, about 4:1 and about 30:1, or about 6:1 to about 20:1). In some embodiments, the weight of hydrophobic drug is determined by reverse-phase high performance liquid chromatography (RP-HPLC). In some embodiments, the weight of bioactive polypeptide is determined by size exclusion chromatography (SEC) or an enzyme-linked immunosorbent assay (ELISA). ELISA allows for a direct measurement of the bioactive polypeptide associated with the nanoparticle, and further allows for distinguishing between functional bioactive polypeptide and nonfunctional (e.g., denatured) bioactive polypeptide.

The weight ratio of the albumin to the hydrophobic drug in nanoparticles in the compositions may be optimized based on presence of a hydrophobic drug, an albumin, a bioactive polypeptide, another therapeutic agent, or combinations thereof. In some embodiments, the weight ratio of the albumin to the hydrophobic drug in nanoparticles in a composition is about 1:1 to about 50:1, about 1:1 to about 20:1, about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, or about 1:1 to about 2:1. In some embodiments, the weight ratio of the albumin to the hydrophobic drug in nanoparticles in a composition is less than about 18:1, 15:1, or 10:1. In some embodiments, the weight ratio of the albumin to the hydrophobic drug in nanoparticles in a composition is about 1:1 to about 18:1, about 2:1 to about 15:1, about 3:1 to about 13:1, about 4:1 to about 12:1, about 5:1 to about 10:1. In some embodiments, the weight ratio of the albumin to the hydrophobic drug in nanoparticles in a composition is about 1:2, 1:3, 1:4, 1:5, 1:6, 1:7, 1:8, 1:9, 1:10, 1:11, 1:12, 1:13, 1:14, or 1:15. In some embodiments, the weight of albumin is determined by size exclusion chromatography (SEC). In some embodiments, the weight of hydrophobic drug is determined by reverse-phase high performance liquid chromatography (RP-HPLC).

The weight ratio of a bioactive polypeptide to the albumin in nanoparticles in compositions may be optimized based on presence of a hydrophobic drug, an albumin, a bioactive polypeptide, another therapeutic agent, or combinations thereof. In some embodiments, the weight ratio of the bioactive polypeptide to the albumin in nanoparticles in a composition is about 1:1 to about 1:1000, about 1:1 to about 1:800, about 1:1 to about 1:600, about 1:1 to about 1:500, about 1:1 to about 1:400, about 1:1 to about 1:300, about 1:1 to about 1:250, about 2:1 to about 1:200, about 2:1 to about 1:150, about 4:1 to about 1:100, or about 4:1 to about 1:50. In some embodiments, the weight of albumin is determined by size exclusion chromatography (SEC). In some embodiments, the weight of the bioactive polypeptide is determined by size exclusion chromatography (SEC) or by an enzyme-linked immunosorbent assay (ELISA).

In some embodiments, the composition comprises nanoparticles with an average diameter of no greater than about 1000 nanometers (nm), such as no greater than about any of 900, 800, 700, 600, 500, 400, 300, 200, and 100 nm. In some embodiments, the average diameter of the nanoparticles is no greater than about 200 nm. In some embodiments, the average diameter of the nanoparticles is no greater than about 150 nm. In some embodiments, the average diameter of the nanoparticles is no greater than about 100 nm. In some embodiments, the average diameter of the nanoparticles is about 20 to about 400 nm. In some embodiments, the average diameter of the nanoparticles is about 40 to about 200 nm. In some embodiments, the nanoparticles are sterile-filterable. Average diameter of the nanoparticles can be measured by Dynamic Light Scattering (DLS).

In some embodiments, the nanoparticles in the composition described herein have an average diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least about any one of 60%, 70%, 80%, 90%, 95%, or 99%) of the nanoparticles in a composition have a diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least any one of 60%, 70%, 80%, 90%, 95%, or 99%) of the nanoparticles in a composition fall within the range of about 20 to about 400 nm, including for example about 20 to about 200 nm, about 40 to about 200 nm, about 30 to about 180 nm, and any one of about 40 to about 150, about 50 to about 120, and about 60 to about 100 nm.

Exemplary embodiments of nanoparticles containing a hydrophobic drug, albumin, and a bioactive polypeptide are further detailed below. Such nanoparticles can include a bioactive polypeptide that is embedded into the nanoparticle (such as embedded into the surface or core of the nanoparticle), or nanoparticles that include a bioactive polypeptide that is conjugated (for example, through a covalent or non-covalent crosslinker) to albumin in the nanoparticle. The association of a bioactive polypeptide and an albumin on the nanoparticle may be non-covalent or covalent. In some embodiments, the nanoparticle comprises a hydrophobic drug associated with an albumin, wherein a bioactive polypeptide is associated with the albumin non-covalently. In some embodiments, the bioactive polypeptide is embedded into the surface of the nanoparticle. In some embodiments, the nanoparticle comprises a solid core of a hydrophobic drug coated with an albumin, wherein a bioactive polypeptide is associated with the albumin non-covalently. In some embodiments, the bioactive polypeptide is associated with the albumin on the nanoparticle non-covalently. In some embodiments, the albumin is a derivatized albumin, wherein the albumin is derivatized with a moiety that non-covalently binds to a bioactive polypeptide. In some embodiments, the bioactive polypeptide comprises a moiety that non-covalently binds to an albumin. In some embodiments, the bioactive polypeptide comprises a moiety that non-covalently binds to a derivatized albumin. The features described above for the nanoparticles can be applied to the specific embodiments described below, as would be understood by a person of ordinary skill in the art in view of the present disclosure.

Nanoparticles with Embedded Bioactive Polypeptides

Contemplated within the scope of the invention are nanoparticles comprising a hydrophobic drug, wherein the hydrophobic drug is associated (such as adsorbed or coated) with an albumin or wherein the hydrophobic drug is associated (such as adsorbed or coated) with an albumin and a bioactive polypeptide (such as an antibody or a fragment thereof). In certain embodiments, the bioactive polypeptide is included in one or more nanoparticle precursors, as discussed in further detail below. During the manufacturing process, at least a portion of the bioactive polypeptide associates with the nanoparticle such that the bioactive polypeptide is embedded in the surface of the nanoparticle or the hydrophobic core of the nanoparticle.

For example, in some embodiments, the nanoparticle comprises a hydrophobic drug associated with an albumin. In some embodiments, the nanoparticle comprises a solid core of a hydrophobic drug associated with an albumin. In some embodiments, the nanoparticle comprises a hydrophobic drug coated with an albumin. In some embodiments, the nanoparticle comprises a solid core of a hydrophobic drug coated with an albumin. In some embodiments, the nanoparticle comprises a hydrophobic drug substantially coated with an albumin. In some embodiments, the nanoparticle comprises a solid core of a hydrophobic drug substantially coated with an albumin. In some embodiments, the nanoparticle comprises a hydrophobic drug coated with an albumin and a bioactive polypeptide. In some embodiments, the nanoparticle comprises a solid core of a hydrophobic drug coated with an albumin and a bioactive polypeptide. In some embodiments, the nanoparticle comprises a solid core of a hydrophobic drug, wherein a bioactive polypeptide is associated with the surface of the solid core of hydrophobic drug, and wherein the solid core of hydrophobic drug is coated with an albumin. In some embodiments, the nanoparticle comprises a solid core of a hydrophobic drug, wherein a portion of a bioactive polypeptide is embedded in the solid core of hydrophobic drug, and wherein the solid core of hydrophobic drug is coated with an albumin.

In some embodiments, about 1% or more (such as about 2%, 3%, 4%, 5%, 10%, 15%, 20%, or 25% or more) of the bioactive polypeptide associated with the nanoparticles is embedded in the solid core of the nanoparticles. In some embodiments, about 25% or less (such as about 20%, 15%, 10%, 5%, 4%, 3%, 2%, or 1% or less) of the bioactive polypeptide is embedded in the solid core of the nanoparticles. In some embodiments, between about 1% and about 25% (such as between about 1% and about 2%, about 2% and about 3%, about 3% and about 4%, about 4% and about 5%, about 5% and about 10%, about 10% and about 15%, about 15% and about 20%, or about 20% and about 25%) of the bioactive polypeptide is embedded in the solid core of the nanoparticles.

In some embodiments, the nanoparticle comprises a hydrophobic drug associated with an albumin, wherein a bioactive polypeptide is associated with the albumin on the nanoparticle. As described herein, the contact (such as association) between a bioactive polypeptide and an albumin may be, for example, at an outer albumin surface of a nanoparticle, within an albumin coating of a nanoparticle, at an inner albumin surface of a nanoparticle (such as the interface between a solid core of a hydrophobic drug and a coating of an albumin), or combinations thereof. In some embodiments, the nanoparticle comprises a hydrophobic drug coated with an albumin, wherein a bioactive polypeptide is associated with the albumin on the nanoparticle. In some embodiments, the nanoparticle comprises a solid core of a hydrophobic drug coated with an albumin, wherein a bioactive polypeptide is associated with the albumin on the nanoparticle.

In some embodiments, the bioactive polypeptide is associated with a hydrophobic drug and an albumin. Thus, for example, in some embodiments, the nanoparticle comprises a solid core of a hydrophobic drug coated with an albumin, wherein at least a portion of the bioactive polypeptide is associated with the solid core of hydrophobic drug, and wherein at least a portion of the bioactive polypeptide is associated with the albumin. In some embodiments, the nanoparticle comprises a solid core of a hydrophobic drug coated with an albumin, wherein at least a portion of the bioactive polypeptide is embedded in the solid core of hydrophobic drug, and wherein at least a portion of the bioactive polypeptide is associated with the albumin. In some embodiments, the nanoparticle comprises a solid core of a hydrophobic drug coated with an albumin, wherein at least a portion of the bioactive polypeptides is partially embedded in the solid core of hydrophobic drug and partially associated with the albumin. In some embodiments, the nanoparticle comprises a solid core of a hydrophobic drug coated with an albumin, wherein a hydrophobic portion of at least one bioactive polypeptide is embedded in the solid core of hydrophobic drug, and wherein a second portion of the same bioactive polypeptide is associated with the albumin. In some embodiments, the nanoparticle comprises a solid core of a hydrophobic drug coated with an albumin, wherein a hydrophobic portion of a bioactive polypeptide is embedded in the solid core of hydrophobic drug, and wherein a hydrophilic portion of the same bioactive polypeptide is associated with the albumin

Nanoparticles with Conjugated Bioactive Polypeptides

In certain embodiments, the bioactive polypeptide (such as an antibody or a fragment thereof) is conjugated to the nanoparticle (e.g., the albumin component of the nanoparticle). In some embodiments, the association of a bioactive polypeptide and an albumin is a direct association (such as a bioactive polypeptide directly binding to an albumin). For example, in certain embodiments, the bioactive polypeptide directly binds the albumin, for example through the formation of a disulfide bond. In some embodiments, conjugation occurs through a crosslinker, which can covalently bond to the bioactive polypeptide and the albumin. In some embodiments, the association of a bioactive polypeptide and an albumin is a covalent association (such as use of a chemical linker to conjugate a bioactive polypeptide and an albumin) In certain embodiments, the crosslinker includes a first component that covalently binds to the albumin and a second component that covalently binds to the polypeptide, and the first component and the second component specifically bind through a non-covalent interaction.

In some embodiments, the nanoparticle comprises a hydrophobic drug associated with an albumin, wherein a bioactive polypeptide is associated with the albumin covalently (i.e., the bioactive polypeptide is conjugated to the albumin) In some embodiments, the nanoparticle comprises a solid core of a hydrophobic drug coated with an albumin, wherein a bioactive polypeptide is associated with the albumin covalently. In some embodiments, the bioactive polypeptide is associated with an albumin on the nanoparticle covalently.

In some embodiments, the bioactive polypeptide is associated with an albumin on the nanoparticle via direct covalent binding. For example, in some embodiments, the bioactive polypeptide is associated with an albumin on the nanoparticle via formation of a disulfide bond between the bioactive polypeptide and the albumin. In some embodiments, the bioactive polypeptide is associated with an albumin on the nanoparticle via a free thiol on the albumin (such as Cys34). In some embodiments, the bioactive polypeptide is associated with an albumin on the nanoparticle via formation of a disulfide bond between the bioactive polypeptide and a free thiol on the albumin (such as Cys34). In some embodiments, the bioactive polypeptide comprises a free thiol, such as a free cysteine, that covalently binds (via a disulfide bond) to a free thiol, such as free cysteine (e.g., Cys34) on the albumin. In some embodiments, the albumin is a derivatized albumin, and the thiol on the albumin is derived (for example, derived from an amine through the use of a thiolating agent, such as 2-iminothiolane).

In some embodiments, the bioactive polypeptide is associated with an albumin on the nanoparticle via a chemical crosslinker (such as a non-zero-length crosslinker or a crosslinker of any suitable length). In some embodiments, the crosslinker is a monofunctional crosslinker. In some embodiments, the crosslinker is a bifunctional crosslinker. In some embodiments, the bioactive polypeptide is associated with more than one albumin on the nanoparticle via more than one chemical crosslinker. Crosslinkers suitable for association (such as covalent conjugation or formation of a complex) of two or more proteins via covalent attachment or formation of a complex to an amino acid residue or other functional group associated with the protein, such as a glycan, are known in the art. For example, in some embodiments, the crosslinker may be attached to a free thiol on an albumin (such as Cys34 or a derivatized thiol), wherein another terminal end of the crosslinker is available to bind to a bioactive polypeptide. In some embodiments, the crosslinker may be attached to a free thiol on an albumin, wherein another terminal end of the crosslinker is bound to a bioactive polypeptide. In some embodiments, the crosslinker may be attached to a free thiol on an albumin (such as Cys34), wherein another terminal end of the crosslinker is available to bind or is bound to a crosslinker attached to a bioactive polypeptide. In some embodiments, the crosslinker may be attached to a free amine on an albumin (such as a lysine residue). In some embodiments, the crosslinker may be attached to a free lysine on an albumin, wherein another terminal end of the crosslinker is bound to a bioactive polypeptide. In some embodiments, the crosslinker may be attached to a free lysine on an albumin, wherein another terminal end of the crosslinker is available to bind to a crosslinker attached to a bioactive polypeptide. In some embodiments, the crosslinker may be attached to a free lysine on an albumin, wherein another terminal end of the crosslinker is available to bind or is bound to a crosslinker attached to a bioactive polypeptide.

In some embodiments, the crosslinker comprises a maleimide functional group (such as maleimidopropionic acid (MPA) or gamma-maleimide-butyralamide (GMBA)). In some embodiments, the crosslinker comprises a succinimidyl ester group, such as N-hydroxysuccinimide (NHS) ester. In some embodiments, the length of the crosslinker is determined by a polymer, such as a polyethylene glycol (PEG), which bridges the chemical reactive groups of the crosslinker. For example, in some embodiments, the crosslinker is NHS-(polyethylene glycol)ₙ-maleimide (SM(PEG)ₙ), wherein n is two or more. For example, in some embodiments the crosslinker is (SM(PEG)₂), (SM(PEG)₄), (SM(PEG)₆), (SM(PEG)₈), (SM(PEG)₁₂), or (SM(PEG)₂₄). In some embodiments, the crosslinker is succinimidyl 4-(N-maleimidomethyl)cyclohexane-1-carboxylate (SMCC).

In some embodiments, the crosslinker comprises a boron moiety, such as boronate ester (which may be derived from a boronic acid). Boronic acid can react with a carbohydrate, such as a glycan on a glycosylated bioactive protein (such as a glycosylated antibody) to form a boronate ester. In some embodiments, the crosslinker comprises a boronic acid and a N-hydroxysuccinimide (NHS) ester. In some embodiments, the crosslinker comprises a chemical bridge linking the boronic acid and the NHS ester, wherein the bridge determines the length of the crosslinker. An exemplary crosslinker can be formed, for example, by combining 4-(2-carboxyethyl)benzeneboronic acid, N-hydroxysuccinimide (NHS), and NN-dicyclohexylcarbodiimide in DMF to form an activated ester crosslinker. The NHS moiety can react with the albumin, and the boronic acid moiety can react with glycans on the bioactive polypeptide.

In some embodiments, the crosslinker is a click chemistry (e.g., a copper-free click chemistry) crosslinker. For example, in some embodiments, the crosslinker comprises a triazole moiety, which can be formed by reacting a cyclooctene derivative moiety (such as dibenzocyclooctyne (DBCO) moiety) with an azide through a strain-promoted alkyne azide cycloaddition reaction. The albumin or the antibody can be covalently linked to the cyclooctene derivative moiety, for example by reacting with NHS bridged to the cycooctene derivative moiety (e.g., DBCO-PEGₙ-NHS, wherein n is 2 or larger), and the other crosslinked entity (i.e., the albumin or the antibody) can be functionalized with an azide.

In some embodiments, the crosslinker comprises a first component covalently attached to the albumin, and a second component covalently attached to the bioactive polypeptide, wherein the first component and the second component specifically bind to one another. For example, the first component or the second component can be a single stranded polynucleotide, such as DNA, or a synthetic polymer, e.g., a morpholino. In some embodiments, the crosslinker comprises two single stranded polynucleotide strands that are substantially complementary, wherein one single stranded polynucleotide is conjugated to a bioactive polypeptide and the other single stranded polynucleotide is conjugated to an albumin. For example, in some embodiments, the first component or the second component comprises a single stranded DNA comprising a plurality of “CA” repeats, such as molecule according to SEQ ID NO: 1 (5′-CACACACACACACACACACA-3′), and other component (i.e., the first component or the second component) comprises a single stranded DNA molecule comprising a plurality of “GT” repeats, such as a DNA molecule according to SEQ ID NO: 2 (5′-GTGTGTGTGTGTGTG-3′). Once the albumin and the bioactive polypeptide are combined, the DNA strands specifically bind, thereby conjugating the albumin to the bioactive polypeptide.

The length of the crosslinker when conjugated to an albumin and a bioactive polypeptide may be any suitable length. In some embodiments, the length of the crosslinker accounts for two initially separate crosslinkers that have been conjugated, for instance, a crosslinker attached to an albumin is associated or conjugated with a crosslinker attached to a bioactive polypeptide, e.g., via click chemistry or complementary DNA base pairing. In some embodiments, the length of the crosslinker is about 200 angstroms or less, such as any of about 175, 150, 125, 100, 90, 80, 70, 60, 50, 40, 30, 20, or 10 angstroms or less. In some embodiments, the length of the crosslinker is about 10 angstroms or more, such as any of about 20, 30, 40, 50, 60, 70, 80, 90, 100, 125, 150, 175, 200 angstroms or more. In some embodiments, the length of the crosslinker is about 8.3 angstroms, about 17.6 angstroms, about 24.6 angstroms, about 32.5 angstroms, about 39.2 angstroms, about 53.4 angstroms, or about 95.2 angstroms.

In some of the above embodiments, the albumin on the nanoparticle is a derivatized albumin (such as albumin derivatized with a chemical crosslinker). In some embodiments, the amount of a derivatized albumin on the nanoparticle is less than about 50%, 25%, 20%, 15%, 10%, or 5% of the total albumin on the nanoparticle. In some embodiments, the amount of a derivatized albumin on the nanoparticle is less than about 5%, 4%, 3%, 2%, or 1% of the total albumin on the nanoparticle. In some embodiments, the amount of derivatized albumin on the nanoparticle is about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the total albumin on the nanoparticle. In some embodiments, the amount of a derivatized albumin on the nanoparticle is between about 1% to about 3%, about 1% to about 5%, about 1% to about 7%, about 1% to about 10%, about 3% to about 7%, about 3% to about 10%, or about 5% to about 15% of the total albumin on the nanoparticle.

In some embodiments, the bioactive polypeptide is conjugated with one or more crosslinker. In some embodiments, the bioactive polypeptide is conjugated with 1 to 20 crosslinkers, such as any of 1 to 10, 1 to 5, 1 to 4, 1 to 3, 2 to 5, 3 to 5, and 2 to 4 crosslinkers. In some embodiments, the bioactive polypeptide is conjugated with less than 10 crosslinkers, such as any of less than 9, 8, 7, 6, 5, 4, 3, and 2 crosslinkers.

In some embodiments, the average number of crosslinkers per bioactive polypeptide in a nanoparticle composition is about 1, 2, 3, 4, 5, 6, 7, 8, 9, or 10 crosslinkers. In some embodiments, the average number of crosslinkers per bioactive polypeptide in a nanoparticle composition is about 1 to 10 crosslinkers, such as any of 1 to 5, 1 to 4, 1 to 3, 2 to 5, 3 to 5, and 2 to 4 crosslinkers. In some embodiments, the average number of crosslinkers per bioactive polypeptide in a nanoparticle composition is less than about 10 crosslinkers, such as any of less than about 9, 8, 7, 6, 5, 4, 3, and 2 crosslinkers.

In some embodiments, the bioactive polypeptide is conjugated to one or more albumin. In some embodiments, the bioactive polypeptide is conjugated to 1 to 10 albumin, such as any of 1 to 3, 1 to 4, 1 to 5, 1 to 6, 2 to 4, and 2 to 5 albumins. In some embodiments, the bioactive polypeptide is conjugated with less than 10 albumins, such as any of less than 9, 8, 7, 6, 5, 4, 3, and 2 albumin.

In some embodiments, the average number of conjugated albumin per bioactive polypeptide in a nanoparticle composition is about 1, 2, 3, 4, 5, 6, 7, 8, 9, or 10. In some embodiments, the average number of conjugated albumin per bioactive polypeptide in a nanoparticle composition is about 1 to 10, such as any of 1 to 5, 1 to 4, 1 to 3, 2 to 5, 3 to 5, and 2 to 4. In some embodiments, the average number of conjugated albumin per bioactive polypeptide in a nanoparticle composition is less than about 10, such as any of less than about 9, 8, 7, 6, 5, 4, 3, and 2.

Nanoparticle Compositions

It is contemplated herein that the composition comprising nanoparticles may comprise any combination of nanoparticles described herein. Furthermore, in some embodiments, the nanoparticles may comprise any combination of features described herein.

As described herein, in some embodiments, the composition comprises an albumin in both a nanoparticle and a non-nanoparticle portion of the composition. In some embodiments, the compositions described herein further comprise an albumin not associated with nanoparticles in the composition. The amount of an albumin in the composition described herein will vary depending on other components in the composition (such as a hydrophobic drug or a bioactive polypeptide). In some embodiments, the composition comprises an albumin in an amount that is sufficient to stabilize a hydrophobic drug in an aqueous suspension, for example, in the form of a stable colloidal suspension (such as a stable suspension of nanoparticles). In some embodiments, the albumin is in an amount that reduces the sedimentation rate of a hydrophobic drug in an aqueous medium. For particle-containing compositions, the amount of the albumin also depends on the size and density of nanoparticles. In some embodiments, at least about 50%, 60%, 70%, 80%, 90%, 95%, or 99% of the albumin in a composition is in a non-nanoparticle portion of the composition.

A hydrophobic drug is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as human). Stability of the suspension is generally (but not necessarily) evaluated at a storage temperature (such as room temperature (such as 20-25° C.) or refrigerated conditions (such as 4° C.)). For example, a suspension is stable at a storage temperature if it exhibits no flocculation or particle agglomeration visible to the naked eye or when viewed under the optical microscope at 400 times magnification, at about fifteen minutes after preparation of the suspension. Stability can also be evaluated under accelerated testing conditions, such as at a temperature that is 40° C. or higher than about 40° C.

The use of an albumin in a non-nanoparticle portion of the composition can avoid the use of toxic solvents (or surfactants) for solubilizing the hydrophobic drug and/or nanoparticles, and thereby can reduce one or more side effects of administration of the hydrophobic drugs into an individual (such as a human). Thus, in some embodiments, the composition described herein is substantially free (such as free) of surfactants, such as Cremophor (including Cremophor EL® (BASF)). In some embodiments, the composition is substantially free (such as free) of surfactants (for example polysorbate such as polysorbate 20 or polysorbate 80). A composition is “substantially free of Cremophor” or “substantially free of surfactant” if the amount of Cremophor or surfactant in the composition is less than about 0.02%. “Substantially free of Cremophor” or “substantially free of surfactant” refers to having less than about 0.01% Cremophor or surfactant. In some embodiments, the compositions have less than about 0.005%, less than about 0.0001%, less than about 0.00005%, or less than about 0.00001% Cremophor or surfactant.

In some embodiments, the albumin is present in a composition in an amount that is sufficient to stabilize a hydrophobic drug in an aqueous suspension at a certain concentration. For example, the concentration of a hydrophobic drug in the composition is about 0.1 to about 100 mg/ml, including for example any of about 0.1 to about 50 mg/ml, about 0.1 to about 20 mg/ml, about 1 to about 10 mg/ml, about 2 mg/ml to about 8 mg/ml, about 4 to about 6 mg/ml, about 5 mg/ml. In some embodiments, the concentration of a hydrophobic drug is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor, polysorbate 20, or polysorbate 80), so that the composition is free or substantially free of surfactant (such as Cremophor, polysorbate 20, or polysorbate 80).

In some embodiments, the composition is substantially free of (or free of) sodium phosphate. “Substantially free of sodium phosphate” as used herein refers to having less than about 0.1 mg/mL of sodium phosphate. In some embodiments, the composition has less than about 0.05 mg/mL, less than about 0.01 mg/mL, less than about 0.005 mg/mL, less than about 0.001 mg/mL, or less than about 0.0001 mg/mL of sodium phosphate. In some embodiments, the composition comprises sodium phosphate.

In some embodiments, the composition is substantially free of (or free of) trehalose. “Substantially free of trehalose” as used herein refers to having less than about 1 mg/mL of trehalose In some embodiments, the composition has less than about 0.5 mg/mL, less than about 0.1 mg/mL, less than about 0.05 mg/mL, less than about 0.01 mg/mL, or less than about 0.001 mg/mL of trehalose. In some embodiments, the composition comprises trehalose.

In some embodiments, the nanoparticle composition, in liquid form, comprises from about 0.1% to about 50% (w/v) (e.g. about 0.5% (w/v), about 5% (w/v), about 10% (w/v), about 15% (w/v), about 20% (w/v), about 30% (w/v), about 40% (w/v), or about 50% (w/v)) of an albumin. In some embodiments, the nanoparticle composition, in liquid form, comprises about 0.5% to about 10% (w/v) of an albumin.

In some embodiments, the composition comprises a hydrophobic drug in both a nanoparticle and a non-nanoparticle portion of the composition. In some embodiments, no greater than 1%, 2%, 3%, 4%, 5%, 10%, or 20% of the hydrophobic drug in a composition is in a non-nanoparticle portion of the composition. In some embodiments, at least about 50%, 60%, 70%, 80%, 90%, 95%, or 99% of the hydrophobic drug in a composition is in nanoparticle portion of the composition.

In some embodiments, the composition comprises a bioactive polypeptide in both a nanoparticle and a non-nanoparticle portion of the composition. In some embodiments, the composition further comprises bioactive polypeptide not associated with the nanoparticles. In some embodiments, no greater than 1%, 2%, 3%, 4%, 5%, 10%, or 20% of the bioactive polypeptide in a composition is in a non-nanoparticle portion of the composition. In some embodiments, at least about 50%, 60%, 70%, 80%, 90%, 95%, or 99% of the bioactive polypeptide in a composition is in nanoparticle portion of the composition.

The weight ratio of the albumin to the hydrophobic drug in compositions may be optimized based on presence of a hydrophobic drug, an albumin, a bioactive polypeptide, another therapeutic agent, or combinations thereof. In some embodiments, the weight ratio of the albumin to the hydrophobic drug in a composition is about 1:1 to about 50:1, about 1:1 to about 20:1, about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, or about 1:1 to about 2:1. In some embodiments, the weight ratio of the albumin to the hydrophobic drug in a composition is less than about 18:1, 15:1, or 10:1. In some embodiments, the weight ratio of the albumin to the hydrophobic drug in a composition is about 1:1 to about 18:1, about 2:1 to about 15:1, about 3:1 to about 13:1, about 4:1 to about 12:1, about 5:1 to about 10:1. In some embodiments, the weight ratio of the albumin to the hydrophobic drug in a composition is about 1:2, 1:3, 1:4, 1:5, 1:6, 1:7, 1:8, 1:9, 1:10, 1:11, 1:12, 1:13, 1:14, or 1:15. In some embodiments, the weight of albumin is determined by size exclusion chromatography (SEC). In some embodiments, the weight of hydrophobic drug is determined by reverse-phase high performance liquid chromatography (RP-HPLC).

The weight ratio of the hydrophobic drug to the bioactive polypeptide in the compositions may be optimized based on presence of a hydrophobic drug, an albumin, a bioactive polypeptide, another therapeutic agent, or combinations thereof. In some embodiments, the weight ratio of the hydrophobic drug to the bioactive polypeptide in the composition is between about 1:1 and 200:1 (such as between 1:1 and about 100:1, about 1:1 and about 80:1, about 1:1 and about 60:1, about 1:1 and about 50:1, about 2:1 and about 40:1, about 4:1 and about 30:1, or about 6:1 to about 20:1). In some embodiments, the weight of hydrophobic drug is determined by reverse-phase high performance liquid chromatography (RP-HPLC). In some embodiments, the weight of bioactive polypeptide is determined by size exclusion chromatography (SEC) or an enzyme-linked immunosorbent assay (ELISA).

The weight ratio of the bioactive polypeptide to the albumin in compositions may be optimized based on presence of a hydrophobic drug, an albumin, a bioactive polypeptide, another therapeutic agent, or combinations thereof. In some embodiments, the weight ratio of the bioactive polypeptide to the albumin in the composition is about 1:1 to about 1:1000, about 1:1 to about 1:800, about 1:1 to about 1:600, about 1:1 to about 1:500, about 1:1 to about 1:400, about 1:1 to about 1:300, about 1:1 to about 1:250, about 2:1 to about 1:200, about 2:1 to about 1:150, about 4:1 to about 1:100, or about 4:1 to about 1:50. In some embodiments, the weight of albumin is determined by size exclusion chromatography (SEC). In some embodiments, the weight of the bioactive polypeptide is determined by size exclusion chromatography (SEC) or by an enzyme-linked immunosorbent assay (ELISA).

Hydrophobic Drugs

Hydrophobic drugs described herein can be, for example, drugs with solubility in water (pH 7) less than about 1 mg/ml at about 25° C., including for example drugs with solubility less than about any of 0.5, 0.2, 0.1, 0.05, 0.02, or 0.01 mg/ml. In some embodiments, the hydrophobic drug is an antineoplastic agent. In some embodiments, the hydrophobic drug is a chemotherapeutic agent. Suitable hydrophobic drugs include, but are not limited to, taxanes (such as paclitaxel, docetaxel, ortataxel, and other taxanes), limus drugs (such as sirolimus), 17-allylamino geldanamycin (17-AAG), or thiocolchicine dimer (such as IDN5404).

In some embodiments, the hydrophobic drug is a taxane. In some embodiments, the taxane is paclitaxel. In some embodiments, the hydrophobic drug is paclitaxel.

In some embodiments, the hydrophobic drug is a limus drug, which includes rapamycin (sirolimus) and its analogues. Examples of limus drugs include, but are not limited to, temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the limus drug is selected from the group consisting of temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the hydrophobic drug is rapamycin (sirolimus).

Thus, in some embodiments, the composition comprises nanoparticles comprising (a) a taxane, (b) an albumin (such as a human albumin), and (c) a bioactive polypeptide. In some embodiments, the composition comprises nanoparticles comprising (a) a taxane, (b) an albumin (such as a human albumin), and (c) a bioactive polypeptide, wherein the taxane is coated with the albumin. In some embodiments, the composition comprises nanoparticles comprising (a) a taxane, (b) an albumin (such as a human albumin), and (c) a bioactive polypeptide, wherein the taxane is coated with the albumin, and wherein the bioactive polypeptide is associated with the taxane. In some embodiments, the composition comprises nanoparticles comprising (a) a taxane, (b) an albumin (such as a human albumin), and (c) a bioactive polypeptide, wherein the taxane is coated with the albumin, and wherein the bioactive polypeptide is associated with the albumin. In some embodiments, the composition comprises nanoparticles comprising (a) a taxane, (b) an albumin (such as a human albumin), and (c) a bioactive polypeptide, wherein the taxane is coated with the albumin, and wherein the bioactive polypeptide is associated with the taxane and the albumin. In some embodiments, the composition comprises nanoparticles comprising (a) paclitaxel, (b) an albumin (such as a human albumin), and (c) a bioactive polypeptide. In some embodiments, the composition comprises nanoparticles comprising (a) paclitaxel, (b) an albumin (such as a human albumin), and (c) a bioactive polypeptide, wherein paclitaxel is coated with the albumin. In some embodiments, the composition comprises nanoparticles comprising (a) paclitaxel, (b) an albumin (such as a human albumin), and (c) a bioactive polypeptide, wherein paclitaxel is coated with the albumin, and wherein the bioactive polypeptide is associated with paclitaxel. In some embodiments, the composition comprises nanoparticles comprising (a) paclitaxel, (b) an albumin (such as a human albumin), and (c) a bioactive polypeptide, wherein paclitaxel is coated with the albumin, and wherein the bioactive polypeptide is associated with the albumin. In some embodiments, the composition comprises nanoparticles comprising (a) paclitaxel, (b) an albumin (such as a human albumin), and (c) a bioactive polypeptide, wherein paclitaxel is coated with the albumin, and wherein the bioactive polypeptide is associated with paclitaxel and the albumin.

In some embodiments, the composition comprises nanoparticles comprising (a) a taxane, (b) an albumin (such as a human albumin), and (c) an antibody. In some embodiments, the composition comprises nanoparticles comprising (a) a taxane, (b) an albumin (such as a human albumin), and (c) an antibody, wherein the taxane is coated with the albumin. In some embodiments, the composition comprises nanoparticles comprising (a) a taxane, (b) an albumin (such as a human albumin), and (c) an antibody, wherein the taxane is coated with the albumin, and wherein the bioactive polypeptide is associated with the taxane. In some embodiments, the composition comprises nanoparticles comprising (a) a taxane, (b) an albumin (such as a human albumin), and (c) an antibody, wherein the taxane is coated with the albumin, and wherein the bioactive polypeptide is associated with the albumin. In some embodiments, the composition comprises nanoparticles comprising (a) a taxane, (b) an albumin (such as a human albumin), and (c) an antibody, wherein the taxane is coated with the albumin, and wherein the antibody is associated with the taxane and the albumin. In some embodiments, the composition comprises nanoparticles comprising (a) paclitaxel, (b) an albumin (such as a human albumin), and (c) an antibody. In some embodiments, the composition comprises nanoparticles comprising (a) paclitaxel, (b) an albumin (such as a human albumin), and (c) an antibody, wherein paclitaxel is coated with the albumin. In some embodiments, the composition comprises nanoparticles comprising (a) paclitaxel, (b) an albumin (such as a human albumin), and (c) an antibody, wherein paclitaxel is coated with the albumin, and wherein the antibody is associated with paclitaxel. In some embodiments, the composition comprises nanoparticles comprising (a) paclitaxel, (b) an albumin (such as a human albumin), and (c) an antibody, wherein paclitaxel is coated with the albumin, and wherein the antibody is associated with the albumin. In some embodiments, the composition comprises nanoparticles comprising (a) paclitaxel, (b) an albumin (such as a human albumin), and (c) an antibody, wherein paclitaxel is coated with the albumin, and wherein the antibody is associated with paclitaxel and the albumin.

Thus, in some embodiments, the composition comprises nanoparticles comprising (a) a limus drug, (b) an albumin (such as a human albumin), and (c) a bioactive polypeptide. In some embodiments, the composition comprises nanoparticles comprising (a) a limus drug, (b) an albumin (such as a human albumin), and (c) a bioactive polypeptide, wherein the limus drug is coated with the albumin. In some embodiments, the composition comprises nanoparticles comprising (a) a limus drug, (b) an albumin (such as a human albumin), and (c) a bioactive polypeptide, wherein the limus drug is coated with the albumin, and wherein the bioactive polypeptide is associated with the limus drug. In some embodiments, the composition comprises nanoparticles comprising (a) a limus drug, (b) an albumin (such as a human albumin), and (c) a bioactive polypeptide, wherein the limus drug is coated with the albumin, and wherein the bioactive polypeptide is associated with the albumin. In some embodiments, the composition comprises nanoparticles comprising (a) a limus drug, (b) an albumin (such as a human albumin), and (c) a bioactive polypeptide, wherein the limus drug is coated with the albumin, and wherein the bioactive polypeptide is associated with the limus drug and the albumin. In some embodiments, the composition comprises nanoparticles comprising (a) rapamycin, (b) an albumin (such as a human albumin), and (c) a bioactive polypeptide. In some embodiments, the composition comprises nanoparticles comprising (a) rapamycin, (b) an albumin (such as a human albumin), and (c) a bioactive polypeptide, wherein rapamycin is coated with the albumin. In some embodiments, the composition comprises nanoparticles comprising (a) rapamycin, (b) an albumin (such as a human albumin), and (c) a bioactive polypeptide, wherein rapamycin is coated with the albumin, and wherein the bioactive polypeptide is associated with rapamycin. In some embodiments, the composition comprises nanoparticles comprising (a) rapamycin, (b) an albumin (such as a human albumin), and (c) a bioactive polypeptide, wherein rapamycin is coated with the albumin, and wherein the bioactive polypeptide is associated with the albumin. In some embodiments, the composition comprises nanoparticles comprising (a) rapamycin, (b) an albumin (such as a human albumin), and (c) a bioactive polypeptide, wherein rapamycin is coated with the albumin, and wherein the bioactive polypeptide is associated with rapamycin and the albumin.

Thus, in some embodiments, the composition comprises nanoparticles comprising (a) a limus drug, (b) an albumin (such as a human albumin), and (c) an antibody. In some embodiments, the composition comprises nanoparticles comprising (a) a limus drug, (b) an albumin (such as a human albumin), and (c) an antibody, wherein the limus drug is coated with the albumin. In some embodiments, the composition comprises nanoparticles comprising (a) a limus drug, (b) an albumin (such as a human albumin), and (c) an antibody, wherein the limus drug is coated with the albumin, and wherein the antibody is associated with the limus drug. In some embodiments, the composition comprises nanoparticles comprising (a) a limus drug, (b) an albumin (such as a human albumin), and (c) an antibody, wherein the limus drug is coated with the albumin, and wherein the antibody is associated with the albumin. In some embodiments, the composition comprises nanoparticles comprising (a) a limus drug, (b) an albumin (such as a human albumin), and (c) an antibody, wherein the limus drug is coated with the albumin, and wherein the antibody is associated with the limus drug and the albumin. In some embodiments, the composition comprises nanoparticles comprising (a) rapamycin, (b) an albumin (such as a human albumin), and (c) an antibody. In some embodiments, the composition comprises nanoparticles comprising (a) rapamycin, (b) an albumin (such as a human albumin), and (c) an antibody, wherein rapamycin is coated with the albumin. In some embodiments, the composition comprises nanoparticles comprising (a) rapamycin, (b) an albumin (such as a human albumin), and (c) an antibody, wherein rapamycin is coated with the albumin, and wherein the antibody is associated with rapamycin. In some embodiments, the composition comprises nanoparticles comprising (a) rapamycin, (b) an albumin (such as a human albumin), and (c) an antibody, wherein rapamycin is coated with the albumin, and wherein the antibody is associated with the albumin. In some embodiments, the composition comprises nanoparticles comprising (a) rapamycin, (b) an albumin (such as a human albumin), and (c) an antibody, wherein rapamycin is coated with the albumin, and wherein the antibody is associated with rapamycin and the albumin

Bioactive Polypeptides

In some embodiments, the bioactive polypeptide is an antibody or a fragment thereof. In some embodiments, the bioactive polypeptide is an antibody or a fragment thereof specifically recognizing an antigen.

In some embodiments, the bioactive polypeptide is selected from the group consisting of: alemtuzumab, bevacizumab, blinatumomab, brentuximab, cetuximab, denosumab, dinutuximab, durvalumab, ipilimumab, nivolumab, obinutuzumab, ofatumumab, panitumumab, pembrolizumab, pertuzumab, trastuzumab, durvalumab, and rituximab. In some embodiments, the bioactive polypeptide is BGB-A317 (BeiGene). In some embodiments, the bioactive polypeptide is tocilizumab.

In some embodiments, the bioactive polypeptides of the compositions described herein (such as the bioactive polypeptides on nanoparticles) are able to trigger an immunological response in an individual (such as a human). The compositions described herein may be optimized to balance the ADCC and CDC effect in an individual. In some embodiments, the bioactive polypeptide triggers an antibody-dependent cell-mediated (ADCC) effect in an individual. In some embodiments, the bioactive polypeptide triggers a complement dependent cytotoxicity (CDC) effect in an individual. In some embodiments, the composition comprising nanoparticles triggers an ADCC effect in an individual. In some embodiments, the composition comprising nanoparticles triggers a CDC effect in an individual. In some embodiments, the composition comprising nanoparticles triggers an ADCC and CDC effect in an individual.

In some embodiments, the bioactive polypeptide specifically recognizes (such as binds to) an antigen. In some embodiments, the bioactive polypeptide is an antibody that specifically binds to alpha-fetoprotein (AFP), carcinoembryonic antigen (CEA), cancer antigen 125 (CA-125), mucin 1 (MUC1), epithelial tumor antigen (ETA), melanoma-associate antigen (MAGE), programmed cell death protein 1 (PD-1), programmed death ligand 1 (PD-L1), tyrosinase, epidermal growth factor receptor (EGFR), vascular endothelial growth factor (VEGF), NY-ESO-1, gp100, BCR-ABL, EGFR, PSA, PMSA, HER2/neu, hTERT, MART1, TRP-1, TRP-2, ras, BRAF, BRCA1, BRCA2, Flt-3, IL-6-receptor, or Smad4. In some embodiments, the antigen is a tumor antigen. Tumor-associated antigens include, but are not limited to, tumor components that may serve as a basis for targeting a cancer tissue (such as a cancer cell) or tumor-associated tissue (such as tumor-associated stroma). For example, tumor-associated antigens include, but are not limited to, alpha-fetoprotein (AFP), carcinoembryonic antigen (CEA), cancer antigen 125 (CA-125), mucin 1 (MUC1), epithelial tumor antigen (ETA), melanoma-associate antigen (MAGE), programmed cell death protein 1 (PD-1), programmed death ligand 1 (PD-L1), tyrosinase, epidermal growth factor receptor (EGFR), vascular endothelial growth factor (VEGF), NY-ESO-1, gp100, BCR-ABL, EGFR, PSA, PMSA, HER2/neu, hTERT, MART1, TRP-1, TRP-2, ras, BRAF, BRCA1, BRCA2, Flt-3, and Smad4.

In some embodiments, the bioactive polypeptide comprises a site for chemical conjugation. In some embodiments, the bioactive polypeptide comprises a site for association of a crosslinker, such as an amino acid residue or a glycan structure. In some embodiments, the bioactive polypeptide further comprises a chemical linker. In some embodiments, the bioactive polypeptide is a derivatized bioactive polypeptide (such as an antibody comprising a chemical crosslinker moiety). In some embodiments, the derivatized bioactive polypeptide is a bioactive polypeptide conjugated with a chemical crosslinker (or portion thereof) reactive (such as specifically reactive) to another chemical crosslinker (or portion thereof) conjugated to an albumin. For example, in some embodiments, the derivatized bioactive polypeptide is conjugated with a chemical crosslinker comprising an alkyne moiety and the derivatized albumin is conjugated with a chemical crosslinker comprising an azide moiety. In some embodiments, the derivatized bioactive polypeptide is conjugated with a chemical crosslinker comprising an azide moiety and the derivatized albumin is conjugated with a chemical crosslinker comprising an alkyne moiety. Other pairs of crosslinking moieties useful for associating a bioactive polypeptide and an albumin include, but are not limited to, a strained alkyne and an azide, a strained alkyne and a nitrone (such as a 1,3-nitrone), a strained alkene and an azide, a strained alkene and a tetrazine, and a strained alkene and a tetrazole. In some embodiments, substantially all of the bioactive polypeptide in a composition is derivatized (such as conjugated) with a chemical crosslinker (or portion thereof). In some embodiments, at least about 90%, 91%, 92%, 93%, 94%, 95%, 96%, 97%, 98%, or 99% of the bioactive polypeptide in a composition is derivatized (such as conjugated) with a chemical crosslinker (or portion thereof). In some embodiments, the derivatized bioactive polypeptide specifically crosslinks to a derivatized albumin, thereby forming a bioactive polypeptide-albumin conjugate. Conjugation (crosslinking) can occur, for example, prior to combining the aqueous solution comprising the derivatized albumin and the organic solution. In some embodiments, conjugation occurs by combining the derivatized bioactive polypeptide with nanoparticles comprising derivatized albumin. In some embodiments, conjugation occurs by combining the derivatized bioactive polypeptide with isolated nanoparticles. In some embodiments, conjugation occurs by combining the derivatized bioactive polypeptide with isolated nanoparticles comprising derivatized albumin.

In some embodiments, the composition comprises nanoparticles comprising (a) a hydrophobic drug (such as a taxane or a limus drug), (b) an albumin (such as a human albumin), and (c) bevacizumab. In some embodiments, the composition comprises nanoparticles comprising (a) a hydrophobic drug (such as a taxane or a limus drug), (b) an albumin (such as a human albumin), and (c) bevacizumab, wherein the hydrophobic drug is coated (such as substantially coated) with the albumin. In some embodiments, the composition comprises nanoparticles comprising (a) a hydrophobic drug (such as a taxane or a limus drug), (b) an albumin (such as a human albumin), and (c) bevacizumab, wherein the hydrophobic drug is coated (such as substantially coated) with the albumin, and wherein bevacizumab is associated with the hydrophobic drug. In some embodiments, the composition comprises nanoparticles comprising (a) a hydrophobic drug (such as a taxane or a limus drug), (b) an albumin (such as a human albumin), and (c) bevacizumab, wherein the hydrophobic drug is coated (such as substantially coated) with the albumin, and wherein bevacizumab is associated with a solid core of the hydrophobic drug. In some embodiments, the composition comprises nanoparticles comprising (a) a hydrophobic drug (such as a taxane or a limus drug), (b) an albumin (such as a human albumin), and (c) bevacizumab, wherein the hydrophobic drug is coated (such as substantially coated) with the albumin, and wherein bevacizumab is embedded in a solid core of the hydrophobic drug. In some embodiments, the composition comprises nanoparticles comprising (a) a hydrophobic drug (such as a taxane or a limus drug), (b) an albumin (such as a human albumin), and (c) bevacizumab, wherein the hydrophobic drug is coated with the albumin, and wherein bevacizumab is associated with the albumin on the nanoparticles. In some embodiments, the composition comprises nanoparticles comprising (a) a hydrophobic drug (such as a taxane or a limus drug), (b) an albumin (such as a human albumin), and (c) bevacizumab, wherein the hydrophobic drug is coated with the albumin, and wherein bevacizumab is associated with the albumin on the nanoparticles non-covalently. In some embodiments, the composition comprises nanoparticles comprising (a) a hydrophobic drug (such as a taxane or a limus drug), (b) an albumin (such as a human albumin), and (c) bevacizumab, wherein the hydrophobic drug is coated with the albumin, and wherein bevacizumab is associated with the albumin on the nanoparticles covalently (such as via a disulfide bond or a chemical crosslink). In some embodiments, the composition comprises nanoparticles comprising (a) a hydrophobic drug (such as a taxane or a limus drug), (b) an albumin (such as a human albumin), and (c) bevacizumab, wherein the hydrophobic drug is coated with the albumin, and wherein bevacizumab is associated with the hydrophobic drug (such as a solid core of the hydrophobic drug) and the albumin on the nanoparticle. In some embodiments, the composition comprises nanoparticles comprising (a) paclitaxel, (b) an albumin (such as a human albumin), and (c) bevacizumab. In some embodiments, the composition comprises nanoparticles comprising (a) paclitaxel, (b) an albumin (such as a human albumin), and (c) bevacizumab, wherein paclitaxel is coated (such as substantially coated) with the albumin. In some embodiments, the composition comprises nanoparticles comprising (a) paclitaxel, (b) an albumin (such as a human albumin), and (c) bevacizumab, wherein paclitaxel is coated (such as substantially coated) with the albumin, and wherein bevacizumab is associated with paclitaxel. In some embodiments, the composition comprises nanoparticles comprising (a) paclitaxel, (b) an albumin (such as a human albumin), and (c) bevacizumab, wherein paclitaxel is coated (such as substantially coated) with the albumin, and wherein bevacizumab is associated with a solid core of paclitaxel. In some embodiments, the composition comprises nanoparticles comprising (a) paclitaxel, (b) an albumin (such as a human albumin), and (c) bevacizumab, wherein paclitaxel is coated (such as substantially coated) with the albumin, and wherein bevacizumab is embedded in a solid core of paclitaxel. In some embodiments, the composition comprises nanoparticles comprising (a) paclitaxel, (b) an albumin (such as a human albumin), and (c) bevacizumab, wherein paclitaxel is coated with the albumin, and wherein bevacizumab is associated with the albumin on the nanoparticles. In some embodiments, the composition comprises nanoparticles comprising (a) paclitaxel, (b) an albumin (such as a human albumin), and (c) bevacizumab, wherein paclitaxel is coated with the albumin, and wherein bevacizumab is associated with the albumin on the nanoparticles non-covalently. In some embodiments, the composition comprises nanoparticles comprising (a) paclitaxel, (b) an albumin (such as a human albumin), and (c) bevacizumab, wherein paclitaxel is coated with the albumin, and wherein bevacizumab is associated with the albumin on the nanoparticles covalently (such as via a disulfide bond or a chemical crosslink). In some embodiments, the composition comprises nanoparticles comprising (a) paclitaxel, (b) an albumin (such as a human albumin), and (c) bevacizumab, wherein paclitaxel is coated with the albumin, and wherein bevacizumab is associated with paclitaxel (such as a solid core of paclitaxel) and the albumin on the nanoparticle. In some embodiments, the composition comprises nanoparticles comprising (a) rapamycin, (b) an albumin (such as a human albumin), and (c) bevacizumab. In some embodiments, the composition comprises nanoparticles comprising (a) rapamycin, (b) an albumin (such as a human albumin), and (c) bevacizumab, wherein rapamycin is coated (such as substantially coated) with the albumin. In some embodiments, the composition comprises nanoparticles comprising (a) rapamycin, (b) an albumin (such as a human albumin), and (c) bevacizumab, wherein rapamycin is coated (such as substantially coated) with the albumin, and wherein bevacizumab is associated with rapamycin. In some embodiments, the composition comprises nanoparticles comprising (a) rapamycin, (b) an albumin (such as a human albumin), and (c) bevacizumab, wherein rapamycin is coated (such as substantially coated) with the albumin, and wherein bevacizumab is associated with a solid core of rapamycin. In some embodiments, the composition comprises nanoparticles comprising (a) rapamycin, (b) an albumin (such as a human albumin), and (c) bevacizumab, wherein rapamycin is coated (such as substantially coated) with the albumin, and wherein bevacizumab is embedded in a solid core of rapamycin. In some embodiments, the composition comprises nanoparticles comprising (a) rapamycin, (b) an albumin (such as a human albumin), and (c) bevacizumab, wherein rapamycin is coated with the albumin, and wherein bevacizumab is associated with the albumin on the nanoparticles. In some embodiments, the composition comprises nanoparticles comprising (a) rapamycin, (b) an albumin (such as a human albumin), and (c) bevacizumab, wherein rapamycin is coated with the albumin, and wherein bevacizumab is associated with the albumin on the nanoparticles non-covalently. In some embodiments, the composition comprises nanoparticles comprising (a) rapamycin, (b) an albumin (such as a human albumin), and (c) bevacizumab, wherein rapamycin is coated with the albumin, and wherein bevacizumab is associated with the albumin on the nanoparticles covalently (such as via a disulfide bond or a chemical crosslink). In some embodiments, the composition comprises nanoparticles comprising (a) rapamycin, (b) an albumin (such as a human albumin), and (c) bevacizumab, wherein rapamycin is coated with the albumin, and wherein bevacizumab is associated with rapamycin (such as a solid core of rapamycin) and the albumin on the nanoparticle. In some embodiments, the average diameter of nanoparticles in a composition, as measured by Dynamic Light Scattering, is no greater than about 200 nm.

In some embodiments, the composition comprises nanoparticles comprising (a) a hydrophobic drug (such as a taxane or a limus drug), (b) an albumin (such as a human albumin), and (c) cetuximab. In some embodiments, the composition comprises nanoparticles comprising (a) a hydrophobic drug (such as a taxane or a limus drug), (b) an albumin (such as a human albumin), and (c) cetuximab, wherein the hydrophobic drug is coated (such as substantially coated) with the albumin. In some embodiments, the composition comprises nanoparticles comprising (a) a hydrophobic drug (such as a taxane or a limus drug), (b) an albumin (such as a human albumin), and (c) cetuximab, wherein the hydrophobic drug is coated (such as substantially coated) with the albumin, and wherein cetuximab is associated with the hydrophobic drug. In some embodiments, the composition comprises nanoparticles comprising (a) a hydrophobic drug (such as a taxane or a limus drug), (b) an albumin (such as a human albumin), and (c) cetuximab, wherein the hydrophobic drug is coated (such as substantially coated) with the albumin, and wherein cetuximab is associated with a solid core of the hydrophobic drug. In some embodiments, the composition comprises nanoparticles comprising (a) a hydrophobic drug (such as a taxane or a limus drug), (b) an albumin (such as a human albumin), and (c) cetuximab, wherein the hydrophobic drug is coated (such as substantially coated) with the albumin, and wherein cetuximab is embedded in a solid core of the hydrophobic drug. In some embodiments, the composition comprises nanoparticles comprising (a) a hydrophobic drug (such as a taxane or a limus drug), (b) an albumin (such as a human albumin), and (c) cetuximab, wherein the hydrophobic drug is coated with the albumin, and wherein cetuximab is associated with the albumin on the nanoparticles. In some embodiments, the composition comprises nanoparticles comprising (a) a hydrophobic drug (such as a taxane or a limus drug), (b) an albumin (such as a human albumin), and (c) cetuximab, wherein the hydrophobic drug is coated with the albumin, and wherein cetuximab is associated with the albumin on the nanoparticles non-covalently. In some embodiments, the composition comprises nanoparticles comprising (a) a hydrophobic drug (such as a taxane or a limus drug), (b) an albumin (such as a human albumin), and (c) cetuximab, wherein the hydrophobic drug is coated with the albumin, and wherein cetuximab is associated with the albumin on the nanoparticles covalently (such as via a disulfide bond or a chemical crosslink). In some embodiments, the composition comprises nanoparticles comprising (a) a hydrophobic drug (such as a taxane or a limus drug), (b) an albumin (such as a human albumin), and (c) cetuximab, wherein the hydrophobic drug is coated with the albumin, and wherein cetuximab is associated with the hydrophobic drug (such as a solid core of the hydrophobic drug) and the albumin on the nanoparticle. In some embodiments, the composition comprises nanoparticles comprising (a) paclitaxel, (b) an albumin (such as a human albumin), and (c) cetuximab. In some embodiments, the composition comprises nanoparticles comprising (a) paclitaxel, (b) an albumin (such as a human albumin), and (c) cetuximab, wherein paclitaxel is coated (such as substantially coated) with the albumin. In some embodiments, the composition comprises nanoparticles comprising (a) paclitaxel, (b) an albumin (such as a human albumin), and (c) cetuximab, wherein paclitaxel is coated (such as substantially coated) with the albumin, and wherein cetuximab is associated with paclitaxel. In some embodiments, the composition comprises nanoparticles comprising (a) paclitaxel, (b) an albumin (such as a human albumin), and (c) cetuximab, wherein paclitaxel is coated (such as substantially coated) with the albumin, and wherein cetuximab is associated with a solid core of paclitaxel. In some embodiments, the composition comprises nanoparticles comprising (a) paclitaxel, (b) an albumin (such as a human albumin), and (c) cetuximab, wherein paclitaxel is coated (such as substantially coated) with the albumin, and wherein cetuximab is embedded in a solid core of paclitaxel. In some embodiments, the composition comprises nanoparticles comprising (a) paclitaxel, (b) an albumin (such as a human albumin), and (c) cetuximab, wherein paclitaxel is coated with the albumin, and wherein cetuximab is associated with the albumin on the nanoparticles. In some embodiments, the composition comprises nanoparticles comprising (a) paclitaxel, (b) an albumin (such as a human albumin), and (c) cetuximab, wherein paclitaxel is coated with the albumin, and wherein cetuximab is associated with the albumin on the nanoparticles non-covalently. In some embodiments, the composition comprises nanoparticles comprising (a) paclitaxel, (b) an albumin (such as a human albumin), and (c) cetuximab, wherein paclitaxel is coated with the albumin, and wherein cetuximab is associated with the albumin on the nanoparticles covalently (such as via a disulfide bond or a chemical crosslink). In some embodiments, the composition comprises nanoparticles comprising (a) paclitaxel, (b) an albumin (such as a human albumin), and (c) cetuximab, wherein paclitaxel is coated with the albumin, and wherein cetuximab is associated with paclitaxel (such as a solid core of paclitaxel) and the albumin on the nanoparticle. In some embodiments, the composition comprises nanoparticles comprising (a) rapamycin, (b) an albumin (such as a human albumin), and (c) cetuximab. In some embodiments, the composition comprises nanoparticles comprising (a) rapamycin, (b) an albumin (such as a human albumin), and (c) cetuximab, wherein rapamycin is coated (such as substantially coated) with the albumin. In some embodiments, the composition comprises nanoparticles comprising (a) rapamycin, (b) an albumin (such as a human albumin), and (c) cetuximab, wherein rapamycin is coated (such as substantially coated) with the albumin, and wherein cetuximab is associated with rapamycin. In some embodiments, the composition comprises nanoparticles comprising (a) rapamycin, (b) an albumin (such as a human albumin), and (c) cetuximab, wherein rapamycin is coated (such as substantially coated) with the albumin, and wherein cetuximab is associated with a solid core of rapamycin. In some embodiments, the composition comprises nanoparticles comprising (a) rapamycin, (b) an albumin (such as a human albumin), and (c) cetuximab, wherein rapamycin is coated (such as substantially coated) with the albumin, and wherein cetuximab is embedded in a solid core of rapamycin. In some embodiments, the composition comprises nanoparticles comprising (a) rapamycin, (b) an albumin (such as a human albumin), and (c) cetuximab, wherein rapamycin is coated with the albumin, and wherein cetuximab is associated with the albumin on the nanoparticles. In some embodiments, the composition comprises nanoparticles comprising (a) rapamycin, (b) an albumin (such as a human albumin), and (c) cetuximab, wherein rapamycin is coated with the albumin, and wherein cetuximab is associated with the albumin on the nanoparticles non-covalently. In some embodiments, the composition comprises nanoparticles comprising (a) rapamycin, (b) an albumin (such as a human albumin), and (c) cetuximab, wherein rapamycin is coated with the albumin, and wherein cetuximab is associated with the albumin on the nanoparticles covalently (such as via a disulfide bond or a chemical crosslink). In some embodiments, the composition comprises nanoparticles comprising (a) rapamycin, (b) an albumin (such as a human albumin), and (c) cetuximab, wherein rapamycin is coated with the albumin, and wherein cetuximab is associated with rapamycin (such as a solid core of rapamycin) and the albumin on the nanoparticle. In some embodiments, the average diameter of nanoparticles in a composition, as measured by Dynamic Light Scattering, is no greater than about 200 nm.

In some embodiments, the composition comprises nanoparticles comprising (a) a hydrophobic drug (such as a taxane or a limus drug), (b) an albumin (such as a human albumin), and (c) ipilimumab. In some embodiments, the composition comprises nanoparticles comprising (a) a hydrophobic drug (such as a taxane or a limus drug), (b) an albumin (such as a human albumin), and (c) ipilimumab, wherein the hydrophobic drug is coated (such as substantially coated) with the albumin. In some embodiments, the composition comprises nanoparticles comprising (a) a hydrophobic drug (such as a taxane or a limus drug), (b) an albumin (such as a human albumin), and (c) ipilimumab, wherein the hydrophobic drug is coated (such as substantially coated) with the albumin, and wherein ipilimumab is associated with the hydrophobic drug. In some embodiments, the composition comprises nanoparticles comprising (a) a hydrophobic drug (such as a taxane or a limus drug), (b) an albumin (such as a human albumin), and (c) ipilimumab, wherein the hydrophobic drug is coated (such as substantially coated) with the albumin, and wherein ipilimumab is associated with a solid core of the hydrophobic drug. In some embodiments, the composition comprises nanoparticles comprising (a) a hydrophobic drug (such as a taxane or a limus drug), (b) an albumin (such as a human albumin), and (c) ipilimumab, wherein the hydrophobic drug is coated (such as substantially coated) with the albumin, and wherein ipilimumab is embedded in a solid core of the hydrophobic drug. In some embodiments, the composition comprises nanoparticles comprising (a) a hydrophobic drug (such as a taxane or a limus drug), (b) an albumin (such as a human albumin), and (c) ipilimumab, wherein the hydrophobic drug is coated with the albumin, and wherein ipilimumab is associated with the albumin on the nanoparticles. In some embodiments, the composition comprises nanoparticles comprising (a) a hydrophobic drug (such as a taxane or a limus drug), (b) an albumin (such as a human albumin), and (c) ipilimumab, wherein the hydrophobic drug is coated with the albumin, and wherein ipilimumab is associated with the albumin on the nanoparticles non-covalently. In some embodiments, the composition comprises nanoparticles comprising (a) a hydrophobic drug (such as a taxane or a limus drug), (b) an albumin (such as a human albumin), and (c) ipilimumab, wherein the hydrophobic drug is coated with the albumin, and wherein ipilimumab is associated with the albumin on the nanoparticles covalently (such as via a disulfide bond or a chemical crosslink). In some embodiments, the composition comprises nanoparticles comprising (a) a hydrophobic drug (such as a taxane or a limus drug), (b) an albumin (such as a human albumin), and (c) ipilimumab, wherein the hydrophobic drug is coated with the albumin, and wherein ipilimumab is associated with the hydrophobic drug (such as a solid core of the hydrophobic drug) and the albumin on the nanoparticle. In some embodiments, the composition comprises nanoparticles comprising (a) paclitaxel, (b) an albumin (such as a human albumin), and (c) ipilimumab. In some embodiments, the composition comprises nanoparticles comprising (a) paclitaxel, (b) an albumin (such as a human albumin), and (c) ipilimumab, wherein paclitaxel is coated (such as substantially coated) with the albumin. In some embodiments, the composition comprises nanoparticles comprising (a) paclitaxel, (b) an albumin (such as a human albumin), and (c) ipilimumab, wherein paclitaxel is coated (such as substantially coated) with the albumin, and wherein ipilimumab is associated with paclitaxel. In some embodiments, the composition comprises nanoparticles comprising (a) paclitaxel, (b) an albumin (such as a human albumin), and (c) ipilimumab, wherein paclitaxel is coated (such as substantially coated) with the albumin, and wherein ipilimumab is associated with a solid core of paclitaxel. In some embodiments, the composition comprises nanoparticles comprising (a) paclitaxel, (b) an albumin (such as a human albumin), and (c) ipilimumab, wherein paclitaxel is coated (such as substantially coated) with the albumin, and wherein ipilimumab is embedded in a solid core of paclitaxel. In some embodiments, the composition comprises nanoparticles comprising (a) paclitaxel, (b) an albumin (such as a human albumin), and (c) ipilimumab, wherein paclitaxel is coated with the albumin, and wherein ipilimumab is associated with the albumin on the nanoparticles. In some embodiments, the composition comprises nanoparticles comprising (a) paclitaxel, (b) an albumin (such as a human albumin), and (c) ipilimumab, wherein paclitaxel is coated with the albumin, and wherein ipilimumab is associated with the albumin on the nanoparticles non-covalently. In some embodiments, the composition comprises nanoparticles comprising (a) paclitaxel, (b) an albumin (such as a human albumin), and (c) ipilimumab, wherein paclitaxel is coated with the albumin, and wherein ipilimumab is associated with the albumin on the nanoparticles covalently (such as via a disulfide bond or a chemical crosslink). In some embodiments, the composition comprises nanoparticles comprising (a) paclitaxel, (b) an albumin (such as a human albumin), and (c) ipilimumab, wherein paclitaxel is coated with the albumin, and wherein ipilimumab is associated with paclitaxel (such as a solid core of paclitaxel) and the albumin on the nanoparticle. In some embodiments, the composition comprises nanoparticles comprising (a) rapamycin, (b) an albumin (such as a human albumin), and (c) ipilimumab. In some embodiments, the composition comprises nanoparticles comprising (a) rapamycin, (b) an albumin (such as a human albumin), and (c) ipilimumab, wherein rapamycin is coated (such as substantially coated) with the albumin. In some embodiments, the composition comprises nanoparticles comprising (a) rapamycin, (b) an albumin (such as a human albumin), and (c) ipilimumab, wherein rapamycin is coated (such as substantially coated) with the albumin, and wherein ipilimumab is associated with rapamycin. In some embodiments, the composition comprises nanoparticles comprising (a) rapamycin, (b) an albumin (such as a human albumin), and (c) ipilimumab, wherein rapamycin is coated (such as substantially coated) with the albumin, and wherein ipilimumab is associated with a solid core of rapamycin. In some embodiments, the composition comprises nanoparticles comprising (a) rapamycin, (b) an albumin (such as a human albumin), and (c) ipilimumab, wherein rapamycin is coated (such as substantially coated) with the albumin, and wherein ipilimumab is embedded in a solid core of rapamycin. In some embodiments, the composition comprises nanoparticles comprising (a) rapamycin, (b) an albumin (such as a human albumin), and (c) ipilimumab, wherein rapamycin is coated with the albumin, and wherein ipilimumab is associated with the albumin on the nanoparticles. In some embodiments, the composition comprises nanoparticles comprising (a) rapamycin, (b) an albumin (such as a human albumin), and (c) ipilimumab, wherein rapamycin is coated with the albumin, and wherein ipilimumab is associated with the albumin on the nanoparticles non-covalently. In some embodiments, the composition comprises nanoparticles comprising (a) rapamycin, (b) an albumin (such as a human albumin), and (c) ipilimumab, wherein rapamycin is coated with the albumin, and wherein ipilimumab is associated with the albumin on the nanoparticles covalently (such as via a disulfide bond or a chemical crosslink). In some embodiments, the composition comprises nanoparticles comprising (a) rapamycin, (b) an albumin (such as a human albumin), and (c) ipilimumab, wherein rapamycin is coated with the albumin, and wherein ipilimumab is associated with rapamycin (such as a solid core of rapamycin) and the albumin on the nanoparticle. In some embodiments, the average diameter of nanoparticles in a composition, as measured by Dynamic Light Scattering, is no greater than about 200 nm.

In some embodiments, the composition comprises nanoparticles comprising (a) a hydrophobic drug (such as a taxane or a limus drug), (b) an albumin (such as a human albumin), and (c) nivolumab. In some embodiments, the composition comprises nanoparticles comprising (a) a hydrophobic drug (such as a taxane or a limus drug), (b) an albumin (such as a human albumin), and (c) nivolumab, wherein the hydrophobic drug is coated (such as substantially coated) with the albumin. In some embodiments, the composition comprises nanoparticles comprising (a) a hydrophobic drug (such as a taxane or a limus drug), (b) an albumin (such as a human albumin), and (c) nivolumab, wherein the hydrophobic drug is coated (such as substantially coated) with the albumin, and wherein nivolumab is associated with the hydrophobic drug. In some embodiments, the composition comprises nanoparticles comprising (a) a hydrophobic drug (such as a taxane or a limus drug), (b) an albumin (such as a human albumin), and (c) nivolumab, wherein the hydrophobic drug is coated (such as substantially coated) with the albumin, and wherein nivolumab is associated with a solid core of the hydrophobic drug. In some embodiments, the composition comprises nanoparticles comprising (a) a hydrophobic drug (such as a taxane or a limus drug), (b) an albumin (such as a human albumin), and (c) nivolumab, wherein the hydrophobic drug is coated (such as substantially coated) with the albumin, and wherein nivolumab is embedded in a solid core of the hydrophobic drug. In some embodiments, the composition comprises nanoparticles comprising (a) a hydrophobic drug (such as a taxane or a limus drug), (b) an albumin (such as a human albumin), and (c) nivolumab, wherein the hydrophobic drug is coated with the albumin, and wherein nivolumab is associated with the albumin on the nanoparticles. In some embodiments, the composition comprises nanoparticles comprising (a) a hydrophobic drug (such as a taxane or a limus drug), (b) an albumin (such as a human albumin), and (c) nivolumab, wherein the hydrophobic drug is coated with the albumin, and wherein nivolumab is associated with the albumin on the nanoparticles non-covalently. In some embodiments, the composition comprises nanoparticles comprising (a) a hydrophobic drug (such as a taxane or a limus drug), (b) an albumin (such as a human albumin), and (c) nivolumab, wherein the hydrophobic drug is coated with the albumin, and wherein nivolumab is associated with the albumin on the nanoparticles covalently (such as via a disulfide bond or a chemical crosslink). In some embodiments, the composition comprises nanoparticles comprising (a) a hydrophobic drug (such as a taxane or a limus drug), (b) an albumin (such as a human albumin), and (c) nivolumab, wherein the hydrophobic drug is coated with the albumin, and wherein nivolumab is associated with the hydrophobic drug (such as a solid core of the hydrophobic drug) and the albumin on the nanoparticle. In some embodiments, the composition comprises nanoparticles comprising (a) paclitaxel, (b) an albumin (such as a human albumin), and (c) nivolumab. In some embodiments, the composition comprises nanoparticles comprising (a) paclitaxel, (b) an albumin (such as a human albumin), and (c) nivolumab, wherein paclitaxel is coated (such as substantially coated) with the albumin. In some embodiments, the composition comprises nanoparticles comprising (a) paclitaxel, (b) an albumin (such as a human albumin), and (c) nivolumab, wherein paclitaxel is coated (such as substantially coated) with the albumin, and wherein nivolumab is associated with paclitaxel. In some embodiments, the composition comprises nanoparticles comprising (a) paclitaxel, (b) an albumin (such as a human albumin), and (c) nivolumab, wherein paclitaxel is coated (such as substantially coated) with the albumin, and wherein nivolumab is associated with a solid core of paclitaxel. In some embodiments, the composition comprises nanoparticles comprising (a) paclitaxel, (b) an albumin (such as a human albumin), and (c) nivolumab, wherein paclitaxel is coated (such as substantially coated) with the albumin, and wherein nivolumab is embedded in a solid core of paclitaxel. In some embodiments, the composition comprises nanoparticles comprising (a) paclitaxel, (b) an albumin (such as a human albumin), and (c) nivolumab, wherein paclitaxel is coated with the albumin, and wherein nivolumab is associated with the albumin on the nanoparticles. In some embodiments, the composition comprises nanoparticles comprising (a) paclitaxel, (b) an albumin (such as a human albumin), and (c) nivolumab, wherein paclitaxel is coated with the albumin, and wherein nivolumab is associated with the albumin on the nanoparticles non-covalently. In some embodiments, the composition comprises nanoparticles comprising (a) paclitaxel, (b) an albumin (such as a human albumin), and (c) nivolumab, wherein paclitaxel is coated with the albumin, and wherein nivolumab is associated with the albumin on the nanoparticles covalently (such as via a disulfide bond or a chemical crosslink). In some embodiments, the composition comprises nanoparticles comprising (a) paclitaxel, (b) an albumin (such as a human albumin), and (c) nivolumab, wherein paclitaxel is coated with the albumin, and wherein nivolumab is associated with paclitaxel (such as a solid core of paclitaxel) and the albumin on the nanoparticle. In some embodiments, the composition comprises nanoparticles comprising (a) rapamycin, (b) an albumin (such as a human albumin), and (c) nivolumab. In some embodiments, the composition comprises nanoparticles comprising (a) rapamycin, (b) an albumin (such as a human albumin), and (c) nivolumab, wherein rapamycin is coated (such as substantially coated) with the albumin. In some embodiments, the composition comprises nanoparticles comprising (a) rapamycin, (b) an albumin (such as a human albumin), and (c) nivolumab, wherein rapamycin is coated (such as substantially coated) with the albumin, and wherein nivolumab is associated with rapamycin. In some embodiments, the composition comprises nanoparticles comprising (a) rapamycin, (b) an albumin (such as a human albumin), and (c) nivolumab, wherein rapamycin is coated (such as substantially coated) with the albumin, and wherein nivolumab is associated with a solid core of rapamycin. In some embodiments, the composition comprises nanoparticles comprising (a) rapamycin, (b) an albumin (such as a human albumin), and (c) nivolumab, wherein rapamycin is coated (such as substantially coated) with the albumin, and wherein nivolumab is embedded in a solid core of rapamycin. In some embodiments, the composition comprises nanoparticles comprising (a) rapamycin, (b) an albumin (such as a human albumin), and (c) nivolumab, wherein rapamycin is coated with the albumin, and wherein nivolumab is associated with the albumin on the nanoparticles. In some embodiments, the composition comprises nanoparticles comprising (a) rapamycin, (b) an albumin (such as a human albumin), and (c) nivolumab, wherein rapamycin is coated with the albumin, and wherein nivolumab is associated with the albumin on the nanoparticles non-covalently. In some embodiments, the composition comprises nanoparticles comprising (a) rapamycin, (b) an albumin (such as a human albumin), and (c) nivolumab, wherein rapamycin is coated with the albumin, and wherein nivolumab is associated with the albumin on the nanoparticles covalently (such as via a disulfide bond or a chemical crosslink). In some embodiments, the composition comprises nanoparticles comprising (a) rapamycin, (b) an albumin (such as a human albumin), and (c) nivolumab, wherein rapamycin is coated with the albumin, and wherein nivolumab is associated with rapamycin (such as a solid core of rapamycin) and the albumin on the nanoparticle. In some embodiments, the average diameter of nanoparticles in a composition, as measured by Dynamic Light Scattering, is no greater than about 200 nm.

In some embodiments, the composition comprises nanoparticles comprising (a) a hydrophobic drug (such as a taxane or a limus drug), (b) an albumin (such as a human albumin), and (c) panitumumab. In some embodiments, the composition comprises nanoparticles comprising (a) a hydrophobic drug (such as a taxane or a limus drug), (b) an albumin (such as a human albumin), and (c) panitumumab, wherein the hydrophobic drug is coated (such as substantially coated) with the albumin. In some embodiments, the composition comprises nanoparticles comprising (a) a hydrophobic drug (such as a taxane or a limus drug), (b) an albumin (such as a human albumin), and (c) panitumumab, wherein the hydrophobic drug is coated (such as substantially coated) with the albumin, and wherein panitumumab is associated with the hydrophobic drug. In some embodiments, the composition comprises nanoparticles comprising (a) a hydrophobic drug (such as a taxane or a limus drug), (b) an albumin (such as a human albumin), and (c) panitumumab, wherein the hydrophobic drug is coated (such as substantially coated) with the albumin, and wherein panitumumab is associated with a solid core of the hydrophobic drug. In some embodiments, the composition comprises nanoparticles comprising (a) a hydrophobic drug (such as a taxane or a limus drug), (b) an albumin (such as a human albumin), and (c) panitumumab, wherein the hydrophobic drug is coated (such as substantially coated) with the albumin, and wherein panitumumab is embedded in a solid core of the hydrophobic drug. In some embodiments, the composition comprises nanoparticles comprising (a) a hydrophobic drug (such as a taxane or a limus drug), (b) an albumin (such as a human albumin), and (c) panitumumab, wherein the hydrophobic drug is coated with the albumin, and wherein panitumumab is associated with the albumin on the nanoparticles. In some embodiments, the composition comprises nanoparticles comprising (a) a hydrophobic drug (such as a taxane or a limus drug), (b) an albumin (such as a human albumin), and (c) panitumumab, wherein the hydrophobic drug is coated with the albumin, and wherein panitumumab is associated with the albumin on the nanoparticles non-covalently. In some embodiments, the composition comprises nanoparticles comprising (a) a hydrophobic drug (such as a taxane or a limus drug), (b) an albumin (such as a human albumin), and (c) panitumumab, wherein the hydrophobic drug is coated with the albumin, and wherein panitumumab is associated with the albumin on the nanoparticles covalently (such as via a disulfide bond or a chemical crosslink). In some embodiments, the composition comprises nanoparticles comprising (a) a hydrophobic drug (such as a taxane or a limus drug), (b) an albumin (such as a human albumin), and (c) panitumumab, wherein the hydrophobic drug is coated with the albumin, and wherein panitumumab is associated with the hydrophobic drug (such as a solid core of the hydrophobic drug) and the albumin on the nanoparticle. In some embodiments, the composition comprises nanoparticles comprising (a) paclitaxel, (b) an albumin (such as a human albumin), and (c) panitumumab. In some embodiments, the composition comprises nanoparticles comprising (a) paclitaxel, (b) an albumin (such as a human albumin), and (c) panitumumab, wherein paclitaxel is coated (such as substantially coated) with the albumin. In some embodiments, the composition comprises nanoparticles comprising (a) paclitaxel, (b) an albumin (such as a human albumin), and (c) panitumumab, wherein paclitaxel is coated (such as substantially coated) with the albumin, and wherein panitumumab is associated with paclitaxel. In some embodiments, the composition comprises nanoparticles comprising (a) paclitaxel, (b) an albumin (such as a human albumin), and (c) panitumumab, wherein paclitaxel is coated (such as substantially coated) with the albumin, and wherein panitumumab is associated with a solid core of paclitaxel. In some embodiments, the composition comprises nanoparticles comprising (a) paclitaxel, (b) an albumin (such as a human albumin), and (c) panitumumab, wherein paclitaxel is coated (such as substantially coated) with the albumin, and wherein panitumumab is embedded in a solid core of paclitaxel. In some embodiments, the composition comprises nanoparticles comprising (a) paclitaxel, (b) an albumin (such as a human albumin), and (c) panitumumab, wherein paclitaxel is coated with the albumin, and wherein panitumumab is associated with the albumin on the nanoparticles. In some embodiments, the composition comprises nanoparticles comprising (a) paclitaxel, (b) an albumin (such as a human albumin), and (c) panitumumab, wherein paclitaxel is coated with the albumin, and wherein panitumumab is associated with the albumin on the nanoparticles non-covalently. In some embodiments, the composition comprises nanoparticles comprising (a) paclitaxel, (b) an albumin (such as a human albumin), and (c) panitumumab, wherein paclitaxel is coated with the albumin, and wherein panitumumab is associated with the albumin on the nanoparticles covalently (such as via a disulfide bond or a chemical crosslink). In some embodiments, the composition comprises nanoparticles comprising (a) paclitaxel, (b) an albumin (such as a human albumin), and (c) panitumumab, wherein paclitaxel is coated with the albumin, and wherein panitumumab is associated with paclitaxel (such as a solid core of paclitaxel) and the albumin on the nanoparticle. In some embodiments, the composition comprises nanoparticles comprising (a) rapamycin, (b) an albumin (such as a human albumin), and (c) panitumumab. In some embodiments, the composition comprises nanoparticles comprising (a) rapamycin, (b) an albumin (such as a human albumin), and (c) panitumumab, wherein rapamycin is coated (such as substantially coated) with the albumin. In some embodiments, the composition comprises nanoparticles comprising (a) rapamycin, (b) an albumin (such as a human albumin), and (c) panitumumab, wherein rapamycin is coated (such as substantially coated) with the albumin, and wherein panitumumab is associated with rapamycin. In some embodiments, the composition comprises nanoparticles comprising (a) rapamycin, (b) an albumin (such as a human albumin), and (c) panitumumab, wherein rapamycin is coated (such as substantially coated) with the albumin, and wherein panitumumab is associated with a solid core of rapamycin. In some embodiments, the composition comprises nanoparticles comprising (a) rapamycin, (b) an albumin (such as a human albumin), and (c) panitumumab, wherein rapamycin is coated (such as substantially coated) with the albumin, and wherein panitumumab is embedded in a solid core of rapamycin. In some embodiments, the composition comprises nanoparticles comprising (a) rapamycin, (b) an albumin (such as a human albumin), and (c) panitumumab, wherein rapamycin is coated with the albumin, and wherein panitumumab is associated with the albumin on the nanoparticles. In some embodiments, the composition comprises nanoparticles comprising (a) rapamycin, (b) an albumin (such as a human albumin), and (c) panitumumab, wherein rapamycin is coated with the albumin, and wherein panitumumab is associated with the albumin on the nanoparticles non-covalently. In some embodiments, the composition comprises nanoparticles comprising (a) rapamycin, (b) an albumin (such as a human albumin), and (c) panitumumab, wherein rapamycin is coated with the albumin, and wherein panitumumab is associated with the albumin on the nanoparticles covalently (such as via a disulfide bond or a chemical crosslink). In some embodiments, the composition comprises nanoparticles comprising (a) rapamycin, (b) an albumin (such as a human albumin), and (c) panitumumab, wherein rapamycin is coated with the albumin, and wherein panitumumab is associated with rapamycin (such as a solid core of rapamycin) and the albumin on the nanoparticle. In some embodiments, the average diameter of nanoparticles in a composition, as measured by Dynamic Light Scattering, is no greater than about 200 nm.

In some embodiments, the composition comprises nanoparticles comprising (a) a hydrophobic drug (such as a taxane or a limus drug), (b) an albumin (such as a human albumin), and (c) rituximab. In some embodiments, the composition comprises nanoparticles comprising (a) a hydrophobic drug (such as a taxane or a limus drug), (b) an albumin (such as a human albumin), and (c) rituximab, wherein the hydrophobic drug is coated (such as substantially coated) with the albumin. In some embodiments, the composition comprises nanoparticles comprising (a) a hydrophobic drug (such as a taxane or a limus drug), (b) an albumin (such as a human albumin), and (c) rituximab, wherein the hydrophobic drug is coated (such as substantially coated) with the albumin, and wherein rituximab is associated with the hydrophobic drug. In some embodiments, the composition comprises nanoparticles comprising (a) a hydrophobic drug (such as a taxane or a limus drug), (b) an albumin (such as a human albumin), and (c) rituximab, wherein the hydrophobic drug is coated (such as substantially coated) with the albumin, and wherein rituximab is associated with a solid core of the hydrophobic drug. In some embodiments, the composition comprises nanoparticles comprising (a) a hydrophobic drug (such as a taxane or a limus drug), (b) an albumin (such as a human albumin), and (c) rituximab, wherein the hydrophobic drug is coated (such as substantially coated) with the albumin, and wherein rituximab is embedded in a solid core of the hydrophobic drug. In some embodiments, the composition comprises nanoparticles comprising (a) a hydrophobic drug (such as a taxane or a limus drug), (b) an albumin (such as a human albumin), and (c) rituximab, wherein the hydrophobic drug is coated with the albumin, and wherein rituximab is associated with the albumin on the nanoparticles. In some embodiments, the composition comprises nanoparticles comprising (a) a hydrophobic drug (such as a taxane or a limus drug), (b) an albumin (such as a human albumin), and (c) rituximab, wherein the hydrophobic drug is coated with the albumin, and wherein rituximab is associated with the albumin on the nanoparticles non-covalently. In some embodiments, the composition comprises nanoparticles comprising (a) a hydrophobic drug (such as a taxane or a limus drug), (b) an albumin (such as a human albumin), and (c) rituximab, wherein the hydrophobic drug is coated with the albumin, and wherein rituximab is associated with the albumin on the nanoparticles covalently (such as via a disulfide bond or a chemical crosslink). In some embodiments, the composition comprises nanoparticles comprising (a) a hydrophobic drug (such as a taxane or a limus drug), (b) an albumin (such as a human albumin), and (c) rituximab, wherein the hydrophobic drug is coated with the albumin, and wherein rituximab is associated with the hydrophobic drug (such as a solid core of the hydrophobic drug) and the albumin on the nanoparticle. In some embodiments, the composition comprises nanoparticles comprising (a) paclitaxel, (b) an albumin (such as a human albumin), and (c) rituximab. In some embodiments, the composition comprises nanoparticles comprising (a) paclitaxel, (b) an albumin (such as a human albumin), and (c) rituximab, wherein paclitaxel is coated (such as substantially coated) with the albumin. In some embodiments, the composition comprises nanoparticles comprising (a) paclitaxel, (b) an albumin (such as a human albumin), and (c) rituximab, wherein paclitaxel is coated (such as substantially coated) with the albumin, and wherein rituximab is associated with paclitaxel. In some embodiments, the composition comprises nanoparticles comprising (a) paclitaxel, (b) an albumin (such as a human albumin), and (c) rituximab, wherein paclitaxel is coated (such as substantially coated) with the albumin, and wherein rituximab is associated with a solid core of paclitaxel. In some embodiments, the composition comprises nanoparticles comprising (a) paclitaxel, (b) an albumin (such as a human albumin), and (c) rituximab, wherein paclitaxel is coated (such as substantially coated) with the albumin, and wherein rituximab is embedded in a solid core of paclitaxel. In some embodiments, the composition comprises nanoparticles comprising (a) paclitaxel, (b) an albumin (such as a human albumin), and (c) rituximab, wherein paclitaxel is coated with the albumin, and wherein rituximab is associated with the albumin on the nanoparticles. In some embodiments, the composition comprises nanoparticles comprising (a) paclitaxel, (b) an albumin (such as a human albumin), and (c) rituximab, wherein paclitaxel is coated with the albumin, and wherein rituximab is associated with the albumin on the nanoparticles non-covalently. In some embodiments, the composition comprises nanoparticles comprising (a) paclitaxel, (b) an albumin (such as a human albumin), and (c) rituximab, wherein paclitaxel is coated with the albumin, and wherein rituximab is associated with the albumin on the nanoparticles covalently (such as via a disulfide bond or a chemical crosslink). In some embodiments, the composition comprises nanoparticles comprising (a) paclitaxel, (b) an albumin (such as a human albumin), and (c) rituximab, wherein paclitaxel is coated with the albumin, and wherein rituximab is associated with paclitaxel (such as a solid core of paclitaxel) and the albumin on the nanoparticle. In some embodiments, the composition comprises nanoparticles comprising (a) rapamycin, (b) an albumin (such as a human albumin), and (c) rituximab. In some embodiments, the composition comprises nanoparticles comprising (a) rapamycin, (b) an albumin (such as a human albumin), and (c) rituximab, wherein rapamycin is coated (such as substantially coated) with the albumin. In some embodiments, the composition comprises nanoparticles comprising (a) rapamycin, (b) an albumin (such as a human albumin), and (c) rituximab, wherein rapamycin is coated (such as substantially coated) with the albumin, and wherein rituximab is associated with rapamycin. In some embodiments, the composition comprises nanoparticles comprising (a) rapamycin, (b) an albumin (such as a human albumin), and (c) rituximab, wherein rapamycin is coated (such as substantially coated) with the albumin, and wherein rituximab is associated with a solid core of rapamycin. In some embodiments, the composition comprises nanoparticles comprising (a) rapamycin, (b) an albumin (such as a human albumin), and (c) rituximab, wherein rapamycin is coated (such as substantially coated) with the albumin, and wherein rituximab is embedded in a solid core of rapamycin. In some embodiments, the composition comprises nanoparticles comprising (a) rapamycin, (b) an albumin (such as a human albumin), and (c) rituximab, wherein rapamycin is coated with the albumin, and wherein rituximab is associated with the albumin on the nanoparticles. In some embodiments, the composition comprises nanoparticles comprising (a) rapamycin, (b) an albumin (such as a human albumin), and (c) rituximab, wherein rapamycin is coated with the albumin, and wherein rituximab is associated with the albumin on the nanoparticles non-covalently. In some embodiments, the composition comprises nanoparticles comprising (a) rapamycin, (b) an albumin (such as a human albumin), and (c) rituximab, wherein rapamycin is coated with the albumin, and wherein rituximab is associated with the albumin on the nanoparticles covalently (such as via a disulfide bond or a chemical crosslink). In some embodiments, the composition comprises nanoparticles comprising (a) rapamycin, (b) an albumin (such as a human albumin), and (c) rituximab, wherein rapamycin is coated with the albumin, and wherein rituximab is associated with rapamycin (such as a solid core of rapamycin) and the albumin on the nanoparticle. In some embodiments, the average diameter of nanoparticles in a composition, as measured by Dynamic Light Scattering, is no greater than about 200 nm.

In some embodiments, the composition comprises nanoparticles comprising (a) a hydrophobic drug (such as a taxane or a limus drug), (b) an albumin (such as a human albumin), and (c) durvalumab. In some embodiments, the composition comprises nanoparticles comprising (a) a hydrophobic drug (such as a taxane or a limus drug), (b) an albumin (such as a human albumin), and (c) durvalumab, wherein the hydrophobic drug is coated (such as substantially coated) with the albumin. In some embodiments, the composition comprises nanoparticles comprising (a) a hydrophobic drug (such as a taxane or a limus drug), (b) an albumin (such as a human albumin), and (c) durvalumab, wherein the hydrophobic drug is coated (such as substantially coated) with the albumin, and wherein durvalumab is associated with the hydrophobic drug. In some embodiments, the composition comprises nanoparticles comprising (a) a hydrophobic drug (such as a taxane or a limus drug), (b) an albumin (such as a human albumin), and (c) durvalumab, wherein the hydrophobic drug is coated (such as substantially coated) with the albumin, and wherein durvalumab is associated with a solid core of the hydrophobic drug. In some embodiments, the composition comprises nanoparticles comprising (a) a hydrophobic drug (such as a taxane or a limus drug), (b) an albumin (such as a human albumin), and (c) durvalumab, wherein the hydrophobic drug is coated (such as substantially coated) with the albumin, and wherein durvalumab is embedded in a solid core of the hydrophobic drug. In some embodiments, the composition comprises nanoparticles comprising (a) a hydrophobic drug (such as a taxane or a limus drug), (b) an albumin (such as a human albumin), and (c) durvalumab, wherein the hydrophobic drug is coated with the albumin, and wherein durvalumab is associated with the albumin on the nanoparticles. In some embodiments, the composition comprises nanoparticles comprising (a) a hydrophobic drug (such as a taxane or a limus drug), (b) an albumin (such as a human albumin), and (c) durvalumab, wherein the hydrophobic drug is coated with the albumin, and wherein durvalumab is associated with the albumin on the nanoparticles non-covalently. In some embodiments, the composition comprises nanoparticles comprising (a) a hydrophobic drug (such as a taxane or a limus drug), (b) an albumin (such as a human albumin), and (c) durvalumab, wherein the hydrophobic drug is coated with the albumin, and wherein durvalumab is associated with the albumin on the nanoparticles covalently (such as via a disulfide bond or a chemical crosslink). In some embodiments, the composition comprises nanoparticles comprising (a) a hydrophobic drug (such as a taxane or a limus drug), (b) an albumin (such as a human albumin), and (c) durvalumab, wherein the hydrophobic drug is coated with the albumin, and wherein durvalumab is associated with the hydrophobic drug (such as a solid core of the hydrophobic drug) and the albumin on the nanoparticle. In some embodiments, the composition comprises nanoparticles comprising (a) paclitaxel, (b) an albumin (such as a human albumin), and (c) durvalumab. In some embodiments, the composition comprises nanoparticles comprising (a) paclitaxel, (b) an albumin (such as a human albumin), and (c) durvalumab, wherein paclitaxel is coated (such as substantially coated) with the albumin. In some embodiments, the composition comprises nanoparticles comprising (a) paclitaxel, (b) an albumin (such as a human albumin), and (c) durvalumab, wherein paclitaxel is coated (such as substantially coated) with the albumin, and wherein durvalumab is associated with paclitaxel. In some embodiments, the composition comprises nanoparticles comprising (a) paclitaxel, (b) an albumin (such as a human albumin), and (c) durvalumab, wherein paclitaxel is coated (such as substantially coated) with the albumin, and wherein durvalumab is associated with a solid core of paclitaxel. In some embodiments, the composition comprises nanoparticles comprising (a) paclitaxel, (b) an albumin (such as a human albumin), and (c) durvalumab, wherein paclitaxel is coated (such as substantially coated) with the albumin, and wherein durvalumab is embedded in a solid core of paclitaxel. In some embodiments, the composition comprises nanoparticles comprising (a) paclitaxel, (b) an albumin (such as a human albumin), and (c) durvalumab, wherein paclitaxel is coated with the albumin, and wherein durvalumab is associated with the albumin on the nanoparticles. In some embodiments, the composition comprises nanoparticles comprising (a) paclitaxel, (b) an albumin (such as a human albumin), and (c) durvalumab, wherein paclitaxel is coated with the albumin, and wherein durvalumab is associated with the albumin on the nanoparticles non-covalently. In some embodiments, the composition comprises nanoparticles comprising (a) paclitaxel, (b) an albumin (such as a human albumin), and (c) durvalumab, wherein paclitaxel is coated with the albumin, and wherein durvalumab is associated with the albumin on the nanoparticles covalently (such as via a disulfide bond or a chemical crosslink). In some embodiments, the composition comprises nanoparticles comprising (a) paclitaxel, (b) an albumin (such as a human albumin), and (c) durvalumab, wherein paclitaxel is coated with the albumin, and wherein durvalumab is associated with paclitaxel (such as a solid core of paclitaxel) and the albumin on the nanoparticle. In some embodiments, the composition comprises nanoparticles comprising (a) rapamycin, (b) an albumin (such as a human albumin), and (c) durvalumab. In some embodiments, the composition comprises nanoparticles comprising (a) rapamycin, (b) an albumin (such as a human albumin), and (c) durvalumab, wherein rapamycin is coated (such as substantially coated) with the albumin. In some embodiments, the composition comprises nanoparticles comprising (a) rapamycin, (b) an albumin (such as a human albumin), and (c) durvalumab, wherein rapamycin is coated (such as substantially coated) with the albumin, and wherein durvalumab is associated with rapamycin. In some embodiments, the composition comprises nanoparticles comprising (a) rapamycin, (b) an albumin (such as a human albumin), and (c) durvalumab, wherein rapamycin is coated (such as substantially coated) with the albumin, and wherein durvalumab is associated with a solid core of rapamycin. In some embodiments, the composition comprises nanoparticles comprising (a) rapamycin, (b) an albumin (such as a human albumin), and (c) durvalumab, wherein rapamycin is coated (such as substantially coated) with the albumin, and wherein durvalumab is embedded in a solid core of rapamycin. In some embodiments, the composition comprises nanoparticles comprising (a) rapamycin, (b) an albumin (such as a human albumin), and (c) durvalumab, wherein rapamycin is coated with the albumin, and wherein durvalumab is associated with the albumin on the nanoparticles. In some embodiments, the composition comprises nanoparticles comprising (a) rapamycin, (b) an albumin (such as a human albumin), and (c) durvalumab, wherein rapamycin is coated with the albumin, and wherein durvalumab is associated with the albumin on the nanoparticles non-covalently. In some embodiments, the composition comprises nanoparticles comprising (a) rapamycin, (b) an albumin (such as a human albumin), and (c) durvalumab, wherein rapamycin is coated with the albumin, and wherein durvalumab is associated with the albumin on the nanoparticles covalently (such as via a disulfide bond or a chemical crosslink). In some embodiments, the composition comprises nanoparticles comprising (a) rapamycin, (b) an albumin (such as a human albumin), and (c) durvalumab, wherein rapamycin is coated with the albumin, and wherein durvalumab is associated with rapamycin (such as a solid core of rapamycin) and the albumin on the nanoparticle. In some embodiments, the average diameter of nanoparticles in a composition, as measured by Dynamic Light Scattering, is no greater than about 200 nm.

Thus, in some embodiments, the composition comprises nanoparticles comprising (a) a hydrophobic drug (such as a taxane or a limus drug), (b) an albumin (such as a human albumin), and (c) BGB-A317. In some embodiments, the composition comprises nanoparticles comprising (a) a hydrophobic drug (such as a taxane or a limus drug), (b) an albumin (such as a human albumin), and (c) BGB-A317, wherein the hydrophobic drug is coated (such as substantially coated) with the albumin. In some embodiments, the composition comprises nanoparticles comprising (a) a hydrophobic drug (such as a taxane or a limus drug), (b) an albumin (such as a human albumin), and (c) BGB-A317, wherein the hydrophobic drug is coated (such as substantially coated) with the albumin, and wherein BGB-A317 is associated with the hydrophobic drug. In some embodiments, the composition comprises nanoparticles comprising (a) a hydrophobic drug (such as a taxane or a limus drug), (b) an albumin (such as a human albumin), and (c) BGB-A317, wherein the hydrophobic drug is coated (such as substantially coated) with the albumin, and wherein BGB-A317 is associated with a solid core of the hydrophobic drug. In some embodiments, the composition comprises nanoparticles comprising (a) a hydrophobic drug (such as a taxane or a limus drug), (b) an albumin (such as a human albumin), and (c) BGB-A317, wherein the hydrophobic drug is coated (such as substantially coated) with the albumin, and wherein BGB-A317 is embedded in a solid core of the hydrophobic drug. In some embodiments, the composition comprises nanoparticles comprising (a) a hydrophobic drug (such as a taxane or a limus drug), (b) an albumin (such as a human albumin), and (c) BGB-A317, wherein the hydrophobic drug is coated with the albumin, and wherein BGB-A317 is associated with the albumin on the nanoparticles. In some embodiments, the composition comprises nanoparticles comprising (a) a hydrophobic drug (such as a taxane or a limus drug), (b) an albumin (such as a human albumin), and (c) BGB-A317, wherein the hydrophobic drug is coated with the albumin, and wherein BGB-A317 is associated with the albumin on the nanoparticles non-covalently. In some embodiments, the composition comprises nanoparticles comprising (a) a hydrophobic drug (such as a taxane or a limus drug), (b) an albumin (such as a human albumin), and (c) BGB-A317, wherein the hydrophobic drug is coated with the albumin, and wherein BGB-A317 is associated with the albumin on the nanoparticles covalently (such as via a disulfide bond or a chemical crosslink). In some embodiments, the composition comprises nanoparticles comprising (a) a hydrophobic drug (such as a taxane or a limus drug), (b) an albumin (such as a human albumin), and (c) BGB-A317, wherein the hydrophobic drug is coated with the albumin, and wherein BGB-A317 is associated with the hydrophobic drug (such as a solid core of the hydrophobic drug) and the albumin on the nanoparticle. In some embodiments, the composition comprises nanoparticles comprising (a) paclitaxel, (b) an albumin (such as a human albumin), and (c) BGB-A317. In some embodiments, the composition comprises nanoparticles comprising (a) paclitaxel, (b) an albumin (such as a human albumin), and (c) BGB-A317, wherein paclitaxel is coated (such as substantially coated) with the albumin. In some embodiments, the composition comprises nanoparticles comprising (a) paclitaxel, (b) an albumin (such as a human albumin), and (c) BGB-A317, wherein paclitaxel is coated (such as substantially coated) with the albumin, and wherein BGB-A317 is associated with paclitaxel. In some embodiments, the composition comprises nanoparticles comprising (a) paclitaxel, (b) an albumin (such as a human albumin), and (c) BGB-A317, wherein paclitaxel is coated (such as substantially coated) with the albumin, and wherein BGB-A317 is associated with a solid core of paclitaxel. In some embodiments, the composition comprises nanoparticles comprising (a) paclitaxel, (b) an albumin (such as a human albumin), and (c) BGB-A317, wherein paclitaxel is coated (such as substantially coated) with the albumin, and wherein BGB-A317 is embedded in a solid core of paclitaxel. In some embodiments, the composition comprises nanoparticles comprising (a) paclitaxel, (b) an albumin (such as a human albumin), and (c) BGB-A317, wherein paclitaxel is coated with the albumin, and wherein BGB-A317 is associated with the albumin on the nanoparticles. In some embodiments, the composition comprises nanoparticles comprising (a) paclitaxel, (b) an albumin (such as a human albumin), and (c) BGB-A317, wherein paclitaxel is coated with the albumin, and wherein BGB-A317 is associated with the albumin on the nanoparticles non-covalently. In some embodiments, the composition comprises nanoparticles comprising (a) paclitaxel, (b) an albumin (such as a human albumin), and (c) BGB-A317, wherein paclitaxel is coated with the albumin, and wherein BGB-A317 is associated with the albumin on the nanoparticles covalently (such as via a disulfide bond or a chemical crosslink). In some embodiments, the composition comprises nanoparticles comprising (a) paclitaxel, (b) an albumin (such as a human albumin), and (c) BGB-A317, wherein paclitaxel is coated with the albumin, and wherein BGB-A317 is associated with paclitaxel (such as a solid core of paclitaxel) and the albumin on the nanoparticle. In some embodiments, the composition comprises nanoparticles comprising (a) rapamycin, (b) an albumin (such as a human albumin), and (c) BGB-A317. In some embodiments, the composition comprises nanoparticles comprising (a) rapamycin, (b) an albumin (such as a human albumin), and (c) BGB-A317, wherein rapamycin is coated (such as substantially coated) with the albumin. In some embodiments, the composition comprises nanoparticles comprising (a) rapamycin, (b) an albumin (such as a human albumin), and (c) BGB-A317, wherein rapamycin is coated (such as substantially coated) with the albumin, and wherein BGB-A317 is associated with rapamycin. In some embodiments, the composition comprises nanoparticles comprising (a) rapamycin, (b) an albumin (such as a human albumin), and (c) BGB-A317, wherein rapamycin is coated (such as substantially coated) with the albumin, and wherein BGB-A317 is associated with a solid core of rapamycin. In some embodiments, the composition comprises nanoparticles comprising (a) rapamycin, (b) an albumin (such as a human albumin), and (c) BGB-A317, wherein rapamycin is coated (such as substantially coated) with the albumin, and wherein BGB-A317 is embedded in a solid core of rapamycin. In some embodiments, the composition comprises nanoparticles comprising (a) rapamycin, (b) an albumin (such as a human albumin), and (c) BGB-A317, wherein rapamycin is coated with the albumin, and wherein BGB-A317 is associated with the albumin on the nanoparticles. In some embodiments, the composition comprises nanoparticles comprising (a) rapamycin, (b) an albumin (such as a human albumin), and (c) BGB-A317, wherein rapamycin is coated with the albumin, and wherein BGB-A317 is associated with the albumin on the nanoparticles non-covalently. In some embodiments, the composition comprises nanoparticles comprising (a) rapamycin, (b) an albumin (such as a human albumin), and (c) BGB-A317, wherein rapamycin is coated with the albumin, and wherein BGB-A317 is associated with the albumin on the nanoparticles covalently (such as via a disulfide bond or a chemical crosslink). In some embodiments, the composition comprises nanoparticles comprising (a) rapamycin, (b) an albumin (such as a human albumin), and (c) BGB-A317, wherein rapamycin is coated with the albumin, and wherein BGB-A317 is associated with rapamycin (such as a solid core of rapamycin) and the albumin on the nanoparticle. In some embodiments, the average diameter of nanoparticles in a composition, as measured by Dynamic Light Scattering, is no greater than about 200 nm.

Thus, in some embodiments, the composition comprises nanoparticles comprising (a) a hydrophobic drug (such as a taxane or a limus drug), (b) an albumin (such as a human albumin), and (c) tocilizumab. In some embodiments, the composition comprises nanoparticles comprising (a) a hydrophobic drug (such as a taxane or a limus drug), (b) an albumin (such as a human albumin), and (c) tocilizumab, wherein the hydrophobic drug is coated (such as substantially coated) with the albumin. In some embodiments, the composition comprises nanoparticles comprising (a) a hydrophobic drug (such as a taxane or a limus drug), (b) an albumin (such as a human albumin), and (c) tocilizumab, wherein the hydrophobic drug is coated (such as substantially coated) with the albumin, and wherein tocilizumab is associated with the hydrophobic drug. In some embodiments, the composition comprises nanoparticles comprising (a) a hydrophobic drug (such as a taxane or a limus drug), (b) an albumin (such as a human albumin), and (c) tocilizumab, wherein the hydrophobic drug is coated (such as substantially coated) with the albumin, and wherein tocilizumab is associated with a solid core of the hydrophobic drug. In some embodiments, the composition comprises nanoparticles comprising (a) a hydrophobic drug (such as a taxane or a limus drug), (b) an albumin (such as a human albumin), and (c) tocilizumab, wherein the hydrophobic drug is coated (such as substantially coated) with the albumin, and wherein tocilizumab is embedded in a solid core of the hydrophobic drug. In some embodiments, the composition comprises nanoparticles comprising (a) a hydrophobic drug (such as a taxane or a limus drug), (b) an albumin (such as a human albumin), and (c) tocilizumab, wherein the hydrophobic drug is coated with the albumin, and wherein tocilizumab is associated with the albumin on the nanoparticles. In some embodiments, the composition comprises nanoparticles comprising (a) a hydrophobic drug (such as a taxane or a limus drug), (b) an albumin (such as a human albumin), and (c) tocilizumab, wherein the hydrophobic drug is coated with the albumin, and wherein tocilizumab is associated with the albumin on the nanoparticles non-covalently. In some embodiments, the composition comprises nanoparticles comprising (a) a hydrophobic drug (such as a taxane or a limus drug), (b) an albumin (such as a human albumin), and (c) tocilizumab, wherein the hydrophobic drug is coated with the albumin, and wherein tocilizumab is associated with the albumin on the nanoparticles covalently (such as via a disulfide bond or a chemical crosslink). In some embodiments, the composition comprises nanoparticles comprising (a) a hydrophobic drug (such as a taxane or a limus drug), (b) an albumin (such as a human albumin), and (c) tocilizumab, wherein the hydrophobic drug is coated with the albumin, and wherein tocilizumab is associated with the hydrophobic drug (such as a solid core of the hydrophobic drug) and the albumin on the nanoparticle. In some embodiments, the composition comprises nanoparticles comprising (a) paclitaxel, (b) an albumin (such as a human albumin), and (c) tocilizumab. In some embodiments, the composition comprises nanoparticles comprising (a) paclitaxel, (b) an albumin (such as a human albumin), and (c) tocilizumab, wherein paclitaxel is coated (such as substantially coated) with the albumin. In some embodiments, the composition comprises nanoparticles comprising (a) paclitaxel, (b) an albumin (such as a human albumin), and (c) tocilizumab, wherein paclitaxel is coated (such as substantially coated) with the albumin, and wherein tocilizumab is associated with paclitaxel. In some embodiments, the composition comprises nanoparticles comprising (a) paclitaxel, (b) an albumin (such as a human albumin), and (c) tocilizumab, wherein paclitaxel is coated (such as substantially coated) with the albumin, and wherein tocilizumab is associated with a solid core of paclitaxel. In some embodiments, the composition comprises nanoparticles comprising (a) paclitaxel, (b) an albumin (such as a human albumin), and (c) tocilizumab, wherein paclitaxel is coated (such as substantially coated) with the albumin, and wherein tocilizumab is embedded in a solid core of paclitaxel. In some embodiments, the composition comprises nanoparticles comprising (a) paclitaxel, (b) an albumin (such as a human albumin), and (c) tocilizumab, wherein paclitaxel is coated with the albumin, and wherein tocilizumab is associated with the albumin on the nanoparticles. In some embodiments, the composition comprises nanoparticles comprising (a) paclitaxel, (b) an albumin (such as a human albumin), and (c) tocilizumab, wherein paclitaxel is coated with the albumin, and wherein tocilizumab is associated with the albumin on the nanoparticles non-covalently. In some embodiments, the composition comprises nanoparticles comprising (a) paclitaxel, (b) an albumin (such as a human albumin), and (c) tocilizumab, wherein paclitaxel is coated with the albumin, and wherein tocilizumab is associated with the albumin on the nanoparticles covalently (such as via a disulfide bond or a chemical crosslink). In some embodiments, the composition comprises nanoparticles comprising (a) paclitaxel, (b) an albumin (such as a human albumin), and (c) tocilizumab, wherein paclitaxel is coated with the albumin, and wherein tocilizumab is associated with paclitaxel (such as a solid core of paclitaxel) and the albumin on the nanoparticle. In some embodiments, the composition comprises nanoparticles comprising (a) rapamycin, (b) an albumin (such as a human albumin), and (c) tocilizumab. In some embodiments, the composition comprises nanoparticles comprising (a) rapamycin, (b) an albumin (such as a human albumin), and (c) tocilizumab, wherein rapamycin is coated (such as substantially coated) with the albumin. In some embodiments, the composition comprises nanoparticles comprising (a) rapamycin, (b) an albumin (such as a human albumin), and (c) tocilizumab, wherein rapamycin is coated (such as substantially coated) with the albumin, and wherein tocilizumab is associated with rapamycin. In some embodiments, the composition comprises nanoparticles comprising (a) rapamycin, (b) an albumin (such as a human albumin), and (c) tocilizumab, wherein rapamycin is coated (such as substantially coated) with the albumin, and wherein tocilizumab is associated with a solid core of rapamycin. In some embodiments, the composition comprises nanoparticles comprising (a) rapamycin, (b) an albumin (such as a human albumin), and (c) tocilizumab, wherein rapamycin is coated (such as substantially coated) with the albumin, and wherein tocilizumab is embedded in a solid core of rapamycin. In some embodiments, the composition comprises nanoparticles comprising (a) rapamycin, (b) an albumin (such as a human albumin), and (c) tocilizumab, wherein rapamycin is coated with the albumin, and wherein tocilizumab is associated with the albumin on the nanoparticles. In some embodiments, the composition comprises nanoparticles comprising (a) rapamycin, (b) an albumin (such as a human albumin), and (c) tocilizumab, wherein rapamycin is coated with the albumin, and wherein tocilizumab is associated with the albumin on the nanoparticles non-covalently. In some embodiments, the composition comprises nanoparticles comprising (a) rapamycin, (b) an albumin (such as a human albumin), and (c) tocilizumab, wherein rapamycin is coated with the albumin, and wherein tocilizumab is associated with the albumin on the nanoparticles covalently (such as via a disulfide bond or a chemical crosslink). In some embodiments, the composition comprises nanoparticles comprising (a) rapamycin, (b) an albumin (such as a human albumin), and (c) tocilizumab, wherein rapamycin is coated with the albumin, and wherein tocilizumab is associated with rapamycin (such as a solid core of rapamycin) and the albumin on the nanoparticle. In some embodiments, the average diameter of nanoparticles in a composition, as measured by Dynamic Light Scattering, is no greater than about 200 nm.

Other Components in the Composition Comprising Nanoparticles or Manufacturing Precursors

The compositions described herein may be used in pharmaceutical compositions or formulations, by combining the compositions described herein with a pharmaceutical acceptable carrier, excipients, stabilizing agents, bulking agent, and/or other agents, which are known in the art for use in the methods of treatment, methods of administration, and dosage regimen described herein. The pharmaceutical acceptable agents can also be included in any of the manufacturing precursor solutions, including aqueous solutions comprising albumin or bioactive polypeptides, or aqueous solutions added to crude mixtures, emulsions, or nanoparticle suspensions at various points during the manufacturing process.

As used herein, “pharmaceutically acceptable” or “pharmacologically compatible” refers to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration. For example, in some embodiments, the pharmaceutically acceptable material is an excipient, stabilizer, antimicrobial, or bulking agent.

To increase the stability of nanoparticles in a composition described herein, material may be added to increase the negative zeta potential of the nanoparticles, such as certain negatively charged components. Such negatively charged components include, but are not limited to bile salts, bile acids, glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid, and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine, stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearoylphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

Suitable pharmaceutically acceptable carriers include sterile water; saline, dextrose; dextrose in water or saline; condensation products of castor oil and ethylene oxide combining about 30 to about 35 moles of ethylene oxide per mole of castor oil; liquid acid; lower alkanols; oils such as corn oil; peanut oil, sesame oil and the like, with emulsifiers such as mono- or di-glyceride of a fatty acid, or a phosphatide, e.g., lecithin, and the like; glycols; polyalkylene glycols; aqueous media in the presence of a suspending agent, for example, sodium carboxymethylcellulose; sodium alginate; poly(vinylpyrolidone); and the like, alone, or with suitable dispensing agents such as lecithin; polyoxyethylene stearate; and the like. The carrier may also contain adjuvants such as preserving stabilizing, wetting, emulsifying agents and the like together with the penetration enhancer. The final form may be sterile and may also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

The compositions described herein may include other agents, excipients, or stabilizers to improve properties of the composition. Examples of suitable excipients and diluents include, but are not limited to, lactose, dextrose, sucrose, trehalose (including α,α-trehalose), sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, and mineral oil. The formulations can additionally include lubricating agents, wetting agents, emulsifying and suspending agents, and preserving agents. Examples of emulsifying agents include tocopherol esters such as tocopheryl polyethylene glycol succinate and the like, Pluronic®, emulsifiers based on polyoxy ethylene compounds, Span 80 and related compounds and other emulsifiers known in the art and approved for use in animals or human dosage forms. The compositions can be formulated so as to provide rapid, sustained or delayed release of the active ingredient after administration to the patient by employing procedures well known in the art.

In some embodiments, the composition is formulated to have a pH in the range of about 4.5 to about 9.0, including for example pH ranges of any one of about 5.0 to about 8.0, about 6.5 to about 7.5, and about 6.5 to about 7.0. In some embodiments, the pH of the composition is formulated to no less than about 6, including for example no less than about any one of 6.5, 7, or 8 (e.g., about 8). In some embodiments compositions further include buffers, such as Tris, phosphates (such as sodium phosphates or potassium phosphates), citrates, succinates, histine, or acetates. The composition can also be made to be isotonic with blood by the addition of a suitable tonicity modifier, such as glycerol.

In some embodiments, the nanoparticle composition is suitable for administration to a human. In some embodiments, the nanoparticle composition is suitable for administration to a mammal such as, in the veterinary context, domestic pets and agricultural animals. There are a wide variety of suitable formulations of the composition (see, e.g., U.S. Pat. Nos. 5,916,596 and 6,096,331, which are incorporated by reference). The following formulations and methods are merely exemplary and are in no way limiting.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the composition is substantially free (such as free) of an undesirable component found in a pharmaceutical formulation of a bioactive polypeptide. For example, in some embodiments, the composition is substantially free (such as free) of a surfactant (such as polysorbate 20 or polysorbate 80). In some embodiments, the composition is substantially free (such as free) of polysorbate 20. In some embodiments, the composition is substantially free (such as free) of polysorbate 80. In some embodiments, the composition is substantially free (such as free) of a buffer salt. In some embodiments, the composition comprises a surfactant (such as polysorbate 20 or polysorbate 80). In some embodiments, the composition comprises polysorbate 20. In some embodiments, the composition comprises polysorbate 80. In some embodiments, the composition comprises a buffer salt.

Methods of Making Nanoparticle Formulations

The present application also provides methods of making the nanoparticle compositions described herein. Nanoparticles containing a hydrophobic drug and albumin can be prepared under conditions of high shear forces (e.g., sonication, high pressure homogenization, or the like). These methods are disclosed in, for example, U.S. Pat. Nos. 5,916,596; 6,096,331; 6,749,868; 6,537,579; and PCT Application Pub. Nos. WO98/14174; WO99/00113; WO07/027941; and WO07/027819. The contents of these publications, particularly with respect the method of making nanoparticle compositions, are hereby incorporated by reference in their entireties. These methods can be modified as described herein to make nanoparticles comprising a hydrophobic drug, albumin (such as derivatized albumin), and bioactive polypeptide (such as an antibody). In some embodiments, at least a portion of the bioactive polypeptide is conjugated to an albumin polypeptide (i.e., a bioactive polypeptide-albumin conjugate). In some embodiments, the bioactive polypeptide or bioactive polypeptide-albumin conjugate is added at one or more steps during the manufacturing process. In some embodiments, the bioactive polypeptide is conjugated to pre-formed nanoparticles containing a hydrophobic drug an albumin.

In one aspect, there is provided a method of making a composition comprising nanoparticles comprising a hydrophobic drug, an albumin, and a bioactive polypeptide, the method comprising: i) subjecting a mixture of an organic solution and an aqueous solution to high-pressure homogenization, thereby forming an emulsion, wherein the organic solution comprises the hydrophobic drug dissolved in one or more organic solvents, and wherein the aqueous solution comprises the albumin and the bioactive polypeptide; and ii) removing at least a portion of the one or more organic solvents from the emulsion (for example, by evaporation), thereby forming the composition. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises adding bioactive polypeptide to the composition after removing the organic solvents. In some embodiments, the method further comprises filling the composition into one or more vials. In some embodiments, the method further comprises lyophilizing the composition.

In some embodiments, there is provided a method of making a composition comprising nanoparticles comprising a hydrophobic drug, an albumin, and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)), the method comprising: i) subjecting a mixture of an organic solution and an aqueous solution to high-pressure homogenization, thereby forming an emulsion, wherein the organic solution comprises the hydrophobic drug dissolved in one or more organic solvents, and wherein the aqueous solution comprises the albumin and the antibody; and ii) removing at least a portion of the one or more organic solvents from the emulsion (for example, by evaporation), thereby forming the composition. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises adding antibody to the composition after removing the organic solvents. In some embodiments, the method further comprises filling the composition into one or more vials. In some embodiments, the method further comprises lyophilizing the composition.

In some embodiments, there is provided a method of making a composition comprising nanoparticles comprising a taxane (such as paclitaxel), an albumin, and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)), the method comprising: i) subjecting a mixture of an organic solution and an aqueous solution to high-pressure homogenization, thereby forming an emulsion, wherein the organic solution comprises the taxane (such as paclitaxel) dissolved in one or more organic solvents, and wherein the aqueous solution comprises the albumin and the antibody; and ii) removing at least a portion of the one or more organic solvents from the emulsion (for example, by evaporation), thereby forming the composition. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises adding an antibody to the composition after removing the organic solvents. In some embodiments, the method further comprises filling the composition into one or more vials. In some embodiments, the method further comprises lyophilizing the composition.

In another aspect, there is provided a method of making a composition comprising nanoparticles comprising a hydrophobic drug, an albumin and a bioactive polypeptide, the method comprising: i) subjecting a mixture of an organic solution and an aqueous solution to high-pressure homogenization, thereby forming an emulsion, wherein the organic solution comprises the hydrophobic drug dissolved in one or more organic solvents, and wherein the aqueous solution comprises the albumin; ii) adding the bioactive polypeptide to the emulsion; and iii) removing at least a portion of the one or more organic solvents from the emulsion (such as by evaporation), thereby forming the composition. In some embodiments, the method comprises preventing any incubation time between adding the bioactive polypeptide to the emulsion and initiating removal of the one or more organic solvents. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises adding bioactive polypeptide to the composition after removing the organic solvents. In some embodiments, the method further comprises filling the composition into one or more vials. In some embodiments, the method further comprises lyophilizing the composition.

In some embodiments, there is provided a method of making a composition comprising nanoparticles comprising a hydrophobic drug, an albumin and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)), the method comprising: i) subjecting a mixture of an organic solution and an aqueous solution to high-pressure homogenization, thereby forming an emulsion, wherein the organic solution comprises the hydrophobic drug dissolved in one or more organic solvents, and wherein the aqueous solution comprises the albumin; ii) adding the antibody to the emulsion; and iii) removing at least a portion of the one or more organic solvents from the emulsion (such as by evaporation), thereby forming the composition. In some embodiments, the method comprises preventing any incubation time between adding the bioactive polypeptide to the emulsion and initiating removal of the one or more organic solvents. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises adding an antibody to the composition after removing the organic solvents. In some embodiments, the method further comprises filling the composition into one or more vials. In some embodiments, the method further comprises lyophilizing the composition.

In another embodiment, there is provided a method of making a composition comprising nanoparticles comprising a taxane (such as paclitaxel), an albumin, and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)), the method comprising: i) subjecting a mixture of an organic solution and an aqueous solution to high-pressure homogenization, thereby forming an emulsion, wherein the organic solution comprises the taxane (such as paclitaxel) dissolved in one or more organic solvents, and wherein the aqueous solution comprises the albumin; ii) adding the antibody to the emulsion; and iii) removing at least a portion of the one or more organic solvents from the emulsion (such as by evaporation), thereby forming the composition. In some embodiments, the method comprises preventing any incubation time between adding the bioactive polypeptide to the emulsion and initiating removal of the one or more organic solvents. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises adding an antibody to the composition after removing the organic solvents. In some embodiments, the method further comprises filling the composition into one or more vials. In some embodiments, the method further comprises lyophilizing the composition.

In another aspect, there is provided a method of making a composition comprising nanoparticles comprising a hydrophobic drug, an albumin and a bioactive polypeptide, the method comprising i) subjecting a mixture of an organic solution and an aqueous solution to high pressure homogenization, thereby forming an emulsion, wherein the organic solution comprises the hydrophobic drug dissolved in one or more organic solvents, and wherein the aqueous solution comprises the albumin; ii) removing at least a portion of the one or more organic solvents from the emulsion (such as by evaporation) to obtain a post-evaporated suspension, and iii) adding the bioactive polypeptide to the post-evaporated suspension, thereby forming the composition. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises adding bioactive polypeptide to the composition after removing the organic solvents. In some embodiments, the method further comprises filling the composition into one or more vials. In some embodiments, the method further comprises lyophilizing the composition.

In another aspect, there is provided a method of making a composition comprising nanoparticles comprising a hydrophobic drug, an albumin and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)), the method comprising i) subjecting a mixture of an organic solution and an aqueous solution to high pressure homogenization, thereby forming an emulsion, wherein the organic solution comprises the hydrophobic drug dissolved in one or more organic solvents, and wherein the aqueous solution comprises the albumin; ii) removing at least a portion of the one or more organic solvents from the emulsion (such as by evaporation) to obtain a post-evaporated suspension, and iii) adding the antibody to the post-evaporated suspension, thereby forming the composition. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises adding an antibody to the composition after removing the organic solvents. In some embodiments, the method further comprises filling the composition into one or more vials. In some embodiments, the method further comprises lyophilizing the composition.

In another aspect, there is provided a method of making a composition comprising nanoparticles comprising a taxane (such as paclitaxel), an albumin and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)), the method comprising i) subjecting a mixture of an organic solution and an aqueous solution to high pressure homogenization, thereby forming an emulsion, wherein the organic solution comprises the taxane (such as paclitaxel) dissolved in one or more organic solvents, and wherein the aqueous solution comprises the albumin; ii) removing at least a portion of the one or more organic solvents from the emulsion (such as by evaporation) to obtain a post-evaporated suspension, and iii) adding the antibody to the post-evaporated suspension, thereby forming the composition. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises adding an antibody to the composition after removing the organic solvents. In some embodiments, the method further comprises filling the composition into one or more vials. In some embodiments, the method further comprises lyophilizing the composition.

In another aspect, there is provided a method of making a composition comprising nanoparticles comprising a hydrophobic drug, an albumin and a bioactive polypeptide, the method comprising i) subjecting a mixture of an organic solution and an aqueous solution to high pressure homogenization, thereby forming an emulsion, wherein the organic solution comprises the hydrophobic drug dissolved in one or more organic solvents, and wherein the aqueous solution comprises the albumin; ii) removing at least a portion but not all of the one or more organic solvents from the emulsion (such as by evaporation) to obtain an emulsion-suspension intermediate, iii) adding the bioactive polypeptide to the emulsion-suspension intermediate, and iv) removing an additional portion of the one or more organic solvents from the emulsion-suspension intermediate comprising the bioactive polypeptide (such as by evaporation), thereby forming the composition. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises adding bioactive polypeptide to the composition after removing the organic solvents. In some embodiments, the method further comprises filling the composition into one or more vials. In some embodiments, the method further comprises lyophilizing the composition.

In another aspect, there is provided a method of making a composition comprising nanoparticles comprising a hydrophobic drug, an albumin and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)), the method comprising i) subjecting a mixture of an organic solution and an aqueous solution to high pressure homogenization, thereby forming an emulsion, wherein the organic solution comprises the hydrophobic drug dissolved in one or more organic solvents, and wherein the aqueous solution comprises the albumin; ii) removing at least a portion but not all of the one or more organic solvents from the emulsion (such as by evaporation) to obtain an emulsion-suspension intermediate, iii) adding the bioactive polypeptide to the emulsion-suspension intermediate, and iv) removing an additional portion of the one or more organic solvents from the emulsion-suspension intermediate comprising the bioactive polypeptide (such as by evaporation), thereby forming the composition. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises adding an antibody to the composition after removing the organic solvents. In some embodiments, the method further comprises filling the composition into one or more vials. In some embodiments, the method further comprises lyophilizing the composition.

In another aspect, there is provided a method of making a composition comprising nanoparticles comprising a taxane (such as paclitaxel), an albumin and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)), the method comprising i) subjecting a mixture of an organic solution and an aqueous solution to high pressure homogenization, thereby forming an emulsion, wherein the organic solution comprises the taxane (such as paclitaxel) dissolved in one or more organic solvents, and wherein the aqueous solution comprises the albumin; ii) removing at least a portion but not all of the one or more organic solvents from the emulsion (such as by evaporation) to obtain an emulsion-suspension intermediate, iii) adding the bioactive polypeptide to the emulsion-suspension intermediate, and iv) removing an additional portion of the one or more organic solvents from the emulsion-suspension intermediate comprising the bioactive polypeptide (such as by evaporation), thereby forming the composition. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises adding an antibody to the composition after removing the organic solvents. In some embodiments, the method further comprises filling the composition into one or more vials. In some embodiments, the method further comprises lyophilizing the composition.

In another aspect, there is provided a method of making a composition comprising nanoparticles comprising a hydrophobic drug, an albumin and a bioactive polypeptide, the method comprising: i) subjecting a mixture of an organic solution and an aqueous solution to high pressure homogenization, thereby forming an emulsion, wherein the organic solution comprises the hydrophobic drug dissolved in one or more organic solvents, and wherein the aqueous solution comprises the albumin, wherein at least a portion of the albumin is conjugated to the bioactive polypeptide; and ii) removing at least a portion of the one or more organic solvents from the emulsion (such as by evaporation), thereby forming the composition. In some embodiments, the bioactive polypeptide is covalently conjugated to the albumin, for example through a disulfide bond or a chemical crosslinker, such a crosslinker comprising a maleimide functional group and/or a NHS moiety, an SMCC crosslinker, a crosslinker comprising a boronate ester, or a moiety derived from click chemistry (such as copper-free click chemistry, such as a triazole moiety). In some embodiments, the albumin and the bioactive polypeptide are non-covalently conjugated. For example, in some embodiments the crosslinker comprises a first component covalently attached to the albumin, and a second component covalently attached to the bioactive polypeptide, wherein the first component and the second component specifically bind to one another (such as complementary nucleic acids molecules). In some embodiments, the method further comprises replacing the bioactive polypeptide-conjugated albumin not associated with the nanoparticles with unconjugated albumin, for example by dialysis, buffer exchange (such as tangential-flow filtration), or by separating the nanoparticles from the bioactive polypeptide-conjugated albumin not associated with the nanoparticles by centrifugation and resuspending the nanoparticles with a solution comprising unconjugated albumin. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises adding bioactive polypeptide to the composition after removing the organic solvents. In some embodiments, the method further comprises filling the composition into one or more vials. In some embodiments, the method further comprises lyophilizing the composition.

In some embodiments, there is provided a method of making a composition comprising nanoparticles comprising a hydrophobic drug, an albumin and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)), the method comprising: i) subjecting a mixture of an organic solution and an aqueous solution to high pressure homogenization, thereby forming an emulsion, wherein the organic solution comprises the hydrophobic drug dissolved in one or more organic solvents, and wherein the aqueous solution comprises the albumin, wherein at least a portion of the albumin is conjugated to the antibody; and ii) removing at least a portion of the one or more organic solvents from the emulsion (such as by evaporation), thereby forming the composition. In some embodiments, the bioactive polypeptide is covalently conjugated to the albumin, for example through a disulfide bond or a chemical crosslinker, such a crosslinker comprising a maleimide functional group and/or a NHS moiety, an SMCC crosslinker, a crosslinker comprising a boronate ester, or a moiety derived from click chemistry (such as copper-free click chemistry, such as a triazole moiety). In some embodiments, the albumin and the bioactive polypeptide are non-covalently conjugated. For example, in some embodiments the crosslinker comprises a first component covalently attached to the albumin, and a second component covalently attached to the bioactive polypeptide, wherein the first component and the second component specifically bind to one another (such as complementary nucleic acids molecules). In some embodiments, the method further comprises replacing the antibody-conjugated albumin not associated with the nanoparticles with unconjugated albumin, for example by dialysis, buffer exchange (such as tangential-flow filtration), or by separating the nanoparticles from the antibody-conjugated albumin not associated with the nanoparticles by centrifugation and resuspending the nanoparticles with a solution comprising unconjugated albumin. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises adding antibody to the composition after removing the organic solvents. In some embodiments, the method further comprises filling the composition into one or more vials. In some embodiments, the method further comprises lyophilizing the composition.

In another aspect, there is provided a method of making a composition comprising nanoparticles comprising a taxane (such as paclitaxel), an albumin and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)), the method comprising: i) subjecting a mixture of an organic solution and an aqueous solution to high pressure homogenization, thereby forming an emulsion, wherein the organic solution comprises the taxane (such as paclitaxel) dissolved in one or more organic solvents, and wherein the aqueous solution comprises the albumin, wherein at least a portion of the albumin is conjugated to the antibody; and ii) removing at least a portion of the one or more organic solvents from the emulsion (such as by evaporation), thereby forming the composition. In some embodiments, the bioactive polypeptide is covalently conjugated to the albumin, for example through a disulfide bond or a chemical crosslinker, such a crosslinker comprising a maleimide functional group and/or a NHS moiety, an SMCC crosslinker, a crosslinker comprising a boronate ester, or a moiety derived from click chemistry (such as copper-free click chemistry, such as a triazole moiety). In some embodiments, the albumin and the bioactive polypeptide are non-covalently conjugated. For example, in some embodiments the crosslinker comprises a first component covalently attached to the albumin, and a second component covalently attached to the bioactive polypeptide, wherein the first component and the second component specifically bind to one another (such as complementary nucleic acids molecules). In some embodiments, the method further comprises replacing the antibody-conjugated albumin not associated with the nanoparticles with unconjugated albumin, for example by dialysis, buffer exchange (such as tangential-flow filtration), or by separating the nanoparticles from the antibody-conjugated albumin not associated with the nanoparticles by centrifugation and resuspending the nanoparticles with a solution comprising unconjugated albumin. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises adding an antibody to the composition after removing the organic solvents. In some embodiments, the method further comprises filling the composition into one or more vials. In some embodiments, the method further comprises lyophilizing the composition.

In another aspect, there is provided a method of making a composition comprising nanoparticles comprising a hydrophobic drug, an albumin, and a bioactive polypeptide, the method comprising: i) subjecting a mixture of an organic solution and an aqueous solution to high pressure homogenization, thereby forming an emulsion, wherein the organic solution comprises the hydrophobic drug dissolved in one or more organic solvents, and wherein the aqueous solution comprises the albumin, wherein at least a portion of the albumin is derivatized; ii) removing at least a portion of the one or more organic solvents from the emulsion (such as by evaporation) to obtain a post-evaporated suspension; and iii) adding the bioactive polypeptide to the post-evaporated suspension. In some embodiments, the bioactive polypeptide is derivatized. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises adding bioactive polypeptide to the composition after removing the organic solvents. In some embodiments, the method further comprises filling the composition into one or more vials. In some embodiments, the method further comprises lyophilizing the composition.

In another aspect, there is provided a method of making a composition comprising nanoparticles comprising a hydrophobic drug, an albumin, and a bioactive polypeptide, the method comprising: i) subjecting a mixture of an organic solution and an aqueous solution to high pressure homogenization, thereby forming an emulsion, wherein the organic solution comprises the hydrophobic drug dissolved in one or more organic solvents, and wherein the aqueous solution comprises the albumin, wherein at least a portion of the albumin is derivatized; ii) removing at least a portion of the one or more organic solvents from the emulsion (such as by evaporation) to obtain a post-evaporated suspension comprising the nanoparticles; iii) replacing the derivatized albumin not associated with the nanoparticles with non-derivatized albumin; and iv) adding the bioactive polypeptide to the nanoparticles. In some embodiments, the bioactive polypeptide is derivatized. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises filling the composition into one or more vials. In some embodiments, the method further comprises lyophilizing the composition.

In another aspect, there is provided a method of making a composition comprising nanoparticles comprising a hydrophobic drug, an albumin, and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)), the method comprising: i) subjecting a mixture of an organic solution and an aqueous solution to high pressure homogenization, thereby forming an emulsion, wherein the organic solution comprises the hydrophobic drug dissolved in one or more organic solvents, and wherein the aqueous solution comprises the albumin, wherein at least a portion of the albumin is derivatized; ii) removing at least a portion of the one or more organic solvents from the emulsion (such as by evaporation) to obtain a post-evaporated suspension comprising the nanoparticles; iii) replacing the derivatized albumin not associated with the nanoparticles with non-derivatized albumin; and iv) adding the antibody to the nanoparticles. In some embodiments, the antibody is derivatized. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises filling the composition into one or more vials. In some embodiments, the method further comprises lyophilizing the composition.

In another aspect, there is provided a method of making a composition comprising nanoparticles comprising a taxane (such as paclitaxel), an albumin, and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)), the method comprising: i) subjecting a mixture of an organic solution and an aqueous solution to high pressure homogenization, thereby forming an emulsion, wherein the organic solution comprises the taxane (such as paclitaxel) dissolved in one or more organic solvents, and wherein the aqueous solution comprises the albumin, wherein at least a portion of the albumin is derivatized; ii) removing at least a portion of the one or more organic solvents from the emulsion (such as by evaporation) to obtain a post-evaporated suspension comprising the nanoparticles; iii) replacing the derivatized albumin not associated with the nanoparticles with non-derivatized albumin; and iv) adding the antibody to the nanoparticles. In some embodiments, the antibody is derivatized. In some embodiments, the method further comprises adding albumin to the emulsion prior to removing the organic solvents. In some embodiments, the method further comprises adding albumin to the composition after removing the organic solvents. In some embodiments, the method further comprises sterile filtering the composition after removing the organic solvents. In some embodiments, the method further comprises filling the composition into one or more vials. In some embodiments, the method further comprises lyophilizing the composition.

The hydrophobic drug is dissolved in an organic solvent (or a mixture of organic solvents) to form an organic solution comprising the hydrophobic drug. Suitable organic solvents include, for example, alkanes, cycloalkanes, ketones, alcohols, esters, ethers, chlorinated solvents, and other solvents known in the art. In some embodiments, the organic solution includes a water miscible organic solvent, a water immiscible organic solvent, or a mixture of a water miscible and a water immiscible organic solvent. In some embodiments the ratio of water miscible to water immiscible organic solvent in the organic solution is between about 20:1 and about 1:20 (for example, between about 20:1 and about 15:1, about 15:1 and about 12:1, about 12:1 and about 10:1, about 10:1 and about 8:1, about 8:1 and about 6:1, about 6:1 and about 4:1, about 4:1 and about 2:1, about 2:1 and about 1:1, about 1:1 and about 1:2, about 1:2 and about 1:4, about 1:4 and about 1:6, about 1:6 and about 1:8, about 1:8 and about 1:10, abut 1:10 and about 1:12, about 1:12 and about 1:15, or about 1:15 and about 1:20). Exemplary organic solvents include, for example, chloroform, dichloromethane, methylene chloride, ethyl acetate, ethanol, t-butanol, methanol, isopropanol, propanol, n-butanol, tetrahydrofuran, cyclohexane, dioxane, acetonitrile, acetone, dimethyl sulfoxide, dimethyl formamide, methyl pyrrolidinone. In some embodiments, the hydrophobic drug is dissolved in the organic solvent at a concentration of about 1 mg/mL to about 200 mg/mL (such as about 1 mg/mL to about 5 mg/mL, about 5 mg/mL to about 10 mg/mL, about 10 mg/mL to about 25 mg/mL, about 25 mg/mL to about 50 mg/mL, about 50 mg/mL to about 100 mg/mL, about 100 mg/mL to about 150 mg/mL, or about 150 mg/mL to about 200 mg/mL).

The organic solution comprising the hydrophobic drug is combined with an aqueous solution. In some embodiments, the aqueous solution comprises albumin (such as recombinant albumin) dissolved in water. The albumin can be, for example, human albumin. In some embodiments, the aqueous solution further comprises one or more salts, buffers, or stabilizers. In some embodiments, the aqueous solution is substantially free (such as free) of a surfactant (such as polysorbate). In some embodiments, the pH of the aqueous solution is between about 5 and about 8. In some embodiments, the concentration of the albumin (including the albumin portion of any bioactive polypeptide-albumin conjugate) in the aqueous solution is between about 0.5 mg/mL and about 250 mg/mL (such as between about 0.5 mg/mL and about 1 mg/mL, between about 1 mg/mL and about 5 mg/mL, between about 5 mg/mL and about 10 mg/mL, between about 10 mg/mL and about 25 mg/mL, between about 25 mg/mL and about 50 mg/mL, between about 50 mg/mL and about 100 mg/mL, between about 100 mg/mL and about 150 mg/mL, between about 150 mg/mL and about 200 mg/mL, or between about 200 mg/mL and about 250 mg/mL). In some embodiments, the aqueous solution comprises the bioactive polypeptide or the bioactive polypeptide-albumin conjugate. That is, the aqueous solution can comprise i) albumin, ii) the bioactive polypeptide, iii) the bioactive polypeptide-albumin conjugate, or iv) a combination of two or more. In some embodiments, the albumin is derivatized, for example by including a crosslinking moiety (such as an amine-reactive succinimidyl ester or a maleimide moiety). In some embodiments, the concentration of the bioactive polypeptide (including the bioactive polypeptide portion of the bioactive-albumin conjugate) in the aqueous solution is between about 0.5 mg/mL and about 30 mg/mL (such as between about 0.5 mg/mL and about 1 mg/mL, between about 1 mg/mL and about 5 mg/mL, between about 5 mg/mL and about 10 mg/mL, between about 10 mg/mL and about 20 mg/mL, or between about 20 mg/mL and about 30 mg/mL). In some embodiments, the aqueous solution comprises albumin and bioactive polypeptide at a w/w ratio (albumin:bioactive polypeptide) of about 1:1 to about 20:1.

In some embodiments, the bioactive polypeptide (or bioactive polypeptide-albumin conjugate) is provided in a separate aqueous solution (that is, an aqueous solution separate from the aqueous solution comprising the albumin or the aqueous solution comprising the albumin and the bioactive polypeptide). The separate aqueous solution (which can be referred to as a “bioactive polypeptide solution”) comprises the bioactive polypeptide (or the bioactive polypeptide-albumin conjugate, or a combination thereof) dissolved in water. In some embodiments, the bioactive polypeptide solution further comprises one or more salts, buffers, or stabilizers. In some embodiments, the bioactive polypeptide solution is substantially free (such as free) of a surfactant (such as polysorbate). In some embodiments, the pH of the bioactive polypeptide solution is between about 4 and about 8. In some embodiments, the concentration of the bioactive polypeptide (including the bioactive polypeptide portion of the bioactive polypeptide-albumin conjugate) in the bioactive polypeptide solution is about 0.5 mg/mL and about 30 mg/mL (such as between about 0.5 mg/mL and about 1 mg/mL, between about 1 mg/mL and about 5 mg/mL, between about 5 mg/mL and about 10 mg/mL, between about 10 mg/mL and about 20 mg/mL, or between about 20 mg/mL and about 30 mg/mL).

In some embodiments, the organic solution and aqueous solution (which may or may not include the bioactive polypeptide or the bioactive polypeptide-albumin conjugate) are mixed, for example using a high-shear mixer (such as a rotor-stator mixer), to form a crude mixture. For example, in some embodiments, the aqueous solution and the organic solution comprising the hydrophobic drug are combined, and the combined aqueous solution and organic solution are mixed to form the crude mixture. In some embodiments, an aqueous solution is mixed with a high-shear mixer, and an organic solution is added to the aqueous solution as the aqueous solution is being mixed to form the crude mixture. In some embodiments, the aqueous solution, the bioactive polypeptide solution, and the organic solution are combined, and the combined aqueous solution, bioactive polypeptide solution, organic solution are mixed to form the crude mixture. In some embodiments, the aqueous solution is mixed with a high-shear mixer, and the bioactive polypeptide solution and the organic solution are combined with the aqueous solution while the aqueous solution is being mixed. In some embodiments, the bioactive polypeptide solution is mixed with a high-shear mixer, and the aqueous solution and the organic solution can be combined with the bioactive polypeptide solution as the bioactive polypeptide solution is being mixed.

In some embodiments, albumin or bioactive polypeptide (or a bioactive polypeptide-albumin conjugate) can be added to the crude mixture. For example, in some embodiments, additional aqueous solution (comprising one or more of albumin, bioactive polypeptide, and/or bioactive-polypeptide conjugate) is added to the crude mixture, for example after the completion of mixing of the organic solution and the first aqueous solution. In some embodiments, the additional aqueous solution is mixed with the crude mixture, which may be performed using a high-shear mixer or a low-shear mixer. In some embodiments, the additional aqueous solution is combined with the crude mixture to adjust the concentration of the albumin (including the albumin portion of any bioactive polypeptide-albumin conjugate) in the crude mixture to between about 0.5 mg/mL and about 250 mg/mL (such as between about 0.5 mg/mL and about 1 mg/mL, between about 1 mg/mL and about 5 mg/mL, between about 5 mg/mL and about 10 mg/mL, between about 10 mg/mL and about 25 mg/mL, between about 25 mg/mL and about 50 mg/mL, between about 50 mg/mL and about 100 mg/mL, between about 100 mg/mL and about 150 mg/mL, between about 150 mg/mL and about 200 mg/mL, or between about 200 mg/mL and about 250 mg/mL). In some embodiments, the additional aqueous solution is combined with the crude mixture to adjust the concentration of the bioactive polypeptide (including the bioactive polypeptide portion of any bioactive polypeptide-albumin conjugate) in the crude mixture to between about 0.5 mg/mL and about 30 mg/mL (such as between about 0.5 mg/mL and about 1 mg/mL, between about 1 mg/mL and about 5 mg/mL, between about 5 mg/mL and about 10 mg/mL, between about 10 mg/mL and about 20 mg/mL, or between about 20 mg/mL and about 30 mg/mL). In some embodiments, the additional aqueous solution is combined with the crude mixture to adjust the ratio (w/w) of albumin to bioactive polypeptide to between 1:1 and about 1000:1. In some embodiments, the additional aqueous solution is combined with the crude mixture to adjust the ratio (w/w) of albumin to hydrophobic drug to about 2.5:1 to about 20:1.

The crude mixture of organic solution and aqueous solution (wherein the crude mixture may or may not include the additional aqueous solution added after the formation of the crude mixture) is subjected to high-pressure homogenization to form an emulsion. In some embodiments, the emulsion is cycled through the high-pressure homogenizer for between about 2 to about 100 cycles, such as about 5 to about 50 cycles or about 8 to about 20 cycles (e.g., about any one of 8, 10, 12, 14, 16, 18 or 20 cycles).

In some embodiments, additional albumin or bioactive polypeptide (or a bioactive polypeptide-albumin conjugate) can be added to the emulsion. For example, in some embodiments, additional aqueous solution (comprising one or more of albumin, bioactive polypeptide, and/or bioactive-polypeptide conjugate) is added to the emulsion. In some embodiments, the additional aqueous solution is added to the emulsion between passes through the high-pressure homogenizer. In some embodiments, the additional aqueous solution is added to the emulsion after the completion of the homogenization process. In some embodiments, the additional aqueous solution is mixed with the emulsion, which may be performed using a high-shear mixer or a low-shear mixer. In some embodiments, the additional aqueous solution is added to the emulsion to adjust the concentration of the albumin (including the albumin portion of any bioactive polypeptide-albumin conjugate) in the emulsion to between about 0.5 mg/mL and about 250 mg/mL (such as between about 0.5 mg/mL and about 1 mg/mL, between about 1 mg/mL and about 5 mg/mL, between about 5 mg/mL and about 10 mg/mL, between about 10 mg/mL and about 25 mg/mL, between about 25 mg/mL and about 50 mg/mL, between about 50 mg/mL and about 100 mg/mL, between about 100 mg/mL and about 150 mg/mL, between about 150 mg/mL and about 200 mg/mL, or between about 200 mg/mL and about 250 mg/mL). In some embodiments, the additional aqueous solution is combined with the emulsion to adjust the concentration of the bioactive polypeptide (including the bioactive polypeptide portion of any bioactive polypeptide-albumin conjugate) in the emulsion to between about 0.5 mg/mL and about 30 mg/mL (such as between about 0.5 mg/mL and about 1 mg/mL, between about 1 mg/mL and about 5 mg/mL, between about 5 mg/mL and about 10 mg/mL, between about 10 mg/mL and about 20 mg/mL, or between about 20 mg/mL and about 30 mg/mL). In some embodiments, the additional aqueous solution is combined with the emulsion to adjust the ratio (w/w) of albumin to bioactive polypeptide to between 1:1 and about 1000:1. In some embodiments, the additional aqueous solution is combined with the emulsion to adjust the ratio (w/w) of albumin to hydrophobic drug to about 2.5:1 to about 50:1.

At least a portion organic solvent (such as substantially all of the organic solvent) can be removed by evaporation utilizing suitable equipment known for this purpose, including, but not limited to, rotary evaporators, falling film evaporators, wiped film evaporators, spray driers, and the like. Evaporation of the organic solvent results in the formation of nanoparticles, which, if sufficient water remains after evaporation, can be in the form of a nanoparticle suspension (and can be referred to a “post-evaporated suspension”). The solvent may be removed at reduced pressure (such as at about any one of 25 mm Hg, 30 mm Hg, 40 mm Hg, 50 mm Hg, 100 mm Hg, 200 mm Hg, or 300 mm Hg). The amount of time used to remove the solvent under reduced pressure may be adjusted based on the volume of the formulation. For example, for a formulation produced on a 300 mL scale, the solvent can be removed at about 1 to about 300 mm Hg (e.g., about any one of 5-100 mm Hg, 10-50 mm Hg, 20-40 mm Hg, or 25 mm Hg) for about 5 to about 60 minutes (e.g., about any one of 7, 8, 9, 10, 11, 12, 13, 14, 15 16, 18, 20, 25, or 30 minutes).

In some embodiments, additional albumin and/or bioactive polypeptide (or a bioactive polypeptide-albumin conjugate) can be added to the nanoparticles (such as the post-evaporation suspension). For example, in some embodiments, additional aqueous solution (comprising one or more of albumin, bioactive polypeptide, and/or bioactive-polypeptide conjugate) is added to the nanoparticles. In some embodiments, the additional aqueous solution is added to the post-evaporation suspension to adjust the concentration of the albumin (including the albumin portion of any bioactive polypeptide-albumin conjugate) in the nanoparticle suspension to between about 0.5 mg/mL and about 250 mg/mL (such as between about 0.5 mg/mL and about 1 mg/mL, between about 1 mg/mL and about 5 mg/mL, between about 5 mg/mL and about 10 mg/mL, between about 10 mg/mL and about 25 mg/mL, between about 25 mg/mL and about 50 mg/mL, between about 50 mg/mL and about 100 mg/mL, between about 100 mg/mL and about 150 mg/mL, between about 150 mg/mL and about 200 mg/mL, or between about 200 mg/mL and about 250 mg/mL). In some embodiments, the additional aqueous solution is combined with the nanoparticle suspension to adjust the concentration of the bioactive polypeptide (including the bioactive polypeptide portion of any bioactive polypeptide-albumin conjugate) in the nanoparticle suspension to between about 0.5 mg/mL and about 30 mg/mL (such as between about 0.5 mg/mL and about 1 mg/mL, between about 1 mg/mL and about 5 mg/mL, between about 5 mg/mL and about 10 mg/mL, between about 10 mg/mL and about 20 mg/mL, or between about 20 mg/mL and about 30 mg/mL). In some embodiments, the additional aqueous solution is combined with the nanoparticle suspension to adjust the ratio (w/w) of albumin to bioactive polypeptide to between 1:1 and about 1000:1. In some embodiments, the additional aqueous solution is combined with the nanoparticle suspension to adjust the ratio (w/w) of albumin to hydrophobic drug to about 2.5:1 to about 50:1. In some embodiments, the nanoparticles are formulated for administration, for example by adding one or more excipients (such as stabilizers, buffers, bulking agents, antimicrobial agents, osmolytes, or reconstitution enhancers) to the nanoparticles (such as the post-evaporation suspension). The one or more excipients may be added to the nanoparticles separately from the aqueous solution, or may be included in the aqueous solution. In some embodiments, the pH of the nanoparticle suspension is adjusted to between about 4 and about 9 (such as between about 4 and about 5, between about 5 and about 6, between about 6 and about 7, between about 7 and about 8, or between about 8 and about 9). The post-evaporation suspension can incubate with the albumin or bioactive polypeptide (or bioactive polypeptide-albumin conjugate) for a desired amount of time (such as about 15 minutes to about 48 hours). In some embodiments, incubation occurs at about 3° C. to about 30° C. This incubation period allows bioactive polypeptide (or bioactive polypeptide-albumin conjugate) to associate with the nanoparticle. For example, in some embodiments, a portion of the albumin associated with the nanoparticle is derivatized, and the incubation period allows the derivatized albumin to conjugate to bioactive polypeptide.

In some embodiments, the albumin is conjugated to the bioactive polypeptide to form the bioactive polypeptide-albumin conjugate. Conjugation of the bioactive polypeptide to the albumin can occur at various points during the manufacturing process. For example, in some embodiments, the bioactive polypeptide is conjugated to the albumin prior to combining the aqueous solution with the organic solution. In some embodiments, the bioactive polypeptide is conjugated to the albumin when the polypeptide is combined with the nanoparticle suspension. In some embodiments, the albumin (or a portion of the albumin) is derivatized, which can provide for conjugation to the bioactive polypeptide. In some embodiments, the bioactive polypeptide is derivatized, which can provide for conjugation to the albumin. In some embodiments, the albumin (or a portion of the albumin) and the bioactive polypeptide are derivatized to provide for conjugation.

The bioactive polypeptide can be conjugated to the albumin, for example, using click chemistry or other known crosslinkers. For example, in some embodiments, the albumin or the bioactive polypeptide is derivatized with a strained alkene or strained alkyne, and the albumin and the bioactive polypeptide can be conjugated through cycloaddition reaction. In some embodiments, a lysine residue on the albumin or the bioactive polypeptide is derivatized, for example with an amine-reactive succinimidyl ester (such as N-hydroxysuccinimide ester (NHS-ester)), an isocyanate, or an isothiocyanate. In some embodiments, a cysteine residue on the albumin or the bioactive polypeptide is derivatized, for example with a maleimide or an iodoacetamide. Cysteine 34 of albumin is an exemplary cysteine that may be derivatized. Other methods of conjugation are known in the art. The derivatized albumin can be incubated with the bioactive polypeptide, or the derivatized bioactive polypeptide can be incubated with the albumin, to form the bioactive polypeptide-albumin conjugate. The bioactive polypeptide-albumin conjugate can be formed before the bioactive polypeptide-albumin conjugate is added to the aqueous solution, crude mixture, emulsion, or past-evaporation suspension. For example, the bioactive polypeptide-albumin conjugate can be separate from non-conjugated albumin or bioactive polypeptide before the bioactive polypeptide-albumin conjugate is included the nanoparticle manufacturing process. The bioactive polypeptide-albumin conjugate can also or alternatively be formed after the formation of the nanoparticles. For example, in some embodiments, derivatized albumin is included in the aqueous solution, the crude mixture, or the emulsion, and the bioactive polypeptide is added to the nanoparticle suspension. Derivatized albumin associated with the nanoparticle can react with the bioactive polypeptide to form the bioactive polypeptide-albumin conjugate. In some embodiments, derivatized bioactive polypeptide is added to the aqueous solution comprising albumin, the crude mixture, the emulsion, the post-evaporation suspension, or other nanoparticle suspension and reacts with albumin to form the bioactive polypeptide-albumin conjugate.

In some embodiments, bioactive polypeptide, bioactive polypeptide-albumin conjugate, or derivatized albumin (if present) that is not associated nanoparticles is removed from the nanoparticle suspension. In some embodiments, the bioactive polypeptide-albumin conjugate or the derivatized albumin is replaced by non-derivatized or unconjugated albumin. For example, in some embodiments, the nanoparticle suspension is centrifuged, the supernate is removed, and the nanoparticles are suspended in a fresh aqueous solution (which can non-derivatized or unconjugated albumin) In some embodiments, nanoparticle suspension is dialyzed to remove the bioactive polypeptide, bioactive polypeptide-albumin conjugate, or derivatized albumin (if present) that is not associated nanoparticles, which can be replaced by non-derivatized or unconjugated albumin. In some embodiments, the bioactive polypeptide, bioactive polypeptide-albumin conjugate, or derivatized albumin (if present) that is not associated nanoparticles is removed by buffer exchange (for example, by tangential-flow filtration), which can be replaced by non-derivatized or unconjugated albumin.

In another aspect, there is provided a method of making a composition comprising nanoparticles comprising a hydrophobic drug, an albumin, and a bioactive polypeptide conjugated to the albumin, comprising conjugating the bioactive polypeptide to nanoparticles comprising the hydrophobic drug and albumin. In some embodiments, the method comprises covalently crosslinking the bioactive polypeptide to the albumin. In some embodiments, the bioactive polypeptide is covalently conjugated to the albumin, for example through a disulfide bond or a chemical crosslinker, such a crosslinker comprising a maleimide functional group and/or a NHS moiety, an SMCC crosslinker, a crosslinker comprising a boronate ester, or a moiety derived from click chemistry (such as copper-free click chemistry, such as a triazole moiety). In some embodiments, the method comprises non-covalently crosslinking the bioactive polypeptide to the albumin, wherein the albumin is covalently bound to a first component of a crosslinker and the bioactive polypeptide is covalently bound to a second component of a crosslinker, wherein the first component of the crosslinker specifically binds to the second component of the crosslinker (such as nucleic acids molecules that are at least partially complementary).

In another aspect, there is provided a method of making a composition comprising nanoparticles comprising a hydrophobic drug, an albumin, and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)) conjugated to the albumin, comprising conjugating the antibody to nanoparticles comprising the hydrophobic drug and albumin. In some embodiments, the method comprises covalently crosslinking the antibody to the albumin. In some embodiments, the antibody is covalently conjugated to the albumin, for example through a disulfide bond or a chemical crosslinker, such a crosslinker comprising a maleimide functional group and/or a NHS moiety, an SMCC crosslinker, a crosslinker comprising a boronate ester, or a moiety derived from click chemistry (such as copper-free click chemistry, such as a triazole moiety). In some embodiments, the method comprises non-covalently crosslinking the antibody to the albumin, wherein the albumin is covalently bound to a first component of a crosslinker and the antibody is covalently bound to a second component of a crosslinker, wherein the first component of the crosslinker specifically binds to the second component of the crosslinker (such as nucleic acids molecules that are at least partially complementary).

In another aspect, there is provided a method of making a composition comprising nanoparticles comprising a taxane (such as paclitaxel), an albumin, and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)) conjugated to the albumin, comprising conjugating the antibody to nanoparticles comprising the taxane and albumin. In some embodiments, the method comprises covalently crosslinking the antibody to the albumin. In some embodiments, the antibody is covalently conjugated to the albumin, for example through a disulfide bond or a chemical crosslinker, such a crosslinker comprising a maleimide functional group and/or a NHS moiety, an SMCC crosslinker, a crosslinker comprising a boronate ester, or a moiety derived from click chemistry (such as copper-free click chemistry, such as a triazole moiety). In some embodiments, the method comprises non-covalently crosslinking the antibody to the albumin, wherein the albumin is covalently bound to a first component of a crosslinker and the antibody is covalently bound to a second component of a crosslinker, wherein the first component of the crosslinker specifically binds to the second component of the crosslinker (such as nucleic acids molecules that are at least partially complementary).

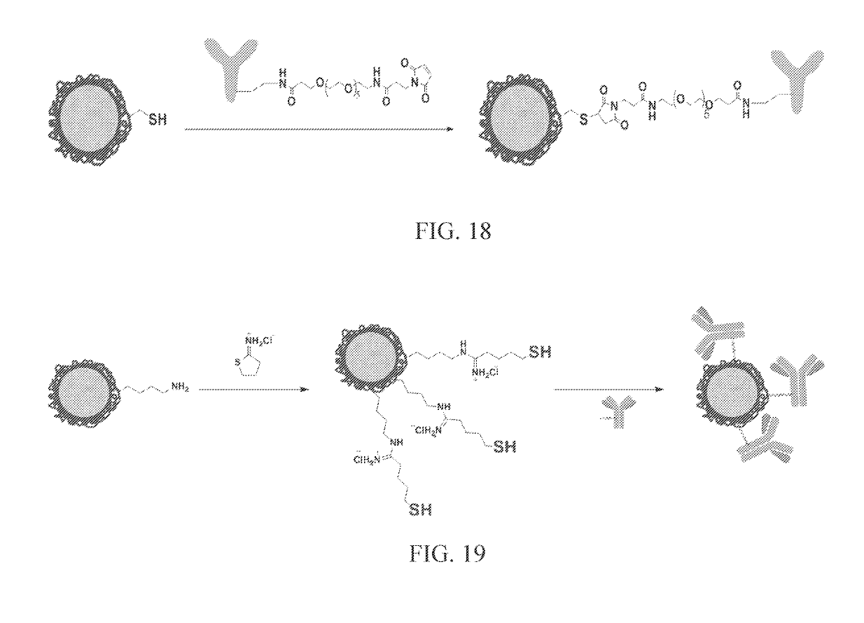

In another aspect, there is provided a method of making a composition comprising nanoparticles comprising a hydrophobic drug, an albumin, and a bioactive polypeptide conjugated to the albumin, comprising i) functionalizing the bioactive polypeptide with a crosslinker, and ii) combining the activated bioactive polypeptide with nanoparticles comprising the hydrophobic drug and albumin. FIG. 18 illustrates an example of such a method. In some embodiments, the crosslinker comprises a maleimide functional group and/or a NHS moiety, an SMCC crosslinker, a boronic acid, a click chemistry crosslinking reagent.

In another aspect, there is provided a method of making a composition comprising nanoparticles comprising a hydrophobic drug, an albumin, and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)) conjugated to the albumin, comprising i) functionalizing the antibody with a crosslinker, and ii) combining the activated antibody with nanoparticles comprising the hydrophobic drug and albumin. In some embodiments, the crosslinker comprises a maleimide functional group and/or a NHS moiety, an SMCC crosslinker, a boronic acid, a click chemistry crosslinking reagent.

In another aspect, there is provided a method of making a composition comprising nanoparticles comprising a taxane (such as paclitaxel), an albumin, and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)) conjugated to the albumin, comprising i) functionalizing the antibody with a crosslinker, and ii) combining the activated antibody with nanoparticles comprising the taxane and albumin. In some embodiments, the crosslinker comprises a maleimide functional group and/or a NHS moiety, an SMCC crosslinker, a boronic acid, a click chemistry crosslinking reagent.

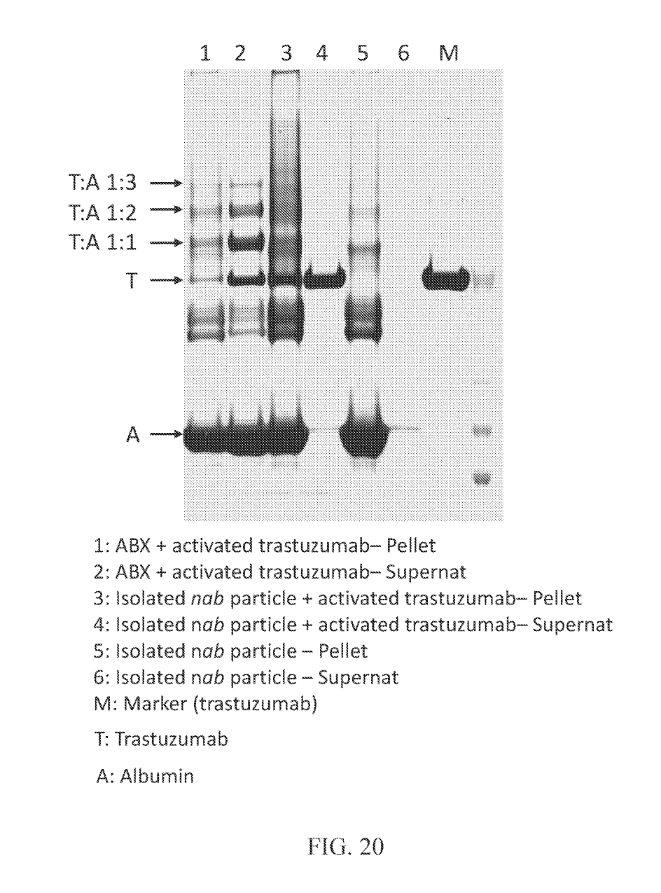

In another aspect, there is provided a method of making a composition comprising nanoparticles comprising a hydrophobic drug, an albumin, and a bioactive polypeptide conjugated to the albumin, comprising i) functionalizing the bioactive polypeptide with a crosslinker, ii) derivatizing at least a portion of the albumin associated with a surface of nanoparticles comprising the albumin and the hydrophobic drug, and iii) combining the activated bioactive polypeptide with the nanoparticles comprising the derivatized albumin FIGS. 19, 21, and 23 illustrate exemplary methods of derivatizing albumin associated with a surface of a nanoparticle, and conjugating functionalized bioactive polypeptide with the derivatized albumin. In some embodiments, derivatizing the albumin comprises thiolating the albumin, for example by combining the nanoparticles comprising the albumin and the hydrophobic drug with a thiolating agent (such as 2-iminothiolane). In some embodiments, the crosslinker comprises a maleimide functional group and/or a NHS moiety, an SMCC crosslinker, a boronic acid, a click chemistry crosslinking reagent.

In another aspect, there is provided a method of making a composition comprising nanoparticles comprising a hydrophobic drug, an albumin, and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)) conjugated to the albumin, comprising i) functionalizing the antibody with a crosslinker, ii) derivatizing at least a portion of the albumin associated with a surface of nanoparticles comprising the albumin and the hydrophobic drug, and iii) combining the activated antibody with the nanoparticles comprising the derivatized albumin. In some embodiments, derivatizing the albumin comprises thiolating the albumin, for example by combining the nanoparticles comprising the albumin and the hydrophobic drug with a thiolating agent (such as 2-iminothiolane). In some embodiments, the crosslinker comprises a maleimide functional group and/or a NHS moiety, an SMCC crosslinker, a boronic acid, a click chemistry crosslinking reagent.

In another aspect, there is provided a method of making a composition comprising nanoparticles comprising a taxane (paclitaxel), an albumin, and an antibody (such as an anti-VEGF antibody (e.g., an anti-VEGF-A antibody, such as bevacizumab), an anti-HER2 antibody (e.g., trastuzumab), and anti-PD-1 antibody (such as BGB-A317), or an anti-IL-6-receptor antibody (such as tocilizumab)) conjugated to the albumin, comprising i) functionalizing the antibody with a crosslinker, ii) derivatizing at least a portion of the albumin associated with a surface of nanoparticles comprising the albumin and the taxane, and iii) combining the activated antibody with the nanoparticles comprising the derivatized albumin. In some embodiments, derivatizing the albumin comprises thiolating the albumin, for example by combining the nanoparticles comprising the albumin and the hydrophobic drug with a thiolating agent (such as 2-iminothiolane). In some embodiments, the crosslinker comprises a maleimide functional group and/or a NHS moiety, an SMCC crosslinker, a boronic acid, a click chemistry crosslinking reagent.

In some embodiments, the nanoparticle suspension is filtered through one or more filter, which may sterilize the nanoparticle suspension (i.e., sterile filtration). The nanoparticle suspension may be serially filtered through multiple filters.

In some embodiments, the nanoparticles are dispensed into vials. In some embodiments, the vials are sealed. In some embodiments, the vials are single use vails. In some embodiments, the vials are multiple use vials. The nanoparticle suspension can also be lyophilized, either inside or outside the vials. In some embodiments, the lyophilized nanoparticles are reconstituted in an aqueous solution (such as water or saline). In some embodiments, the aqueous solution comprises one or more of albumin, the bioactive polypeptide, and/or a bioactive polypeptide-albumin conjugate. In some embodiments, the reconstituted nanoparticles can be incubated in the aqueous solution, can be filtered, the bioactive polypeptide or bioactive polypeptide-albumin conjugate can be removed, or can be re-lyophilized, for example as described above. In some embodiments, the reconstituted nanoparticles are administered to a subject.

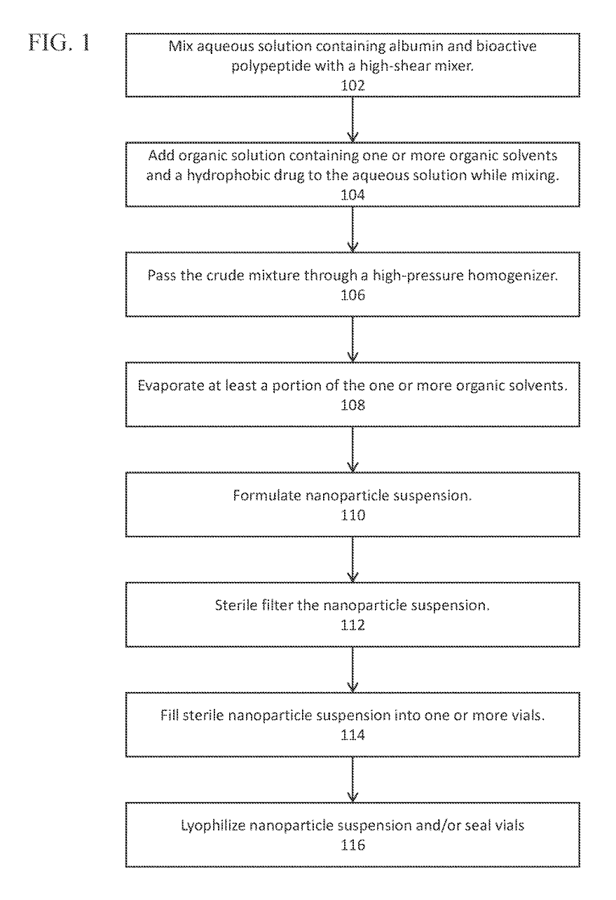

The following embodiments are exemplary methods for the manufacture of the nanoparticles described herein and should not be considered limiting. FIG. 1 is a flow chart illustrating one embodiment of a method of making the nanoparticles described herein. An aqueous solution containing albumin and a bioactive polypeptide (such as an antibody) dissolved in water is transferred to a vessel and mixed with a high-shear mixer at step 102. An organic solution containing one or more organic solvents (such as a water-miscible solvent and a water-immiscible solvent) and a hydrophobic drug (such as a taxane, such as paclitaxel) is added to the vessel containing the aqueous solution while the aqueous solution is being mixed with the high-shear mixer at step 104, thereby forming a crude mixture. At step 106, the crude mixture is homogenized by passing the crude mixture through a high-pressure homogenizer, thereby forming an emulsion. Optionally, the emulsion is passed through the high-pressure homogenizer two or more times. At step 108, at least a portion of the one or more organic solvents is removed from the emulsion by evaporation, thereby forming a post-evaporation nanoparticle suspension. Optionally, at step 110, the post-evaporation nanoparticle suspension is formulated, for example by adding an aqueous solution containing albumin or one or more excipients. The formulated nanoparticle suspension is optionally sterile filtered at step 112, and the sterile nanoparticle suspension is optionally filled into one or more vials at step 114. Optionally, at step 116, the vials are lyophilized and/or sealed.

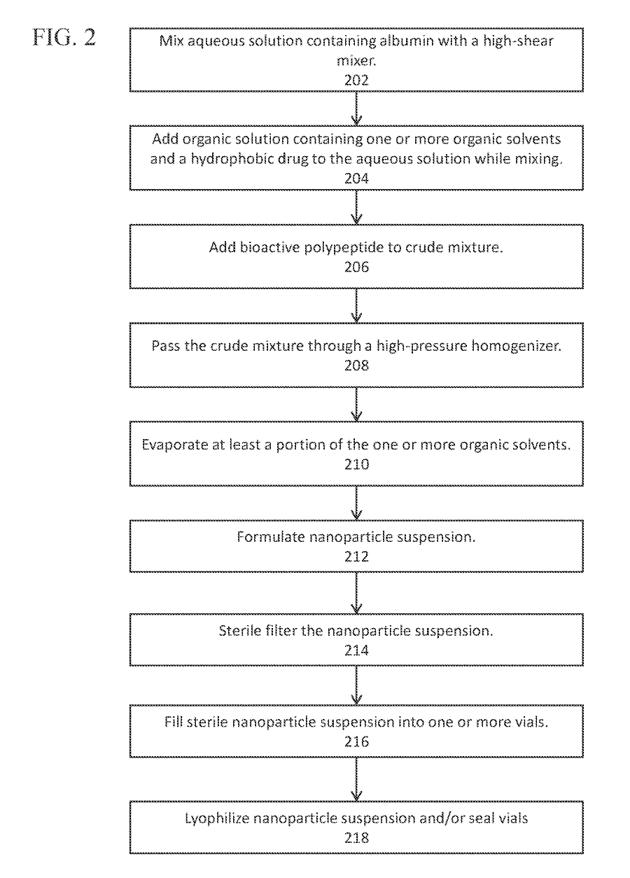

FIG. 2 is a flow chart illustrating another embodiment of a method of making the nanoparticles described herein. An aqueous solution containing albumin dissolved in water is transferred to a vessel and mixed with a high-shear mixer at step 202. An organic solution containing one or more organic solvents (such as a water-miscible solvent and a water-immiscible solvent) and a hydrophobic drug (such as a taxane, such as paclitaxel) is added to the vessel containing the aqueous solution while the aqueous solution is being mixed with the high-shear mixer at step 204, thereby forming a crude mixture. At step 206, a bioactive polypeptide (which can be contained in a second aqueous solution) is added to the crude mixture. At step 208, the crude mixture is homogenized by passing the crude mixture through a high-pressure homogenizer, thereby forming an emulsion. Optionally, the emulsion is passed through the high-pressure homogenizer two or more times. At step 210, at least a portion of the one or more organic solvents is removed from the emulsion by evaporation, thereby forming a post-evaporation nanoparticle suspension. Optionally, at step 212, the post-evaporation nanoparticle suspension is formulated, for example by adding an aqueous solution containing albumin or one or more excipients. The formulated nanoparticle suspension is optionally sterile filtered at step 214, and the sterile nanoparticle suspension is optionally filled into one or more vials at step 216. Optionally, at step 218, the vials are lyophilized and/or sealed.

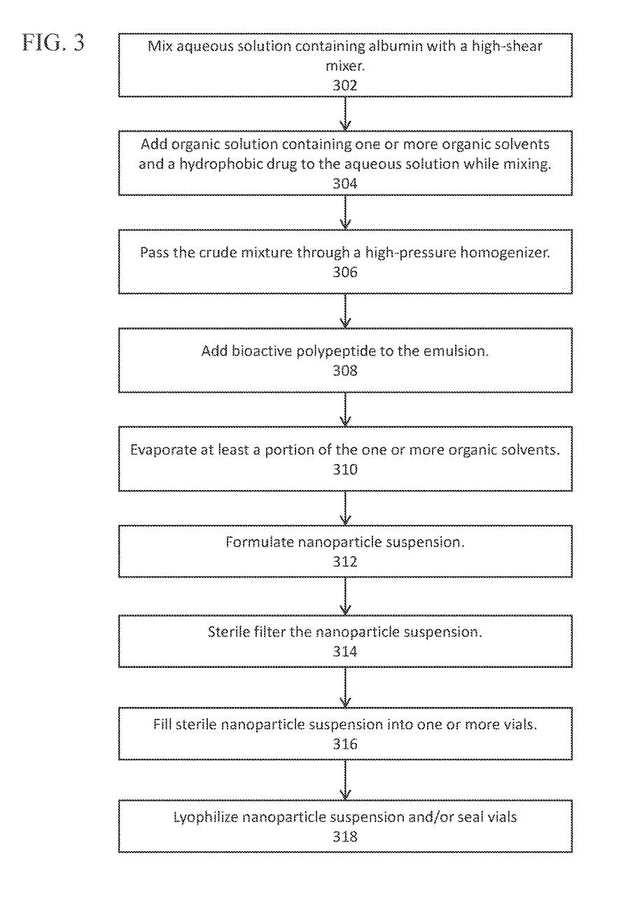

FIG. 3 is a flow chart illustrating another embodiment of a method of making the nanoparticles described herein. An aqueous solution containing albumin dissolved in water is transferred to a vessel and mixed with a high-shear mixer at step 302. An organic solution containing one or more organic solvents (such as a water-miscible solvent and a water-immiscible solvent) and a hydrophobic drug (such as a taxane, such as paclitaxel) is added to the vessel containing the aqueous solution while the aqueous solution is being mixed with the high-shear mixer at step 304, thereby forming a crude mixture. At step 306, the crude mixture is homogenized by passing the crude mixture through a high-pressure homogenizer, thereby forming an emulsion. Optionally, the emulsion is passed through the high-pressure homogenizer two or more times. At step 308, a bioactive polypeptide (which can be contained in a second aqueous solution) is added to the emulsion. At step 310, at least a portion of the one or more organic solvents is removed from the emulsion by evaporation, thereby forming a post-evaporation nanoparticle suspension. Optionally, at step 312, the post-evaporation nanoparticle suspension is formulated, for example by adding an aqueous solution containing albumin or one or more excipients. The formulated nanoparticle suspension is optionally sterile filtered at step 314, and the sterile nanoparticle suspension is optionally filled into one or more vials at step 316. Optionally, at step 318, the vials are lyophilized and/or sealed.

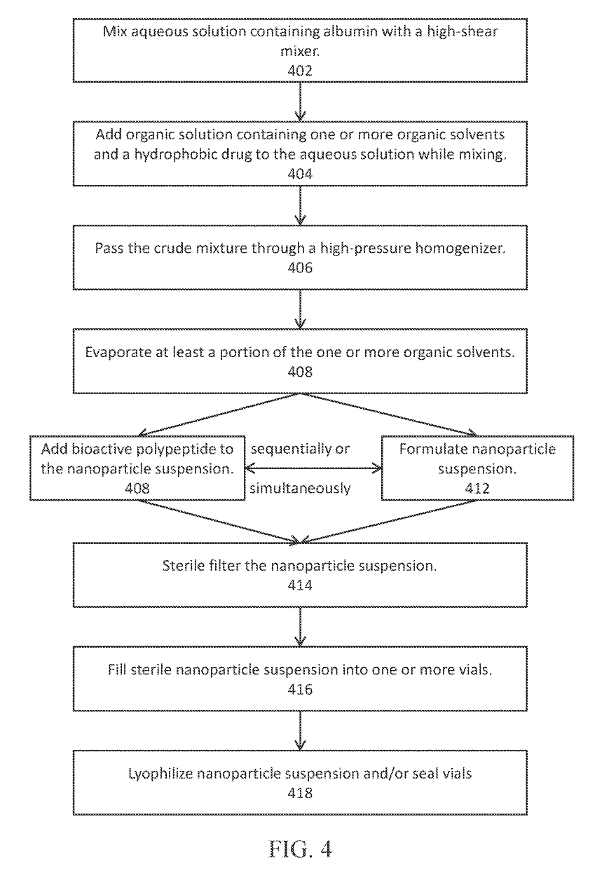

FIG. 4 is a flow chart illustrating another embodiment of a method of making the nanoparticles described herein. An aqueous solution containing albumin dissolved in water is transferred to a vessel and mixed with a high-shear mixer at step 402. An organic solution containing one or more organic solvents (such as a water-miscible solvent and a water-immiscible solvent) and a hydrophobic drug (such as a taxane, such as paclitaxel) is added to the vessel containing the aqueous solution while the aqueous solution is being mixed with the high-shear mixer at step 404, thereby forming a crude mixture. At step 406, the crude mixture is homogenized by passing the crude mixture through a high-pressure homogenizer, thereby forming an emulsion. Optionally, the emulsion is passed through the high-pressure homogenizer two or more times. At step 408, at least a portion of the one or more organic solvents is removed from the emulsion by evaporation, thereby forming a post-evaporation nanoparticle suspension. At step 410, a bioactive polypeptide (which can be contained in a second aqueous solution) is added to the post-evaporation nanoparticle suspension. In some embodiments, the bioactive polypeptide and the nanoparticle suspension are incubated for a period of time. Optionally, at step 412, the post-evaporation nanoparticle suspension is formulated, for example by adding an aqueous solution containing albumin or one or more excipients. In some embodiments, steps 410 and 412 are combined in a single step. For example, the bioactive polypeptide can be added to the nanoparticle suspension simultaneously to adding albumin or one or more excipients. The bioactive polypeptide, albumin, or excipients can be combined in the same aqueous solution or in different aqueous solutions. The formulated nanoparticle suspension is optionally sterile filtered at step 414, and the sterile nanoparticle suspension is optionally filled into one or more vials at step 416. Optionally, at step 418, the vials are lyophilized and/or sealed.

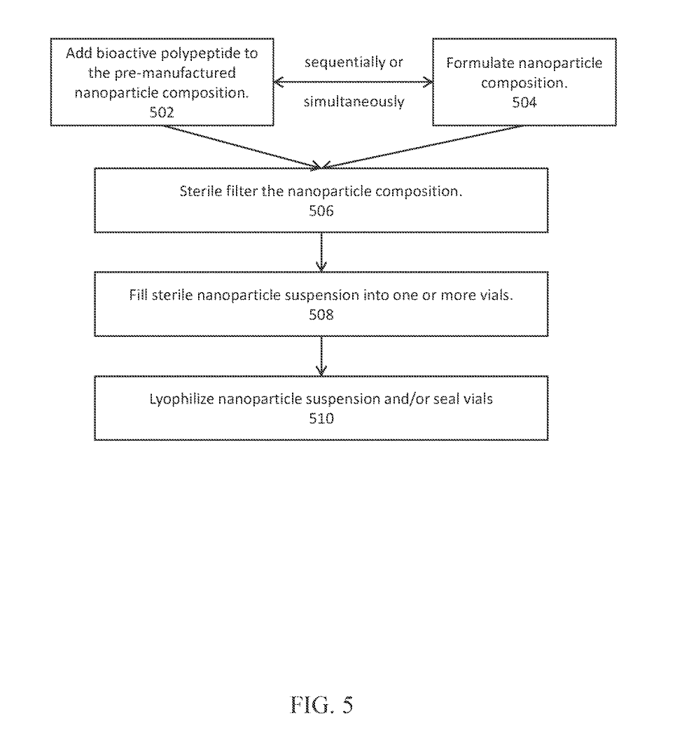

FIG. 5 is a flow chart illustrating another embodiment of a method of making the nanoparticles described herein. At step 502, a bioactive polypeptide (for example, in an aqueous solution) is combined with a composition comprising nanoparticles comprising albumin and a hydrophobic drug. The pre-made nanoparticles can be, for example, from an earlier-manufactured filtered nanoparticle suspension or a lyophilized nanoparticle composition (which may or may not be reconstituted). Exemplary pre-made nanoparticles are Abraxane® (Nab-paclitaxel). In some embodiments, an aqueous solution comprising the bioactive polypeptide is used to suspend a lyophilized nanoparticle composition. Optionally, at step 504, the nanoparticle suspension containing is formulated, for example by adding an aqueous solution containing albumin or one or more excipients. In some embodiments, steps 502 and 504 are combined in a single step. For example, the bioactive polypeptide can be added to the nanoparticle suspension simultaneously to adding albumin or one or more excipients. The bioactive polypeptide, albumin, or excipients can be combined in the same aqueous solution or in different aqueous solutions. The formulated nanoparticle suspension is optionally sterile filtered at step 506, and the sterile nanoparticle suspension is optionally filled into one or more vials at step 508. Optionally, at step 510, the vials are lyophilized and/or sealed.

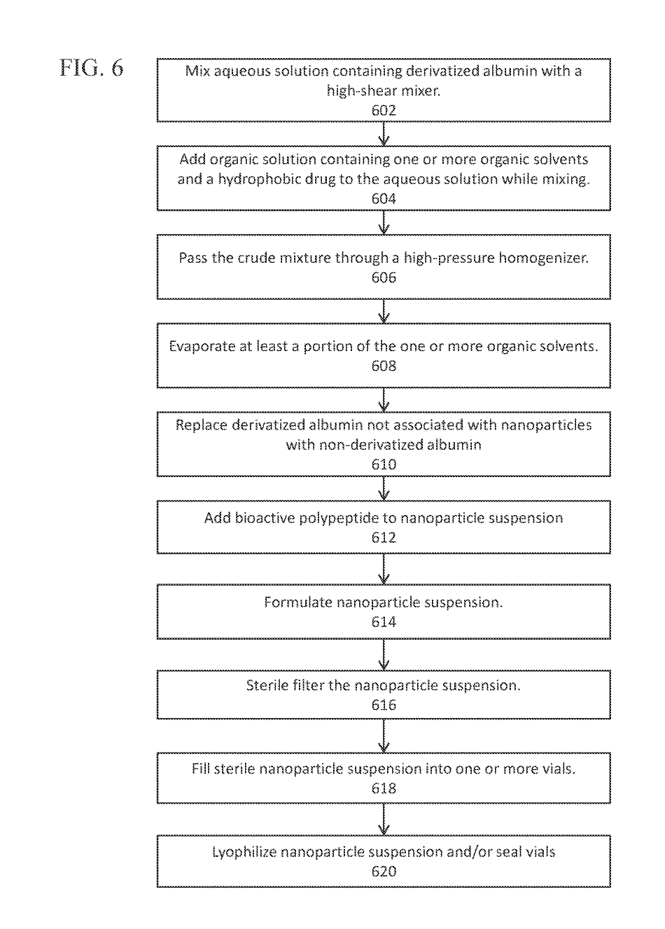

FIG. 6 is a flow chart illustrating another embodiment of a method of making the nanoparticles described herein. An aqueous solution containing derivatized albumin dissolved in water is transferred to a vessel and mixed with a high-shear mixer at step 602. In some embodiments, the aqueous solution further comprises non-derivatized albumin. An organic solution containing one or more organic solvents (such as a water-miscible solvent and a water-immiscible solvent) and a hydrophobic drug (such as a taxane, such as paclitaxel) is added to the vessel containing the aqueous solution while the aqueous solution is being mixed with the high-shear mixer at step 604, thereby forming a crude mixture. At step 606, the crude mixture is homogenized by passing the crude mixture through a high-pressure homogenizer, thereby forming an emulsion. Optionally, the emulsion is passed through the high-pressure homogenizer two or more times. At step 608, at least a portion of the one or more organic solvents is removed from the emulsion by evaporation, thereby forming a post-evaporation nanoparticle suspension. Derivatized albumin that is not associated with nanoparticles can be replaced with non-derivatized albumin at step 610, for example by dialysis, centrifugation and resuspension of the nanoparticles, or tangential flow filtration. At step 612, a bioactive polypeptide (which can be contained in a second aqueous solution) is added to the nanoparticle suspension. In some embodiments, the bioactive polypeptide is derivatized (depending on the method of conjugation). Optionally, at step 614, the suspension is formulated, for example by adding an aqueous solution containing albumin or one or more excipients. In some embodiments, steps 610 and 614, or steps 612 and 614, are combined in a single step. For example, the bioactive polypeptide can be added to the nanoparticle suspension simultaneously to adding albumin or one or more excipients. The bioactive polypeptide, albumin, or excipients can be combined in the same aqueous solution or in different aqueous solutions. The formulated nanoparticle suspension is optionally sterile filtered at step 616, and the sterile nanoparticle suspension is optionally filled into one or more vials at step 618. Optionally, at step 620, the vials are lyophilized and/or sealed.

Methods of Use

The compositions described herein may be used to treat diseases associated with cellular proliferation or hyperproliferation, such as cancers.

Thus, in some embodiments, there is provided methods of treating a disease (such as a cancer) in an individual in need thereof comprising administering to the individual an effective amount of a composition comprising nanoparticles comprising (a) a hydrophobic drug, (b) an albumin, and (c) a bioactive polypeptide. In some embodiments, the nanoparticles comprise a solid core of the hydrophobic drug coated with the albumin. In some embodiments, the bioactive polypeptide is associated with the surface of the solid core of the hydrophobic drug. In some embodiments, a portion of the bioactive polypeptide is embedded in the solid core of the hydrophobic drug. In some embodiments, the bioactive polypeptide is associated with the albumin on the nanoparticles. In some embodiments, at least about 75% of the bioactive polypeptide in the composition is associated with the nanoparticles. In some embodiments, the nanoparticles comprise at least about 100 bioactive polypeptides. In some embodiments, the weight ratio of the hydrophobic drug and the bioactive polypeptide in the nanoparticles in the composition is about 4:1. In some embodiments, the weight ratio of the albumin and the hydrophobic drug in the nanoparticles in the composition is less than about 1:1 to about 9:1. In some embodiments, the average diameter of the nanoparticles as measured by Dynamic Light Scattering (DLS) is no greater than about 200 nm. In some embodiments, the composition further comprises bioactive polypeptide not associated with the nanoparticles. In some embodiments, the hydrophobic drug is a taxane. In some embodiments, the taxane is paclitaxel. In some embodiments, the hydrophobic drug is a limus drug. In some embodiments, the limus drug is rapamycin. In some embodiments, the bioactive polypeptide is a therapeutic antibody. In some embodiments, the bioactive polypeptide is selected from the group consisting of: bevacizumab, cetuximab, ipilimumab, nivolumab, panitumumab, and rituximab.

Cancers to be treated by compositions described herein include, but are not limited to, carcinoma, lymphoma, blastoma, sarcoma, and leukemia. Examples of cancers that can be treated by compositions described herein include, but are not limited to, squamous cell cancer, lung cancer (including small cell lung cancer, non-small cell lung cancer, adenocarcinoma of the lung, and squamous carcinoma of the lung, including squamous NSCLC), cancer of the peritoneum, hepatocellular cancer, gastric or stomach cancer (including gastrointestinal cancer), pancreatic cancer (such as advanced pancreatic cancer), glioblastoma, cervical cancer, ovarian cancer, liver cancer (such as hepatocellular carcinoma), bladder cancer, hepatoma, breast cancer, colon cancer, melanoma, endometrial or uterine carcinoma, salivary gland carcinoma, kidney or renal cancer, liver cancer, prostate cancer (such as advanced prostate cancer), vulva! cancer, thyroid cancer, hepatic carcinoma, head and neck cancer, colorectal cancer, rectal cancer, soft-tissue sarcoma, Kaposi's sarcoma, B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's macroglobulinemia), chronic lymphocytic leukemia (CLL), acute lymphoblastic leukemia (ALL), myeloma, Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome. In some embodiments, there is provided a method of treating metastatic cancer (that is, cancer that has metastasized from the primary tumor). In some embodiments, there is provided a method of reducing cell proliferation and/or cell migration. In some embodiments, there is provided a method of treating hyperplasia, for example hyperplasia in the vascular system that can result in restenosis or hyperplasia that can result in arterial or venous hypertension.

In some embodiments, there are provided methods of treating cancer at an advanced stage(s). In some embodiments, there are provided methods of treating breast cancer (which may be HER2 positive or HER2 negative), including, for example, advanced breast cancer, stage IV breast cancer, locally advanced breast cancer, and metastatic breast cancer. In some embodiments, the cancer is lung cancer, including, for example, non-small cell lung cancer (NSCLC, such as advanced NSCLC), small cell lung cancer (SCLC, such as advanced SCLC), and advanced solid tumor malignancy in the lung. In some embodiments, the cancer is ovarian cancer, head and neck cancer, gastric malignancies, melanoma (including metastatic melanoma), colorectal cancer, pancreatic cancer, and solid tumors (such as advanced solid tumors). In some embodiments, the cancer is any of (and in some embodiments selected from the group consisting of) breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, gliomas, glioblastomas, neuroblastomas, and multiple myeloma. In some embodiments, the cancer is a solid tumor.

In some embodiments, the cancer to be treated is breast cancer, such as metastatic breast cancer. In some embodiments, the cancer to be treated is lung cancer, such as non-small cell lung cancer, including advanced stage non-small cell lung cancer. In some embodiments, the cancer to be treated is pancreatic cancer, such as early stage pancreatic cancer or advanced or metastatic pancreatic cancer. In some embodiments, the cancer to be treated is melanoma, such as stage III or IV melanoma.

In some embodiments, the individual being treated for a proliferative disease has been identified as having one or more of the conditions described herein. Identification of the conditions as described herein by a skilled physician is routine in the art (e.g., via blood tests, X-rays, CT scans, endoscopy, biopsy, angiography, CT-angiography, etc.) and may also be suspected by the individual or others, for example, due to tumor growth, hemorrhage, ulceration, pain, enlarged lymph nodes, cough, jaundice, swelling, weight loss, cachexia, sweating, anemia, paraneoplastic phenomena, thrombosis, etc. In some embodiments, the individual has been identified as susceptible to one or more of the conditions as described herein. The susceptibility of an individual may be based on any one or more of a number of risk factors and/or diagnostic approaches appreciated by the skilled artisan, including, but not limited to, genetic profiling, family history, medical history (e.g., appearance of related conditions), lifestyle or habits.

In some embodiments, the methods and/or compositions used herein reduce the severity of one or more symptoms associated with proliferative disease (e.g., cancer) by at least about any one of 10%, 20%, 30%, 40%, 50%, 60%, 70%, 80%, 90%, 95%, or 100% compared to the corresponding symptom in the same individual prior to treatment or compared to the corresponding symptom in other individuals not receiving the methods and/or compositions.

In some embodiments, the composition (such as pharmaceutical composition) described herein is used in combination with another administration modality or treatment.

Combination Treatments

The compositions described herein may be used as a component of a combination treatment to treat diseases associated with cellular proliferation or hyperproliferation, such as cancers.

Thus, in some embodiments, there is provided methods of treating a disease (such as a cancer) in an individual in need thereof comprising administering to the individual: (1) an effective amount of a composition comprising nanoparticles comprising (a) a hydrophobic drug, (b) an albumin, and (c) a bioactive polypeptide; and (2) an effective amount of one or more additional therapeutic agents. In some embodiments, the compositions described herein comprise (1) nanoparticles comprising (a) a hydrophobic drug, (b) an albumin, and (c) a bioactive polypeptide; and (2) one or more additional therapeutic agens.

In some embodiments, the one or more additional therapeutic agent is a water-soluble agent. In some embodiments, the other therapeutic agent is a chemotherapeutic agent. In some embodiments, the other therapeutic agent is a platinum-based agent. In some embodiments, the platinum-based agent is carboplatin. In some embodiments, the platinum-based agent is cisplatin. In some embodiments, the other therapeutic agent is an antimetabolite. In some embodiments, the other therapeutic agent is gemcitabine. In some embodiments, the other therapeutic agent is durvalumab. In some embodiments, the other therapeutic agent is capecitabine. In some embodiments, the other therapeutic agent is 5-fluorouracil, leucovorin, irinotecan, and/or oxaliplatin. In some embodiments, the other therapeutic agent is ipafricept. In some embodiments, the other therapeutic agent is vantictumab. In some embodiments, the other therapeutic agent is PEGPH20. In some embodiments, the other therapeutic agent is nivolumab. In some embodiments, the other therapeutic agent is necitumumab.

Thus, in some embodiments, there is provided methods of treating a disease (such as a cancer) in an individual in need thereof comprising administering to the individual: (1) an effective amount of a composition comprising nanoparticles comprising (a) a hydrophobic drug, (b) an albumin, and (c) a bioactive polypeptide; and (2) an effective amount of another therapeutic agent. In some embodiments, the nanoparticles comprise a solid core of the hydrophobic drug coated with the albumin. In some embodiments, the bioactive polypeptide is associated with the surface of the solid core of the hydrophobic drug. In some embodiments, a portion of the bioactive polypeptide is embedded in the solid core of the hydrophobic drug. In some embodiments, the bioactive polypeptide is associated with the albumin on the nanoparticles. In some embodiments, at least about 75% of the bioactive polypeptide in the composition is associated with the nanoparticles. In some embodiments, the nanoparticles comprise at least about 100 bioactive polypeptides. In some embodiments, the weight ratio of the hydrophobic drug and the bioactive polypeptide in the nanoparticles in the composition is about 4:1. In some embodiments, the weight ratio of the albumin and the hydrophobic drug in the nanoparticles in the composition is less than about 1:1 to about 9:1. In some embodiments, the average diameter of the nanoparticles as measured by Dynamic Light Scattering is no greater than about 200 nm. In some embodiments, the composition further comprises bioactive polypeptide not associated with the nanoparticles. In some embodiments, the hydrophobic drug is a taxane. In some embodiments, the taxane is paclitaxel. In some embodiments, the hydrophobic drug is a limus drug. In some embodiments, the limus drug is rapamycin. In some embodiments, the bioactive polypeptide is a therapeutic antibody. In some embodiments, the bioactive polypeptide is selected from the group consisting of: bevacizumab, cetuximab, ipilimumab, nivolumab, panitumumab, and rituximab. In some embodiments, the other therapeutic agent is a chemotherapeutic agent. In some embodiments, the other therapeutic agent is a platinum-based agent. In some embodiments, the platinum-based agent is carboplatin. In some embodiments, the platinum-based agent is cisplatin. In some embodiments, the other therapeutic agent is gemcitabine.

Dosing and Method of Administering the Nanoparticle Compositions

The dose of the composition administered to an individual (such as a human) may vary with the particular composition, the mode of administration, and the type of disease being treated. In some embodiments, the amount of nanoparticle is effective to result in an objective response (such as a partial response or a complete response). In some embodiments, the amount of the composition is sufficient to result in a complete response in an individual. In some embodiments, the amount of the composition is sufficient to result in a partial response in an individual. In some embodiments, the amount of the composition administered (for example when administered alone) is sufficient to produce an overall response rate of more than about any of 40%, 50%, 60%, or 64% among a population of individuals treated with the composition. Responses of an individual to the treatment of the methods described herein can be determined, for example, based on RECIST levels.

In some embodiments, the amount of the composition is sufficient to prolong progress-free survival of an individual. In some embodiments, the amount of the composition is sufficient to prolong overall survival of an individual. In some embodiments, the amount of the composition (for example when administered alone) is sufficient to produce clinical benefit of more than about any of 50%, 60%, 70%, or 77% among a population of individuals treated with the composition.

In some embodiments, the amount of the composition, first therapy, second therapy, or combination therapy is an amount sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of cancer cells, or tumor growth rate in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

In some embodiments, the amount of the hydrophobic drug (e.g., a taxane such as paclitaxel) in the composition is below the level that induces a toxicological effect (i.e., an effect above a clinically acceptable level of toxicity) or is at a level where a potential side effect can be controlled or tolerated when the composition is administered to the individual.

In some embodiments, the amount of the hydrophobic drug (e.g., a taxane such as paclitaxel) in the composition is included in any of the following ranges: about 0.1 mg to about 500 mg, about 0.1 mg to about 2.5 mg, about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount of the hydrophobic drug (e.g., a taxane such as paclitaxel) in the effective amount of the composition (e.g., a unit dosage form) is in the range of about 5 mg to about 500 mg, such as about 30 mg to about 300 mg or about 50 mg to about 200 mg. In some embodiments, the concentration of the hydrophobic drug (e.g., a taxane such as paclitaxel) in the composition is dilute (about 0.1 mg/ml) or concentrated (about 100 mg/ml), including for example any of about 0.1 to about 50 mg/ml, about 0.1 to about 20 mg/ml, about 1 to about 10 mg/ml, about 2 mg/ml to about 8 mg/ml, about 4 to about 6 mg/ml, or about 5 mg/ml. In some embodiments, the concentration of the hydrophobic drug (e.g., a taxane such as paclitaxel) is at least about any of 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, or 50 mg/ml.

Exemplary effective amounts of a hydrophobic drug (e.g., a taxane such as paclitaxel) in the composition include, but are not limited to, at least about any of 25 mg/m², 30 mg/m², 50 mg/m², 60 mg/m², 75 mg/m², 80 mg/m², 90 mg/m², 100 mg/m², 120 mg/m², 125 mg/m², 150 mg/m², 160 mg/m², 175 mg/m², 180 mg/m², 200 mg/m², 210 mg/m², 220 mg/m², 250 mg/m², 260 mg/m², 300 mg/m², 350 mg/m², 400 mg/m², 500 mg/m², 540 mg/m², 750 mg/m², 1000 mg/m², or 1080 mg/m² of the hydrophobic drug. In various embodiments, the composition includes less than about any of 350 mg/m², 300 mg/m², 250 mg/m², 200 mg/m², 150 mg/m², 120 mg/m², 100 mg/m², 90 mg/m², 50 mg/m², or 30 mg/m² of a hydrophobic drug (e.g., a taxane such as paclitaxel). In some embodiments, the amount of the hydrophobic drug (e.g., a taxane such as paclitaxel) per administration is less than about any of 25 mg/m², 22 mg/m², 20 mg/m², 18 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the effective amount of the hydrophobic drug (e.g., a taxane such as paclitaxel) in the composition is included in any of the following ranges: about 1 to about 5 mg/m², about 5 to about 10 mg/m², about 10 to about 25 mg/m², about 25 to about 50 mg/m², about 50 to about 75 mg/m², about 75 to about 100 mg/m², about 100 to about 125 mg/m², about 125 to about 150 mg/m², about 150 to about 175 mg/m², about 175 to about 200 mg/m², about 200 to about 225 mg/m², about 225 to about 250 mg/m², about 250 to about 300 mg/m², about 300 to about 350 mg/m², or about 350 to about 400 mg/m². In some embodiments, the effective amount of the hydrophobic drug (e.g., a taxane such as paclitaxel) in the composition is about 5 to about 300 mg/m², such as about 100 to about 150 mg/m², about 120 mg/m², about 130 mg/m², or about 140 mg/m².

In some embodiments of any of the above aspects, the effective amount of the hydrophobic drug (e.g., a taxane such as paclitaxel) in the composition includes at least about any of 1 mg/kg, 2.5 mg/kg, 3.5 mg/kg, 5 mg/kg, 6.5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, or 60 mg/kg. In various embodiments, the effective amount of the hydrophobic drug (e.g., a taxane such as paclitaxel) in the composition includes less than about any of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, or 1 mg/kg of the hydrophobic drug (e.g., a taxane such as paclitaxel).

In some embodiments, the amount of the bioactive polypeptide (e.g., a therapeutic antibody) in the composition is included in any of the following ranges: about 0.1 mg to about 500 mg, about 0.1 mg to about 2.5 mg, about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount of the bioactive polypeptide (e.g., a therapeutic antibody) in the effective amount of the composition (e.g., a unit dosage form) is in the range of about 5 mg to about 500 mg, such as about 30 mg to about 300 mg or about 50 mg to about 200 mg. In some embodiments, the concentration of the bioactive polypeptide (e.g., a therapeutic antibody) in the composition is dilute (about 0.1 mg/ml) or concentrated (about 100 mg/ml), including for example any of about 0.1 to about 50 mg/ml, about 0.1 to about 20 mg/ml, about 1 to about 10 mg/ml, about 2 mg/ml to about 8 mg/ml, about 4 to about 6 mg/ml, or about 5 mg/ml. In some embodiments, the concentration of the bioactive polypeptide (e.g., a therapeutic antibody) is at least about any of 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, or 50 mg/ml.

Exemplary effective amounts of a bioactive polypeptide (e.g., a therapeutic antibody) in the composition include, but are not limited to, at least about any of 25 mg/m², 30 mg/m², 50 mg/m², 60 mg/m², 75 mg/m², 80 mg/m², 90 mg/m², 100 mg/m², 120 mg/m², 125 mg/m², 150 mg/m², 160 mg/m², 175 mg/m², 180 mg/m², 200 mg/m², 210 mg/m², 220 mg/m², 250 mg/m², 260 mg/m², 300 mg/m², 350 mg/m², 400 mg/m², 500 mg/m², 540 mg/m², 750 mg/m², 1000 mg/m², or 1080 mg/m² of the bioactive polypeptide. In various embodiments, the composition includes less than about any of 350 mg/m², 300 mg/m², 250 mg/m², 200 mg/m², 150 mg/m², 120 mg/m², 100 mg/m², 90 mg/m², 50 mg/m², or 30 mg/m² of a bioactive polypeptide (e.g., a therapeutic antibody). In some embodiments, the amount of the bioactive polypeptide (e.g., a therapeutic antibody) per administration is less than about any of 25 mg/m², 22 mg/m², 20 mg/m², 18 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the effective amount of the bioactive polypeptide (e.g., a therapeutic antibody) in the composition is included in any of the following ranges: about 1 to about 5 mg/m², about 5 to about 10 mg/m², about 10 to about 25 mg/m², about 25 to about 50 mg/m², about 50 to about 75 mg/m², about 75 to about 100 mg/m², about 100 to about 125 mg/m², about 125 to about 150 mg/m², about 150 to about 175 mg/m², about 175 to about 200 mg/m², about 200 to about 225 mg/m², about 225 to about 250 mg/m², about 250 to about 300 mg/m², about 300 to about 350 mg/m², or about 350 to about 400 mg/m². In some embodiments, the effective amount of the bioactive polypeptide (e.g., a therapeutic antibody) in the composition is about 5 to about 300 mg/m², such as about 100 to about 150 mg/m², about 120 mg/m², about 130 mg/m², or about 140 mg/m².

In some embodiments of any of the above aspects, the effective amount of the bioactive polypeptide (e.g., a therapeutic antibody) in the composition includes at least about any of 1 mg/kg, 2.5 mg/kg, 3.5 mg/kg, 5 mg/kg, 6.5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, or 60 mg/kg. In various embodiments, the effective amount of the bioactive polypeptide (e.g., a therapeutic antibody) in the composition includes less than about any of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, or 1 mg/kg of the bioactive polypeptide.

In some embodiments, the amount of the bioactive polypeptide in a composition that is associated with a non-nanoparticle portion of the composition is optimized for a disease (such as a cancer) and/or individual being treated. In some embodiments, the amount of the bioactive polypeptide in a composition that is associated with a nanoparticle portion of the composition is optimized for a disease (such as a cancer) and/or individual being treated.

In some embodiments, the ratio of a hydrophobic drug and a bioactive polypeptide in a composition is optimized for a disease (such as a cancer) and/or individual being treated.

In some embodiments, the amount of the composition is close to a maximum tolerated dose (MTD) of the composition following the same dosing regimen. In some embodiments, the amount of the composition is more than about any of 80%, 90%, 95%, or 98% of the MTD.

Exemplary dosing frequencies for the administration of the compositions described herein include, but are not limited to, daily, every two days, every three days, every four days, every five days, every six days, weekly without break, three out of four weeks, once every three weeks, once every two weeks, or two out of three weeks. In some embodiments, the composition is administered about once every 2 weeks, once every 3 weeks, once every 4 weeks, once every 6 weeks, or once every 8 weeks. In some embodiments, the composition is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

In some embodiments, the dosing frequency is once every two days for one time, two times, three times, four times, five times, six times, seven times, eight times, nine times, ten times, and eleven times. In some embodiments, the dosing frequency is once every two days for five times. In some embodiments, the composition is administered over a period of at least ten days, wherein the interval between each administration is no more than about two days, and wherein the dose of the composition at each administration is about 0.25 mg/m² to about 250 mg/m², about 0.25 mg/m² to about 150 mg/m², about 0.25 mg/m² to about 75 mg/m², such as about 0.25 mg/m² to about 25 mg/m², or about 25 mg/m² to about 50 mg/m², as measured by the amount of a hydrophobic drug in the composition.

The administration of the composition can be extended over an extended period of time, such as from about a month up to about seven years. In some embodiments, the composition is administered over a period of at least about any of 2, 3, 4, 5, 6, 7, 8, 9, 10, 11, 12, 18, 24, 30, 36, 48, 60, 72, or 84 months.

In some embodiments, the dosage of a hydrophobic drug (e.g., a taxane such as paclitaxel) in a composition can be in the range of 5-400 mg/m² when given on a 3 week schedule, or 5-250 mg/m² (such as 80-150 mg/m², for example 100-120 mg/m²) when given on a weekly schedule. For example, the amount of a hydrophobic drug (e.g., a taxane such as paclitaxel) is about 60 to about 300 mg/m² (e.g., about 260 mg/m²) on a three week schedule.

Other exemplary dosing schedules for the administration of the composition include, but are not limited to, 100 mg/m², weekly, without break; 75 mg/m² weekly, 3 out of four weeks; 100 mg/m², weekly, 3 out of 4 weeks; 125 mg/m², weekly, 3 out of 4 weeks; 125 mg/m², weekly, 2 out of 3 weeks; 130 mg/m², weekly, without break; 175 mg/m², once every 2 weeks; 260 mg/m², once every 2 weeks; 260 mg/m², once every 3 weeks; 180-300 mg/m², every three weeks; 60-175 mg/m², weekly, without break; 20-150 mg/m² twice a week; and 150-250 mg/m² twice a week, as measured by the amount of a hydrophobic drug in the composition.

The dosing frequency of the nanoparticle composition may be adjusted over the course of a treatment based on the judgment of the administering physician.

In some embodiments, the individual is treated for at least about any of one, two, three, four, five, six, seven, eight, nine, or ten treatment cycles.

The compositions described herein allow infusion of the composition to an individual over an infusion time that is shorter than about 24 hours. For example, in some embodiments, the composition is administered over an infusion period of less than about any of 24 hours, 12 hours, 8 hours, 5 hours, 3 hours, 2 hours, 1 hour, 30 minutes, 20 minutes, or 10 minutes. In some embodiments, the composition is administered over an infusion period of about 30 minutes.

Other exemplary doses of a hydrophobic drug in the composition include, but are not limited to, about any of 50 mg/m², 60 mg/m², 75 mg/m², 80 mg/m², 90 mg/m², 100 mg/m², 120 mg/m², 160 mg/m², 175 mg/m², 200 mg/m², 210 mg/m², 220 mg/m², 260 mg/m², and 300 mg/m², as measured by the amount of the hydrophobic drug in the composition. For example, in some embodiments, the dosage of paclitaxel in a composition can be in the range of about 100-400 mg/m² when given on a 3 week schedule, or about 50-250 mg/m² when given on a weekly schedule.

The compositions described herein can be administered to an individual (such as human) via various routes, including, for example, intravenously, intraarterially, intraperitoneally, intravesicularly, subcutaneously, intrathecally, intrapulmonarily, intramuscularly, intratracheally, intraocularly, transdermally, intradermally, orally, intraportally, intrahepatically, hepatic arterial infusion, or by inhalation. In some embodiments, sustained continuous release formulation of the composition may be used. In some embodiments, the composition is administered intravenously. In some embodiments, the composition is administered intraportally. In some embodiments, the composition is administered intraarterially. In some embodiments, the composition is administered intraperitoneally. In some embodiments, the composition is administered intrahepatically.

Modes of Administration of Combination Treatments

The dosing regimens for compositions described herein apply to both monotherapy and combination treatment settings. The modes of administration for combination therapy methods are further described below.

In some embodiments, the composition and the other therapeutic agent (including the specific chemotherapeutic agents described herein) are administered simultaneously. When the drugs are administered simultaneously, the drug in the composition and the other therapeutic agent may be contained in the same composition (e.g., a composition comprising both the nanoparticle composition and the other therapeutic agent) or in separate compositions (e.g., the composition and the other therapeutic agent are contained in separate compositions).

In some embodiments, the composition and the other therapeutic agent are administered sequentially. Either the composition or the other therapeutic agent may be administered first. In some embodiments, the composition and the other therapeutic agent are contained in separate compositions, which may be contained in the same or different packages.

In some embodiments, the administration of the composition and the other therapeutic agent are concurrent, i.e., the administration period of the composition and that of the other therapeutic agent overlap with each other. In some embodiments, the composition is administered for at least one cycle (for example, at least any of 2, 3, or 4 cycles) prior to the administration of the other therapeutic agent. In some embodiments, the other therapeutic agent is administered for at least any of one, two, three, or four weeks. In some embodiments, the administrations of the composition and the other therapeutic agent are initiated at about the same time (for example, within any one of 1, 2, 3, 4, 5, 6, or 7 days). In some embodiments, the administrations of the composition and the other therapeutic agent are terminated at about the same time (for example, within any one of 1, 2, 3, 4, 5, 6, or 7 days). In some embodiments, the administration of the other therapeutic agent continues (for example for about any one of 1, 2, 3, 4, 5, 6, 7, 8, 9, 10, 11, or 12 months) after the termination of the administration of the composition. In some embodiments, the administration of the other therapeutic agent is initiated after (for example after about any one of 1, 2, 3, 4, 5, 6, 7, 8, 9, 10, 11, or we months) the initiation of the administration of the composition. In some embodiments, the administrations of the composition and the other therapeutic agent are initiated and terminated at about the same time. In some embodiments, the administrations of the composition and the other therapeutic agent are initiated at about the same time and the administration of the other therapeutic agent continues (for example for about any one of 1, 2, 3, 4, 5, 6, 7, 8, 9, 10, 11, or 12 months) after the termination of the administration of the composition. In some embodiments, the administration of the composition and the other therapeutic agent stop at about the same time and the administration of the other therapeutic agent is initiated after (for example after about any one of 1, 2, 3, 4, 5, 6, 7, 8, 9, 10, 11, or we months) the initiation of the administration of the composition.

In some embodiments, the administration of the composition and the other therapeutic agent are non-concurrent. For example, in some embodiments, the administration of the composition is terminated before the other therapeutic agent is administered. In some embodiments, the administration of the other therapeutic agent is terminated before the composition is administered. The time period between these two non-concurrent administrations can range from about two to eight weeks, such as about four weeks.

The dosing frequency of the composition and the other therapeutic agent may be adjusted over the course of a treatment, based on the judgment of the administering physician. When administered separately, the composition and the other therapeutic agent can be administered at different dosing frequency or intervals. For example, the composition can be administered weekly, while another agent can be administered more or less frequently. In some embodiments, sustained continuous release formulation of the composition and/or other agent may be used. Various formulations and devices for achieving sustained release are known in the art. A combination of the administration configurations described herein can also be used.

The composition and the other therapeutic agent can be administered using the same route of administration or different routes of administration. In some embodiments (for both simultaneous and sequential administrations), the composition and the other therapeutic agent are administered at a predetermined ratio. For example, in some embodiments, the ratio by weight of the hydrophobic drug in the composition and the other therapeutic agent is about 1 to 1. In some embodiments, the weight ratio may be between about 0.001 to about 1 and about 1000 to about 1, or between about 0.01 to about 1 and 100 to about 1. In some embodiments, the ratio by weight of the hydrophobic drug in the composition and the other therapeutic agent is less than about any of 100:1, 50:1, 30:1, 10:1, 9:1, 8:1, 7:1, 6:1, 5:1, 4:1, 3:1, 2:1, and 1:1 In some embodiments, the ratio by weight of the hydrophobic drug in the composition and the other therapeutic agent is more than about any of 1:1, 2:1, 3:1, 4:1, 5:1, 6:1, 7:1, 8:1, 9:1, 30:1, 50:1, 100:1. Other ratios are contemplated.

The doses required for the hydrophobic drug, bioactive polypeptide, and/or the other therapeutic agent may (but not necessarily) be lower than what is normally required when each agent is administered alone or when bioactive polypeptide is not a part of the hydrophobic drug nanoparticle composition. Thus, in some embodiments, the subtherapeutic amount of the hydrophobic drug in the composition and/or the other therapeutic agent is administered. “Subtherapeutic amount” or “subtherapeutic level” refer to an amount that is less than the therapeutic amount, that is, less than the amount normally used when the hydrophobic drug and/or bioactive polypeptide in the composition and/or the other therapeutic agent are administered alone. The reduction may be reflected in terms of the amount administered at a given administration and/or the amount administered over a given period of time (reduced frequency).

In some embodiments, other chemotherapeutic agent is administered so as to allow reduction of the normal dose of the hydrophobic drug and/or bioactive polypeptide in the composition required to effect the same degree of treatment by at least about any of 5%, 10%, 20%, 30%, 50%, 60%, 70%, 80%, 90%, or more. In some embodiments, enough hydrophobic drug and/or bioactive polypeptide in the composition is administered so as to allow reduction of the normal dose of the other therapeutic agent required to effect the same degree of treatment by at least about any of 5%, 10%, 20%, 30%, 50%, 60%, 70%, 80%, 90%, or more.

In some embodiments, the dose of both the hydrophobic drug and/or bioactive polypeptide in the composition and the other therapeutic agent are reduced as compared to the corresponding normal dose of each when administered alone. In some embodiments, both the hydrophobic drug and/or the bioactive polypeptide in the composition and the other therapeutic agent are administered at a subtherapeutic, i.e., reduced, level. In some embodiments, the dose of the composition and/or the other therapeutic agent is substantially less than the established maximum toxic dose (MTD). For example, the dose of the nanoparticle composition and/or the other therapeutic agent is less than about 50%, 40%, 30%, 20%, or 10% of the MTD.

A combination of the administration configurations described herein can be used. The combination therapy methods described herein may be performed alone or in conjunction with another therapy, such as chemotherapy, radiation therapy, surgery, hormone therapy, gene therapy, immunotherapy, chemoimmunotherapy, hepatic artery-based therapy, cryotherapy, ultrasound therapy, liver transplantation, local ablative therapy, radiofrequency ablation therapy, photodynamic therapy, and the like. Additionally, a person having a greater risk of developing a disease (such as a cancer) may receive treatments to inhibit and/or delay the development of the disease.

The other therapeutic agent described herein can be administered to an individual (such as human) via various routes, such as parenterally, including intravenous, intra-arterial, intraperitoneal, intrapulmonary, oral, inhalation, intravesicular, intramuscular, intra-tracheal, subcutaneous, intraocular, intrathecal, or transdermal. In some embodiments, the other therapeutic agent is administrated intravenously. In some embodiments, the nanoparticle composition is administered orally.

The dosing frequency of the other therapeutic agent can be the same or different from that of the composition. Exemplary frequencies are provided above. As further example, the other therapeutic agent can be administered three times a day, two times a day, daily, 6 times a week, 5 times a week, 4 times a week, 3 times a week, two times a week, weekly. In some embodiments, the other therapeutic agent is administered twice daily or three times daily. Exemplary amounts of the other therapeutic agent include, but are not limited to, any of the following ranges: about 1 to about 2000 mg, about 500 to about 1500 mg, about 700 to about 1200 mg, about 800 to about 1000 mg, about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. For example, the other therapeutic agent can be administered at a dose of about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg).

In some embodiments, for example when a platinum-based agent is administered, the other therapeutic agent is administered at a dose of less than about 8, 7, 6, 5, 4, 3, 2, or 1, AUC. In some embodiments, the other therapeutic agent is administered at a dose of about 1, 2, 3, 4, 5, 6, 7, or 8 AUC. In some embodiments, the other therapeutic agent is administered at a dose of about 4 AUC. In some embodiments, the other therapeutic agent is administered at a dose of about 5 AUC. In some embodiments, the other therapeutic agent is administered at a dose of about 6 AUC. In some embodiments, the other therapeutic agent is administered at a dose of about 4 to about 7 AUC, about 5 to about 6 AUC, about 3 to about 6 AUC, about 4 to about 5 AUC, or about 5 to about 7 AUC.

In some embodiments, the appropriate doses of other therapeutic agents are approximately those already employed in clinical therapies wherein the other therapeutic agent are administered alone or in combination with other therapeutic agents.

Pharmaceutical Composition and Unit Dosages

The compositions described herein may be used in pharmaceutical compositions or formulations, by combining the compositions described herein with a pharmaceutical acceptable carrier, excipients, stabilizing agents and/or other agents, which are known in the art, for use in the methods of treatment, methods of administration, and dosage regimes described herein. Further provided are unit dosages of the compositions described herein.

In some embodiments, the albumin allows the composition to be administered to an individual (such as human) without significant side effects. In some embodiments, the albumin (such as human serum albumin) is in an amount that is effective to reduce one or more side effects of administration of the hydrophobic drug or bioactive polypeptide to an individual (such as a human). The term “reducing one or more side effects of administration” refers to reduction, alleviation, elimination, or avoidance of one or more undesirable effects caused by administration of an agent, such as a hydrophobic drug or a bioactive polypeptide, as well as side effects caused by delivery vehicles (such as surfactants and solvents that render agents suitable for injection) used for deliver. Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with a hydrophobic drug and/or bioactive polypeptide can be reduced.

The compositions described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

The compositions described herein may be present in a sealed vial. In some embodiments, the sealed vial is for single use. In some embodiments, the sealed vial is for multiple uses.

Also provided are unit dosage forms comprising the compositions and formulations described herein. These unit dosage forms can be stored in a suitable packaging in single or multiple unit dosages and may also be further sterilized and sealed. In some embodiments, the composition (such as pharmaceutical composition) also includes one or more other compounds (or pharmaceutically acceptable salts thereof) that are useful for treating cancer. In various variations, the amount of hydrophobic drug in the composition is included in any one of the following ranges: about 5 to about 50 mg, about 20 to about 50 mg, about 50 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount of hydrophobic drug in the composition (e.g., a dosage or unit dosage form) is in the range of about 5 mg to about 500 mg, such as about 30 mg to about 300 mg or about 50 mg to about 200 mg, of the derivative. In some embodiments, the carrier is suitable for parental administration (e.g., intravenous administration).

In some embodiments, there is provided a dosage form (e.g., a unit dosage form) for the treatment of cancer comprising any one of the compositions (such as pharmaceutical compositions) described herein. In some embodiments, there are provided articles of manufacture comprising the compositions, formulations, and unit dosages described herein in suitable packaging for use in the methods of treatment, methods of administration, and dosage regimes described herein. Suitable packaging for compositions described herein are known in the art, and include, for example, vials (such as sealed vials), vessels (such as sealed vessels), ampules, bottles, jars, flexible packaging (e.g., sealed Mylar or plastic bags), and the like. These articles of manufacture may further be sterilized and/or sealed.

Kits, Medicines, Medicaments, and Compositions

The invention also provides kits, medicines, medicaments, and compositions for use in any of the methods described herein.

Kits of the invention include one or more containers comprising the composition or compositions described herein (or unit dosage forms and/or articles of manufacture) and/or another therapeutic agent (such as the agents described herein), and in some embodiments, further comprise instructions for use in accordance with any of the methods described herein. The kit may further comprise a description of selection an individual suitable or treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises: (1) a composition comprising nanoparticles comprising (a) a hydrophobic drug, (b) an albumin, and (c) a bioactive polypeptide; and (2) instructions for administering the composition for treatment of a disease (such as a cancer). In some embodiments, the kit comprises: (1) a composition comprising nanoparticles comprising (a) a hydrophobic drug, (b) an albumin, and (c) a bioactive polypeptide; (2) an effective amount of another therapeutic agent; and (3) instructions for administering the composition and the other therapeutic agent for treatment of a disease (such as a cancer). The composition(s) and the other therapeutic agent(s) can be present in separate containers or in a single container.

The kits of the invention are in suitable packaging. Suitable packaging include, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the compositions described herein generally include information as to dosage, dosing schedule, and route of administration for the intended treatment. The containers may be unit doses, bulk packages (e.g., multi-dose packages) or sub-unit doses. For example, kits may be provided that contain sufficient dosages of the composition as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the compositions described herein and instructions for use and packaged in quantities sufficient for storage and use in pharmacies, for example, hospital pharmacies and compounding pharmacies.

Also provided are medicines, medicament, combinations, and compositions for use in the methods described herein. In some embodiments, there is provided a medicine (or composition) for use in treating a disease (such as a cancer) comprising nanoparticles comprising (a) a hydrophobic drug, (b) an albumin, and (c) a bioactive polypeptide. In some embodiments, there is provided a medicine (or composition) for use in treating a disease (such as a cancer) comprising nanoparticles comprising (a) a hydrophobic drug, (b) an albumin, and (c) a bioactive polypeptide, wherein the medicine (or composition) is further administered with another therapeutic agent. In some embodiments, there is provided use of a composition comprising nanoparticles comprising (a) a hydrophobic drug, (b) an albumin, and (c) a bioactive polypeptide in the manufacture of a medicament for a disease (such as a cancer) in an individual. In some embodiments, there is provided use of a composition comprising nanoparticles comprising (a) a hydrophobic drug, (b) an albumin, and (c) a bioactive polypeptide in the manufacture of a medicament for a disease (such as a cancer) in an individual, wherein the medicament is further administered with another therapeutic agent. In some embodiments, there is provided use of: (1) a composition comprising nanoparticles comprising (a) a hydrophobic drug, (b) an albumin, and (c) a bioactive polypeptide, and (2) another therapeutic agent in the manufacture of a medicament combination for a disease (such as a cancer) in an individual. In some embodiments, there is provided a combination comprising: (1) a composition comprising nanoparticles comprising (a) a hydrophobic drug, (b) an albumin, and (c) a bioactive polypeptide, and (2) another therapeutic agent, for use in treating a disease (such as a cancer) in an individual in need thereof.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXEMPLARY EMBODIMENTS

The following exemplary embodiments are for illustrative purposes only and are not intended to limit the scope of the invention.

### Embodiment 1

A composition comprising nanoparticles comprising (a) a hydrophobic drug, (b) an albumin, and (c) a bioactive polypeptide.

### Embodiment 2

The composition of embodiment 1, wherein the bioactive polypeptide is conjugated to the albumin.

### Embodiment 3

The composition of embodiment 2, wherein the bioactive polypeptide is covalently crosslinked to the albumin.

### Embodiment 4

The composition of embodiment 3, wherein the bioactive polypeptide is covalently crosslinked to the albumin through a chemical crosslinker.

### Embodiment 5

The composition of embodiment 3, wherein the bioactive polypeptide is covalently crosslinked to the albumin through a disulfide bond.

### Embodiment 6

The composition of embodiment 2, wherein the bioactive polypeptide is conjugated to the albumin through a non-covalent crosslinker.

### Embodiment 7

The composition of embodiment 6, wherein the bioactive polypeptide comprises a first component of the non-covalent crosslinker and the albumin comprises a second component of the non-covalent crosslinker, and wherein the first component specifically binds to the second component.

### Embodiment 8

The composition of embodiment 7, wherein the non-covalent crosslinker comprises nucleic acid molecules, wherein at least a portion of the nucleic acid molecules are complementary.

### Embodiment 9

The composition of any one of embodiments 1-8, wherein the nanoparticles comprise a solid core of the hydrophobic drug coated with the albumin.

### Embodiment 10

The composition of embodiment 1 or 9, wherein the bioactive polypeptide is associated with the surface of the solid core of the hydrophobic drug.

### Embodiment 11

The composition of embodiment 9 or 10, wherein a portion of the bioactive polypeptide is embedded in the solid core of the hydrophobic drug.

### Embodiment 12

The composition of any one of embodiments 1-11, wherein the bioactive polypeptide is associated with the albumin on the nanoparticles.

### Embodiment 13

The composition of embodiment 12, wherein the bioactive polypeptide is embedded in the surface of the nanoparticles.

### Embodiment 14

The composition of embodiment 12 or 13, wherein the bioactive polypeptide is associated with the albumin on the nanoparticles non-covalently.

### Embodiment 15

The composition of any one of embodiments 12-14, wherein the bioactive polypeptide is associated with the albumin on the nanoparticles covalently.

### Embodiment 16

The composition of any one of embodiments 1-15, wherein at least 75% of the bioactive polypeptide in the composition is associated with the nanoparticles.

### Embodiment 17

The composition of any one of embodiments 1-16, wherein nanoparticles comprise at least about 100 bioactive polypeptides.

### Embodiment 18

The composition of any one of embodiments 1-17, wherein the weight ratio of the hydrophobic drug to the bioactive polypeptide in the nanoparticles in the composition is about 1:1 to about 100:1.

### Embodiment 19

The composition of any one of embodiments 1-18, wherein the weight ratio of the albumin to the bioactive polypeptide in the nanoparticles is about 1:1 to about 1000:1.

### Embodiment 20

The composition of embodiment 18 or 19, wherein:

the weight of the hydrophobic drug is determined by reverse-phase high performance liquid chromatography (HPLC), and the weight of the bioactive polypeptide and the albumin is determined by size exclusion chromatography (SEC); or

the weight of the hydrophobic drug is determined by reverse-phase high performance liquid chromatography (HPLC), the weight of the albumin is determined by size exclusion chromatography (SEC), and the weight of bioactive polypeptide is determined by an enzyme-linked immunosorbent assay (ELISA).

### Embodiment 21

The composition of any one of embodiments 1-20, wherein the composition further comprises bioactive polypeptide not associated with the nanoparticles.

### Embodiment 22

The composition of any one of embodiments 1-21, wherein the bioactive polypeptide is an antibody or fragment thereof.

### Embodiment 23

The composition of embodiment 22, wherein the antibody or fragment thereof specifically binds a tumor-associated antigen.

### Embodiment 24

The composition of any embodiment 22 or 23, wherein the antibody or fragment thereof is selected from the group consisting of a full length antibody, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding fragment thereof, a bifunctional hybrid antibody, and a single chain antibody.

### Embodiment 25

The composition of embodiment 24, wherein the antibody or fragment thereof is an Fc fragment.

### Embodiment 26

The composition of any one of embodiments 1-13, wherein the bioactive polypeptide is bevacizumab, trastuzumab, BGB-A317, or tocilizumab.

### Embodiment 27

A composition comprising nanoparticles comprising (a) a hydrophobic drug, and (b) an albumin derivatized with a crosslinker moiety.

### Embodiment 28

The composition of embodiment 27, wherein the nanoparticles comprise a solid core of the hydrophobic drug coated with the albumin.

### Embodiment 29

The composition of any one of embodiments 1-19, wherein the weight ratio of the albumin to the hydrophobic drug in the nanoparticles in the composition is about 1:1 to about 20:1.

### Embodiment 30

The composition of embodiment 29, wherein the weight of the hydrophobic drug is determined by reverse-phase high performance liquid chromatography (HPLC), and the weight of the albumin is determined by size exclusion chromatography (SEC).

### Embodiment 31

The composition of any one of embodiments 1-30, wherein at least about 40% of the albumin in the nanoparticle portion of the composition is crosslinked by disulfide bonds.

### Embodiment 32

The composition of any one of embodiments 1-31, wherein the average diameter of the nanoparticles as measured by dynamic light scattering is no greater than about 200 nm.

### Embodiment 33

The composition of any one of embodiments 1-32, wherein the composition further comprises albumin not associated with the nanoparticles.

### Embodiment 34

The composition of any one of embodiments 1-33, wherein the hydrophobic drug is a taxane.

### Embodiment 35

The composition of any one of embodiments 1-34, wherein the hydrophobic drug is paclitaxel.

### Embodiment 36

The composition of any one of embodiments 1-33, wherein the hydrophobic drug is a limus drug.

### Embodiment 37

The composition of any one of embodiments 1-33 and 36, wherein the hydrophobic drug is rapamycin.

### Embodiment 38

A method of making a composition comprising nanoparticles comprising a hydrophobic drug, an albumin and a bioactive polypeptide, the method comprising:

i) subjecting a mixture of an organic solution and an aqueous solution to high pressure homogenization, thereby forming an emulsion,
wherein the organic solution comprises the hydrophobic drug dissolved in one or more organic solvents, and
wherein the aqueous solution comprises the albumin and the bioactive polypeptide; and

ii) removing at least a portion of the one or more organic solvents from the emulsion, thereby forming the composition.

### Embodiment 39

The method of embodiment 38, wherein the bioactive polypeptide is conjugated to the albumin in the aqueous solution.

### Embodiment 40

A method of making a composition comprising nanoparticles comprising a hydrophobic drug, an albumin and a bioactive polypeptide, the method comprising:

i) subjecting a mixture of an organic solution and an aqueous solution to high pressure homogenization, thereby forming an emulsion,
wherein the organic solution comprises a hydrophobic drug dissolved in one or more organic solvents, and
wherein the aqueous solution comprises the albumin;

ii) adding the bioactive polypeptide to the emulsion; and

iii) removing at least a portion of the one or more organic solvents from the emulsion, thereby forming the composition.

### Embodiment 41

A method of making a composition comprising nanoparticles comprising a hydrophobic drug, an albumin and a bioactive polypeptide, the method comprising:

i) subjecting a mixture of an organic solution and an aqueous solution to high pressure homogenization, thereby forming an emulsion,
wherein the organic solution comprises a hydrophobic drug dissolved in one or more organic solvents, and
wherein the aqueous solution comprises the albumin;

ii) removing at least a portion of the one or more organic solvents from the emulsion to obtain a post-evaporated suspension, and

iii) adding the bioactive polypeptide to the post-evaporated suspension, thereby forming the composition.

### Embodiment 42

A method of making a composition comprising nanoparticles comprising a hydrophobic drug, an albumin and a bioactive polypeptide, the method comprising:

i) subjecting a mixture of an organic solution and an aqueous solution to high pressure homogenization, thereby forming an emulsion,
wherein the organic solution comprises a hydrophobic drug dissolved in one or more organic solvents, and
wherein the aqueous solution comprises the albumin;

ii) removing at least a portion of but not all of the one or more organic solvents from the emulsion to obtain an emulsion-suspension intermediate;

iii) adding the bioactive polypeptide to the emulsion-suspension intermediate; and

iv) removing an additional portion of the one or more organic solvents from the emulsion-suspension intermediate comprising the bioactive polypeptide, thereby forming the composition.

### Embodiment 43

A method of making a composition comprising nanoparticles comprising a hydrophobic drug, an albumin and a bioactive polypeptide, the method comprising:

i) subjecting a mixture of an organic solution and an aqueous solution to high pressure homogenization, thereby forming an emulsion,
wherein the organic solution comprises a hydrophobic drug dissolved in one or more organic solvents, and
wherein the aqueous solution comprises the albumin, wherein the albumin is derivatized with a crosslinker moiety;

ii) removing at least a portion of the one or more organic solvents from the emulsion to obtain a post-evaporated suspension, and

iii) adding the bioactive polypeptide to the post-evaporated suspension, wherein the bioactive polypeptide is derivatized with a crosslinker moiety, thereby forming the composition.

### Embodiment 44

The method of embodiment 43, further comprising replacing the derivatized albumin not associated with the nanoparticles with non-derivatized albumin.

### Embodiment 45

The method of embodiment 44, wherein the replacement is by dialysis.

### Embodiment 46

The method of embodiment 44, wherein the replacement is by buffer exchange.

### Embodiment 47

The method of embodiment 44, wherein the replacement is by separating the nanoparticles from the derivatized albumin not associated with the nanoparticles by centrifugation and resuspending the nanoparticles with a solution comprising non-derivatized albumin.

### Embodiment 48

A method of making a composition comprising nanoparticles comprising a hydrophobic drug, an albumin and a bioactive polypeptide, the method comprising:

i) subjecting a mixture of an organic solution and an aqueous solution to high pressure homogenization, thereby forming an emulsion,
wherein the organic solution comprises a hydrophobic drug dissolved in one or more organic solvents, and
wherein the aqueous solution comprises the albumin, wherein at least a portion of the albumin is conjugated to the bioactive polypeptide; and

ii) removing at least a portion of the one or more organic solvents from the emulsion, thereby forming the composition.

### Embodiment 49

The method of embodiment 48, further comprising replacing the bioactive polypeptide-conjugated albumin not associated with the nanoparticles with unconjugated albumin.

### Embodiment 50

The method of embodiment 48, wherein the replacement is by dialysis.

### Embodiment 51

The method of embodiment 48, wherein the replacement is by buffer exchange.

### Embodiment 52

The method of embodiment 48, wherein the replacement is by separating the nanoparticles from the bioactive polypeptide-conjugated albumin not associated with the nanoparticles by centrifugation and resuspending the nanoparticles with a solution comprising unconjugated albumin.

### Embodiment 53

A method of making a composition comprising nanoparticles comprising a hydrophobic drug, an albumin, and a bioactive polypeptide conjugated to the albumin, comprising conjugating the bioactive polypeptide to nanoparticles comprising the hydrophobic drug and albumin.

### Embodiment 54

The method of any one of embodiments 38-53, further comprising adding albumin to the emulsion prior to the removal of the organic solvents.

### Embodiment 55

The method of any one of embodiments 38-54, further comprising adding albumin to the composition after removal of the organic solvents.

### Embodiment 56

The method of any one of embodiments 38-55, further comprising adding bioactive polypeptide to the composition after removal of the organic solvents.

### Embodiment 57

The method of any one of embodiments 38-56, further comprising sterile filtering the composition formed after removal of the organic solvents.

### Embodiment 58

The method of any one of embodiments 38-57, wherein the hydrophobic drug is a taxane.

### Embodiment 59

The method of any one of embodiments 38-58, wherein the hydrophobic drug is paclitaxel.

### Embodiment 60

The method of any one of embodiments 38-59, wherein the hydrophobic drug is a limus drug.

### Embodiment 61

The method of any one of embodiments 38-57 and 60, wherein the hydrophobic drug is rapamycin.

### Embodiment 62

The method of any one of embodiments 38-61, wherein the bioactive polypeptide is an antibody or fragment thereof.

### Embodiment 63

The method of embodiment 62, wherein the antibody or fragment there of specifically binds a tumor-associated antigen.

### Embodiment 64

The method of embodiment 62 or 63, wherein the antibody or fragment thereof is selected from the group consisting of a full length antibody, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding fragment thereof, a bifunctional hybrid antibody, and a single chain antibody.

### Embodiment 65

The method of embodiment 64, wherein the antibody or fragment thereof is an Fc fragment.

### Embodiment 66

The method of any one of embodiments 38-65, wherein the bioactive polypeptide is bevacizumab, trastuzumab, BGB-A317, or tocilizumab.

### Embodiment 67

A composition obtained by the method of any one of embodiments 37-67.

### Embodiment 68

The composition of any one of embodiments 1-37 and 67, wherein the composition is substantially free of surfactants.

### Embodiment 69

The composition of any one of embodiments 1-37, 67, and 68, wherein the composition is an aqueous suspension.

### Embodiment 70

The composition of any one of embodiments 1-37, 67, and 68, wherein the composition is a dry composition.

### Embodiment 71

The composition of embodiment 70, wherein the composition is lyophilized.

### Embodiment 72

The composition according to any one of embodiments 1-37 and 67-71, further comprising one or more additional therapeutic agents.

### Embodiment 73

A pharmaceutical composition comprising the composition of any one of embodiments 1-37 and 67-72, and a pharmaceutically acceptable excipient.

### Embodiment 74

A sealed vial comprising the composition of any one of embodiments 1-37 and 67-73.

### Embodiment 75

The sealed vial of embodiment 74, wherein the sealed vial is for single use.

### Embodiment 76

The sealed vial of embodiment 74, wherein the sealed vial is for multiple uses.

### Embodiment 77

An emulsion comprising: a) a dispersed organic phase comprising nanodroplets comprising one or more organic solvents and a hydrophobic drug, and b) a continuous aqueous phase comprising an albumin and a bioactive polypeptide.

### Embodiment 78

The emulsion of embodiment 77, wherein at least a portion of the albumin is conjugated to the bioactive polypeptide.

### Embodiment 79

The emulsion of embodiment 77 or 78, wherein the weight ratio of the albumin to the bioactive polypeptide in the emulsion is about 1:1 to about 1000:1.

### Embodiment 80

The emulsion of any one of embodiments 77-79, wherein the weight ratio of the hydrophobic drug to the bioactive polypeptide in the emulsion about 1:1 to about 100:1.

### Embodiment 81

An emulsion comprising: a) a dispersed organic phase comprising nanodroplets comprising one or more of the one or more organic solvents and a hydrophobic drug, and b) a continuous aqueous phase comprising an albumin derivatized with a crosslinker moiety.

### Embodiment 82

The emulsion of any one of embodiments 77-81, wherein the weight ratio of the albumin to the hydrophobic drug in the emulsion is about 1:1 to about 20:1.

### Embodiment 83

A crude mixture comprising: a) an organic solution comprising one or more organic solvents and a hydrophobic drug, and b) a continuous aqueous phase comprising an albumin derivatized with a crosslinker moiety.

### Embodiment 84

The crude mixture of embodiment 84, wherein the weight ratio of the albumin to the hydrophobic drug in the crude mixture is about 1:1 to about 20:1.

### Embodiment 85

A method of treating a disease in an individual, comprising administering to the individual an effective amount of the composition of any one of embodiments 1-37 and 67-73.

### Embodiment 86

The method of embodiment 85, further comprising administering to the individual an effective amount of another therapeutic agent.

### Embodiment 87

The method of embodiment 85 or 86, where the disease is a cancer.

### Embodiment 88

The method of any one of embodiments 85-87, wherein the composition is administered to the individual intravenously.

### Embodiment 89

The method of any one of embodiments 85-88, wherein the individual is human.

### EXAMPLES

### Example 1: Effect of Avastin® Excipients on Nab-Paclitaxel Compositions

Excipients contained within Avastin® (bevacizumab) include sodium phosphate buffer, pH 6.2; α,α-trehalose; and polysorbate 20. The stability of nanoparticles comprising albumin and paclitaxel in the presence of these excipients at various temperatures and pH was determined by measuring particle size distribution by Dynamic Light Scattering and, in some cases, filterability using a 0.2 μm membrane. Pre-manufactured nanoparticles comprising albumin and paclitaxel (namely, Abraxane®) were reconstituted at a paclitaxel concentration of 10 mg/mL and subjected to the following conditions.

1. Abraxane® was reconstituted to 10 mg/mL with normal saline solution and the pH was adjusted to 7. After 24 hours at room temperature, the nanoparticles were stable (as judged by no significant alteration in particle size distribution as determined by dynamic light scattering, as well as by visual and microscopic observation of particulates, aggregates and sedimentation.

2. Abraxane® was reconstituted to 10 mg/mL with normal saline solution and the pH was adjusted to 5. After 24 hours at room temperature, the nanoparticles were not stable, most likely due to aggregation near the isoelectric point (pI) of albumin near 5.

3. Abraxane® was reconstituted to 10 mg/mL with 20% of Avastin® buffer (5.8 mg/mL sodium phosphate (monobasic, monohydrate), 1.2 mg/mL sodium phosphate (dibasic, anhydrous), pH 6.2, 60 mg/mL α,α-trehalsoe), but excluding polysorbate 20) and 80% normal saline, and the pH was adjusted to 7. After 24 hours at room temperature, the nanoparticles were stable with no significant alteration in particle size distribution. The sodium phosphate and α,α-trehalose were concluded to not have a significant impact on nanoparticle stability.

4. Abraxane® was reconstituted to 10 mg/mL with 20% of Avastin® buffer (containing sodium phosphate buffer and α,α-trehalose, but excluding polysorbate 20) and 80% normal saline, and the pH was adjusted to 5. After 24 hours at room temperature, the nanoparticles were not stable.

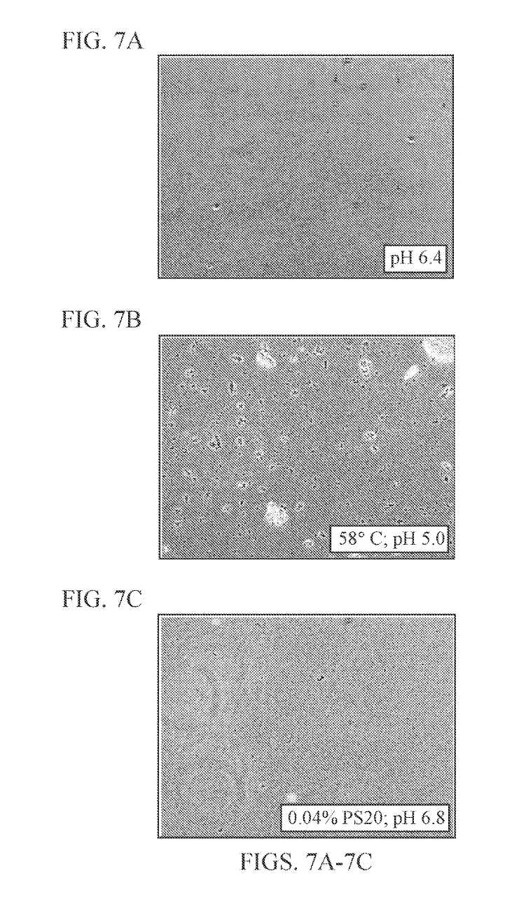

5. Abraxane® was reconstituted to 10 mg/mL with 20% of Avastin® buffer (containing sodium phosphate buffer and α,α-trehalose, but excluding polysorbate 20) and 80% normal saline, and the pH was adjusted to 5. After 24 hours at 58° C., the nanoparticles were not stable, as nanoparticle aggregates were detected. See FIG. 7B.

6. Abraxane® was reconstituted to 10 mg/mL with 100% of Avastin® buffer (containing sodium phosphate buffer and α,α-trehalose, but excluding polysorbate 20), and without adjusting the pH (the pH was measured as 6.4). After 24 hours at room temperature, the nanoparticles were stable with no significant alteration in particle size distribution. See FIG. 7A The sodium phosphate and α,α-trehalose were concluded to not have a significant impact on nanoparticle stability.

7. Abraxane® was reconstituted to 10 mg/mL with 100% of Avastin® buffer (containing each of sodium phosphate buffer and α,α-trehalose, and polysorbate 20), and without adjusting the pH (the pH was measured as 6.8). The nanoparticles aggregated immediately when viewed by optical microscopy (FIG. 7C), and resulted in microscopic particulates and visible sedimentation of the suspension after incubation for 24 hours at room temperature. Therefore, the polysorbate surfactant itself at the concentration provided in the Avastin® formulation does impact nanoparticle stability.

8. Abraxane® was reconstituted to 10 mg/mL with 100% of Avastin® buffer (containing each of sodium phosphate buffer and α,α-trehalose, and polysorbate 20 (0.04%)), and the pH was adjusted to 5. The nanoparticles aggregated immediately after preparation (mean particle size grew from 143 nm to 159 nm), and the nanoparticles continued to be unstable after 24 hours at room temperature.

These studies indicate that polysorbate 20 present in the Avastin® buffer causes aggregation of the Abraxane® nanoparticles. Further, lowering the pH to 5 results in Abraxane® particle aggregation.

### Example 2: Effect of Nanoparticle Manufacturing Process Steps on the Stability of Bevacizumab without the Presence of Albumin

In some embodiments, nanoparticles comprising albumin and a hydrophobic drug include mixing albumin in an aqueous solution with an organic solution comprising one or more solvents and the hydrophobic drug to form a crude mixture, high-pressure homogenization of the crude mixture to form an emulsion, evaporating the organic solvent to form a post-evaporation nanoparticle suspension, diluting the nanoparticle suspension, and filtering the nanoparticle suspension. The ability of bevacizumab to withstand each of these manufacturing steps in the absence of albumin and polysorbate 20 was determined by subjecting bevacizumab to each manufacturing step. Samples from each manufacturing step were analyzed by size exclusion chromatography (SEC) to determine the amount of remaining bevacizumab and its aggregation state (i.e., by measuring the conversion to high molecular weight species).

400 mg bevacizumab was purified from Avastin® (25 mg/mL bevacizumab, 400 mg/vial) by ionic exchange chromatography to remove polysorbate 20. The 400 mg of bevacizumab was loaded onto a preparative FPLC (ATKA Purifier) with an 85 mL Capto S Impact column. The column was washed with 5 M sodium citrate, pH 5.0, and eluted with 25 mM sodium citrate, pH 5.0, 1 M NaCl. The purified bevacizumab was buffer exchanged twice against 1 L of dialysis buffer (50 mM sodium phosphate buffer, pH 6.2) at 5° C. The dialyzed bevacizumab was formulated by adding 10.059 mL of trehalose stock (50 mM sodium phosphate buffer, pH 6.2 containing 400 mg/mL α,α-trehalose dihydrate). The final composition of the formulated bevacizumab was 5.98 mg/mL (theoretical), 50 mM sodium phosphate, pH 6.2, 60 mg/mL α,α-trehalose dihydrate. Bevacizumab concentration in the formulated bevacizumab was 5.966 mg/mL as determined by A280 using theoretical extinction coefficient of 1.661.

18.4 mL of 2.18 mg/mL bevacizumab solution was prepared by mixing 6.7 mL purified bevacizumab stock solution (5.966 mg/mL) with 11.7 mL water. The bevacizumab solution was mixed using a high-shear mixer set at 5400 rpm. While the bevacizumab solution was being mixed, 1.5 mL of organic solution containing 90% v/v chloroform and 10% v/v ethanol was added. The organic solution and the aqueous solution were mixed for 5 minutes to create a crude mixture, which was sampled and allowed to settle, with the settled supernatant used for a SEC measurement. The crude mixture was transferred to a vessel in a high-pressure homogenizer. The crude mixture was homogenized using the high-pressure homogenizer for several passes, thereby forming an emulsion. The emulsion was sampled and allowed to settle, with the settled supernatant used for a SEC measurement. Approximately 18 mL of the high-pressure homogenized emulsion was transferred to a rotary evaporator equipped with a 2 L round bottom evaporation flask. Vacuum pressure of the evaporation flask was maintained, and the evaporation was partially immersed in the water bath, which was set at 40° C. The organic solvent (and a portion of the water) was evaporated from the emulsion, and foaming inside the flask was controlled by changing the rotation speed of the flask and the pressure as necessary. After approximately 15 minute of evaporation, the resulting volume was about 2 mL. The post-evaporation nanoparticle suspension was transferred to a separate container and 3 mL of water was added. The diluted post-evaporation nanoparticle suspension was sampled for a SEC measurement. Approximately 0.5 mL of the diluted post-evaporation nanoparticle suspension was sterile filtered and sampled for a SEC measurement.

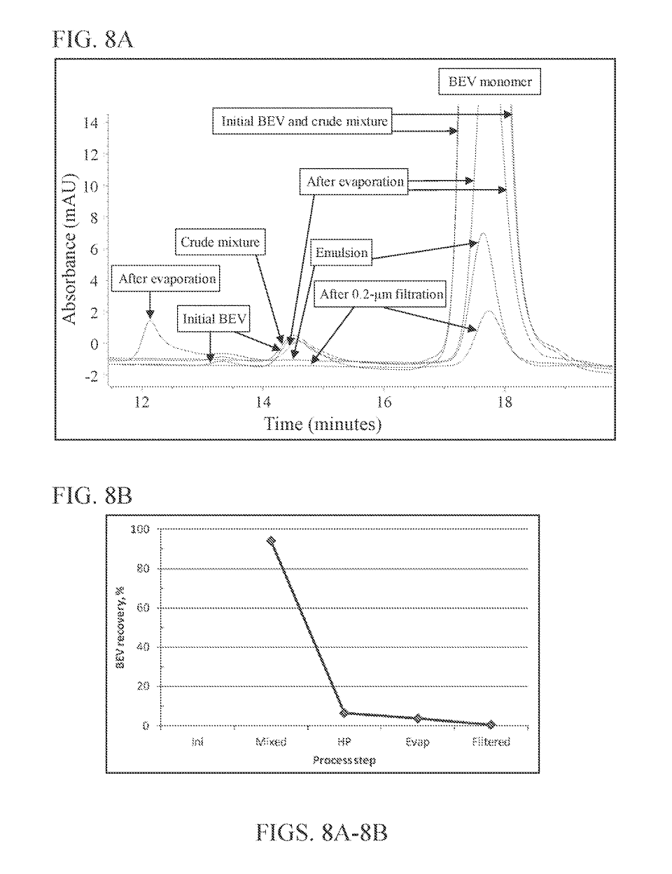

Results from the size exclusion chromatography measurements are shown in FIG. 8A. These measurements include the unprocessed bevacizumab, supernatant from the crude mixture, supernatant from the high-pressure homogenized emulsion, the diluted post-evaporation suspension, and the filtered sample. The late-eluting peak (right-side of the figure) illustrates the bevacizumab monomer, and the earlier-eluting peaks represent higher molecular weight species of bevacizumab. As the processing progressed, the amount of bevacizumab monomer decreased. The fraction of bevacizumab recovery at each step relative to the initial concentration of bevacizumab added to the beginning of the process is shown in FIG. 8B, showing that most of the bevacizumab is degraded or aggregated in the high-pressure homogenization step.

### Example 3: Effect of Albumin on the Stability of Bevacizumab in the Manufacturing Process

The ability of bevacizumab to withstand each of the manufacturing steps in the absence polysorbate 20, but with the inclusion of various amounts of human albumin (HA), was determined by subjecting bevacizumab to each manufacturing step. Bevacizumab was prepared as described in Example 2, except 1%, 2.5%, 5% or 10% human albumin was included in the bevacizumab preparation at the start of the manufacturing process. Samples from each manufacturing step were analyzed by size exclusion chromatography (SEC) to determine the amount of remaining bevacizumab and its aggregation state (i.e., by measuring the conversion to high molecular weight species).

For the 1% HA containing preparation, 0.945 mL of 20% human albumin, 6.9 mL of bevacizumab stock solution (5.966 mg/mL, Example 2), and 11.1 mL of water were combined to make aqueous solution for processing. For the 2.5% HA containing preparation, 2.363 mL of 20% human albumin, 6.9 mL of bevacizumab stock solution (5.966 mg/mL, Example 2), and 9.6 mL of water were combined to make aqueous solution for processing. For the 5% HA containing preparation, 4.85 mL of 20% human albumin, 7 mL of bevacizumab stock solution (5.966 mg/mL, Example 2), and 7.55 mL of water were combined to make aqueous solution for processing. For the 10% HA containing preparation, 9.45 mL of 20% human albumin, 6.9 mL of bevacizumab stock solution (5.966 mg/mL, Example 2), and 2.55 mL of water were combined to make aqueous solution for processing.

The bevacizumab solution was mixed using a high-shear mixer set at 5400 rpm. While the bevacizumab solution was being mixed, 1.5 mL of organic solution containing 90% v/v chloroform and 10% v/v ethanol was added. The organic solution and the aqueous solution were mixed for 5 minutes to create a crude mixture, which was sampled for a SEC measurement. The crude mixture was transferred to a vessel in a high-pressure homogenizer. The crude mixture was homogenized using the high-pressure homogenizer for several passes, thereby forming an emulsion. The emulsion was sampled for a SEC measurement. Approximately 18 mL of the high-pressure homogenized emulsion was transferred to a rotary evaporator equipped with a 2 L round bottom evaporation flask. Vacuum pressure of the evaporation flask was maintained, and the evaporation was partially immersed in the water bath, which was set at 40° C. The organic solvent (and a portion of the water) was evaporated from the emulsion, and foaming inside the flask was controlled by changing the rotation speed of the flask and the pressure as necessary. After approximately 3-5 minutes of evaporation, the resulting volume was measured. This post-evaporation nanoparticle suspension was sampled for SEC measurement. A portion of this suspension was filtered and sampled for SEC measurement.

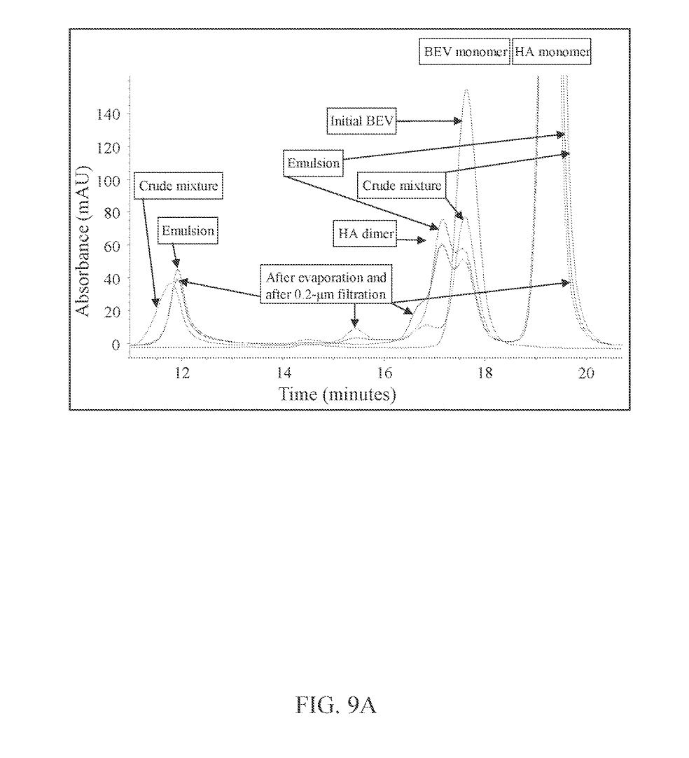

Results from the size exclusion chromatography measurements for the 10% HA solution are shown in FIG. 9A. The latest-eluting peak (about 18.3 minutes) contains the HA monomer, and the peak eluting at about 17.6 minutes contains the bevacizumab monomer. Earlier-eluting peaks represent higher molecular weight species of human albumin and/or bevacizumab. The amount of each species was quantified by the area under the peak.

FIG. 9B shows the amounts of bevacizumab recovered after each step in the process for each of formulations at different HA concentrations, including the formulation containing no HA (Example 2). The 0% and 1% HA containing formulations lose a large proportion of the initial bevacizumab added to the formulation. The majority of the bevacizumab was retained throughout the manufacturing process for each of the 2.5%, 5% and 10% HA containing formulations, indicating that the presence of at least 2.5% HA in the formulations protects the loss of bevacizumab through severe degradation and aggregation.

FIG. 9C shows the amounts of bevacizumab monomer remaining at each step in the process for each of formulations, by integration of only the bevacizumab monomer peak. By the measure of the fraction of unaggregated bevacizumab monomer, there is a monotonic increase in the fraction of bevacizumab not adversely degraded during the processing as the HA concentration in the formulation is increased (the results for the formulation with 0% HA at the later processing steps are inconclusive due to the small quantity of bevacizumab present in those samples. At 10% HA, approximately 100% of the bevacizumab remains as monomer throughout the manufacturing process, demonstrating that this concentration of HA can completely protect bevacizumab from degradation during nanoparticle manufacturing.

### Example 4: Addition of Bevacizumab at Different Stages of the Manufacturing Process

The impact of adding bevacizumab to an emulsion comprising an aqueous phase containing albumin and an organic phase containing organic solvents was compared to the inclusion of the bevacizumab earlier in the nanoparticle manufacturing process. Samples from each manufacturing step were analyzed by size exclusion chromatography (SEC) to determine the amount of remaining bevacizumab and its aggregation state (i.e., by measuring the conversion to high molecular weight species).

18.4 mL of 5% human albumin (HA) solution (the aqueous solution) was prepared by mixing 4.6 mL HA stock solution (20%) and 13.8 mL of water. In the control sample, 4.85 mL of 20% human albumin, 7 mL of bevacizumab stock solution (5.966 mg/mL, Example 2), and 7.55 mL of water were combined to make aqueous solution. The aqueous solution was mixed using a high-shear mixer set at 5400 rpm. While the bevacizumab solution was being mixed, 1.5 mL of organic solution containing 90% v/v chloroform and 10% v/v ethanol was added. The organic solution and the aqueous solution were mixed for 5 minutes to create a crude mixture. The crude mixture was transferred to a vessel in a high-pressure homogenizer. The crude mixture was homogenized for several passes through the high-pressure homogenizer, thereby forming an emulsion. 6.0 mL of bevacizumab stock solution (5.966 mg/mL, Example 2) was added to the crude emulsion, and the emulsion was sampled for a SEC measurement. Approximately 24 mL of the high-pressure homogenized emulsion was transferred to a rotary evaporator equipped with a 2 L round bottom evaporation flask. Vacuum pressure of the evaporation flask was maintained, and the evaporation was partially immersed in the water bath, which was set at 40° C. The organic solvent (and a portion of the water) was evaporated from the emulsion, and foaming inside the flask was controlled by changing the rotation speed of the flask and the pressure as necessary. After approximately 6 minutes of evaporation, the resulting volume was measured as about 3 mL. 2 mL of water was added to the post-evaporation nanoparticle suspension, and the post-evaporation nanoparticle suspension was sampled for SEC measurement. A portion of this suspension was filtered and sampled for SEC measurement.

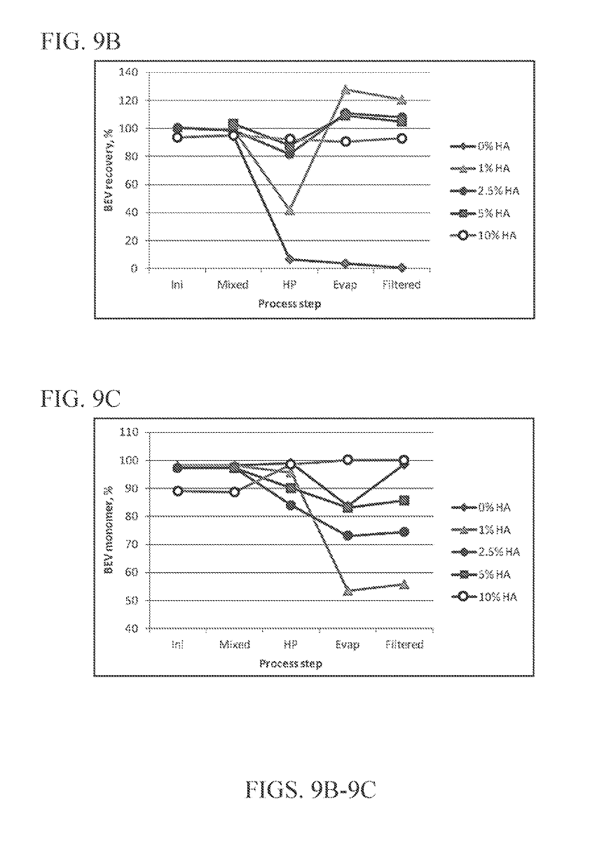

FIG. 10A shows the amounts of bevacizumab remaining at each step in the process for both the formulation where the bevacizumab is added in the initial aqueous solution (control) and where the bevacizumab is added after the emulsion is formed. FIG. 10B shows the amounts of bevacizumab monomer remaining at each step in the process for the two formulations, by integration of only the bevacizumab monomer peak. For the formulation created by adding bevacizumab to the emulsion, over 96% of the bevacizumab monomer is retained at the end of the process, as compared to approximately 85% for the formulation made by adding bevacizumab in the initial aqueous solution (control). This result demonstrates that adding the bevacizumab after the creating of the emulsion can protect bevacizumab from excessive degradation during nanoparticle manufacturing while still providing an opportunity for bevacizumab to contact and become associated with the emulsion droplet surface before completion of particle formation.

### Example 5: Manufacture of Nanoparticles Containing Albumin, Paclitaxel, and Bevacizumab

1.6 mL of a 100 mg/mL solution of paclitaxel in an organic mixture containing 90% v/v chloroform and 10% v/v ethanol was prepared (the “organic solution). Separately, 18.4 mL of an aqueous solution containing 5% HA solution and 2.2 mg/mL bevacizumab was prepared by mixing 4.725 mL HA stock solution, 6.9 mL of bevacizumab stock solution and 7.275 mL water. The aqueous solution was mixed using a high-shear mixer set at 5400 rpm. While mixing the aqueous solution 1.6 mL of the paclitaxel containing organic solution was added. The aqueous solution and the organic solution were mixed for 5 minutes to create a crude mixture. The crude mixture can be sampled for SEC measurement. The crude mixture was transferred to a vessel in a high-pressure homogenizer. The crude mixture was then homogenized by the high-pressure homogenizer for several passes. Approximately 24 mL of the emulsion was transferred to a rotary evaporator equipped with 2 L round bottom flask and water bath set at 40° C. The organic solvents and a portion of the water were evaporated from the emulsion by applying and maintaining a vacuum pressure and partially immersing the rotating round bottom flask in the water bath. Foaming inside the flask was controlled by changing the rotation speed of the flask and the pressure as necessary. After approximately 5 minutes of evaporation, the resulting volume was about 4 mL, and this was transferred to a separate container where 4 mL of water was added. This evaporated suspension can be sampled for SEC measurement. The total remaining volume of this suspension was then sterile filtered. The resulting particle suspension can be sampled for particle size by dynamic light scattering and SEC measurement. The particles were recovered by ultracentrifugation. The amount of bevacizumab associated with the nanoparticles can be determined.

### Example 6: Admixture Manufacture of Nab-Paclitaxel and Bevacizumab

Admixtures of nab-paclitaxel and bevacizumab were formed at various bevacizumab concentrations according to the following protocols.

Batch 1. 1.6 mL of Avastin® (bevacizumab and excipients, 25 mg/mL) and 3.4 mL 0.9% NaCl normal saline (G-Biosciences) was added to a lyophilized nab-paclitaxel composition (100 mg paclitaxel) in a vial. The contents of the vial were reconstituted without mixing for 5 minutes, followed by gentle mixing to assure complete reconstitution. The mixture was incubated at room temperature (˜20° C.) for 1 hour before adding 5 mL normal saline to the vial. The mixture was gently mixed to assure homogeneity. The final concentration of paclitaxel was 10 mg/mL (with 90 mg/mL albumin), and the final concentration of bevacizumab was 4 mg/mL.

Batch 2. 3.2 mL of Avastin® (bevacizumab and excipients, 25 mg/mL) and 1.8 mL 0.9% NaCl normal saline (G-Biosciences) was added to a lyophilized nab-paclitaxel composition (100 mg paclitaxel) in a vial. The contents of the vial were reconstituted without mixing for 5 minutes, followed by gentle mixing to assure complete reconstitution. The mixture was incubated at room temperature (˜20° C.) for 1 hour before adding 5 mL normal saline to the vial. The mixture was gently mixed to assure homogeneity. The final concentration of paclitaxel was 10 mg/mL (with 90 mg/mL albumin), and the final concentration of bevacizumab was 8 mg/mL.

Batch 3. 6 mL of Avastin® (bevacizumab and excipients, 25 mg/mL) was added to a lyophilized nab-paclitaxel composition (100 mg paclitaxel) in a vial. The contents of the vial were reconstituted without mixing for 5 minutes, followed by gentle mixing to assure complete reconstitution. The mixture was incubated at room temperature (˜20° C.) for 1 hour before adding 4 mL 0.9% NaCl normal saline (G-Biosciences) to the vial. The mixture was gently mixed to assure homogeneity. The final concentration of paclitaxel was 10 mg/mL (with 90 mg/mL albumin), and the final concentration of bevacizumab was 15 mg/mL.

Control Batch 4. 5 mL 0.9% NaCl normal saline (G-Biosciences) was added to a lyophilized nab-paclitaxel composition (100 mg paclitaxel) in a vial. The contents of the vial were reconstituted without mixing for 5 minutes, followed by gentle mixing to assure complete reconstitution. The mixture was incubated at room temperature (˜20° C.) for 1 hour before adding 5 mL normal saline to the vial. The mixture was gently mixed to assure homogeneity. The final concentration of paclitaxel was 10 mg/mL.

Samples of each suspension were diluted by a 10-fold using (1) 0.9% NaCl normal saline, (2) water for injection (WFI), (3) phosphate buffered saline (PBS) (Amresco, diluted from 10×PBS), (4) 9:1 mixture of WFI and 10×PBS formed by adding WFI to the sample, incubating for 5 minutes at room temperature, then adding a spike of 10×PBS, or (5) 1:9 mixture of 10×PBS and WFI formed by adding 10×PBS to the sample, incubating for 5 minutes at room temperature, then adding WFI. Particle size and particle size distribution after dilution was measured by dynamic light scattering (DLS) using a Malvern Zetasizer Nano ZS (Malvern Instruments, Westborough, Mass.). Results are shown in Table 1.

TABLE 1
Particle Size and Distribution by DLS after 10-Fold Dilution of Sample

| | Z-Average diameter in nm (PDI) | | | |
|---|---|---|---|---|
| | Batch 1 | Batch 2 | Batch 3 | |
| | 10 mg/mL | 10 mg/mL | 10 mg/mL | |
| | nab-paclitaxel + | nab-paclitaxel + | nab-paclitaxel + | Batch 4 |
| | 4 mg/mL | 8 mg/mL | 15 mg/mL | 10 mg/mL |
| | Avastin ® | Avastin ® | Avastin ® | nab-paclitaxel |
| (1) 0.9% NaCl saline | n.d. | 163.8 nm | 162.5 nm | 158.1 nm |
| | | (0.146) | (0.154) | (0.105) |
| (2) WFI | 164.8 nm | 202.4 nm | 2624 nm | 160.3 nm |
| | (0.093) | (0.254) | (0.423)* | (0.113) |
| (3) PBS | n.d. | 160.4 nm | 162.3 nm | 161.9 nm |
| | | (0.113) | (0.134) | (0.157) |
| (4) 9:1 WFI:10X PBS | n.d. | 170.1 nm | 164.2 nm | 168.1 nm |
| | | (0.155) | (0.127)** | (0.176) |
| (5) 1:9 PBS:WFI | n.d. | n.d. | 165.5 nm | n.d. |
| | | | (0.150) | |

n.d. = not determined.
*= sample becomes opaque with WFI and precipitates during storage.
**= sample becomes opaque with WFI, but low turbidity recovered with addition of 10X PBS.

### Example 7: Bevacizumab Associates with Nab-Paclitaxel after Admixture

6 mL of Avastin® (bevacizumab and excipients, 25 mg/mL) was added to a lyophilized nab-paclitaxel composition (100 mg paclitaxel) in a vial. The contents of the vial were reconstituted without mixing for 5 minutes, followed by gentle mixing to assure complete reconstitution. The mixture was incubated at room temperature (˜20° C.) for 1 hour before adding 4 mL 0.9% NaCl normal saline (G-Biosciences) to the vial. The mixture was gently mixed to assure homogeneity. The final concentration of paclitaxel was 10 mg/mL (with 90 mg/mL albumin), and the final concentration of bevacizumab was 15 mg/mL.

The sample was centrifuged at 39,000 RPM (176,351×g) for 70 minutes at 20° C. (Beckman Optima LE-80 Ultracentrifuge with Ti45 rotor and Teflon inserts). The supernatant was retains (Sample “S”). The pellet was washed with phosphate buffered saline (PBS) five times (Samples “W1” to “W5”). 200 proof ethanol was added to the centrifuge tube and the pellet was sonicated to dissolve the paclitaxel. The sample washed with ethanol was centrifuged at 10,000 RPM (14,029.3×g) for 20 minutes at 20° C., the supernatant decanted, and the sample dried by lyophilization. The lyophilized pellet was resuspended in PBS (Sample “P”).

The retained samples were subjected to an immunoblot analysis. The samples were run on a 4-1% Bis-Tris SDS-PAGE gel in MOPS buffer. Proteins were transferred to a nitrocellulose membrane and stained using anti-HSA (human serum albumin, 1:5000 mouse) and anti-human IgG (1:2000 rabbit) primary antibodies, and IRDye labeled anti-mouse (1:20,000) and anti-rabbit (1:20,000) secondary antibodies.

The immunoblot showed that both bevacizumab and albumin were retained in the pellet after extracting paclitaxel. This indicates association of the bevacizumab with nab-paclitaxel after admixture of Avastin® and nab-paclitaxel.

### Example 8: Admixture Manufacture of Nab-Paclitaxel and Trastuzumab

Admixtures of nab-paclitaxel and trastuzumab were formed at various trastuzumab concentrations according to the following protocols. Herceptin® (trastuzumab and excipients) was reconstituted by adding 20 mL of 0.9% NaCl saline for injection to a vial and allowing the contents to dissolve to provide a 21 mg/mL solution. The vial we then gently mixed to ensure complete reconstitution.

Batch 1.0.476 mL of reconstituted Herceptin® (21 mg/mL) and 9.524 mL of 0.9% NaCl saline for injection was added to a lyophilized nab-paclitaxel composition (100 mg paclitaxel) in a vial. The contents of the vial were allowed to dissolve for 15 minutes before the vial was gently swirled to ensure complete dissolution. The dissolved contents were then allowed to stand for 1 hour at room temperature. 10 mL of 0.9% NaCl saline for injection was then added to the vial. The final concentration of paclitaxel was 5 mg/mL, and the final concentration of trastuzumab was 0.5 mg/mL.

Batch 2. 3.808 mL of reconstituted Herceptin® (21 mg/mL) and 6.192 mL of 0.9% NaCl saline for injection was added to a lyophilized nab-paclitaxel composition (100 mg paclitaxel) in a vial. The contents of the vial were allowed to dissolve for 15 minutes before the vial was gently swirled to ensure complete dissolution. The dissolved contents were then allowed to stand for 1 hour at room temperature. 10 mL of 0.9% NaCl saline for injection was then added to the vial. The final concentration of paclitaxel was 5 mg/mL, and the final concentration of trastuzumab was 4 mg/mL.

Batch 3. 7.14 mL of reconstituted Herceptin® (21 mg/mL) and 2.86 mL of 0.9% NaCl saline for injection was added to a lyophilized nab-paclitaxel composition (100 mg paclitaxel) in a vial. The contents of the vial were allowed to dissolve for 15 minutes before the vial was gently swirled to ensure complete dissolution. The dissolved contents were then allowed to stand for 1 hour at room temperature. 10 mL of 0.9% NaCl saline for injection was then added to the vial. The final concentration of paclitaxel was 5 mg/mL, and the final concentration of trastuzumab was 7.5 mg/mL.

### Example 9: Ionic Strength Impact of Nab-Paclitaxel Admixed with Bevacizumab or Trastuzumab

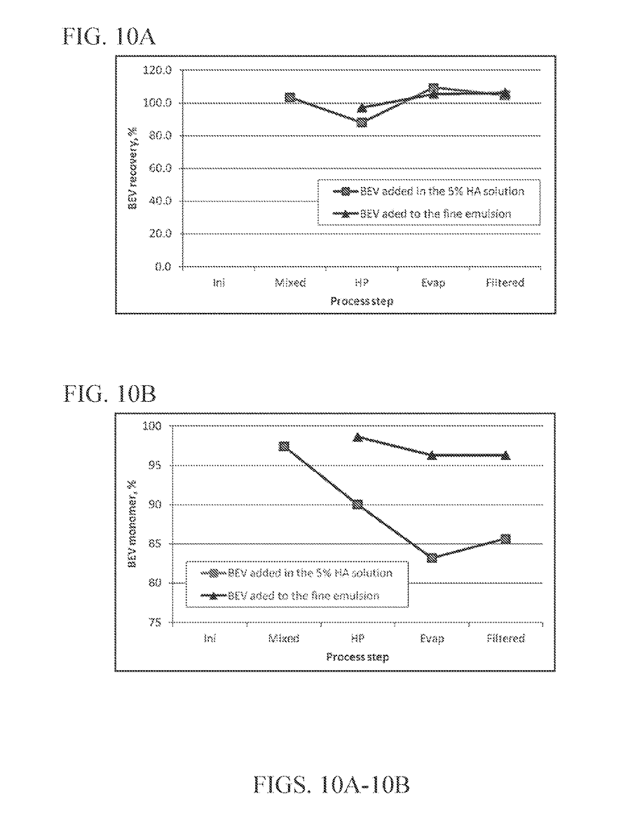

Nab-paclitaxel was admixed with either bevacizumab or trastuzumab according to the protocols described in Examples 6 and 8 (8:10 or 15:10 ratio of antibody to nab-paclitaxel). The nanoparticle suspensions were diluted with either WFI or various concentrations of NaCl saline. Particle size (Z-average diameter in nanometers) after dilution was measured by dynamic light scattering (DLS) using a Malvern Zetasizer Nano ZS (Malvern Instruments, Westborough, Mass.). Results are shown in FIG. 11.

A 10-fold dilution of bevacizumab alone or nab-paclitaxel alone in low ionic strength media does not cause a particle size increase. However, a 10-fold dilution of admixtures of nab-paclitaxel and antibody in low ionic strength media cause an increase in particle size. The particles increase in size in less than 0.15% (25 mM) NaCl for trastuzumab admixed with nab-paclitaxel at a 15:10 ratio of trastuzumab to paclitaxel, and less than 0.075% (12.5 mM) NaCl for trastuzumab admixed with nab-paclitaxel at a 8:10 ratio of trastuzumab to paclitaxel. For bevacizumab admixed with nab-paclitaxel (15:10 ratio of bevacizumab to paclitaxel), the particles increase in size in less than 0.05% (9 mM) NaCl. As can be seen from the results shown in FIG. 11, low ionic strength media (e.g., WFI) causes an increase in particle size and higher ionic strength media decreases particle size until it reaches the size of the control sample. The change in particle size is likely due to electrostatic association of the antibody with free albumin or surface bound albumin on the nab-paclitaxel nanoparticles. The size increase depends on the ionic strength of the medium, the type of antibody (e.g., bevacizumab or trastuzumab), and the concentration of antibody in the admixture. The increase in particle size is reversible if the ionic strength is increased.

### Example 10: Treatment of A375 Mouse Xenografts with of Nab-Paclitaxel Admixed with Bevacizumab

A375 human melanoma cells were subcutaneously injected into mice to generate xenograft models. The tumors were allowed to grow to about 600 mm³ before treatment according to the following protocols. Nine mice were treated for each protocol, except as noted.

Cohort 1—Vehicle control (“Vehicle”). Avastin® buffer was prepared by mixing together in a sterile 30 mL PETG container 960 mg α,α-trehalose dehydrate, 92.8 mg sodium phosphate (monobasic, monohydrate), 19.2 mg sodium phosphate (dibasic, anhydrous), 6.4 mg polysorbate 20, and 16 mL water for injection (WFI). The Avastin® buffer was then passed through a 0.2 μm sterile filter. 96 μL of the Avastin® buffer was mixed with 270 μL of 200 mg/mL human albumin solution and 834 μL of 0.9% NaCl saline for injection. The vehicle control was administered to each mouse at a dosage of 120 μL/mouse on the second day of the experiment.

Cohort 2—nab-paclitaxel at 30 mg/kg paclitaxel dose (“ABX30”). 20 mL 0.9% NaCl saline for injection was used to reconstitute a lyophilized nab-paclitaxel composition (100 mg paclitaxel) to a concentration of 5 mg/mL paclitaxel. Each mouse weighed approximately 0.02 kg, and the reconstituted nab-paclitaxel was administered to each mouse at a dosage of 120 μL/mouse on the second day of the experiment.

Cohort 3—Bevacizumab at 12 mg/kg dose (“BEV12”). 96 μL bevacizumab (Avastin®, 25 mg/mL) was diluted with 904 μL 0.9% NaCl saline for injection to a final concentration of 2.4 mg/mL bevacizumab. Each mouse weight approximately 0.02 kg, and the diluted Avastin® was administered to each mouse at a dosage of 100 μL/mouse on the second day of the experiment.

Cohort 4—BEV12 and ABX30 on same day (“BEV12+ABX30—Same Day”). 100 μL/mouse of diluted Avastin® as prepared for Cohort 3 (“BEV12”) and 120 μL/mouse nab-paclitaxel as prepared for Cohort 2 (“ABX30”) was administered to each mouse on the second day of the experiment.

Cohort 5—BEV12 followed by ABX30 one day later (“BEV12+ABX30—1 Day Apart”); eight mice treated. 100 μL/mouse of diluted Avastin® as prepared for Cohort 3 (“BEV12”) was administered to each mouse on the first day of the experiment. 120 μL/mouse nab-paclitaxel as prepared for Cohort 2 (“ABX30”) was administered to each mouse on the second day of the experiment.

Cohort 6—Nab-paclitaxel (30 mg/kg paclitaxel) admixed with bevacizumab (12 mg/kg) (“AB160”). A lyophilized nab-paclitaxel composition (100 mg paclitaxel) was reconstituted with 1.6 mL bevacizumab (Avastin®, 25 mg/mL) and 3.4 mL 0.9% NaCl saline for injection. The mixture was incubated for 1 hour at room temperature before adding 15 mL 0.9% NaCl saline for injection, resulting in a final concentration of 5 mg/mL paclitaxel and 2 mg/mL bevacizumab. The admixture was administered to each mouse at a dosage of 120 μL/mouse on the second day of the experiment.

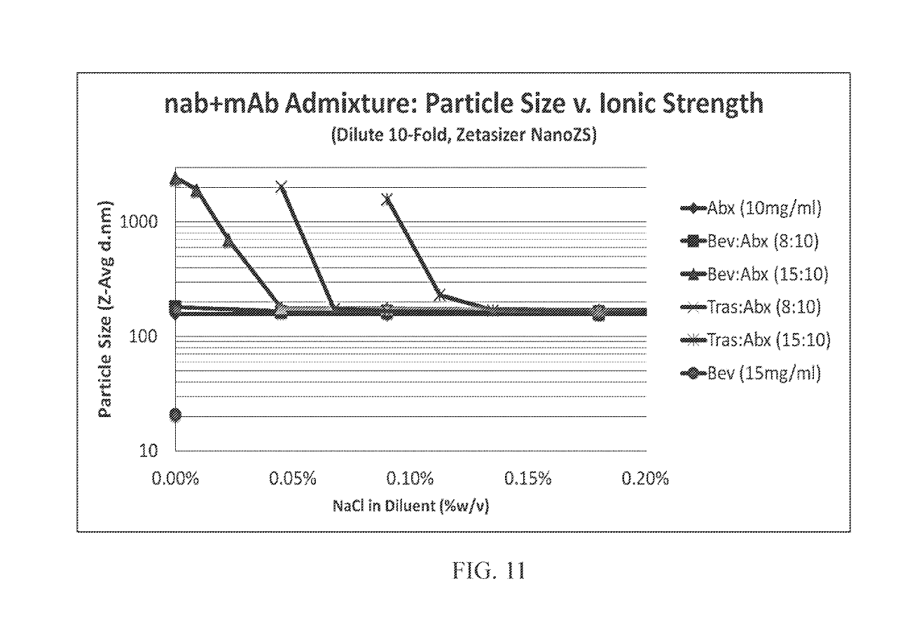

Tumor volume was measured on day seven of the experiment, and percent tumor volume change for each mouse in each cohort is shown in FIG. 12. No significant difference was seen between the admixture of nab-paclitaxel and bevacizumab (“AB160”) and either same day (“BEV12+ABX30—Same Day”) or one day apart (“BEV12+ABX30—1 Day Apart”) administration. However, the admixture of nab-paclitaxel and bevacizumab (“AB160”) did result in statistically significant decrease in tumor growth compared to administration of nab-paclitaxel alone (“ABX30”; p=0.0034), bevacizumab alone (“BEV12”; p=0.006), or the vehicle control (“Vehicle”; p<0.0001). P-values were determined using an un-paired t-test.

Survival time of the treated mice was also recoded. Mice with tumors larger than 2000 mm³ were euthanized. Median survival time and relevant p-values are shown in Table 2. As shown by the p-values, there is no significant difference in the survival of the mice treated with the admixture of nab-paclitaxel and bevacizumab (“AB160”) and the separately administered nab-paclitaxel and bevacizumab (“BEV12+ABX30—Same Day” or “BEV12+ABX30—1 Day Apart”).

TABLE 2
Survival Study of A375 Melanoma Xenograft Model

| | Median | P Values (log-rank test) | | |
|---|---|---|---|---|
| | Survival | vs. | | vs. |
| Cohort | (days) | Vehicle | vs. ABX30 | AB160 |
| Vehicle | 26 | N/A | N/S | <0.001 |
| ABX30 | 26 | N/S | N/A | <0.01 |
| BEV12 | 38 | <0.01 | N/S | N/S |
| BEV12 + | 38 | <0.001 | <0.05 | N/S |
| ABX30 − | | | | |
| Same Day | | | | |
| BEV12 + | 43 | <0.0001 | <0.001 | N/S |
| ABX30 − 1 | | | | |
| Day Apart | | | | |
| AB160 | 42 | <0.001 | <0.01 | N/A |

N/A = not applicable;
N/S = not significant.

### Example 11: Manufacture of Nanoparticles with Embedded Trastuzumab, Paclitaxel, and Albumin

27.6 mL of a 15 mg/mL human serum albumin (HSA) solution (in water) was combined with 2.4 mL of a 200 mg/mL paclitaxel solution (90/10 mixture of CHCl₃ and ethanol) while mixing to form a mixture. The mixture was transferred to an Avestin homogenizer and homogenized at 20-22 kpsi. An additional 5 mL of 15 mg/mL HSA was passed through the homogenizer to collect a final volume of 35 mL. The mixture was passed through the homogenizer for several cycles before an additional 20 mL of 15 mg/mL HSA was added to the homogenizer to chase the emulsion. A total of 40 mL of fine emulsion was collected. 0.714 mL of 21 mg/mL trastuzumab (Herceptin®) and 3.426 mL of 0.9% NaCl was added to the fine emulsion before subjecting the fine emulsion to rotary evaporation. Liquid was removed from the fine emulsion using the rotary evaporator until 9.3 mL of the sample remained as a post-evaporation suspension. The post-evaporation suspension was transferred to a scintillation vial, and the rotary evaporator was twice washed with 1.5 mL aliquots of water for injection (WFI), which were added to the scintillation vial (12.3 mL final volume). The resulting post-evaporation suspension was stored overnight at 5° C.

The post-evaporation suspension was removed from cold storage and equilibrated to room temperature. 12 mL of the post-evaporation suspension was mixed with 10.84 mL of 200 mg/mL HSA and 33.82 mL 0.9% NaCl. The mixture was then filtered before measuring particle size and paclitaxel concentration. Particle size and distribution was measured by dynamic light scattering (DLS) using a Malvern Zetasizer Nano ZS (Malvern Instruments, Westborough, Mass.) by diluting 50 μL of the post-evaporation suspension in 1.5 mL 0.9% NaCl saline. The Z-average diameter of the filtered suspension was 145.5 nm, with a polydispersion index (PDI) of 0.159. Paclitaxel concentration of the filtered suspension was measured by RP-HPLC as 6.21 mg/mL. The filtered suspension was frozen at −80° C. overnight.

The frozen, filtered suspension was removed from cold storage and allowed to thaw and equilibrate to room temperature. 0.829 mL of 21 mg/mL trastuzumab (Herceptin®) and 10.787 mL 0.9% NaCl was added to 48 mL of the filtered suspension to adjust the concentration of the suspension to include 5 mg/mL paclitaxel and 0.5 mg/mL trastuzumab. The final suspension was aliquoted into vials (3 mL per vial) and stored at −80° C. One sample aliquot was not frozen and analyzed for particle size (Z-average diameter of 145.6 nm, by DLS), particle size distribution (PDI of 0.130, by DLS), paclitaxel concentration (5.0 mg/mL by RP-HPLC), trastuzumab concentration (0.53 mg/mL, by SEC-HPLC), and osmolality (266 mOSm).

### Example 12: Manufacture of Nanoparticles with Embedded Trastuzumab, Paclitaxel, and Albumin

Four batches of nab-paclitaxel including embedded trastuzumab were manufactured by including the trastuzumab at different time points during the manufacturing process. Trastuzumab was added (1) to a fine emulsion and incubated 10 minutes prior to processing by rotary evaporation, (2) to a fine emulsion that was immediately processed by rotary evaporation, (3) to the fine emulsion after about 3 minutes of rotary evaporation, or (4) to the to the post-evaporation suspension immediately following rotary evaporation. A control batch (5) of nab-paclitaxel without trastuzumab was also manufactured.

Batch 1. 27.6 mL of a 15 mg/mL human serum albumin (HSA) solution (in water) was combined with 2.4 mL of a 200 mg/mL paclitaxel solution (90/10 mixture of CHCl₃ and ethanol) while mixing to form a mixture. The mixture was transferred to an Avestin homogenizer and homogenized at 20-22 kpsi. The mixture was passed through the homogenizer for several cycles before an additional 20 mL of 15 mg/mL HSA as added to the homogenizer to chase the emulsion. 2.14 mL of 21 mg/mL trastuzumab (Herceptin® in 0.9% NaCl saline) and 2.00 mL 0.9% NaCl saline were added to the emulsion and incubated at room temperature for 10 minutes before processing the emulsion by rotary evaporation. The emulsion was subjected to rotary evaporation to reduce the volume to 5.0 mL. The post-evaporation suspension was then transferred to a scintillation vial. The rotary evaporator flask was washed with 2.5 mL water for injection (WFI), and the wash was added to the vial. The rotary evaporator flask was again washed with 2.5 mL WFI, which was then added to the vial.

Batch 2. 27.6 mL of a 15 mg/mL human serum albumin (HSA) solution (in water) was combined with 2.4 mL of a 200 mg/mL paclitaxel solution (90/10 mixture of CHCl₃ and ethanol) while mixing to form a mixture. The mixture was transferred to an Avestin homogenizer and homogenized at 20-22 kpsi. The mixture was passed through the homogenizer for several cycles before an additional 20 mL of 15 mg/mL HSA as added to the homogenizer to chase the emulsion. 2.14 mL of 21 mg/mL trastuzumab (Herceptin® in 0.9% NaCl saline) and 2.00 mL 0.9% NaCl saline were added to the emulsion, briefly swirled, and immediately processed by rotary evaporation. The emulsion was subjected to rotary evaporation to reduce the volume to 6.0 mL. The post-evaporation suspension was then transferred to a scintillation vial. The rotary evaporator flask was washed with 2.0 mL water for injection (WFI), and the wash was added to the vial. The rotary evaporator flask was again washed with 2.0 mL WFI, which was then added to the vial.

Batch 3. 27.6 mL of a 15 mg/mL human serum albumin (HSA) solution (in water) was combined with 2.4 mL of a 200 mg/mL paclitaxel solution (90/10 mixture of CHCl₃ and ethanol) while mixing to form a mixture. The mixture was transferred to an Avestin homogenizer and homogenized at 20-22 kpsi. The mixture was passed through the homogenizer for several cycles before an additional 20 mL of 15 mg/mL HSA as added to the homogenizer to chase the emulsion. The emulsion was immediately processed using a rotary evaporator for three minutes, before 2.14 mL of 21 mg/mL trastuzumab (Herceptin® in 0.9% NaCl saline) and 2.00 mL 0.9% NaCl saline were added to the emulsion. Once the trastuzumab and the saline were added to the emulsion, processing using the rotary evaporator immediately continued until the volume of the sample was 6.8 mL. The post-evaporation suspension was then transferred to a scintillation vial. The rotary evaporator flask was washed with 1.6 mL water for injection (WFI), and the wash was added to the vial. The rotary evaporator flask was again washed with 1.6 mL WFI, which was then added to the vial.

Batch 4. 27.6 mL of a 15 mg/mL human serum albumin (HSA) solution (in water) was combined with 2.4 mL of a 200 mg/mL paclitaxel solution (90/10 mixture of CHCl₃ and ethanol) while mixing to form a mixture. The mixture was transferred to an Avestin homogenizer and homogenized at 20-22 kpsi. The mixture was passed through the homogenizer for several cycles before an additional 20 mL of 15 mg/mL HSA as added to the homogenizer to chase the emulsion. The emulsion was immediately processed using a rotary evaporator to reduce the volume to about 10 mL. 2.14 mL of 21 mg/mL trastuzumab (Herceptin® in 0.9% NaCl saline) and 2.00 mL 0.9% NaCl saline were added to the post-evaporation solution, which was further processed using the rotary evaporator to reduce the volume to 7.0 mL. The post-evaporation suspension was then transferred to a scintillation vial. The rotary evaporator flask was washed with 1.5 mL water for injection (WFI), and the wash was added to the vial. The rotary evaporator flask was again washed with 1.5 mL WFI, which was then added to the vial.

Batch 5 (control nab-paclitaxel). 27.6 mL of a 15 mg/mL human serum albumin (HSA) solution (in water) was combined with 2.4 mL of a 200 mg/mL paclitaxel solution (90/10 mixture of CHCl₃ and ethanol) while mixing to form a mixture. The mixture was transferred to an Avestin homogenizer and homogenized at 20-22 kpsi. The mixture was passed through the homogenizer for several cycles before an additional 20 mL of 15 mg/mL HSA as added to the homogenizer to chase the emulsion. 4.14 mL of 0.9% NaCl saline was added to the emulsion, and the emulsion was subjected to rotary evaporation until the volume of the resulting post-evaporation suspension was 6.4 mL. The post-evaporation suspension was then transferred to a scintillation vial. The rotary evaporator flask was washed with 1.8 mL water for injection (WFI), and the wash was added to the vial. The rotary evaporator flask was again washed with 1.8 mL WFI, which was then added to the vial.

Average particle diameter and polydispersity index were measured by dynamic light scattering (DLS) for the post-evaporation suspension of each batch (after washing the rotary evaporator and adding the wash to the sample), with results shown in Table 3. The DLS measurements were performed using a Malvern Zetasizer Nano ZS (Malvern Instruments, Westborough, Mass.) by diluting 50 μL of the post-evaporation suspension with 1.5 mL 0.9% NaCl normal saline.

TABLE 3
Particle Size Diameter and Polydispersity Index
of Post-Evaporation Suspension

| | Diameter | Polydispersity |
|---|---|---|
| Batch | (Z-average) | Index |
| 1 (Ab added with 10 minute | 209.7 nm | 0.143 |
| incubation prior to | | |
| evaporation) | | |
| 2 (Ab added immediately prior to | 165.4 nm | 0.111 |
| evaporation) | | |
| 3 (Ab added during evaporation) | 164.6 nm | 0.106 |
| 4 (Ab added to post-evaporation) | 148.7 nm | 0.159 |
| 5 (control) | 147.6 nm | 0.159 |

The particle size of the final batches (measured by DLS) showed a trend of particle sizes being larger for batches with antibody added earlier in the manufacturing process. Compared to the control batch which had no antibody added (Batch 5), all batches containing antibody had a larger particle size. The largest particle size was observed for Batch 1 (antibody added to the emulsion, then 10 minutes of incubation at room temp, before starting rotary evaporation), which might be due to coalescence of the emulsion droplets before rotary evaporation removes the solvent in the droplet and produces solid particles. The other batches (Batches 2, 3, 4) did not include any incubation time before solvent removal by rotary evaporation, and a smaller particle size increase was observed for these batches relative to the control (Batch 1).

The concentration of paclitaxel and trastuzumab in the post-evaporation suspension was determined. Paclitaxel concentration was determined by RP-HPLC, and trastuzumab concentration was determined by SEC-HPLC. Results are shown in Table 4.

TABLE 4
Paclitaxel and Trastuzumab Concentration
in Post-Evaporation Suspension

| | Paclitaxel | Trastuzumab |
|---|---|---|
| Batch | concentration | concentration |
| 1 (Ab added with 10 minute | 24.8 mg/mL | 4.42 mg/mL |
| incubation prior to | | |
| evaporation) | | |
| 2 (Ab added immediately prior | 24.7 mg/mL | 4.17 mg/mL |
| to evaporation) | | |
| 3 (Ab added during | 29.5 mg/mL | 4.39 mg/mL |
| evaporation) | | |
| 4 (Ab added to | 22.0 mg/mL | 4.87 mg/mL |
| post-evaporation) | | |
| 5 (control) | 24.8 mg/mL | 0.00 mg/mL |

2 mL of the post-evaporation suspension was centrifuged to pellet the particles, and the supernatant was removed to separate unbound trastuzumab from the nanoparticles. The pellet was re-suspended in fresh 2 mL phosphate buffered saline (PBS). The concentration of the trastuzumab in the re-suspended nanoparticles was measured by SEC-HPLC and ELISA (Trastuzumab ELISA Assay Kit, #1G-AA105, Eagle Bioscience), with results shown in Table 5. Also shown in Table 5 is the percentage of trastuzumab in the post-evaporation suspension that is associated with the nanoparticles based on the concentration of trastuzumab in the post-evaporation suspension and the concentration of trastuzumab in the washed nanoparticle suspension (average of SEC-HPLC and ELISA measurements. Adding the trastuzumab immediately prior to evaporation and without incubation resulted in the highest portion of the trastuzumab in the suspended associated with the nanoparticles at 5.76%.

TABLE 5
Trastuzumab Concentration in Washed Nanoparticle Suspension

| | | | Percentage |
|---|---|---|---|
| | | | Trastuzumab |
| | Trastuzumab | Trastuzumab | in Suspension |
| | concentration | concentration | Associated with |
| Batch | (SEC-HPLC) | (ELISA) | Nanoparticles |
| 1 (Ab added with 10 | 0.13 mg/mL | 0.15 mg/mL | 3.17% |
| minute incubation | | | |
| prior to evaporation) | | | |
| 2 (Ab added | 0.24 mg/mL | 0.24 mg/mL | 5.76% |
| immediately prior to | | | |
| evaporation) | | | |
| 3 (Ab added during | 0.15 mg/mL | 0.17 mg/mL | 3.64% |
| evaporation) | | | |
| 4 (Ab added to | 0.16 mg/mL | 0.18 mg/mL | 3.49% |
| post-evaporation) | | | |
| 5 (control) | 0.00 mg/mL | 0.00 mg/mL | N/A |

### Example 13: Manufacture of Nanoparticles with Embedded Trastuzumab, Paclitaxel, and Albumin by Adding Trastuzumab to Aqueous Solution Prior to Homogenization

Trastuzumab (Herceptin®) was reconstituted in water for injection (WFI) to a concentration of 21 mg/mL. 7.14 mL of the 21 mg/mL trastuzumab was mixed with 5 mL of 200 mg/mL human serum albumin (HSA), 2.86 mL water, and 5 mL 0.9% NaCl normal saline to form a solution of 50 mg/mL HSA and 7.5 mg/mL trastuzumab. 18.4 mL of the HSA/trastuzumab solution was mixed with 1.6 mL of a 200 mg/mL paclitaxel solution (dissolved in 90/10 CHCl₃/ethanol) to form a mixture. The mixture was transferred to an Avestin homogenizer and homogenized at 20-22 kpsi for several cycles to form a fine emulsion. 15 mL of 50 mg/mL HSA solution was added to the homogenizer to chase the fine emulsion, and 25.5 mL of the fine emulsion was collected. The fine emulsion was immediately transferred to a rotary evaporator to obtain a post-evaporation volume of 7.3 mL.

Dynamic light scattering (DLS) was used to measure the average particle diameter (Z-average) and the polydispersity index (PDI) of the post-evaporation suspension. The DLS measurements were performed using a Malvern Zetasizer Nano ZS (Malvern Instruments, Westborough, Mass.) by diluting 20 μL of the post-evaporation suspension with 1.5 mL 0.9% NaCl normal saline. The average particle diameter for the post-evaporation suspension was determined to be 147.8 nm, and the PDI was determined to be 0.131.

The paclitaxel concentration of the post-evaporation solution was determined by RP-HPLC to be 25.88 mg/mL. The total HSA concentration and trastuzumab concentration were measured by SEC-HPLC as 117 mg/mL and 16.2 mg/mL, respectively.

The post-evaporation suspension was frozen at −20° C. overnight before being thawed an allowed to equilibrate to room temperature. Dynamic light scattering (DLS) was again used to measure the average particle diameter (Z-average) and the polydispersity index (PDI) of the post-evaporation suspension using either 0.9% NaCl normal saline or WFI as a diluent. When 0.9% NaCl normal saline was used as a diluent, the average particle diameter was determined to be 147.8 nm, and the PDI was determined to be 0.145. When WFI was used as a diluent, the average particle diameter was determined to be 2354 nm, and the PDI was determined to be 0.500.

The thawed post-evaporation suspension was diluted with 2.7 mL of WFI to a final volume of 10 mL. 8 mL (divided in two) of the diluted post-evaporation suspension was centrifuged at 39,000 RPM at 20° C. using a Beckman Optima LE-80 centrifuge with a Ti45 rotor and Teflon inserts for 70 minutes to pellet the nanoparticles. The top 3 mL of supernatant was withdrawn as Supernatant 1, and the bottom ˜1 mL of supernatant was withdrawn as Supernatant 2. The pellet was washed three times with 4.0 mL of phosphate buffered saline (PBS) as Wash 1, Wash 2, and Wash 3. The pellet in the first centrifuge tube was resuspended in 3.0 mL ethanol, vortexed, and sonicated to extract the paclitaxel from the pellet. The sample was again centrifuged, and the supernatant was withdrawn as Pellet 1. The pellet in the second centrifuge tube was resuspended in 4.0 mL PBS, vortexed, sonicated, and withdrawn as Pellet 2. The samples were analyzed for paclitaxel content by RP-HPLC, and for HSA or trastuzumab content by SEC-HPLC. These results are shown in Table 6.

TABLE 6
HSA, Trastuzumab (Tz), and Paclitaxel (PTX) in the Post-Evaporation (PE)
Suspension and Associated with the Nanoparticles (NPs) by Centrifuge Analysis

| | Diluted | | | | | | | |
|---|---|---|---|---|---|---|---|---|
| | PE | Supernatant 1 | Supernatant 2 | Wash 1 | Wash 2 | Wash 3 | Pellet 1 | Pellet 2 |
| PTX | 148.7 | 0.7 | 0.8 | 2.8 | 3.2 | 3.1 | 131.4 | n.d. |
| (mg) | | | | | | | | |
| HSA | 574.4* | 365.7 | 178.6 | 5.4 | 1.5 | 1.0 | n.d. | 22.4 |
| (mg) | | | | | | | | |
| Tz | 81.8* | 51.4 | 27.6 | 0.84 | 0.2 | 0.1 | n.d. | 1.8 |
| (mg) | | | | | | | | |

*= Total determined from sum of Supernatant 1, Supernatant 2, Wash 1, Wash 2, Wash 3, and Pellet 2, based on a 8 mL processed sample.

### Example 14: Manufacture of Nanoparticles with Embedded Trastuzumab, Paclitaxel, and Albumin by Adding Trastuzumab to the Emulsion after Homogenization

18.4 mL of 50 mg/mL human serum albumin (HSA) was mixed with 1.6 mL of a 200 mg/mL paclitaxel solution (dissolved in 90/10 CHCl₃/ethanol) to form a mixture. The mixture was transferred to an Avestin homogenizer and homogenized at 20-22 kpsi for several cycles to form a fine emulsion. 15 mL of 50 mg/mL HSA solution was added to the homogenizer to chase the fine emulsion, and 22 mL of the fine emulsion was collected. The collected fine emulsion was immediately transferred to a round bottom flask containing 7.15 mL of 21 mg/mL trastuzumab (Herceptin® dissolved in water for injection (WFI)) and 5 mL 0.9% NaCl normal saline. The round bottom flask was transferred to a rotary evaporator, where the volume of the emulsion was reduced to 10 mL of post-evaporation suspension.

Dynamic light scattering (DLS) was used to measure the average particle diameter (Z-average) and the polydispersity index (PDI) of the post-evaporation suspension. The DLS measurements were performed using a Malvern Zetasizer Nano ZS (Malvern Instruments, Westborough, Mass.). 1.5 mL 0.9% NaCl normal saline diluent was mixed with 20 μL of the post-evaporation suspension in a 1 cm square disposable cuvette. Measurements were taken at 25° C. after a 2 minute equilibration with a detection angle of 173° using an instrument-selected attenuator level and fixed measurement position of 1.15 mm Measurements were taken in triplicate, with 60 second durations for each measurement. Size distributions were calculated using a general analysis model, a particle refractive index of 1.465+0i, a dispersant viscosity of 0.8872 cP, and a dispersant refractive index of 1.330+0i. The average particle diameter (Z-average) for the post-evaporation suspension was determined to be 141.6 nm, and the PDI was determined to be 0.110.

The paclitaxel concentration of the post-evaporation suspension was determined by RP-HPLC to be 18.18 mg/mL. The total HSA concentration and trastuzumab concentration were measured by SEC-HPLC as 76 mg/mL and 15.6 mg/mL, respectively.

The post-evaporation suspension was frozen at −20° C. overnight before being thawed an allowed to equilibrate to room temperature. Dynamic light scattering (DLS) was again used to measure the average particle diameter (Z-average) and the polydispersity index (PDI) of the post-evaporation suspension using either 0.9% NaCl normal saline or WFI as a diluent. When 0.9% NaCl normal saline was used as a diluent, the average particle diameter was determined to be 142.2 nm, and the PDI was determined to be 0.125. When WFI was used as a diluent, the average particle diameter was determined to be 2295 nm, and the PDI was determined to be 0.587.

The thawed post-evaporation suspension was diluted with 2.7 mL WFI to a final volume of 10 mL. 8 mL (divided in two) of the diluted post-evaporation suspension was centrifuged at 39,000 RPM at 20° C. using a Beckman Optima LE-80 centrifuge with a Ti45 rotor and Teflon inserts for 70 minutes to pellet the nanoparticles. The top 3 mL of supernatant was withdrawn as Supernatant 1, and the bottom ˜1 mL of supernatant was withdrawn as Supernatant 2. The pellet was washed three times with 4.0 mL of phosphate buffered saline (PBS) as Wash 1, Wash 2, and Wash 3. The pellet from a first centrifuge tube was resuspended in 3.0 mL ethanol, vortexed, and sonicated to extract the paclitaxel from the pellet. The sample was again centrifuged, and the supernatant was withdrawn as Pellet 1. The pellet from the second centrifuge tube was resuspended in 4.0 mL PBS, vortexed, sonicated, and withdrawn as Pellet 2. The samples were analyzed for paclitaxel content by RP-HPLC, and for HSA or trastuzumab content by SEC-HPLC. These results are shown in Table 7.

TABLE 7
HSA, Trastuzumab (Tz), and Paclitaxel (PTX) in the Post-Evaporation (PE)
Suspension and Associated with the Nanoparticles (NPs) by Centrifuge Analysis

| | PE | Supernatant 1 | Supernatant 2 | Wash 1 | Wash 2 | Wash 3 | Pellet 1 | Pellet 2 |
|---|---|---|---|---|---|---|---|---|
| PTX | 146.4 | 0.7 | 1.2 | 2.9 | 3.1 | 3.3 | 135.6 | n.d. |
| (mg) | | | | | | | | |
| HSA | 589.8* | 386.8 | 168.6 | 5.9 | 1.7 | 0.9 | n.d. | 25.9 |
| (mg) | | | | | | | | |
| Tz | 123.0* | 80.4 | 38.9 | 1.3 | 0.3 | 0.0 | n.d. | 2.1 |
| (mg) | | | | | | | | |

*= Total determined from sum of Supernatant 1, Supernatant 2, Wash 1, Wash 2, Wash 3, and Pellet 2, based on a 8 mL processed sample.

### Example 15: Manufacture of Nanoparticles with Embedded Trastuzumab, Paclitaxel, and Albumin by Adding Trastuzumab to the Post-Evaporation Suspension

18.4 mL of 50 mg/mL human serum albumin (HSA) was mixed with 1.6 mL of a 200 mg/mL paclitaxel solution (dissolved in 90/10 CHCl₃/ethanol) to form a mixture. The mixture was transferred to an Avestin homogenizer and homogenized at 20-22 kpsi for several cycles to form a fine emulsion. 15 mL of 50 mg/mL HSA solution was added to the homogenizer to chase the fine emulsion, and 20 mL of the fine emulsion was collected. The collected fine emulsion was immediately transferred to a round bottom flask and processed using a rotary evaporator until a final post-evaporation suspension volume of 7.9 mL was obtained.

Dynamic light scattering (DLS) was used to measure the average particle diameter (Z-average) and the polydispersity index (PDI) of the post-evaporation suspension. The DLS measurements were performed using a Malvern Zetasizer Nano ZS (Malvern Instruments, Westborough, Mass.) by diluting 150 μL of the post-evaporation suspension with 1.5 mL 0.9% NaCl normal saline. The average particle diameter (Z-average) for the post-evaporation suspension was determined to be 140.2 nm, and the PDI was determined to be 0.088.

The paclitaxel concentration of the post-evaporation suspension was determined by RP-HPLC to be 20.73 mg/mL. The HSA concentration of the post-evaporation suspension was measured by SEC-HPLC as 77.7 mg/mL

The post-evaporation suspension was frozen at −20° C. overnight before being thawed an allowed to equilibrate to room temperature. Dynamic light scattering (DLS) was again used to measure the average particle diameter (Z-average) and the polydispersity index (PDI) of the post-evaporation suspension. The average particle diameter was determined to be 132.3 nm, and the PDI was determined to be 0.094.

The post-evaporation suspension was again frozen at −20° C. overnight before being thawed an allowed to equilibrate to room temperature. A milky-white color to the suspension was observed upon thawing. The paclitaxel concentration of the thawed post-evaporation suspension was determined by RP-HPLC to be 20.70 mg/mL. 3.47 mL of 200 mg/mL HSA and 11.37 mL of 21.5 mg/mL trastuzumab (Herceptin® in 0.9% NaCl normal saline) was added to the 7.9 mL of post-evaporation suspension. The mixture was allowed to incubate at room temperature for one hour. Dynamic light scattering (DLS) was again used to measure the average particle diameter (Z-average) and the polydispersity index (PDI) of the suspension with trastuzumab. The average particle diameter was determined to be 139.9 nm, and the PDI was determined to be 0.105. The paclitaxel concentration of the thawed suspension was determined by RP-HPLC to be 7.2 mg/mL.

The suspension with trastuzumab was filtered using a 1.2 μm filter. Dynamic light scattering (DLS) was again used to measure the average particle diameter (Z-average) and the polydispersity index (PDI) of the filtered suspension. The average particle diameter was determined to be 136.9 nm, and the PDI was determined to be 0.119. The paclitaxel concentration of the thawed suspension was determined by RP-HPLC to be 7.1 mg/mL. The filtered suspension was lyophilized as four 2 mL aliquots using a VirTis Genesis EL25 shelf lyophilizer (SP Industries, Gardiner, N.Y.) and stored at −80° C.

A first lyophilized aliquot of the filtered suspension was removed from −80° C., equilibrated to room temperature and reconstituted with 2 mL 0.9% NaCl normal saline and incubated at room temperature for 24 hours. Dynamic light scattering (DLS) was used to measure the average particle diameter (Z-average) and the polydispersity index (PDI) of the suspension with trastuzumab. The average particle diameter was determined to be 141.4 nm, and the PDI was determined to be 0.081.

A second lyophilized aliquot of the filtered suspension was removed from −80° C., equilibrated to room temperature and reconstituted with 2 mL 0.9% NaCl normal saline. After 10-15 minutes, dynamic light scattering (DLS) was used to measure the average particle diameter (Z-average) and the polydispersity index (PDI) of the suspension with trastuzumab. The average particle diameter was determined to be 141.8 nm, and the PDI was determined to be 0.112.

### Example 16: Manufacture of Nanoparticles with Embedded Trastuzumab, Paclitaxel, and Albumin by Adding Trastuzumab to Aqueous Solution Prior to Homogenization

A first batch of a nanoparticle composition was manufactured by including a solution containing 15 mg/mL human serum albumin (HSA) and 15 mg/mL trastuzumab in the aqueous solution of the initial mixture. A second batch of a nanoparticle composition was manufactured by including a solution containing 30 mg/mL human serum albumin (HSA) and 15 mg/mL trastuzumab in the aqueous solution of the initial mixture. As a control, a third batch of a nanoparticle composition was manufactured using 15 mg/mL HSA and no trastuzumab.

Batch 1. 4.01 mL water for injection (WFI), 1.425 mL of 200 mg/mL human serum albumin (HSA), 13.57 mL of 21 mg/mL trastuzumab (Herceptin® in water for injection (WFI), and 87.3 mg NaCl were combined to obtain 19 mL of a solution containing 15 mg/mL HSA and 15 mg/mL trastuzumab. 1.6 mL of 200 mg/mL paclitaxel (dissolved in 90/10 CHCl₃/ethanol) was added to 18.4 mL of the HSA/trastuzumab solution to form a mixture. The mixture was transferred to an Avestin homogenizer and homogenized at 20-22 kpsi for several cycles to form a fine emulsion. 15 mL of WFI was added to the homogenizer to chase the fine emulsion, and 20 mL of the fine emulsion was collected. The fine emulsion was processed by a rotary evaporator until the volume of the resulting post-evaporation suspension was 5.6 mL. Dynamic light scattering (DLS) was used to measure the average particle diameter (Z-average) and the polydispersity index (PDI) of the post-evaporation suspension. The DLS measurements were performed using a Malvern Zetasizer Nano ZS (Malvern Instruments, Westborough, Mass.) by diluting 50 μL of the post-evaporation suspension with 1.5 mL 0.9% NaCl normal saline. The average particle diameter for the post-evaporation suspension was determined to be 134.8 nm, and the PDI was determined to be 0.084.

Batch 2. 85.5 mg NaCl was dissolved in 2.85 mL water for injection (WFI) to form a 3% NaCl saline solution. 2.85 mL of 200 mg/mL human serum albumin (HSA) and 13.57 mL of 21 mg/mL trastuzumab (Herceptin® in water for injection (WFI) were combined with the 3% NaCl saline solution to obtain 19 mL of a solution containing 30 mg/mL HSA and 15 mg/mL trastuzumab. 1.6 mL of 200 mg/mL paclitaxel (dissolved in 90/10 CHCl₃/ethanol) was added to 18.4 mL of the HSA/trastuzumab solution to form a mixture. The mixture was transferred to an Avestin homogenizer and homogenized at 20-22 kpsi for several cycles to form an emulsion. 15 mL of 0.045% NaCl saline was added to the homogenizer to chase the fine emulsion, and 20 mL of the fine emulsion was collected. The fine emulsion was processed by a rotary evaporator until the volume of the resulting post-evaporation suspension was 5.4 mL. Dynamic light scattering (DLS) was used to measure the average particle diameter (Z-average) and the polydispersity index (PDI) of the post-evaporation suspension as described for Batch 1. The average particle diameter for the post-evaporation suspension was determined to be 153.8 nm, and the PDI was determined to be 0.124.

Batch 3. 18.4 mL of 15 mg/mL HSA was mixed with 1.6 mL of 200 mg/mL paclitaxel (dissolved in 90/10 CHCl₃/ethanol) to form a mixture. The mixture was transferred to an Avestin homogenizer and homogenized at 20-22 kpsi for several cycles to form an emulsion. 15 mL of 15 mg/mL was added to the homogenizer to chase the fine emulsion, and 20 mL of the fine emulsion was collected. The fine emulsion was processed by a rotary evaporator until the volume of the resulting post-evaporation suspension was 7.2 mL. Dynamic light scattering (DLS) was used to measure the average particle diameter (Z-average) and the polydispersity index (PDI) of the post-evaporation suspension as described for Batch 1. The average particle diameter for the post-evaporation suspension was determined to be 142.9 nm, and the PDI was determined to be 0.133.

Paclitaxel concentration of the post-evaporation suspension from Batch 1, Batch 2, and Batch 3 was measured by RP-HPLC. The concentration of paclitaxel in the post-evaporation suspension from Batch 1 was 29.19 mg/mL, from Batch 2 was 35.59 mg/mL, and from Batch 3 was 22.77 mg/mL. The post-evaporation suspension from each batch was diluted with 0.9% NaCl normal saline to reach a paclitaxel concentration of 7.00 mg/mL.

8 mL of the diluted post-evaporation suspension from each batch was divided into two centrifuged tubes. The unused portions were frozen at −20° C. The samples were centrifuged at 39,000 RPM at 20° C. using a Beckman Optima LE-80 centrifuge with a Ti45 rotor and Teflon inserts for 70 minutes to pellet the nanoparticles. The top 3 mL of supernatant was from each sample withdrawn as Supernatant 1, and the bottom ˜1 mL of supernatant was withdrawn as Supernatant 2. The pellets were washed twice with 4.0 mL of phosphate buffered saline (PBS) as Wash 1 and Wash 2. The pellets from the first of the two centrifuge tubes were resuspended in 3.0 mL ethanol, vortexed, and sonicated to extract the paclitaxel from the pellet. The samples were again centrifuged, and the supernatant was withdrawn as Pellet 1. The pellets from the second of the two centrifuge tubes were resuspended in 4.0 mL PBS, vortexed, sonicated, and withdrawn as Pellet 2. The samples were analyzed for paclitaxel content by RP-HPLC, and for HSA or trastuzumab content by SEC-HPLC. These results are shown in Tables 8 and 9.

TABLE 8
Batch 1 - HSA, Trastuzumab (Tz), and Paclitaxel (PTX) in the Post-Evaporation (PE)
Suspension and Associated with the Nanoparticles (NPs) by Centrifuge Analysis

| | Diluted | | | | | | |
|---|---|---|---|---|---|---|---|
| | PE | Supernatant 1 | Supernatant 2 | Wash 1 | Wash 2 | Pellet 1 | Pellet 2 |
| PTX | 56.0* | 0.2 | 0.8 | 0.8 | 1.2 | 64 | n.d. |
| (mg) | | | | | | | |
| HSA | 68.2** | 31.5 | 22.4 | 1.0 | 0.6 | n.d. | 12.7 |
| (mg) | | | | | | | |
| Tz | 87.0** | 48.6 | 36.8 | 0.9 | 0.1 | n.d. | 0.7 |
| (mg) | | | | | | | |

*Based on 7.00 mg/mL Paclitaxel for 8 mL processed sample.
**= Total determined from sum of Supernatant 1, Supernatant 2, Wash 1, Wash 2, and Pellet 2, based on a 8 mL processed sample.

TABLE 9
Batch 2 - HSA, Trastuzumab (Tz), and Paclitaxel (PTX) in the Post-Evaporation (PE)
Suspension and Associated with the Nanoparticles (NPs) by Centrifuge Analysis

| | Diluted | | | | | | |
|---|---|---|---|---|---|---|---|
| | PE | Supernatant 1 | Supernatant 2 | Wash 1 | Wash 2 | Pellet 1 | Pellet 2 |
| PTX | 56.0* | 0.1 | 1.8 | 1.1 | 0.7 | 47.1 | n.d. |
| (mg) | | | | | | | |
| HSA | 93.4** | 52.0 | 33.1 | 1.5 | 0.4 | n.d. | 6.3 |
| (mg) | | | | | | | |
| Tz | 59.7** | 33.5 | 25.1 | 0.8 | 0.0 | n.d. | 0.3 |
| (mg) | | | | | | | |

*Based on 7.00 mg/mL Paclitaxel for 8 mL processed sample.
** = Total determined from sum of Supernatant 1, Supernatant 2, Wash 1, Wash 2, and Pellet 2, based on a 8 mL processed sample.

### Example 17: Manufacture of Nanoparticles with Embedded Trastuzumab, Paclitaxel, and Albumin by Adding Trastuzumab Prior to Lyophilization

36.8 mL of 50 mg/mL human serum albumin (HSA) was mixed with 3.2 mL of a 200 mg/mL paclitaxel solution (dissolved in 90/10 CHCl₃/ethanol) to form a mixture. The mixture was transferred to an Avestin homogenizer and homogenized at 20-22 kpsi for several cycles to form a fine emulsion. 20 mL of 50 mg/mL HSA solution was added to the homogenizer to chase the fine emulsion, and 43 mL of the fine emulsion was collected. The emulsion was immediately transferred to a rotary evaporator, and the volume was reduced to a post-evaporation suspension volume of 11.5 mL. Dynamic light scattering (DLS) was used to measure the average particle diameter (Z-average) and the polydispersity index (PDI) of the post-evaporation suspension. The DLS measurements were performed using a Malvern Zetasizer Nano ZS (Malvern Instruments, Westborough, Mass.) by diluting 50 μL of the post-evaporation suspension with 1.5 mL 0.9% NaCl normal saline. The average particle diameter for the post-evaporation suspension was determined to be 149.1 nm, and the PDI was determined to be 0.151. The paclitaxel concentration of the post-evaporation suspension was determined to be 35.79 mg/mL by RP-HPLC, and the HSA concentration of the post-evaporation suspension was determined to be 126.0 mg/mL by SEC-HPLC. The post-evaporation suspension was further processed in two separate batches, as follows.

Batch 1. 4.60 mL of the post evaporation suspension was mixed with 0.888 mL of 181.63 mg/mL HSA (in water for injection (WFI)) and 10.98 mL of 2.7 mg/mL NaCl to form a final solution of 16.46 mL containing a paclitaxel:albumin ratio of 1:4.5 (and no trastuzumab).

Batch 2. 4.60 mL of the post evaporation suspension was mixed with 0.888 mL of 181.63 mg/mL HSA (in WFI) and 10.98 mL of 22.5 mg/mL trastuzumab (Herceptin® reconstituted in 2.7 mg/mL NaCl) to form a final solution of 16.46 mL containing a paclitaxel:albumin:trastuzumab ratio of 1:4.5:1.5.

4 mL aliquots of batch 1 or batch 2 were dispensed into vials and lyophilized using a VirTis Genesis EL25 shelf lyophilizer (SP Industries, Gardiner, N.Y.).

### Example 18: Manufacture of Nanoparticles with Embedded Trastuzumab, Paclitaxel, and Albumin by Adding Trastuzumab Prior to Lyophilization

36.8 mL of 15 mg/mL human serum albumin (HSA) was mixed with 3.2 mL of a 200 mg/mL paclitaxel solution (dissolved in 90/10 CHCl₃/ethanol) to form a mixture. The mixture was transferred to an Avestin homogenizer and homogenized at 20-22 kpsi for several cycles to form a fine emulsion. 20 mL of 15 mg/mL HSA solution was added to the homogenizer to chase the fine emulsion, and about 40 mL of the fine emulsion was collected. The emulsion was immediately transferred to a rotary evaporator, and the volume was reduced to a post-evaporation suspension volume of 9.9 mL. Dynamic light scattering (DLS) was used to measure the average particle diameter (Z-average) and the polydispersity index (PDI) of the post-evaporation suspension. The DLS measurements were performed using a Malvern Zetasizer Nano ZS (Malvern Instruments, Westborough, Mass.) by diluting 50 μL of the post-evaporation suspension with 1.5 mL 0.9% NaCl normal saline. The average particle diameter for the post-evaporation suspension was determined to be 161.8 nm, and the PDI was determined to be 0.118. The paclitaxel concentration of the post-evaporation suspension was determined to be 42.43 mg/mL by RP-HPLC, and the HSA concentration of the post-evaporation suspension was determined to be 50.33 mg/mL by SEC-HPLC. The post-evaporation suspension was further processed in two separate batches, as follows.

Batch 1. 3.90 mL of the post-evaporation suspension was mixed with 1.62 mL of 32.14 mg/mL HSA (in water for injection (WFI)) and 11.03 mL of 2.7 mg/mL NaCl to form a final solution of 16.55 mL containing a paclitaxel:albumin ratio of 1:4.5 (and no trastuzumab).

Batch 2. 3.90 mL of the post evaporation suspension was mixed with 1.62 mL of 32.14 mg/mL HSA (in WFI) and 11.03 mL of 22.5 mg/mL trastuzumab (Herceptin® reconstituted in 2.7 mg/mL NaCl) to form a final solution of 16.55 mL containing a paclitaxel:albumin:trastuzumab ratio of 1:1.5:1.5.

4 mL aliquots of batch 1 or batch 2 were dispensed into vials and lyophilized using a VirTis Genesis EL25 shelf lyophilizer (SP Industries, Gardiner, N.Y.).

### Example 19: Nanoparticles with Embedded Trastuzumab, Paclitaxel, and Albumin Manufactured by Adding Trastuzumab Prior to Lyophilization Compared to Timecourse Admixtures of Nab-Paclitaxel and Trastuzumab

Lyophilized nanoparticles with the characteristics described in Table 10 were used for this Example.

TABLE 10
Lyophilized Samples for Example 18

| Sample | Manufacture | Paclitaxel | Albumin | Trastuzumab | NaCl | No. Vials |
|---|---|---|---|---|---|---|
| 1 | Example 17, | 40 mg/vial | 180 mg/vial | 0 mg/vial | 7.2 mg/vial | 4 |
| | Batch 1 | | | | | |
| 2 | Example 17, | 40 mg/vial | 180 mg/vial | 60 mg/vial | 7.2 mg/vial | 4 |
| | Batch 2 | | | | | |
| 3 | Example 18, | 40 mg/vial | 60 mg/vial | 0 mg/vial | 7.2 mg/vial | 4 |
| | Batch 1 | | | | | |
| 4 | Example 18, | 40 mg/vial | 60 mg/vial | 60 mg/vial | 7.2 mg/vial | 4 |
| | Batch 2 | | | | | |

4 mL of 15 mg/mL trastuzumab (Herceptin®) dissolved in 5.4 mg/mL NaCl was added to the vials from Sample 1 and Sample 3. The samples were incubated for 10 minutes before characterizing and centrifuging the sample, incubated for 70 minutes before characterizing and centrifuging the sample, or incubated for 250 minutes before characterizing and centrifuging the sample. 4 mL of 7.2 mg/mL NaCl was added to Sample 2 and Sample 4. The samples were incubated at room temperature for 10 minutes before being characterized and centrifuged. Characterization of the sample included determining osmolality by freezing point depression (Advanced Micro Osmometer Model 3300 (Advanced Instruments, Inc.)), average particle size (diameter) and polydispersity index (PDI) by dynamic light scattering (Zetasizer nano ZS, Malvern Instruments, Westborough, Mass.), paclitaxel content (RP-HPLC), and HSA and trastuzumab (Tz) content (SEC-HPLC). Particle size (Z-average diameter), PDI, and osmolality are shown in Table 11.

TABLE 11
Particle Size, PDI, and Osmolality of Reconstituted Samples

| | | Z-Avg | | |
|---|---|---|---|---|
| Sample | Incubation Time | Diameter | PDI | Osmolality |
| 1 | Pre-lyophilization | 149.1 nm | 0.151 | n.d. |
| | 10 min | 146.8 nm | 0.105 | 401 mOsm/kg |
| | 70 min | 149.0 nm | 0.116 | 405 mOsm/kg |
| | 250 min | 149.2 nm | 0.125 | 398 mOsm/kg |
| 2 | Pre-lyophilization | 149.1 nm | 0.151 | n.d. |
| | 10 | 148.9 nm | 0.139 | 401 mOsm/kg |
| 3 | Pre-lyophilization | 161.8 nm | 0.118 | n.d. |
| | 10 min | 162.8 nm | 0.121 | 364 mOsm/kg |
| | 70 min | 164.1 nm | 0.152 | 367 mOsm/kg |
| | 250 min | 162.4 nm | 0.132 | 359 mOsm/kg |
| 4 | Pre-lyophilization | 161.8 nm | 0.118 | n.d. |
| | 10 min | 162.9 | 0.164 | 354 mOsm/kg |

For the centrifugation analysis, 3.6 mL of the samples were centrifuged at 39,000 RPM at 20° C. using a Beckman Optima LE-80 centrifuge with a Ti45 rotor and Teflon inserts for 70 minutes to pellet the nanoparticles. The supernatant was from each sample withdrawn. The pellets were washed twice with 4.0 mL of phosphate buffered saline (PBS) as Wash 1 and Wash 2. The pellet were resuspended in 3.0 mL ethanol, vortexed, and sonicated to extract the paclitaxel from the pellet. The samples were again centrifuged, and the supernatant was withdrawn as Pellet 1. The pellets in the other centrifuge tubes were resuspended in 4.0 mL PBS, vortexed, sonicated, and withdrawn as Pellet 2. The amount of paclitaxel in each fraction was determined by RP-HPLC, and the amount of HSA and trastuzumab (Tz) in each fraction was determined by SEC-HPLC. Results are shown in Table 12.

TABLE 12
Paclitaxel (PTX), Albumin (HSA), and Trastuzumab in Each Fraction of
Centrifugation Analysis

| | | Incubation | | | |
|---|---|---|---|---|---|
| Fraction | Sample | time (min) | PTX (mg) | HSA (mg) | Tz (mg) |
| Pre-centrifuge | 1 | 10 | 32.3 | 145.0 | 62.3* |
| suspension | | 70 | 35.6 | 158.1 | 62.4* |
| | | 250 | 57.0 | 233.1 | 59.8* |
| | 2 | 10 | 32.5 | 142.2 | 61.2* |
| | 3 | 10 | 19.8 | 32.4 | 63.4 |
| | | 70 | 35.4 | 53.7 | 64.9 |
| | | 250 | 59.3 | 83.9 | 61.9 |
| | 4 | 10 | 30.5 | 49.8 | 62.3 |
| Supernatant | 1 | 10 | 0.6 | 137.8 | 64.8 |
| | | 70 | 0.6 | 156.2 | 64.1 |
| | | 250 | 0.4 | 162.5 | 68.6 |
| | 2 | 10 | 1.6 | 147.9 | 61.3 |
| | 3 | 10 | 0.7 | 47.2 | 61.6 |
| | | 70 | 1.8 | 45.6 | 59.7 |
| | | 250 | 0.3 | 49.7 | 69.5 |
| | 4 | 10 | 0.8 | 45.2 | 60.4 |
| Wash 1 | 1 | 10 | 0.5 | 0.8 | 0.4 |
| | | 70 | 0.6 | 0.9 | 0.4 |
| | | 250 | 0.3 | 1.1 | 0.6 |
| | 2 | 10 | 0.8 | 0.8 | 0.3 |
| | 3 | 10 | 1.7 | 0.9 | 0.8 |
| | | 70 | 1.6 | 0.6 | 0.4 |
| | | 250 | 0.3 | 0.6 | 0.9 |
| | 4 | 10 | 1.6 | 0.7 | 0.5 |
| Wash 2 | 1 | 10 | 0.5 | n.d. | n.d. |
| | | 70 | 0.6 | n.d. | n.d. |
| | | 250 | 0.4 | n.d. | n.d. |
| | 2 | 10 | 0.7 | n.d. | n.d. |
| | 3 | 10 | 0.7 | n.d. | n.d. |
| | | 70 | 0.7 | n.d. | n.d. |
| | | 250 | 0.8 | n.d. | n.d. |
| | 4 | 10 | 0.8 | n.d. | n.d. |
| Pellet 1 | 1 | 10 | 32.4 | n.d. | n.d. |
| (Ethanol | | 70 | 34.7 | n.d. | n.d. |
| extraction) | | 250 | 34.7 | n.d. | n.d. |
| | 2 | 10 | 32.2 | n.d. | n.d. |
| | 3 | 10 | 32.4 | n.d. | n.d. |
| | | 70 | 33.1 | n.d. | n.d. |
| | | 250 | 34.2 | n.d. | n.d. |
| | 4 | 10 | 31.2 | n.d. | n.d. |
| Pellet 2 | 1 | 10 | 0.7 | 6.0 | 0.5* |
| | | 70 | 0.8 | 6.3 | 0.4* |
| | | 250 | 0.8 | 6.3 | 0.5* |
| | 2 | 10 | 0.7 | 5.6 | 0.4 |
| | 3 | 10 | 0.5 | 3.9 | 0.3 |
| | | 70 | 0.5 | 3.9 | 0.3 |
| | | 250 | 0.6 | 4.3 | 0.4 |
| | 4 | 10 | 0.5 | 3.8 | 0.3 |

*= Average of two measurements.

### Example 20: Manufacture of Nanoparticles with Embedded Trastuzumab, Paclitaxel, and Albumin by Adding Trastuzumab to Emulsion after Homogenization for In Vivo Administration

27.6 mL of a 15 mg/mL human serum albumin (HSA) solution (in water) was combined with 2.4 mL of a 200 mg/mL paclitaxel solution (90/10 mixture of CHCl₃ and ethanol) while mixing to form a mixture. The mixture was transferred to an Avestin homogenizer and homogenized at 20-22 kpsi. An additional 5 mL of 15 mg/mL HSA was used to wash the container holding the mixture, which was added to the homogenizer. The mixture was passed through the homogenizer for several cycles before an additional 20 mL of 15 mg/mL HSA as added to the homogenizer to chase the emulsion. A total of 42 mL of fine emulsion was collected. 1.2 mL of 21 mg/mL trastuzumab (Herceptin® in 0.9% NaCl saline) and 10 mL of 0.9% NaCl saline was combined with the fine emulsion before subjecting the fine emulsion to rotary evaporation. Once the volume of the post-evaporation suspension was reduced to about 10 mL, the post-evaporation suspension was transferred to a scintillation vial. The rotary evaporator flask was twice washed with 1.5 mL aliquots of water for injection (WFI), which were added to the scintillation vial, generating a final volume of 12.5 mL of post-evaporation suspension.

Dynamic light scattering (DLS) was used to measure the average particle diameter (Z-average) and the polydispersity index (PDI) of the post-evaporation suspension. The DLS measurements were performed using a Malvern Zetasizer Nano ZS (Malvern Instruments, Westborough, Mass.) by diluting 50 μL of the post-evaporation suspension with 1.5 mL 0.9% NaCl normal saline. The average particle diameter (Z-average) for the post-evaporation suspension was determined to be 149.1 nm, and the PDI was determined to be 0.133. The paclitaxel concentration of the post-evaporation suspension was determined to be 28.7 mg/mL by RP-HPLC, and the HSA and trastuzumab concentrations of the post-evaporation suspension were determined to be 41.61 mg/mL and 2.07 mg/mL, respectively, by SEC-HPLC. The post-evaporation suspension was then stored overnight at 5° C.

The post-evaporation was removed from cold storage and allowed to equilibrate to room temperature. 11.27 ml of 200 mg/mL HSA and 34.03 mL of 0.9% NaCl saline was mixed with 12 mL of post-evaporation suspension, resulting in a volume of 57.3 mL. The diluted suspension was filtered using a series of sterile filters, resulting in a post-filtration volume of 49.7 mL. The average particle diameter (Z-average) for the filtered suspension was determined to be 149.2 nm, and the PDI was determined to be 0.13, by DLS. The paclitaxel concentration of the filtered suspension was determined to be 5.693 mg/mL by RP-HPLC.

0.32 mL of 21 mg/mL trastuzumab (Herceptin®) and 6.56 mL 0.9% NaCl saline was added to 49.7 mL of the filtered suspension, resulting in 56.58 mL of a suspension containing 5 mg/mL paclitaxel and 0.5 mg/mL trastuzumab, and gently mixed and incubated for 10 minutes at room temperature. The calibrated, filtered suspension was again analyzed. The average particle diameter (Z-average) was determined to be 149 nm, and the PDI was determined to be 0.13, by DLS. The paclitaxel concentration of the filtered suspension was determined to be 5.4 mg/mL by RP-HPLC. Trastuzumab concentration was determined to be 0.5 mg/mL by SEC-HPLC. The osmolality was determined to be 288 mOsm. The suspension was dispensed into 3 mL aliquots and stored at −80° C.

### Example 21: Conjugation Formulation Characterization Methods

Immunoblots, SDS-PAGE gels, and ELISA assays in the following examples are conducted according to the protocols detailed in this Example, unless otherwise specified.

Native Gel Immunoblot

Materials used in this protocol included: sample buffer: Novex™ Tris-Glycine Native Sample Buffer (2×) (Cat# LC2673); running buffer: Novex™ Tris-Glycine Native Running Buffer (10×) (Cat# LC2672); protein gel: NuPAGE™ 3-8% Tris-Acetate Protein Gels, 1.0 mm, 10-well (Cat#EA0375BOX); and Western Blot kit: Trans-Blot Turbo RTA Mini Nitrocellulose Transfer kit (Cat#1704270).

Samples were diluted with PBS to a concentration range of 0.1-2 mg/μL. The diluted samples were mixed 1:1 (v/v) with tris-glycine native sample buffer and then vortexed briefly. A gel tank with one protein gel was prepared (two protein gels can be used based on the number of samples). The samples were loaded into the wells (about 16-20 μL per well). The gel was run at 200 V, 120 mA for 90 minutes. After running the gel, the gel was removed from its case and washed with water.

The proteins in the gel were transferred to a nitrocellulose membrane. The membrane was washed with TBST and incubated in 20 mL of Licor blocking solution for 1 hour at room temperature. The membrane was then washed three times with TBST and incubated overnight at 4° C. with anti-HSA (1:5000, mouse) and anti-human IgG (1:5000, rabbit) primary antibodies. (Optionally, this incubation could have been performed for 1 hour at room temperature.)

The membrane was washed three times with TBST (each time incubating for 5 minutes). The membrane was incubated with anti-mouse (1:20000, 680 nm) and anti-rabbit (1:20000, 800 nm) for 45 minutes at room temperature.

The membrane was washed three times with TB ST (each time incubating for 5 minutes). The membrane was washed with deionized water briefly and then imaged with Licor.

SDS-PAGE Immunoblot

Materials used in this protocol included: SDS running buffer: LDS sample buffer, Non-reducing (4×) (Cat#84788); gel running buffer: Novex™ MOPS SDS Running Buffer (20×) (Cat# NP0001); gel running buffer: Novex™ Tris-Acetate SDS Running Buffer (20×) (Cat# LA0041); protein gel: NuPAGE™ 3-8% Tris-Acetate Protein Gels, 1.0 mm, 10-well (Cat#EA0375BOX); protein gel: NuPAGE™ 4-12%, 1.0 mm, 10-well (Cat# NP0321PK2); and Western Blot kit: Trans-Blot Turbo RTA Mini Nitrocellulose Transfer kit (Cat#1704270).

Samples were diluted with PBS to a concentration range of 1-2 mg/mL. 250 mM NEM solution was prepared (31 μg NEM in 1 mL water). SDS-PAGE (non-reducing) master mix was prepared (60 μL of 1×SDS running buffer+100 μL 4×SDS load buffer+40 μL 250 mM NEM). The diluted protein samples were mixed 1:1 (v/v) with master mix and then vortexed briefly. The samples were incubated at 70° C. for 10 minutes.

A gel tank with one protein gel was prepared (two protein gels can be used based on the number of samples). The samples were loaded into the wells (about 16-20 μL per well). The gel was run at 200 V, 120 mA for 45 minutes. After running the gel, the gel was removed from its case and washed with water.

The proteins in the gel were transferred to a nitrocellulose membrane. The membrane was washed with TBST and incubated in 20 mL of Licor blocking solution for 1 hour at room temperature. The membrane was then washed three times with TBST and incubated overnight at 4° C. with anti-HSA (1:5000, mouse) and anti-human IgG (1:5000, rabbit) primary antibodies. (Optionally, this incubation could have been performed for 1 hour at room temperature.

The membrane was washed three times with TBST (each time incubating for 5 minutes). The membrane was incubated with anti-mouse (1:20000, 680 nm) and anti-rabbit (1:20000, 800 nm) for 45 minutes at room temperature.

The membrane was washed three times with TBST (each time incubating for 5 minutes). The membrane was washed with deionized water briefly and then imaged with Licor.

ELISA

Materials used in this protocol included: ELISA assay kit (#IG-AA105, Eagle Bioscience).

The provided standard samples were diluted to generate standards at 100 ng/mL, 60 ng/mL, 20 ng/mL, 6 ng/mL, and blank. Mab-conjugated particle solution was directly diluted by the dilution buffer to 1:1000 to 1:10000 according to different formats of samples.

100 μL of Assay Buffer was pipetted into each of the wells to be used. 100 μL of standard or sample was pipetted into the respective wells of the microtiter plate. The plate was covered with an adhesive seal and incubated for 60 minutes at room temperature.

The adhesive seal was removed and the incubation solution was decanted. The plate was washed with 3×300 μL of wash buffer per well. The excess solution was removed by tapping the inverted plate on a paper towel. 100 μL of enzyme conjugate was pipetted into each well. The plate was covered with an adhesive seal and incubated for 30 minutes at room temperature.

The adhesive seal was removed and the incubation solution was decanted. The plate was washed with 3×300 μL of wash buffer per well. The excess solution was removed by tapping the inverted plate on a paper towel. 100 μL of TMB substrate solution was pipetted into each well. The plate was covered with an adhesive seal and incubated in the dark for 20 minutes.

The adhesive seal was removed and 100 μL of stop solution was pipetted into each well. The absorbance was measured (450 nm) after pipetting the stop solution.

### Example 22: Conjugation of Trastuzumab and Free Human Serum Albumin Via SM(PEG)₆ Activated Trastuzumab

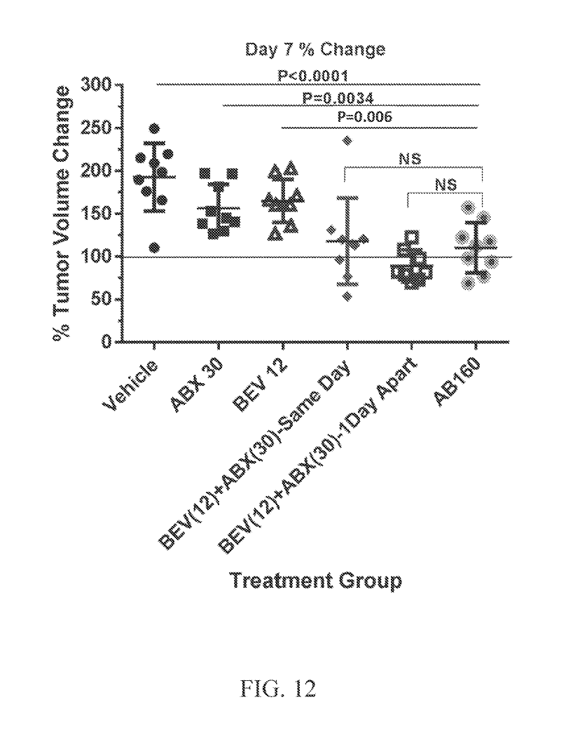

A schematic for conjugation of an antibody and free human serum albumin (HSA) described in this example is shown in FIG. 13. 1 mL of 21 mg/mL Herceptin® in normal saline was aliquoted into a 15 mL spin concentrator (MW cutoff 30,000 KDa). The spin concentrator was filled with 100 mM potassium phosphate buffer (pH 6.6) and centrifuged for 35 minutes at 3750 rpm. The spin concentrator was filled once more with phosphate buffer (pH 6.6) and centrifuged for 35 minutes at 3750 rpm. The resulting trastuzumab solution was pipetted into a Falcon tube and diluted to a total volume of 2 mL. The trastuzumab solution concentration was measured by size exclusion chromatography (SEC), and adjusted to 10.5 mg/mL as needed.

The SM(PEG)₆ package was removed from −20° C. and warmed at room temperature for 1 hour. 122 μL of dimethyl sulfoxide (DMSO) was added to 8.3 mg of SM(PEG)₆ (112 mM final concentration). The linker solution was vortexed until the linker was completely dissolved. 1 mL of the trastuzumab solution was aliquoted into Eppendorf tubes. To each tube, 3.4 μL of the linker solution was slowly added (stoichiometric ratio of linker:trastuzumab was 5:1). While still protecting the solution from light, the linker/trastuzumab solution was reacted on a shaker for 2 hours. 5 mL desalting columns were washed with phosphate buffer (pH 6.6) 4 times (1,000 RCF for 6 minutes for each centrifugation). The linker/trastuzumab solution was filtered through a desalting column to remove any unreacted linker. The activated trastuzumab solution was collected and 0.8 mL aliquots were pipetted into Eppendorf tubes. 200 μL of a 200 mg/mL (3 mM) human serum albumin (HSA) solution was diluted to 4 mL in a 4 mL spin concentrator (MW cutoff 10,000 kDa). The sample was centrifuged for 20 minutes according to manufacturer's instructions. This dilution and centrifugation was repeated once more. The resulting solution was pipetted into a Falcon tube and diluted to a final volume of 0.8 mL. 0.8 mL of activated trastuzumab solution (0.07 mM) was subsequently added to 0.8 mL of HSA solution (0.75 mM). The solution was incubated overnight.

Using a similar protocol, activated trastuzumab was further produced using various linker loading ratios and linker reaction conditions (such as different pH conditions of the conjugation reaction). SEC analysis was performed on an activated trastuzumab crosslinked with SM(PEG)₆ using phosphate buffer (pH 6.6) and 10× linker loading. At pH 6.6 and 10× linker loading, trastuzumab aggregation was less than about 2% of the total antibody amount. SEC analysis was also performed on an activated trastuzumab crosslinked with SM(PEG)₆ using PBS saline (pH 7.4) and 20× linker loading. At pH 7.4 and 20× linker loading, trastuzumab aggregation was about 30% of the total antibody amount.

SEC analysis was performed to compare SM(PEG)₆ activated trastuzumab and unconjugated trastuzumab. Conjugation did not significantly increase the amount of high molecular weight (HMW) species of trastuzumab (trastuzumab: 0.6% HMW of total antibody; SM(PEG)₆ activated trastuzumab: 1.7% HMW of total antibody) when the SM(PEG)₆ and trastuzumab were used at a 10:1 ratio.

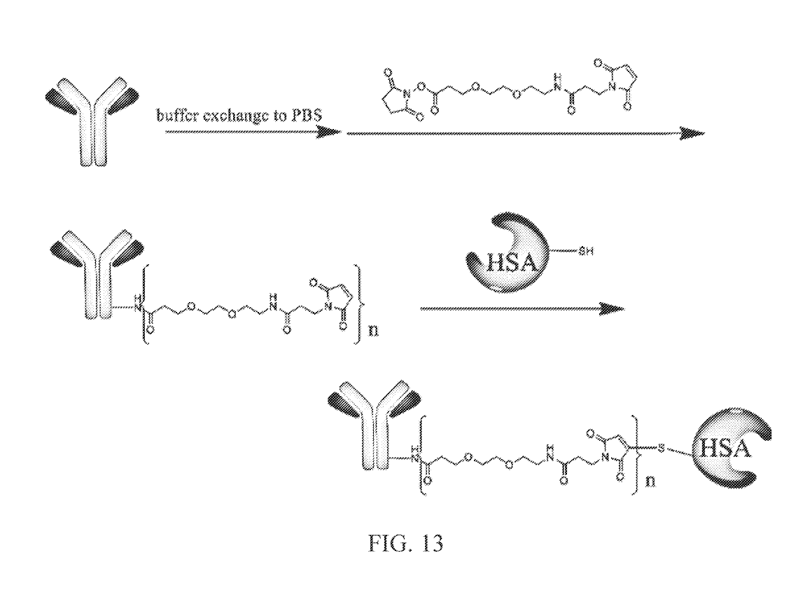

FIGS. 14A-14C show deconvoluted mass spectra of trastuzumab (FIG. 14A), SM(PEG)₆ activated trastuzumab using a trastuzumab:linker ratio of 1:5 (FIG. 14B), and SM(PEG)₆ activated trastuzumab using a trastuzumab:linker ratio of 1:10 (FIG. 14C). The isotope cluster representing trastuzumab without SM(PEG)₆ conjugation is indicated (1:0; trastuzumab:SM(PEG)₆), and it was observed that the N-glycan on trastuzumab was not cleaved (FIG. 14A). Using a trastuzumab:linker ratio of 1:5, the most represented species of activated trastuzumab are 1:0, 1:1, 1:2, and 1:3 (trastuzumab:SM(PEG)₆) (FIG. 14B). Using a trastuzumab:linker ratio of 1:10, the most represented species of activated trastuzumab are 1:1, 1:2, 1:3, and 1:4 (trastuzumab:SM(PEG)₆) (FIG. 14C).

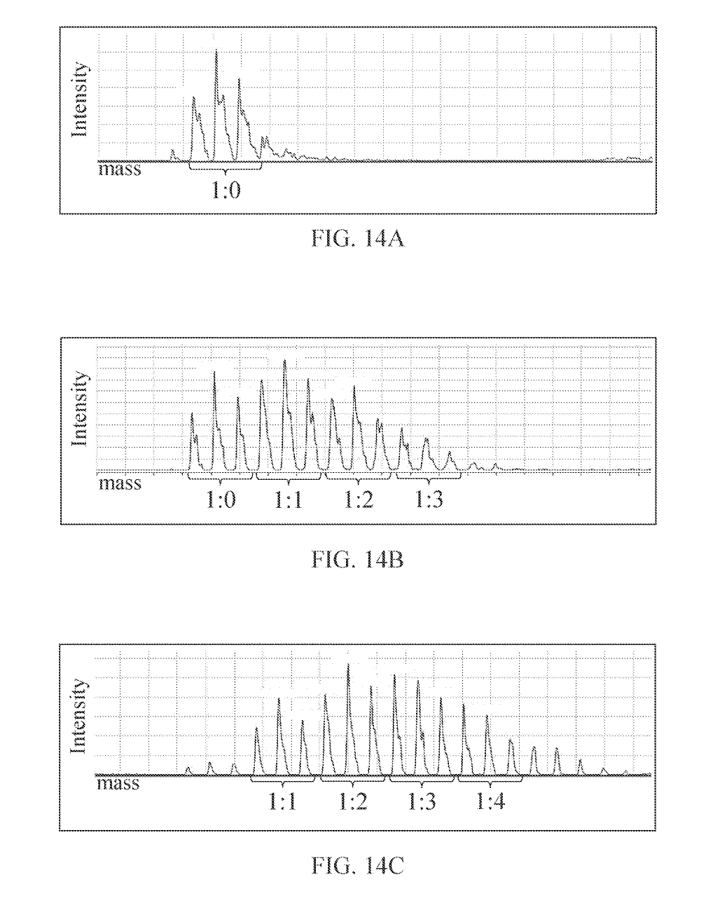

The 1:1 SM(PEG)₆ conjugate of trastuzumab and HSA was isolated using SEC (FIG. 15; showing separated peaks for the conjugate, trastuzumab and HSA). The mass of the conjugate was confirmed as 215498.52 Da using mass spectrometry.

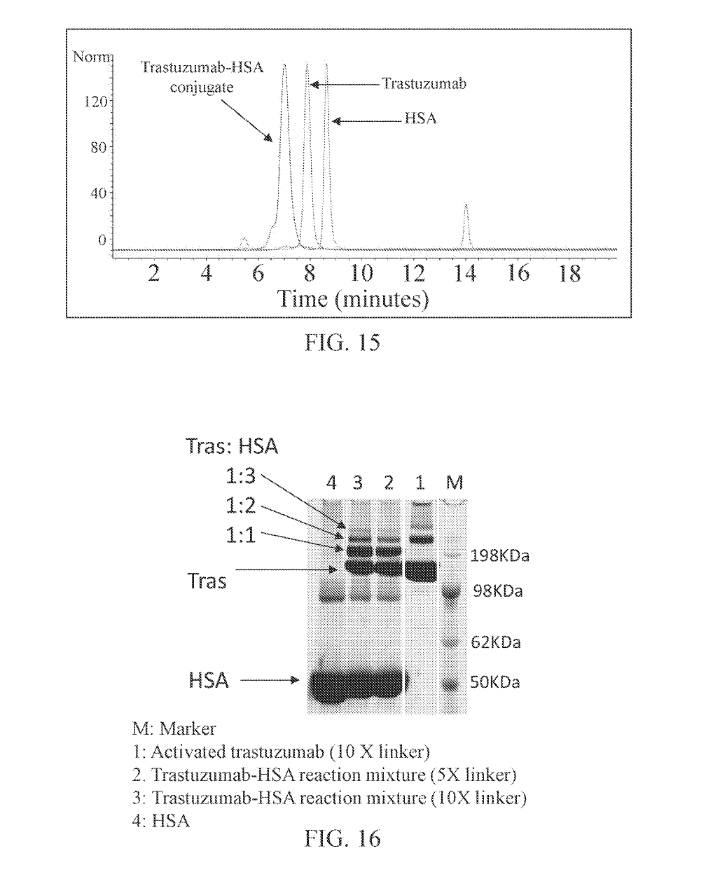

The SM(PEG)₆ trastuzumab-HSA conjugates from 5:1 and 10:1 linker:antibody reaction ratios were confirmed by SDS-PAGE gel, which showed the presence of 1:1 trastuzumab:HSA and higher order conjugates including, e.g., 1:2 and 1:3 (FIG. 16). The trastuzumab:HSA conjugates were confirmed by immunoblot (data not shown).

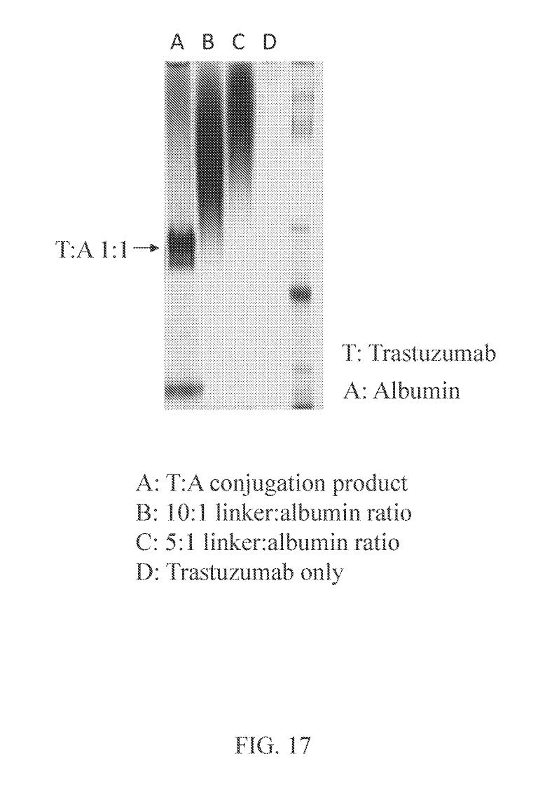

A native gel was performed analyzing samples of SM(PEG)₆ conjugated trastuzumab (FIG. 17). The pI of trastuzumab is 8.5, and trastuzumab will not migrate into the gel unless conjugated (lane D; FIG. 17). In lane A, the 1:1 trastuzumab-HSA conjugate is indicated by an arrow (FIG. 17). Lanes B and C show, respectively, 10:1 linker:antibody ratio and 5:1 linker:antibody ratio (without conjugation to albumin) (FIG. 17).

### Example 23: Conjugation of Trastuzumab and Isolated Nab-Paclitaxel Particles Via SM(PEG)₆ Activated Trastuzumab

A schematic for conjugation of an activated antibody and an isolated nab-paclitaxel particle as described in this Example is shown in FIG. 18. Materials used in this protocol were as follows: (a) nab-paclitaxel (100 mg of paclitaxel vial, Lot #013397); (b) Herceptin® (trastuzumab, 440 mg vial, 21 mg/mL, Lot#3157971); (c) normal saline, USP grade (RMBIO Lot #20607161); (d) MilliQ water, 18.4 MOhms·cm and 4 ppb TOC; (e) scintillation vials (VWR, 20-mL disposable scintillation vials); (f) 15 mL polypropylene conical tubes (Falcon, P/N 352097); (g) Zepa Spin Desalting column 7K MWCO, 5 mLs (Cat #89892, Lot # SC245087); (h) DMSO (Sigma, Cat # D2438-50 mL, Lot # RNBG0012); (i) SM(PEG)₆ (Thermo Scientific, Cat #22105, Lot # SD249151); and (j) phosphate buffered saline tablet (Sigma, Cat # P4417-50TAB, Lot# SLBS4223).

Nab-paclitaxel nanoparticles were isolated from 1 vial of a lyophilized nab-paclitaxel composition (100 mg paclitaxel). 20 mL of normal saline was added the vial to reconstitute to the nanoparticles, and 1.2 mL of the reconstituted nab-paclitaxel formulation was aliquoted into Eppendorf tubes. The Eppendorf tubes were gently mixed for 20 minutes. One Eppendorf tube was used to determine the paclitaxel content using RP-HPLC to measure particle size. The remaining Eppendorf tubes were centrifuged at 21,000 RCF for 80 minutes at 20° C. After centrifugation, the tubes were immediately decanted to remove the supernatant. 0.6 mL of normal saline was added to the remaining pellet to reconstitute the nanoparticles (final concentration of 10 mg/mL of paclitaxel).

A 21 mg/mL Herceptin® (trastuzumab) solution was prepared using normal saline. 1.2 mL of the Herceptin® solution was aliquoted into 2 15 mL spin concentrators. 100 mM potassium phosphate buffer (pH 6.6) was prepared using endotoxin free water and filtered. The spin concentrators were filled with pH 6.6 phosphate buffer and centrifuged for 30 minutes according to the manufacturer's instructions. This was repeated once. The remaining trastuzumab solution was pipetted into a 15 mL Falcon tube and diluted to a total volume of 2.4 mL. The trastuzumab concentration was measured by size exclusion chromatography (SEC). The trastuzumab concentration was adjusted to 10.5 mg/mL, as needed.

The SM(PEG)₆ package was removed from the −20° C. freezer and warmed to room temperature for 1 hour. 2 mL of sterile DMSO solution was aliquoted into an Eppendorf tube. From this Eppendorf tube, 1.51 mL of DMSO was transferred into the vial containing 100 mg of SM(PEG)₆ (112 mM final concentration). The linker solution was vortexed until the linker was completely dissolved in DMSO.

1 mL of the 10.5 mg/mL trastuzumab solution was aliquoted into Eppendorf tubes. To each Eppendorf tube containing 1 mL of trastuzumab solution, 5.1 μL of the linker solution was slowly added (ratio of linker to antibody is 7.5:1; using a ratio above 15:1 caused antibody aggregation). During this procedure, the solution was protected from light. While still protected from light, the linker/antibody solution was reacted on a shaker for 2 hours. The particle size was determined to be 157 nm by dynamic light scattering.

5 mL desalting columns were washed with phosphate buffer (pH 6.6) 4 times (1000G for 5 minutes for each centrifugation). The activated antibody solution was filtered through a desalting column to remove the unreacted linker. The filtrate was collected and pooled. The ratio of trastuzumab and linker was determined by liquid chromatography-mass spectrometry (LC-MS). 600 μL of the activated trastuzumab was slowly added into 600 μL of the nanoparticle solution. The solution was then incubated at 4° C. overnight (shaking or agitation was avoided).

The conjugation solution was taken out of the refrigerator. The solution did not have aggregates on the surface of the Eppendorf tube.

The conjugation solution was centrifuged at 21,000 G for 80 minutes. After centrifugation, the tubes were immediately decanted to remove the supernatant and each tube was washed once with PBS saline.

A 40 mg/mL human serum albumin (HSA) solution was prepared in PBS saline (pH 7.4). 800 μL of 40 mg/mL HSA solution was aliquoted into each tube. The tubes were then sonicated for 20 seconds and then mixed (this step was repeated several times until the nanoparticles are completely resuspended).

The paclitaxel content was measured by RP-HPLC. The resulting solution was then transferred to vials and stored at −80° C.

An immunoblot was performed on samples from isolated nab-paclitaxel nanoparticles and trastuzumab conjugated particles confirming the presence of 1:1 trastuzumab-HSA conjugation as well as higher order conjugates, including 1:2, 1:3, and 1:4. Prior to performing the immunoblot, the particle samples were first dissolved in ethanol to solubilize the paclitaxel and then centrifuged at 10,000 RCF. The resulting pellets were resuspended in PBS for immunoblot analysis.

The albumin isomer profiles (e.g., amount of albumin monomer) of particles from isolated nab-paclitaxel nanoparticles before and after conjugation to trastuzumab were determined using SEC. The ratios of albumin monomer to total albumin for the isolated nab-paclitaxel nanoparticles and the isolated nab-paclitaxel particles conjugated with trastuzumab are provided in Table 13.

TABLE 13
Albumin monomer ratio.

| | Ratio of albumin monomer to total |
|---|---|
| | albumin |
| Isolated particle | 0.558 |
| Isolated particle conjugated | 0.419 |
| with trastuzumab | |

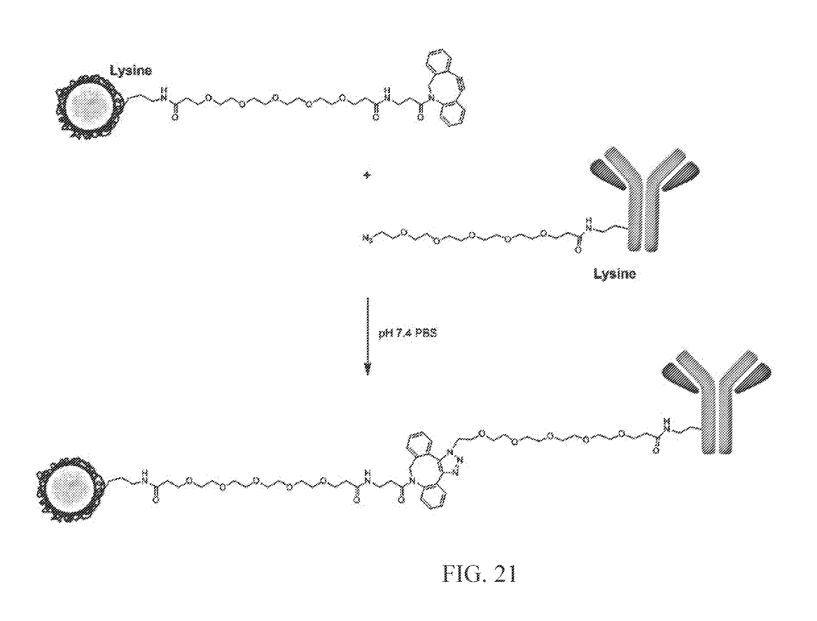

A comparison of conjugating SM(PEG)₆ to an aliquot of isolated nab-paclitaxel nanoparticles and an aliquot of nab-paclitxel formulation (ABX) without particle isolation was performed. A SDS-PAGE gel of samples from the conjugation reaction and standards is shown in FIG. 20, where lanes 1, 2, and 3 show the presence of trastuzumab-HSA conjugates. A comparison of lanes 3 and 5 show the presence of HSA conjugation of trastuzumab using the method described herein, including 1:1 trastuzumab:HSA conjugate as well as higher order conjugates. An immunoblot was performed and confirmed the presence of trastuzumab:HSA conjugates, including higher order trastuzumab:HSA conjugates.

### Example 24: Conjugation of an Antibody and Isolate Nab-Paclitaxel Particles Using Linkers with Different Spacer Lengths

This example demonstrates conjugation of an antibody and isolated nab-paclitaxel particles via SM(PEG)₆ (32.6 angstroms), SM(PEG)₂ (17.5 angstroms), and SMCC (8 angstroms) activated antibody.

Conjugated particles were prepared substantially as described in Example 23, except that the indicated linker was used. 10 mg/mL antibody-linker conjugate (prepared in a 5:1 ratio of linker to antibody) was added to 10 mg/mL of reconstituted nab-paclitaxel in saline. Antibody concentration was measured using ELISA and the paclitaxel content was measured using RP-HPLC after centrifugation and resuspension in 5% HSA. The paclitaxel/trastuzumab (Tz) mass ratio of the nanoparticle formulations are reported in Table 14.

Total free sulfhydryl groups on the nab-paclitaxel particle surface was measured as 0.66 μM for a 4.5 mg/mL nab-paclitaxel suspension. This represent 4% mole ratio of HSA (native free HSA contains 40% free sulfhydryls).

TABLE 14
Trastuzumab concentration and paclitaxel/Tz ratio

| | | Paclitaxel/Tz mass |
|---|---|---|
| Linker | Tz (mg/mL) | ratio |
| SM (PEG)₆ | 0.039 | 114 |
| SM (PEG)₂ | 0.029 | 150 |
| SMCC | 0.038 | 123 |

### Example 25: Conjugation of Trastuzumab and Isolated Nab-Paclitaxel Particles Via Activation of Isolated Nab-Paclitaxel Nanoparticles and Activation of Trastuzumab

A schematic for conjugation of an activated antibody and a thiolated, isolated nab-paclitaxel particle described in this Example is shown in FIG. 19.

To isolate nanoparticles from a nab-paclitaxel formulation, 20 mL of normal saline was added to 1 vial of a lyophilized nab-paclitaxel composition (100 mg of paclitaxel). 1.2 mL of the nab-paclitaxel formulation was aliquoted into 1.5 mL Eppendorf tubes. The Eppendorf tubes were gently mixed for 20 minutes. One Eppendorf tube was used to determine the paclitaxel content using RP-HPLC and to measure particle size. The average particle size was 147 nm. The remaining Eppendorf tubes were centrifuged at 21,000 RCF for 80 minutes at 20° C. After centrifugation, the tubes were immediately decanted to remove the supernatant. 500 μL of sodium acetate (pH 8.0) and 150 mM sodium chloride was added to the resulting pellet to reconstitute the nanoparticles (nab-paclitaxel particle concentration was 10 mg/mL of paclitaxel; contained 2.5 mg/mL (0.0376 mM) of HSA).

Traut's reagent was dissolved in WFI at a concentration of 2 mg/mL (14.5 mM). Depending on the desired degree of thiolation, 15 μL to 60 μL (linker:HSA ratio of 10:1 to 40:1, respectively) of Traut's reagent was slowly added to the isolated nanoparticle solution and reacted for 70 minutes at 4° C. 2.4 μL of 500 mM EDTA (pH 8.0) was added and incubated for 10 minutes at 4° C. The samples were centrifuged at 21,000 RCF for 80 minutes at 4° C. The tubes were removed from the centrifuge, decanted, and washed twice with PBS saline. The isolated activated nab-paclitaxel particles were resuspended in 500 μL PBS saline and sonicated for 1 minute to resuspend the particles. The particle size was measured as 150 nm.

After thiolation of the isolated nab-paclitaxel particles via different stoichiometries of Traut's reagent, the particles were analyzed by Protein Thio Fluorescent Detection Kit (Invitrogen). For nanoparticles with a paclitaxel concentration of 4.6 mg/mL, the concentration of thiol groups was determined (Table 15).

TABLE 15
Thiolation of isolated nab-paclitaxel nanoparticles.

| | Concentration |
|---|---|
| | (uM) |
| ABX Only | 0.66 |
| 10:1 | 8.02 |
| 20:1 | 15.89 |
| 30:1 | 22.53 |
| 40:1 | 30.46 |

A 21 mg/mL Herceptin® (trastuzumab) solution was prepared using normal saline. 1 mL of the Herceptin® solution was aliquoted into each 15 mL spin concentrators. 100 mM potassium phosphate buffer (pH 6.6) was prepared using endotoxin free water and filtered. The spin concentrators were filled with pH 6.6 phosphate buffer and centrifuged for 35 minutes at 3750 rpm. This was repeated once. The remaining solution was pipetted into a 15 mL Falcon tube and diluted to 2.4 mL total volume. The trastuzumab concentration was measured by size exclusion chromatography (SEC). The trastuzumab concentration was adjusted to 10.5 mg/mL, as needed.

The SM(PEG)₆ package was removed from the −20° C. freezer and warmed to room temperature for 1 hour. 2 mL of sterile DMSO solution was aliquoted into an Eppendorf tube. From this Eppendorf tube, 122 μL of DMSO was transferred into a vial containing 8.3 mg of SM(PEG)₆ (112 mM final concentration). The linker solution was vortexed until the linker was completely dissolved in DMSO.

1 mL of the trastuzumab solution was aliquoted into each Eppendorf tube. 3.4 μL of SM(PEG)₆ linker solution was slowly added into each tube containing 1 mL of trastuzumab. During this procedure, the solution was protected from light. While still protected from light, the linker/antibody solution was reacted on a shaker for 2 hours.

A 5 mL desalting column was washed with phosphate buffer (pH 6.6) 4 times (1000 G for 6 minutes for each centrifugation). The activated antibody solution was filtered through a desalting column to remove the unreacted linker. The filtrate was collected and pooled. The ratio of trastuzumab and linker was determined by liquid chromatography-mass spectrometry (LC-MS).

500 μL of the activated trastuzumab was slowly added into 500 μL of the nanoparticle solution. The solution was then incubated at 4° C. overnight (shaking or agitation was avoided).

The conjugation solution was removed from 4° C. The solution did not have aggregates on the surface of the Eppendorf tube. The particle size was measured. The average particle size for 10:1 thiolation linker:HSA was 159 nm, 20:1 thiolation linker:HSA was 166 nm, 30:1 thiolation linker:HSA was 215.9 nm, and 40:1 thiolation linker:HSA was 734.1 nm.

The conjugation solution was centrifuged at 21,000 G for 80 minutes. After centrifugation, the tubes were immediately decanted to remove the supernatant and each tube was washed twice with PBS saline.

A 40 mg/mL human serum albumin (HSA) solution was prepared in PBS saline. 800 μL of 40 mg/mL HSA solution was aliquoted into each tube. The tubes were then sonicated for 20 seconds and mixed (this step was repeated several times until the nanoparticles were completely resuspended).

The paclitaxel content was measured using RP-HPLC. The paclitaxel concentration was then adjusted according to the required concentration. The particle size and paclitaxel concentration for the final formulation was measured. The average particle size for 10:1 thiolation linker:HSA was 159.8 nm, 20:1 thiolation linker:HSA was 171.5 nm, 30:1 thiolation linker:HSA was 175.8 nm, and 40:1 thiolation linker:HSA was 196.4 nm. The resulting solution was then transferred to vials and stored at −80° C.

The final conjugated trastuzumab (Tz) concentration was measured by ELISA, and paclitaxel (PTX) concentration was measured by RP-HPLC. (Table 16).

TABLE 16
Concentration measurements of conjugated particles.

| | Tz conc. | PTX conc. | PTX/Tz |
|---|---|---|---|
| Ratio | (mg/mL) | (mg/mL) | mass ratio |
| No treatment | 0.035 | 4.4 | 110:1 |
| 10:1 | 0.16 | 4.5 | 27:1 |
| 20:1 | 0.21 | 4.1 | 20:1 |
| 30:1 | 0.21 | 3.9 | 19:1 |
| 40:1 | 0.27 | 4.1 | 15:1 |

### Example 26: Conjugation of Trastuzumab and Isolated Nab-Paclitaxel Particle Using Copper-Free Click Chemistry

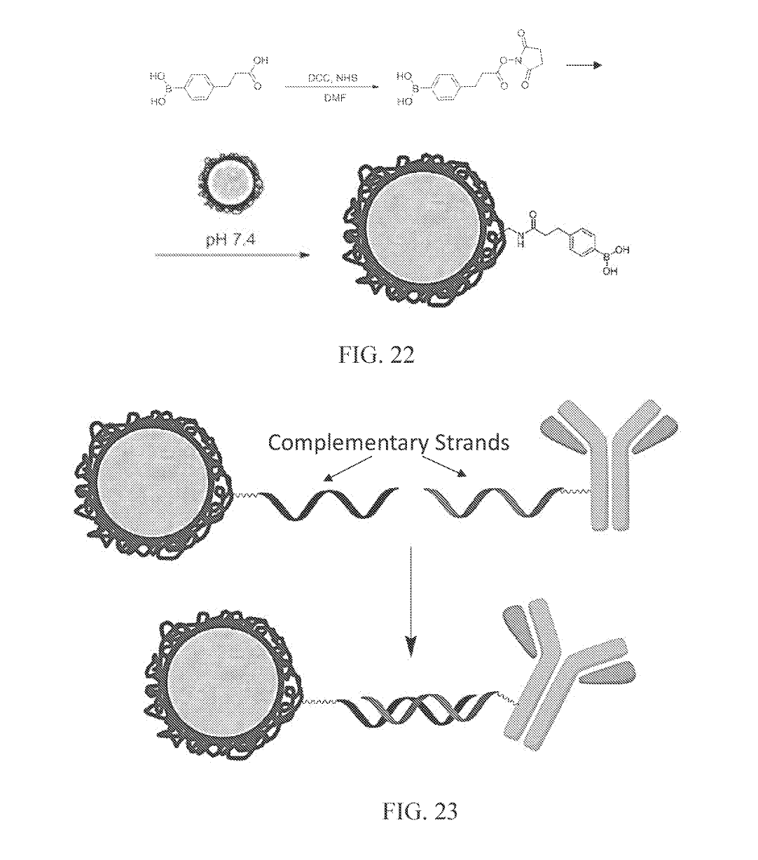

A schematic for conjugation of an antibody and an isolated nab-paclitaxel particle using copper-free click chemistry is shown in FIG. 21.

To isolate nanoparticles from a nab-paclitaxel composition, 20 mL of normal saline was added to 1 vial of lyophilized nab-paclitaxel (100 mg of paclitaxel). 1.2 mL of the nab-paclitaxel formulation was aliquoted into 16 Eppendorf tubes. The Eppendorf tubes were gently mixed for 20 minutes. One Eppendorf tube was used for an HPLC assay to determine the paclitaxel content using RP-HPLC. The remaining Eppendorf tubes were centrifuged at 21,000 RCF for 80 minutes at 20° C. After centrifugation, the tubes were immediately decanted to remove the supernatant. 600 μL of PBS (pH 7.4) was added to the resulting pellet to reconstitute the nanoparticles (nab-paclitaxel concentration of about 10 mg/mL of paclitaxel).

A 29 mM diarylcyclooctyne (DBCO)-PEGS-NHS solution was made by dissolving 3 mg of the linker reagent in 150 μL DMSO. Either 15 μL (20:1; linker:HSA) or 30 μL (40:1; linker:HSA) of DBCO solution was added to each aliquot of isolated nanoparticles, and the suspension was allowed to react for 70 minutes at room temperature. The samples were then centrifuged at 21,000 RCF for 70 minutes at 4° C. After centrifugation, the samples were decanted to remove the supernatant and washed twice with PBS saline. 500 μL PBS saline was added to the pelleted nanoparticles and then the samples were sonicated to resuspended the nanoparticles.

A 21 mg/mL Herceptin® (trastuzumab) solution was prepared using normal saline. 6 mL of the antibody solution was aliquoted into two 15 mL spin concentrators. The spin concentrators were filled with 100 mM potassium phosphate buffer (pH 6.6) and centrifuged for 30 minutes according to manufacturer's instructions. This was repeated once. The remaining trastuzumab solution was pipetted to a 15 mL Falcon tube and diluted to 12 mL of total volume. The trastuzumab concentration was adjusted to 10.5 mg/mL, as needed.

Azido-PEGS-NHS was removed from −20° C. and warmed to room temperature for 1 hour. 4.03 mg of Azido-PEGS-NHS was weighted in a vial and 82 μL of DMSO was added (112 mM solution). The solution was vortexed until the linker was completely dissolved.

1.5 mL of the antibody solution was aliquoted into separate tubes. Either 5.1 μL (5:1 linker:antibody ratio) or 10.2 μL (10:1 linker:antibody ratio) was slowly added to each tube containing antibody solution. The solution was allowed to react on a shaker for 2 hours.

Two 5 mL desalting column were washed with phosphate buffer (pH 6.6) 4 times (1000G for 6 minutes for each centrifugation). The activated antibody solution was filtered through a desalting column to remove the unreacted linker. The filtrate was collected and pooled. The ratio of trastuzumab and linker was determined by liquid chromatography-mass spectrometry (LC-MS).

500 μL of the activated trastuzumab was slowly added into 500 μL of the nanoparticle solution. The solution was incubated at room temperature for 30 minutes and then incubated at 4° C. overnight. The conjugation solution was removed from 4° C. The solution did not have aggregates on the surface of the Eppendorf tube. The particle size was measured.

The conjugation solution was centrifuged at 21,000 G for 80 minutes. After centrifugation, the tubes were immediately decanted to remove the supernatant and each tube was washed twice with PBS saline.

A 40 mg/mL human serum albumin (HSA) solution was prepared in PBS saline. 800 μL of 40 mg/mL HSA solution was aliquoted into each tube. The tubes were then sonicated until the nanoparticles were completely resuspended. The resulting solution was then transferred to vials and stored at −80° C.

Particle size was determined to be 169 nm. Final paclitaxel concentration associated with the particles was determined to be 5.73 mg/mL (RP-HPLC), and final trastuzumab concentration was determined to b 0.11 mg/mL.

### Example 27: Boronic Acid-Modified Nab-Paclitaxel

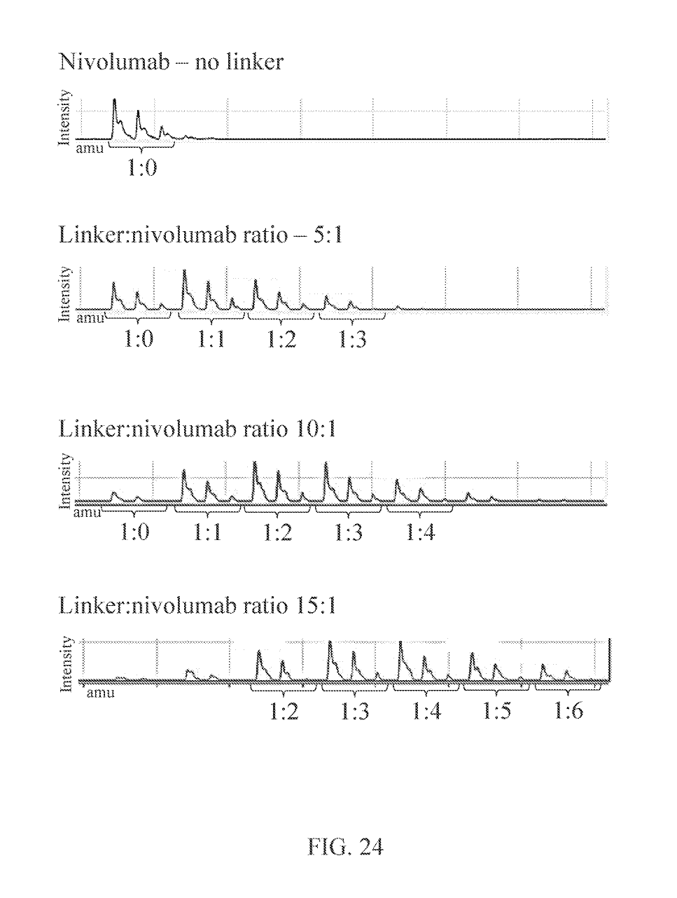

A schematic for boronic acid modification of an isolated nab-paclitaxel particle as described in this Example is shown in FIG. 22. To prepare the activated NHS ester, 4-(2-carboxyethyl)benzeneboronic (16 mg, 82.4 μmol, MW=193.99), N,N′-dicyclohexylcarbodiimide (DCC, 17.04 mg, 82.4 μmol), and N-hydroxysuccinimide (NHS, 9.52 mg, 82.4 μmol) were dissolved in 1 mL of DMF and stirred for 2 hours at room temperature.

The particle was resuspended in 600 μL of PBS buffer (pH 7.4) to make a 10 mg/mL nanoparticle suspension.

Three ratios of activated NHS ester were tested (20:1, 40:1 and 80:1 of NHS ester to surface albumin, assuming 25% of the nanoparticle weight is due to albumin) The concentration required to reach each ratio (20:1, 40:1, and 80:1) were 0.75 mM, 1.5 mM, and 3 mM, respectively. Accordingly, 5.5 μL, 11 μL and 22 of the NHS ester was added into each Eppendorf tube (containing 600 μL of the nanoparticle suspension) to make ratios of 20:1, 40:1, and 80:1. The reaction was incubated at room temperature for 2 hours and then the tubes were centrifuge at 21,000 RCF for 70 minutes. The pellet was washed three times before being resuspended in 500 μL of PBS buffer (pH 7.4).

The amount of boronic acid on the particle surface was measured by reacting with Alizarin Red S and determining the fluorescence signal at 590 nm. The concentration was compared to 4-(2-carboxyethyl)benzeneboronic standards. For the 20:1, 40:1 and 80:1 ratios, the amount of boronic acid was determined as 1.4 mM, 2.0 mM, and 2.6 mM, respectively, per 5 mg/mL of nab-paclitaxel. The unmodified nanoparticles, along with the 20:1 and 40:1 modified nanoparticles were stable, although the 80:1 nanoparticles began to aggregate.

### Example 28: Conjugation of Trastuzumab and Isolated Nab-Paclitaxel Particles Using Complementary DNA

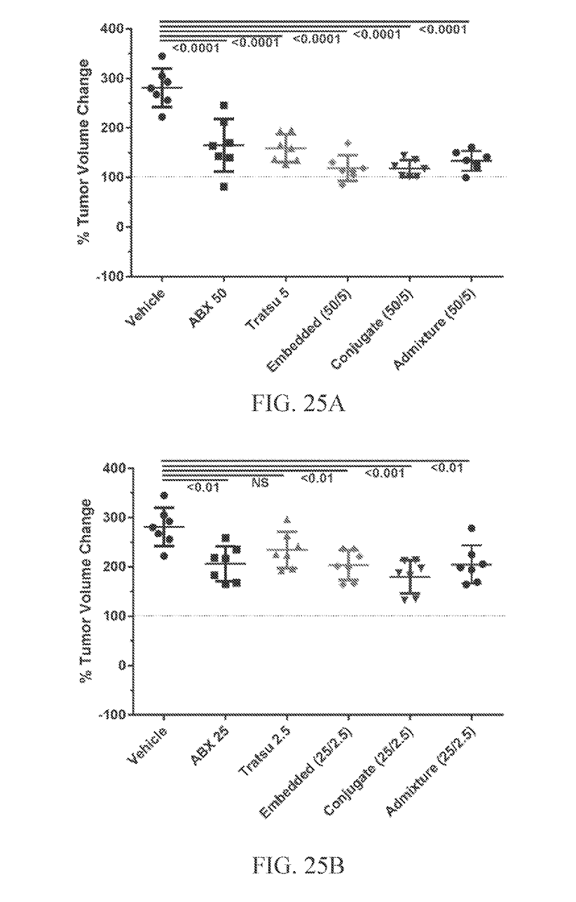

A schematic for conjugation of an activated antibody and an activated isolated nab-paclitaxel particle using complementary DNA is shown in FIG. 23. In brief, a first strand is conjugated to the nab-paclitaxel nanoparticles and a second strand complementary to the first strand is conjugated to the antibody. Mixing of the nanoparticles and the antibody allows the commentary strands to pair, thereby conjugating the antibody to the nab-paclitaxel nanoparticles.

Single stranded DNA (ssDNA) was conjugated to isolated nab-paclitaxel particles from a formulation of a nab-paclitaxel. A 20 mM TCEP solution was made. 3 mg of ssDNA (5ThioMC6-CACACACACACACACACACA; SEQ ID NO: 1; “CA20”) was weighed and dissolved in 1 mL of ddH₂O (447 μM solution of ssDNA). The 20 mM TCEP solution was added to the 447 μM ssDNA solution at a 3:7 volume ratio, mixed, and then reacted for 2 hours at room temperature. The reaction mixture was purified using NAP-10 (GE Healthcare Life Science, Cat: #17-0854-01). The measured concentration of collected CA20 ssDNA was 216 μM, as determined by UV spectroscopy (260 nm).

6.83 mg of SM(PEG)₆ was weighed and dissolve in 0.5 mL of DMSO (22.4 mM SM(PEG)₆ solution). 11 μL of the 22.4 mM SM(PEG)₆ solution was added to 1.2 mL of the CA20 ssDNA solution and reacted for 20 minutes.

600 μL of isolated nab-paclitaxel suspension was aliquoted into Eppendorf tubes. 600 μL of CA20-SM(PEG)₆ was added to each aliquot of nanoparticle solution. The reaction mixture was incubated overnight.

The Eppendorf tubes were centrifuged to pellet the nanoparticles, and the supernatant was decanted. The pellets were washed three times with PBS. 500 μL of PBS was added to each tube and the pellet was resuspended by sonication (5 seconds and repeated until the particles were resuspended). Particle size was determined using Dynamic Light Scattering. The particle size of a nab-paclitaxel standard from a controlled nab-paclitaxel formulation was 152 nm and the CA20 conjugated nab-paclitaxel was 152 nm.

ssDNA was conjugated to trastuzumab from a formulation of Herceptin®. 4 mM TCEP solution was prepared. The ssDNA (5ThioMC6-GTGTGTGTGTGTGTG; SEQ ID NO: 2; “GT15”) was dissolved in ddH₂O to make a 500 mM ssDNA solution. The 4 mM TCEP solution was added to the 500 μM ssDNA solution at a 1:2 volume ratio, mixed, and then reacted for 2 hours at room temperature. The reaction mixture was purified using NAP-10 (GE Healthcare Life Science, Cat: #17-0854-01).

1 mL of Herceptin® (21 mg/mL reconstituted in normal saline) was aliquoted into a 15 mL spin concentrator. A PBS buffer (pH 7.4) was prepared. The spin concentrator was filled with the PBS buffer and centrifuged for 30 minutes according to manufacturer's instructions. The spin concentrator was once again filled with the PBS and centrifuged. The resulting trastuzumab solution was removed and the concentration was adjusted to 10.5 mg/mL with the PBS buffer as needed.

6.8 mg of SM(PEG)₆ was weighed and dissolve in 100 μL of DMSO (112 mM SM(PEG)₆ solution). 2.3 μL of the 112 mM SM(PEG)₆ solution was added to 500 μL of the trastuzumab solution and reacted for 1.5 hours. The unreacted linker was removed using desalting columns.

The deprotected GT15 solution was added to the resulting activated trastuzumab and incubated for 2 hours. The mixture was added into a spin concentrator and filled with PBS buffer (pH 7.4). The mixture was centrifuged and the flow through was analyzed for the presence of ssDNA. The spin concentrator was refilled with PBS and centrifuged five times before ssDNA was not detected in the flow through.

The trastuzumab-GT15 solution and the CA20-nab-paclitaxel solution were mixed in an Eppendorf tube and incubated for 2 hours. The reaction mixture was then centrifuged at 21,000 RCF. The tube was immediately removed from the centrifuge, the supernatant was decanted, and the resulting pellet was washed with PBS three times. The pellet was resuspended in 4% HSA solution in PBS and sonicated to resuspend the particles. The conjugated particles were stored at −80° C.

### Example 29: Conjugation of Bevacizumab and Isolated Nab-Paclitaxel Particles

Materials used in this Example include: (a) nab-paclitaxel, 100 mg of paclitaxel; (b) Avastin (bevacizumab; 25 mg/mL, Lot#3039196); (c) normal saline (RMBIO, USP grade, Lot #20607161); (d) MilliQ water (18.4 MOhms·cm and 4 ppb TOC); (e) scintillation vials (VWR, 20-mL disposable scintillation vials); (f) 15 mL polypropylene conical tubes (Falcon, P/N 352097); (g) Zepa spin desalting column (7K MWCO, 5 mLs, Cat #89892, Lot # SC245087); (h) DMSO (Sigma, Cat # D2438-50 mL, Lot # RNBG0012); (i) SM(PEG)₆ (Thermo Scientific, Cat #22105, Lot # SD249151); and (j) phosphate buffered saline (PBS) tablet (Sigma, Cat # P4417-50TAB, Lot# SLBS4223).

20 mL of normal saline was added to one vial of a lyophilized nab-paclitaxel composition (100 mg paclitaxel). Aliquots of 1.2 mL of the nab-paclitaxel solution were added into four Eppendorf tubes. The tubes were incubated and gently swirled for 20 minutes. One tube was used to conduct a paclitaxel HPLC assay to determine the paclitaxel content using RP-HPLC. The remaining Eppendorf tubes containing nab-paclitaxel suspension were centrifuged at 21,000 RCF for 80 minutes at 20° C. After centrifugation, each tube was immediately taken out and the supernatant of each tube was removed. The pellet of nanoparticles was gently reconstituted with 0.5 mL of normal saline to make a final nanoparticle solution of 12 mg/mL of paclitaxel.

1 mL of Avastin® (25 mg/mL, reconstituted in WFI) was aliquoted into a 15 mL spin concentrator. 100 mM potassium phosphate buffer (pH 6.6) was prepared. The spin concentrator was filled with phosphate buffer (pH 6.6) and centrifuged for 30 minutes according to the manufacturer's instructions. The spin concentrator was once more filled with phosphate buffer (pH 6.6) and centrifuged for 30 minutes according to the manufacturer's instructions. The remaining bevacizumab solution was pipetted into a 15 mL Falcon tube and diluted to 2.5 mL total volume. The concentration was confirmed using size exclusion chromatography (SEC).

The SM(PEG)₆ package was removed from −20° C. and warmed at room temperature for 1 hour. 6.83 mg of SM(PEG)₆ was dissolved in 100 μL DMSO (112 mM linker solution). The linker solution was vortexed until the linker was completely dissolved.

0.5 mL of bevacizumab solution was aliquoted into four Eppendorf tubes. Into two of the Eppendorf tubes, 1.65 μL, and 3.3 μL of the linker solution was slowly added to generate a 5:1 and 10:1 molar ratio of linker to antibody. An admixture control tube was reserved without linker modification. During this procedure, the solution was protected from light. The solution was reacted on a shaker for 2 hours.

Two 5 mL desalting columns were prepared by washing the desalting columns with phosphate buffer (pH 6.6) 4 times (1000G, 5 minutes for each centrifugation). The activated bevacizumab solution was then filtered through the column to remove any unreacted linker.

500 μL of each linker ratio of the activated bevacizumab solution was then slowly added to 500 μL of the nanoparticle solution. 500 μL of the non-activated bevacizumab solution was added to 500 μL of the nanoparticle solution. The resulting solutions were then incubated at 4° C. overnight without shaking or agitation.

The conjugation solutions were removed from 4° C. storage (the solutions did not appear to have aggregates on the surface) and centrifuged at 21,000 G for 80 minutes. The tubes were then immediately removed from the centrifuge and the supernatants were removed. Each tube was washed three times with PBS saline.

A 40 mg/mL human serum albumin (HSA) solution was prepared in PBS saline (pH 7.4). 500 μL of 40 mg/mL HSA solution was added into each tube containing pelleted nanoparticles. The tubes were sonicated for 20 seconds and mixed. The sonication step was repeated several times until the nanoparticles were completely resuspended. Following removal of sample for characterization, the solutions were transferred to vials and stored at −80° C.

The particle sizes of the samples were measured. The average particle sizes were as follows: nab-paclitaxel, 146 nm; bevacizumab admixture control, 148 nm; 5:1 linker:antibody bevacizumab nanoparticles, 152 nm; and 10:1 linker:antibody bevacizumab nanoparticles, 153 nm.

The bevacizumab content of the samples was measured by ELISA and paclitaxel content of the samples was measured by RP-HPLC (Table 16).

TABLE 16
Bevacizumab and paclitaxel concentrations.

| | Bevacizumab | Paclitaxel | Paclitaxel/ |
|---|---|---|---|
| Sample | (mg/mL) | (mg/mL) | Bevacizumab |
| Nab-paclitaxel | 0.0007 | 5.00 | 6971 |
| suspension | | | |
| Admixture | 0.023 | 8.51 | 376 |
| Control | | | |
| 5:1 bevacizumab | 0.155 | 8.92 | 57 |
| 10:1 bevacizumab | 0.144 | 8.76 | 61 |

Immunoblot analysis of the samples was performed and confirmed conjugation of bevacizumab and HSA on particles, including presence of higher order conjugates, from conjugation reactions of a isolated nab-paclitaxel nanoparticles.

### Example 30: Conjugation of Cetuximab and Isolated Nab-Paclitaxel Particles

Materials used in this method include: (a) nab-paclitaxel, 100 mg of paclitaxel; (b) Erbitux® (cetuximab, Lot# IMG395); (c) normal saline (RMBIO, USP grade, Lot #20607161); (d) MilliQ water (18.4 MOhms·cm and 4 ppb TOC); (e) scintillation vials (VWR, 20-mL disposable scintillation vials); (f) 15 mL polypropylene conical tubes (Falcon, P/N 352097); (g) Zepa spin desalting column (7K MWCO, 5 mLs, Cat #89892, Lot # SC245087); (h) DMSO (Sigma, Cat # D2438-50 mL, Lot # RNBG0012); (i) SM(PEG)₆ (Thermo Scientific, Cat #22105, Lot # SD249151); and (j) phosphate buffered saline (PBS) tablet (Sigma, Cat # P4417-50TAB, Lot# SLBS4223).

20 mL of normal saline was added to one vial of a lyophilized nab-paclitaxel composition. Aliquots of 1.2 mL of the nab-paclitaxel suspension were added into four Eppendorf tubes. The tubes were incubated and gently swirled for 20 minutes. One tube was used to conduct a paclitaxel HPLC assay to determine the paclitaxel content using RP-HPLC.

The remaining Eppendorf tubes containing the nab-paclitaxel suspension were centrifuged at 21,000 RCF for 80 minutes at 20° C. After centrifugation, each tube was immediately taken out and the supernatant of each tube was removed. The pellet of nanoparticles was gently reconstituted with 0.5 mL of normal saline to make a final 12 mg/mL nanoparticle solution.

1 mL of Erbitux solution (25 mg/mL, reconstituted in WFI) was aliquoted into a 15 mL spin concentrator. 100 mM potassium phosphate buffer (pH 6.6) was prepared. The spin concentrator was filled with phosphate buffer (pH 6.6) and centrifuged for 30 minutes according to the manufacturer's instructions. The spin concentrator was once more filled with phosphate buffer (pH 6.6) and centrifuged for 30 minutes according to the manufacturer's instructions. The remaining cetuximab solution was pipetted into a 15 mL Falcon tube and diluted to 2.5 mL total volume. The concentration was confirmed using size exclusion chromatography (SEC).

The SM(PEG)₆ package was removed from −20° C. and warmed at room temperature for 1 hour. 6.83 mg of SM(PEG)₆ was dissolved in 100 μL DMSO (112 mM linker solution). The linker solution was vortexed until the linker was completely dissolved.

0.5 mL of cetuximab solution was aliquoted into four Eppendorf tubes. Into two of the Eppendorf tubes, 1.65 μL, and 3.3 μL of the linker solution was slowly added to generate a 5:1 and 10:1 molar ratio of linker to antibody. An admixture control tube was reserved without linker modification. During this procedure, the solution was protected from light. The solution was reacted on a shaker for 2 hours.

Two 5 mL desalting columns were prepared by washing the desalting columns with phosphate buffer (pH 6.6) 4 times (1000G, 5 minutes for each centrifugation). The activated cetuximab solution was then filtered through the column to remove any unreacted linker.

500 μL of each linker ratio of the activated cetuximab solution was then slowly added to 500 μL of the nanoparticle solution. 500 μL of the non-activated cetuximab solution was added to 500 μL of the nanoparticle solution. The resulting solutions were then incubated at 4° C. overnight without shaking or agitation.

The conjugation solutions were taken out of the fridge (the solutions did not appear to have aggregates on the surface) and centrifuged at 21,000 G for 80 minutes. The tubes were then immediately removed from the centrifuged and the supernatants were removed. Each tube was washed three times with PBS saline.

A 40 mg/mL human serum albumin (HSA) solution was prepared in PBS saline (pH 7.4). 500 μL of 40 mg/mL HSA solution was added into each tube containing pelleted nanoparticles. The tubes were sonicated for 20 seconds and mixed. The sonication step was repeated several times until the nanoparticles were completely resuspended.

Following removal of sample for characterization, the solutions were transferred to vials and stored at −80° C.

The particle sizes of the samples were measured. The average particle sizes were as follows: nab-paclitaxel, 146 nm; cetuximab admixture control, 148 nm; 5:1 linker:antibody cetuximab nanoparticles, 149 nm; and 10:1 linker:antibody cetuximab nanoparticles, 147 nm.

The cetuximab content of the samples was measured by ELISA and paclitaxel content of the samples was measured by RP-HPLC (Table 17).

TABLE 17
Cetuximab and paclitaxel concentrations.

| | Cetuximab | Paclitaxel | Paclitaxel/ |
|---|---|---|---|
| Sample | (mg/mL) | (mg/mL) | Cetuximab |
| Nab-paclitaxel | 0.0007 | 5.00 | 6971 |
| Admixture | 0.032 | 8.52 | 269 |
| Control | | | |
| 5:1 cetuximab | 0.044 | 8.28 | 188 |
| 10:1 cetuximab | 0.057 | 8.38 | 148 |

Immunoblot analysis of the samples was performed and confirmed conjugation of cetuximab and HSA on particles, including presence of higher order conjugates, from conjugation reactions of isolated nab-paclitaxel nanoparticles.

### Example 31: Conjugation of Nivolumab and Isolated Nab-Paclitaxel Particles

Materials used in this method include: (a) nab-paclitaxel, 100 mg vial; (b) Opdivo (nivolumab, Lot# AAL6305); (c) normal saline (RMBIO, USP grade, Lot #20607161); (d) MilliQ water (18.4 MOhms·cm and 4 ppb TOC); (e) scintillation vials (VWR, 20-mL disposable scintillation vials); (f) 15 mL polypropylene conical tubes (Falcon, P/N 352097); (g) Zepa spin desalting column (7K MWCO, 5 mLs, Cat #89892, Lot # SC245087); (h) DMSO (Sigma, Cat # D2438-50 mL, Lot # RNBG0012); (i) SM(PEG)₆ (Thermo Scientific, Cat #22105, Lot # SD249151); and (j) phosphate buffered saline (PBS) tablet (Sigma, Cat # P4417-50TAB, Lot# SLBS4223).

20 mL of normal saline was added to 1 vial of a lyophilized nab-paclitaxel composition. Aliquots of 1.2 mL of the nab-paclitaxel suspension were added into four Eppendorf tubes. The tubes were incubated and gently swirled for 20 minutes. One tube was used to conduct measure the paclitaxel content using RP-HPLC.

The remaining Eppendorf tubes containing the nab-paclitaxel suspension were centrifuged at 21,000 RCF for 80 minutes at 20° C. After centrifugation, each tube was immediately taken out and the supernatant of each tube was removed. The pellet of nanoparticles was gently reconstituted with 0.5 mL of normal saline to make a final 12 mg/mL nanoparticle solution.

2 mL of Opdivo (10 mg/mL, reconstituted in WFI) was aliquoted into a 15 mL spin concentrator. 100 mM potassium phosphate buffer (pH 6.6) was prepared. The spin concentrator was filed with phosphate buffer (pH 6.6) and centrifuged for 30 minutes according to the manufacturer's instructions. The spin concentrator was once more filled with phosphate buffer (pH 6.6) and centrifuged for 30 minutes according to the manufacturer's instructions. The remaining nivolumab solution was pipetted into a 15 mL Falcon tube and diluted to 2 ml total volume.

The SM(PEG)₆ package was removed from −20° C. and warmed at room temperature for 1 hour. 6.83 mg of SM(PEG)₆ was dissolved in 100 μL DMSO (112 mM linker solution). The linker solution was vortexed until the linker was completely dissolved.

0.5 mL of nivolumab solution was aliquoted into 4 Eppendorf tubes. Into three of the Eppendorf tubes, 1.7 μL, 3.4 μL, and 5.1 μL of the linker solution was slowly added to generate a 5:1, 10:1, or 15:1 molar ratio of linker to antibody. An admixture control tube was reserved without linker modification. During this procedure, the solution should be protected from light. The solution was reacted on a shaker for 2 hours.

Three 5 mL desalting columns were prepared by washing the desalting columns with phosphate buffer (pH 6.6) 4 times (1000G, 5 minutes for each centrifugation). The activated nivolumab solution was then filtered through the column to remove any unreacted linker.

500 μL of each concentration of the activated nivolumab solution was then slowly added to 500 μL of the nanoparticle solution. 500 μL of the non-activated nivolumab solution was added to 500 μL of the nanoparticle solution. The resulting solutions were then incubated at 4° C. overnight without shaking or agitation.

The conjugation solutions were taken out of the fridge (the solutions did not appear to have aggregates on the surface) and centrifuged at 21,000 G for 80 minutes. The tubes were then immediately removed from the centrifuged and the supernatants were removed. Each tube was washed three times with PBS saline.

A 40 mg/mL human serum albumin (HSA) solution was prepared in PBS saline (pH 7.4). 500 μL of 40 mg/mL HSA solution was added into each tube containing pelleted nanoparticles. The tubes were sonicated for 20 seconds and mixed. The sonication step was repeated several times until the nanoparticles were completely resuspended.

Following removal of sample for characterization, the solutions were transferred to vials and stored at −80° C.

The particle sizes of the samples were measured. The average particle sizes were as follows: nab-paclitaxel, 145 nm; nivolumab admixture control, 145 nm; 5:1 linker:antibody nivolumab nanoparticles, 149 nm; 10:1 linker:antibody nivolumab nanoparticles, 147 nm; and 15:1 linker:antibody nivolumab nanoparticles, 150 nm.

The nivolumab content of the samples was measured by ELISA and paclitaxel content of the samples was measured by RP-HPLC (Table 18).

TABLE 18
Nivolumab and paclitaxel concentrations.

| | Nivolumab | Paclitaxel | Paclitaxel/ |
|---|---|---|---|
| Sample | (mg/mL) | (mg/mL) | Nivolumab |
| nab-paclitaxel | 0.0005 | 5.00 | 10000 |
| Admixture | 0.023 | 9.48 | 411 |
| Control | | | |
| 5:1 nivolumab | 0.042 | 9.20 | 216 |
| 10:1 nivolumab | 0.042 | 9.26 | 221 |
| 15:1 nivolumab | 0.035 | 9.29 | 267 |

Immunoblot analysis of the samples was performed and confirmed conjugation of nivolumab and HSA on particles, including presence of higher order conjugates, from conjugation reactions of isolated nab-paclitaxel nanoparticles.

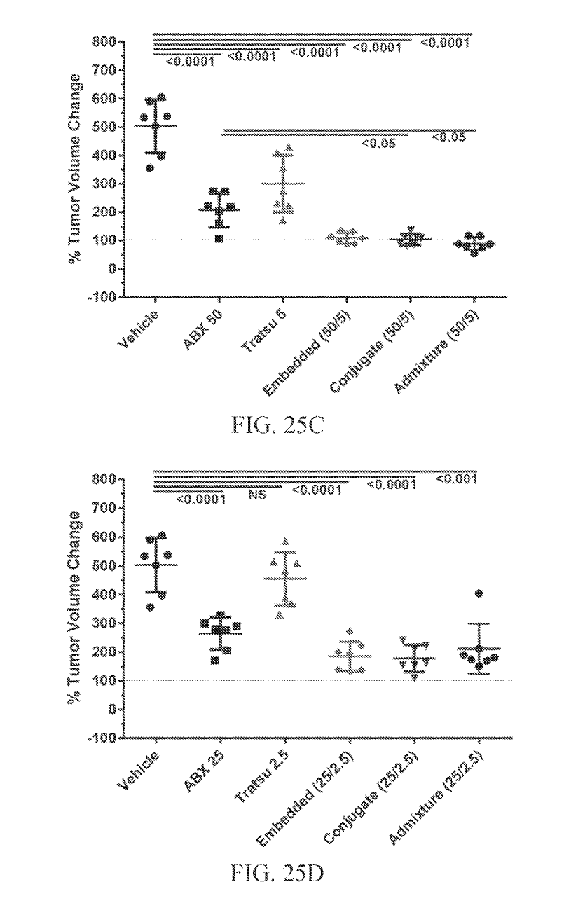

FIG. 24 shows deconvoluted mass spectra from LC-MS analyses of nivolumab and activated nivolumab at linker:antibody reaction ratios of 5:1, 10:1, and 15:1. Increasing reaction ratios of linker:antibody increased the number of linkers conjugated to a nivolumab, as shown under each isotope cluster as antibody:conjugated linker (FIG. 24).

### Example 32: Preparation of Admixed Trastuzumab-Nanoparticle Formulations for In Vitro and In Vivo Studies

Batch 1. 0.39 mL of Herceptin® (Trastuzumab and excipients, 21 mg/mL) and 9.61 mL 0.9% NaCl normal saline (G-Biosciences) was added to a lyophilized nab-paclitaxel composition (100 mg of paclitaxel) in a vial. The contents of the vial were reconstituted without mixing for 5 minutes, followed by gentle mixing to assure complete reconstitution. The mixture was incubated at room temperature (˜20° C.) for 1 hour before adding 10 mL normal saline to the vial. The mixture was gently mixed to assure homogeneity. The final concentration of paclitaxel was 5 mg/mL (with 45 mg/mL albumin), and the final concentration of Trastuzumab was 0.41 mg/mL.

Batch 2. 0.152 mL of Herceptin® (Trastuzumab and excipients, 21 mg/mL) and 9.848 mL 0.9% NaCl normal saline (G-Biosciences) was added a lyophilized nab-paclitaxel composition (100 mg of paclitaxel) in a vial. The contents of the vial were reconstituted without mixing for 5 minutes, followed by gentle mixing to assure complete reconstitution. The mixture was incubated at room temperature (˜20° C.) for 1 hour before adding 10 mL normal saline to the vial. The mixture was gently mixed to assure homogeneity. The final concentration of paclitaxel was 5 mg/mL (with 45 mg/mL albumin), and the final concentration of Trastuzumab was 0.16 mg/mL.

Batch 3. 0.093 mL of Herceptin® (Trastuzumab and excipients, 21 mg/mL) and 9.907 mL 0.9% NaCl normal saline (G-Biosciences) was added to a lyophilized nab-paclitaxel composition (100 mg of paclitaxel) in a vial. The contents of the vial were reconstituted without mixing for 5 minutes, followed by gentle mixing to assure complete reconstitution. The mixture was incubated at room temperature (˜20° C.) for 1 hour before adding 10 mL normal saline to the vial. The mixture was gently mixed to assure homogeneity. The final concentration of paclitaxel was 5 mg/mL (with 45 mg/mL albumin), and the final concentration of Trastuzumab was 0.098 mg/mL.

Batch 4. 0.051 mL of Herceptin® (Trastuzumab and excipients, 21 mg/mL) and 9.949 mL 0.9% NaCl normal saline (G-Biosciences) was added to a lyophilized nab-paclitaxel composition (100 mg of paclitaxel) in a vial. The contents of the vial were reconstituted without mixing for 5 minutes, followed by gentle mixing to assure complete reconstitution. The mixture was incubated at room temperature (˜20° C.) for 1 hour before adding 10 mL normal saline to the vial. The mixture was gently mixed to assure homogeneity. The final concentration of paclitaxel was 5 mg/mL (with 45 mg/mL albumin), and the final concentration of Trastuzumab was 0.054 mg/mL.

Batch 5. 0.476 mL of Herceptin® (Trastuzumab and excipients, 21 mg/mL) and 9.524 mL 0.9% NaCl normal saline (G-Biosciences) was added to a lyophilized nab-paclitaxel composition (100 mg of paclitaxel) in a vial. The contents of the vial were reconstituted without mixing for 5 minutes, followed by gentle mixing to assure complete reconstitution. The mixture was incubated at room temperature (˜20° C.) for 1 hour before adding 10 mL normal saline to the vial. The mixture was gently mixed to assure homogeneity. The final concentration of paclitaxel was 5 mg/mL (with 45 mg/mL albumin), and the final concentration of Trastuzumab was 0.5 mg/mL.

### Example 33: Preparation of Conjugated Trastuzumab-Nanoparticle Formulations for In Vitro and In Vivo Studies

To isolate nab-paclitaxel nanoparticles, 20 mL of normal saline was added to 1 vial containing a lyophilized nab-paclitaxel composition (100 mg of paclitaxel). 1.2 mL of the nab-paclitaxel suspension was aliquoted into 16 of 1.5 mL Eppendorf tubes. The Eppendorf tubes were gently mixing for 20 minutes. One Eppendorf tube was used for an HPLC assay to determine the paclitaxel content and to measure particle size. The average particle size was 147 nm. The remaining Eppendorf tubes were centrifuged at 21,000 RCF for 80 minutes at 20° C. After centrifugation, the tubes were immediately decanted to remove the supernatant. 500 μL of sodium acetate (pH 8.0) and 150 mM sodium chloride was added to the resulting pellet to reconstitute the nanoparticles (nab-paclitaxel particle concentration was 10 mg/mL of paclitaxel).

Traut's reagent was dissolved in WFI at a concentration of 2 mg/mL (14.5 mM). 30 μL (linker:HSA ratio of 20:1) of Traut's reagent was slowly added to the isolated nanoparticle solution and reacted for 70 minutes at 4° C. 2.4 μL of 500 mM EDTA (pH 8.0) was added and incubated for 10 minutes at 4° C. The samples were centrifuged at 21,000 RCF for 80 minutes at 4° C. The tubes were removed from the centrifuge, decanted, and washed twice with PBS saline. The isolated activated nab-paclitaxel particles were resuspended in 500 μL PBS saline and sonicated for 1 minute to resuspend the particles.

A 21 mg/mL Herceptin® (trastuzumab) solution was prepared using normal saline. 6 mL of the Herceptin® solution was aliquoted into 3 of 15 mL spin concentrator.

100 mM potassium phosphate buffer (pH 6.6) was prepared using endotoxin free water and filtered. The spin concentrators were filled with pH 6.6 phosphate buffer and centrifuged for 35 minutes at 3750 rpm. This was repeated once. The remaining solution was pipetted into a 15 mL Falcon tube and diluted to 12 mL total volume. The trastuzumab concentration was measured by size exclusion chromatography (SEC). The trastuzumab concentration was adjusted to 10.5 mg/mL, as needed.

The SM(PEG)₆ package was removed from the −20° C. freezer and warmed to room temperature for 1 hour. 2 mL of sterile DMSO solution was aliquoted into an Eppendorf tube. From this Eppendorf tube, 122 μL of DMSO was transferred into a vial containing 8.3 mg of SM(PEG)₆ (112 mM final concentration). The linker solution was vortexed until the linker was completely dissolved in DMSO.

2 mL of the trastuzumab solution was aliquoted into each of six 15 mL Falcon tube. 6.8 μL of SM(PEG)₆ linker solution was slowly added into each 2 mL of tube containing 1 mL trastuzumab. During this procedure, the solution was protected from light. While still protected from light, the linker/antibody solution was reacted on a shaker for 2 hours.

A 5 mL desalting column was washed with phosphate buffer (pH 6.6) 4 times (1000 G for 6 minutes for each centrifugation). The activated antibody solution was filtered through a desalting column to remove the unreacted linker. The filtrate was collected and pooled. The ratio of trastuzumab and linker was determined by liquid chromatography-mass spectrometry (LC-MS).

500 μL of the activated trastuzumab was slowly added into 500 μL of the nanoparticle solution. The solution was then incubated at 4° C. overnight (shaking or agitation was avoided).

The conjugation solution was removed from 4° C. The solution did not have aggregates on the surface of the Eppendorf tube. The conjugation solution was centrifuged at 21,000 G for 80 minutes. After centrifugation, the tubes were immediately decanted to remove the supernatant and each tube was washed twice with PBS saline.

A 40 mg/mL human serum albumin (HSA) solution was prepared in PBS saline. 500 μL of 40 mg/mL HSA solution was aliquoted into each tube. The tubes were then sonicated for 20 seconds and mixed (this step was repeated several times until the nanoparticles were completely resuspended).

The paclitaxel content was measured by RP-HPLC and was then adjusted to 5.33 mg/mL. The particle size was measured by DLS. The particle size was 175 nm. The resulting solution was then transferred to vials and stored at −80° C.

The Herceptin concentration of the final conjugated particles were analyzed by in vitro cell binding assay. The concentration is 0.43 mg/mL.

### Example 31: Preparation of Conjugated Trastuzumab-Nanoparticle Formulations for In Vitro and In Vivo Studies

To isolate nab-paclitaxel nanoparticles, 20 mL of normal saline was added to 1 vial containing a lyophilized nab-paclitaxel composition (100 mg of paclitaxel). 1.2 mL of the nab-paclitaxel suspension was aliquoted into 16 of 1.5 mL Eppendorf tubes. The Eppendorf tubes were gently mixing for 20 minutes. One Eppendorf tube was used for an HPLC assay to determine the paclitaxel content and to measure particle size. The average particle size was 147 nm. The remaining Eppendorf tubes were centrifuged at 21,000 RCF for 80 minutes at 20° C. After centrifugation, the tubes were immediately decanted to remove the supernatant. 500 μL of sodium acetate (pH 8.0) and 150 mM sodium chloride was added to the resulting pellet to reconstitute the nanoparticles (nab-paclitaxel particle concentration was 10 mg/mL of paclitaxel.

Traut's reagent was dissolved in WFI at a concentration of 2 mg/mL (14.5 mM). 15 μL (linker:HSA ratio of 10:1) of Traut's reagent was slowly added to the isolated nanoparticle solution and reacted for 70 minutes at 4° C. 2.4 μL of 500 mM EDTA (pH 8.0) was added and incubated for 10 minutes at 4° C. The samples were centrifuged at 21,000 RCF for 80 minutes at 4° C. The tubes were removed from the centrifuge, decanted, and washed twice with PBS saline. The isolated activated nab-paclitaxel particles were resuspended in 500 μL PBS saline and sonicated for 1 minute to resuspend the particles.

A 21 mg/mL Herceptin® (trastuzumab) solution was prepared using normal saline. 6 mL of the Herceptin® solution was aliquoted into 3 of 15 mL spin concentrator.

100 mM potassium phosphate buffer (pH 6.6) was prepared using endotoxin free water and filtered. The spin concentrators were filled with pH 6.6 phosphate buffer and centrifuged for 35 minutes at 3750 rpm. This was repeated once. The remaining solution was pipetted into a 15 mL Falcon tube and diluted to 12 mL total volume. The trastuzumab concentration was measured by size exclusion chromatography (SEC). The trastuzumab concentration was adjusted to 10.5 mg/mL, as needed.

The SM(PEG)₆ package was removed from the −20° C. freezer and warmed to room temperature for 1 hour. 2 mL of sterile DMSO solution was aliquoted into an Eppendorf tube. From this Eppendorf tube, 122 μL of DMSO was transferred into a vial containing 8.3 mg of SM(PEG)₆ (112 mM final concentration). The linker solution was vortexed until the linker was completely dissolved in DMSO.

2 mL of the trastuzumab solution was aliquoted into 6 of 15 mL Falcon tube. 6.8 μL of SM(PEG)₆ linker solution was slowly added into each tube containing 1 mL trastuzumab. During this procedure, the solution was protected from light. While still protected from light, the linker/antibody solution was reacted on a shaker for 2 hours.

A 5 mL desalting column was washed with phosphate buffer (pH 6.6) 4 times (1000 G for 6 minutes for each centrifugation). The activated antibody solution was filtered through a desalting column to remove the unreacted linker. The filtrate was collected and pooled. The ratio of trastuzumab and linker was determined by liquid chromatography-mass spectrometry (LC-MS).

500 μL of the activated trastuzumab was slowly added into 500 μL of the nanoparticle solution. The solution was then incubated at 4° C. overnight (shaking or agitation was avoided).

The conjugation solution was removed from 4° C. The solution did not have aggregates on the surface of the Eppendorf tube. The conjugation solution was centrifuged at 21,000 G for 80 minutes. After centrifugation, the tubes were immediately decanted to remove the supernatant and each tube was washed twice with PBS saline.

A 40 mg/mL human serum albumin (HSA) solution was prepared in PBS saline. 500 μL of 40 mg/mL HSA solution was aliquoted into each tube. The tubes were then sonicated for 20 seconds and mixed (this step was repeated several times until the nanoparticles were completely resuspended).

The paclitaxel content was measured by RP-HPLC and was then adjusted to 5.56 mg/mL. The particle size was measured by DLS. The particle size was 164 nm. The resulting solution was then transferred to vials and stored at −80° C.

The Herceptin concentration of the final conjugated particles were analyzed by ELISA. The concentration is 0.18 mg/mL.

### Example 35: Preparation of Conjugated Trastuzumab-Nanoparticle Formulations for In Vitro and In Vivo Studies

To isolate nab-paclitaxel nanoparticles, 20 mL of normal saline was added to 1 vial containing a lyophilized nab-paclitaxel composition (100 mg of paclitaxel). 1.2 mL of the nab-paclitaxel suspension was aliquoted into 25 Eppendorf tubes. The Eppendorf tubes were gently mixing for 20 minutes. One Eppendorf tube was used for an RP-HPLC assay to determine the paclitaxel content and to measure particle size. The remaining Eppendorf tubes were centrifuged at 21,000 RCF for 80 minutes at 20° C. After centrifugation, the tubes were immediately decanted to remove the supernatant. 0.6 mL of normal saline was added to the remaining pellet to reconstitute the nanoparticles (final concentration of 10 mg/mL of paclitaxel). The particle size was measured as 149 nm.

A 21 mg/mL Herceptin® (trastuzumab) solution was prepared using normal saline. 8 mL of the Herceptin® solution was aliquoted into 2 of 15 mL spin concentrators. 100 mM potassium phosphate buffer (pH 6.6) was prepared using endotoxin free water and filtered. The spin concentrators were filled with pH 6.6 phosphate buffer and centrifuged for 30 minutes according to the manufacturer's instructions. This was repeated once. The remaining trastuzumab solution was pipetted into a 15 mL Falcon tube and diluted to a total volume of 2.4 mL. The trastuzumab concentration was measured by size exclusion chromatography (SEC). The trastuzumab concentration was adjusted to 10.5 mg/mL, as needed.

The SM(PEG)₆ package was removed from the −20° C. freezer and warmed to room temperature for 1 hour. 2 mL of sterile DMSO solution was aliquoted into an Eppendorf tube. From this Eppendorf tube, 1.51 mL of DMSO was transferred into the vial containing 100 mg of SM(PEG)₆ (112 mM final concentration). The linker solution was vortexed until the linker was completely dissolved in DMSO.

1 mL of the 10.5 mg/mL trastuzumab solution was aliquoted into 16 Eppendorf tubes. To each Eppendorf tube containing 1 mL of trastuzumab solution, 5.1 μL of the linker solution was slowly added (ratio of linker to antibody is 7.5:1). During this procedure, the solution was protected from light. While still protected from light, the linker/antibody solution was reacted on a shaker for 2 hours.

5 mL desalting columns were washed with phosphate buffer (pH 6.6) 4 times (1000G for 5 minutes for each centrifugation). The activated antibody solution was filtered through a desalting column to remove the unreacted linker. The filtrate was collected and pooled. The ratio of trastuzumab and linker was determined by liquid chromatography-mass spectrometry (LC-MS). 600 μL of the activated trastuzumab was slowly added into 600 μL of the nanoparticle solution. The solution was then incubated at 4° C. overnight (shaking or agitation was avoided).

The conjugation solution was taken out of the fridge. The solution did not have aggregates on the surface of the Eppendorf tube. The particle size was measured as 164 nm

The conjugation solution was centrifuged at 21,000 G for 80 minutes. After centrifugation, the tubes were immediately decanted to remove the supernatant and each tube was washed twice with PBS saline.

A 40 mg/mL human serum albumin (HSA) solution was prepared in PBS saline (pH 7.4). 800 μL of 40 mg/mL HSA solution was aliquoted into each tube. The tubes were then sonicated for 20 seconds and then mixed (this step was repeated several times until the nanoparticles are completely resuspended).

The paclitaxel content was measured by RP-HPLC and was then adjusted by adding solution of 40 mg/mL of HSA and Herceptin to the final of 5.05 mg/mL of paclitaxel and 0.5 mg/mL of Herceptin. The particle size was determined as 157 nm. The resulting solution was then transferred to vials and stored at −80° C.

An immunoblot was performed on isolated nab-paclitaxel nanoparticles and trastuzumab conjugated particles to confirm the antibody conjugation.

### Example 36 In Vitro Efficacy of Admixed, Embedded, and Conjugated Trastuzumab-Nanoparticle Formulations

Various formulations of antibody-nab-paclitaxel were tested for in vitro anti-proliferation effects and inhibition of p(Tyr1248)/Total ErbB2 binding.

Anti-proliferation assay. The growth inhibitory activity in vitro was evaluated using an anti-proliferative assay. 40 μL/well of cells was plated into 384-well plates (Corning, 3712) at their optimized densities and allowed to incubate overnight at 37° C. and 5% CO₂ The following day, cells were treated with test trastuzumab-nanoparticle formulations and placed back into the incubator at 37° C. and 5% CO₂ for 3 days. After 3 days of treatment, cell viability was assessed via the addition of 20 μL/well of Cell Titer-Glo (Promega, G7573). After 30 minutes of incubation, plates were read on the Perkin Elmer Envision for luminescence detection.

Phospho(Tyr1248)/Total ErbB2 Binding Assay. Test trastuzumab-nanoparticle formulations were characterized for ErbB2 binding via a pY1248/total ErbB2 whole cell lysate assay kit (Mesoscale Discovery, K15125D). 25,000 cells/well were plated overnight and allowed to attach to a 96-well culture plate (Corning 3704) at 37° C. and 5% CO₂. The following day, cells were treated with test trastuzumab-nanoparticle formulations for two hours. After two hours of treatment, cells were lysed by adding 65 μL/well of MSD lysis buffer and placed on a rocking shaker at 4° C. for one hour. During this time, the MSD capture plate was blocked with MSD Blocking Buffer A. After one hour, the MSD capture plate was washed with MSD wash buffer. 35 μL/well of cell lysate was then transferred to the MSD capture plate and allowed to incubate for one hour on a rocking shaker at room temperature. Captured protein amounts were then assessed for their Erb2 levels by first discarding the cell lysates and washing the MSD capture plate with MSD wash buffer. 25 μL/well of SULFO-TAG anti-total ErbB2 antibody in antibody dilution buffer was added and then allowed to incubate with the MSD plate on a rocking shaker for one hour at room temperature. After one hour, the MSD plate was washed with MSD wash buffer. 150 μL/well of MSD read buffer was then added and the plate was read immediately on the MSD Sector 5600 instrument.

Data from binding studies is provided in Table 19, and data from proliferation studies is provided in Table 20.

TABLE 19
IC50 for Phospho(Tyr1248)/Total ErbB2 Binding Assay

| | | | BT-474 | |
|---|---|---|---|---|
| | Sample | Paclitaxel:Trastuzumab | IC50 | SK-BR-3 |
| Description | Preparation | Ratio | (nM) | IC50 (nM) |
| Nab-paclitaxel | — | — | >1000 | >1000 |
| SM(PEG)₆ conjugate | Example 23 | 92.5:1 | 0.291 | 0.557 |
| Thiolated conjugate | Example 33 | 12:1 | 0.216 | 0.977 |
| Thiolated conjugate | Example 34 | 31:1 | 0.419 | 0.311 |
| Click conjugate | Example 26 | 51:1 | 0.702 | 0.590 |
| SM(PEG)₆ conjugate with free | Example 35 | 10:1 | 0.441 | 0.301 |
| mAb | | | | |
| Embedded with free mAb | Example 20 | 10:1 | 0.524 | 0.409 |
| Embedded | Example 12, | 5.6:1 | 0.350 | 0.357 |
| | Batch 1 | | | |
| Embedded | Example 12, | 5.9:1 | 0.349 | 0.361 |
| | Batch 2 | | | |
| Embedded | Example 12, | 6.7:1 | 0.456 | 0.370 |
| | Batch 3 | | | |
| Embedded | Example 12, | 4.5:1 | 0.429 | 0.289 |
| | Batch 4 | | | |
| Herceptin ® | — | — | 0.527 | 0.433 |

TABLE 20
Cell Proliferation Assay

| | | | BT- | SK- | CAL- | MDA-MB- | BT- | SK- | CAL- | MDA-MB- |
|---|---|---|---|---|---|---|---|---|---|---|
| | | | 474 | BR-3 | 51 | 468 | 474 | BR-3 | 51 | 468 |
| | Sample | PXT:Tz | GI50 | GI50 | GI50 | GI50 | IC50 | IC50 | IC50 | IC50 |
| Descrip. | Prep | Ratio | (nM) | (nM) | (nM) | (nM) | (nM) | (nM) | (nM) | (nM) |
| Nab-pxt | — | — | 2.715 | 0.184 | 0.149 | 1.032 | 6.092 | 1.228 | 0.181 | 2.105 |
| Admix | Ex. 32, | 12:1 | 2.757 | 0.229 | 0.142 | 1.227 | 5.304 | 1.118 | 0.184 | 2.578 |
| | Batch 1 | | | | | | | | | |
| Admix | Ex. 32, | 31:1 | 0.808 | 0.252 | 0.080 | 0.930 | 1.717 | 0.966 | 0.110 | 2.039 |
| | Batch 2 | | | | | | | | | |
| Admix | Ex. 32, | 51:1 | 0.492 | 0.176 | 0.056 | 0.949 | 1.261 | 0.770 | 0.083 | 2.151 |
| | Batch 3 | | | | | | | | | |
| Admix | Ex. 32, | 92.5:1 | 2.181 | 0.242 | 0.137 | 1.194 | 3.878 | 1.029 | 0.175 | 2.508 |
| | Batch 4 | | | | | | | | | |
| Thiol. | Ex. 33 | 12:1 | 2.264 | 0.208 | 0.101 | 1.176 | 5.148 | 1.129 | 0.130 | 2.177 |
| Conj. | | | | | | | | | | |
| Thiol. | Ex. 34 | 31:1 | 0.924 | 0.150 | 0.085 | 0.752 | 2.528 | 0.797 | 0.111 | 1.735 |
| Conj. | | | | | | | | | | |
| Click | Ex. 26 | 51:1 | 0.632 | 0.152 | 0.085 | 0.745 | 1.391 | 0.659 | 0.109 | 1.654 |
| Conj. | | | | | | | | | | |
| SM- | Ex. 23 | 92.5:1 | 2.780 | 0.268 | 0.171 | 1.618 | 4.967 | 1.439 | 0.215 | 3.085 |
| (PEG)₆ | | | | | | | | | | |
| Conj. | | | | | | | | | | |
| Tz | — | — | >13500 | >13500 | >13500 | >13500 | >13500 | >13500 | >13500 | >13500 |

Ex. = Example;
Tz = trastuzumab;
Pxt = paclitaxel

### Example 37: In Vivo Efficacy of Admixed Embedded, and Conjugated Trastuzumab-Nanoparticle Formulations

This Example demonstrates treatment efficacy of admixed, embedded, and conjugated nab-paclitaxel-trastuzumab formulations administered in BT-474 xenograft mice.

Severe combined immunodeficiency (SCID) mice were subcutaneously implanted with 17β-Estradiol pellets (90 day release, 0.36 mg/pellet) on the day before tumor cell inoculation (day: −1). On day 0, mice were subcutaneously inoculated with BT-474 cells (sourced from ATCC). For the smaller tumor study, around 2 weeks after inoculation with BT-474 cells, tumors were measured and mice were randomized into study groups. For the larger tumor study, around 4 weeks after inoculation with BT-474 cells, tumors were measured and mice were randomized into study groups identified below.

For the smaller tumor study (tumor size of around 150 mm³-180 mm³), 7-8 SCID mice were assigned to each of the following study groups and received a once-weekly administration of: (1) vehicle; (2) nab-paclitaxel: 50 mg/kg (“ABX50”); (3) nab-paclitaxel: 25 mg/kg (“ABX25”); (4) Herceptin®: 5 mg/kg (“Tratsu 5”); (5) Herceptin®: 2.5 mg/kg (“Tratsu 2.5”); (6) nab-paclitaxel-trastuzumab conjugate (50 mg nab-paclitaxel/5 mg trastuzumab per kg) (“Conjugate (50/5)”) (according to Example 35); (7) nab-paclitaxel-trastuzumab conjugate (25 mg nab-paclitaxel/2.5 mg trastuzumab per kg) (“Conjugate (25/2.5)”) (according to Example 35); (8) embedded nab-paclitaxel-trastuzumab (50 mg nab-paclitaxel/5 mg trastuzumab per kg) (“Embedded (50/5)”) (according to Example 20); (9) embedded nab-paclitaxel-trastuzumab (25 mg nab-paclitaxel/2.5 mg trastuzumab per kg) (“Embedded (25/2.5)”) (according to Example 20); (10) admixture of nab-paclitaxel and Herceptin® (50 mg nab-paclitaxel and 5 mg Herceptin® per kg) (“Admixture 50/5”) (according to Example 32, batch 5); and (11) admixture of nab-paclitaxel and Herceptin® (25 mg nab-paclitaxel and 2.5 mg Herceptin® per kg) (“Admixture 25/2.5”) (according to Example 32, batch 5).

For the larger tumor study (tumor size of around 500 mm³-750 mm³), 7 SCID mice were assigned to each of the following study groups and received a once-weekly administration of: (1) vehicle; (2) nab-paclitaxel: 50 mg/kg; (3) Herceptin®: 5 mg/kg; (4) nab-paclitaxel-trastuzumab conjugate (50 mg nab-paclitaxel/5 mg trastuzumab per kg) (according to Example 35); (5) embedded nab-paclitaxel-trastuzumab (50 mg nab-paclitaxel/5 mg trastuzumab per kg) (according to Example 20); and (6) admixture of Abraxane® and Herceptin® (50 mg nab-paclitaxel and 5 mg Herceptin® per kg) (according to Example 32, batch 5).

Animal tumor measurements and body weights were measured twice a week. The last tumor measurements were blinded from the originating group designation.

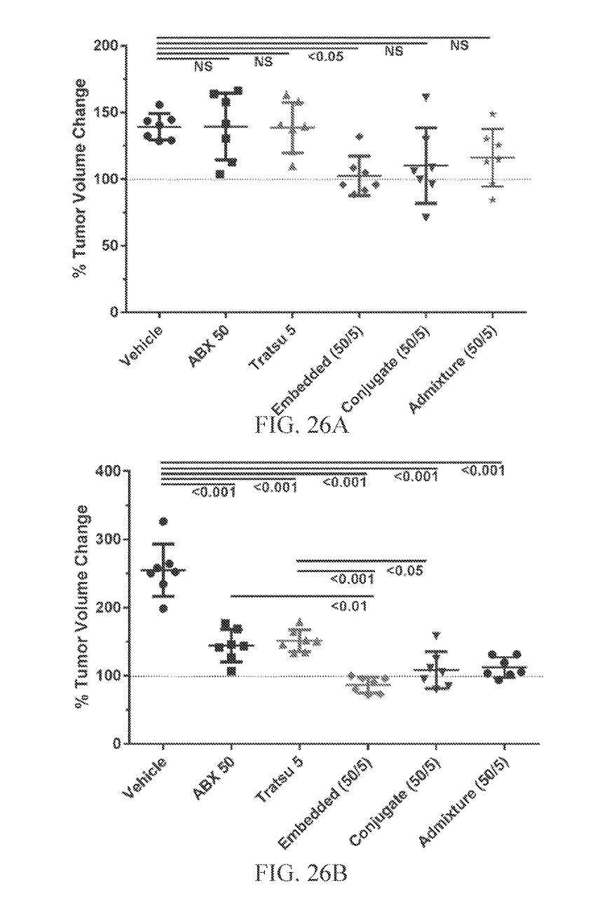

Percent tumor volume change results from the smaller tumor study are shown in FIGS. 25A-25B (7 days after treatment) and FIGS. 25C-25D (14 days after treatment). By day 14, higher dose nab-paclitaxel-trastuzumab conjugate group (50 mg nab-paclitaxel/5 mg trastuzumab per kg) achieved significantly improved antitumor activity compared to single agent nab-paclitaxel (FIG. 25C).

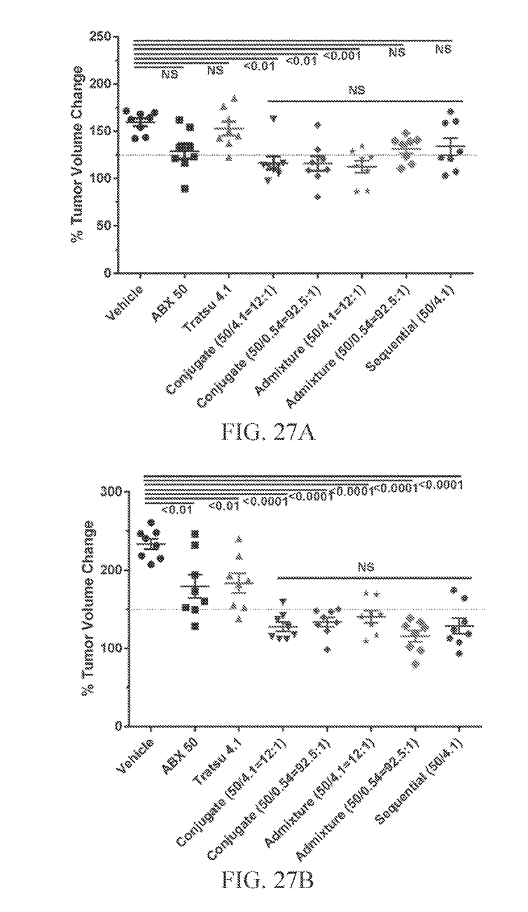

Results from the larger tumor study are shown in FIG. 26A (7 days after treatment) and FIG. 26B (14 days after treatment). By day 14, embedded nab-paclitaxel-trastuzumab (50 mg nab-paclitaxel/5 mg trastuzumab per kg) achieved significantly improved antitumor activity compared to single agent nab-paclitaxel or single agent Trastuzumab. Further, 14 days after treatment, conjugated nab-paclitaxel-trastuzumab (50 mg nab-paclitaxel/5 mg trastuzumab per kg) achieved significantly improved antitumor activity compared to single agent Trastuzumab.

### Example 38: In Vivo Efficacy of Admixed, Embedded, and Conjugated Trastuzumab-Nanoparticle Formulations

This example demonstrates treatment efficacy of admixed, embedded, and conjugated nab-paclitaxel-trastuzumab formulations on a BT-474 xenograft mouse model.

Severe combined immunodeficiency (SCID) mice prepared as described in Example 31. Around 4 weeks after inoculation with BT-474 cells, tumors were measured (approximately 600 mm³) and mice were randomized into study groups identified below.

8 SCID mice were assigned to each of the following study groups and received a once-weekly administration of: (1) vehicle (5% HSA); (2) nab-paclitaxel: 50 mg/kg (“ABX 50”); (3) Herceptin®: 4.1 mg/kg (“Tratsu 4.1”); (4) nab-paclitaxel-trastuzumab conjugate (50 mg nab-paclitaxel/4.1 mg trastuzumab per kg) (“Conjugate 50/4.1=12:1”) (according to Example 33); (5) nab-paclitaxel-trastuzumab conjugate (50 mg nab-paclitaxel/0.54 mg trastuzumab per kg) (“Conjugate 50/0.54=92.5:1)”) (according to Example 23); (6) admixture of nab-paclitaxel and Herceptin® (50 mg nab-paclitaxel and 4.1 mg Herceptin® per kg) (Admixture (50/4.1=12:1)”) (according to Example 36, batch 1); (7) admixture of nab-paclitaxel and Herceptin® (50 mg nab-paclitaxel and 0.54 mg Herceptin® per kg) (“Admixture (50/0.54=92.5:1)”) (according to Example 36, batch 4); and (8) sequential administration of nab-paclitaxel (50 mg/kg) and Herceptin® (4.1 mg/kg) (“Sequential (50/4.1)”).

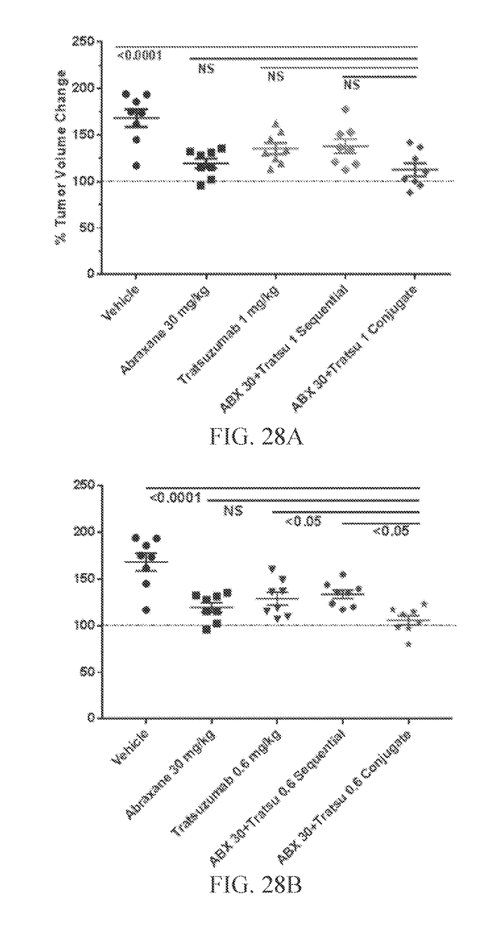

Percent tumor volume change results from the study are shown in FIGS. 27A (7 days after treatment on day 0) and 27B (14 days after treatment on days 0 and 7).

### Example 39: In Vivo Efficacy of Admixed Embedded, and Conjugated Trastuzumab-Nanoparticle Formulations

This example demonstrates treatment efficacy of embedded and conjugated nab-paclitaxel-trastuzumab formulations on a BT-474 xenograft mouse model.

Severe combined immunodeficiency (SCID) mice prepared as described in Example M. Around 4 weeks after inoculation with BT-474 cells, tumors were measured (approximately 600 mm³) and mice were randomized into study groups identified below.

8 SCID mice were assigned to each of the following study groups in the 1 mg/kg Herceptin® study group and received a once-weekly administration of: (1) vehicle (5% HSA); (2) nab-paclitaxel: 30 mg/kg; (3) Herceptin®: 1 mg/kg; (4) sequential administration of nab-paclitaxel (30 mg/kg) and Herceptin® (1 mg/kg); and (5) nab-paclitaxel-trastuzumab conjugate (30 mg nab-paclitaxel/1 mg trastuzumab per kg) (according to Example 34); (6) Herceptin®: 0.6 mg/kg; (7) sequential administration of nab-paclitaxel (30 mg/kg) and Herceptin® (0.6 mg/kg); and (8) nab-paclitaxel-trastuzumab conjugate (30 mg nab-paclitaxel/0.6 mg trastuzumab per kg) (according to Example 26).

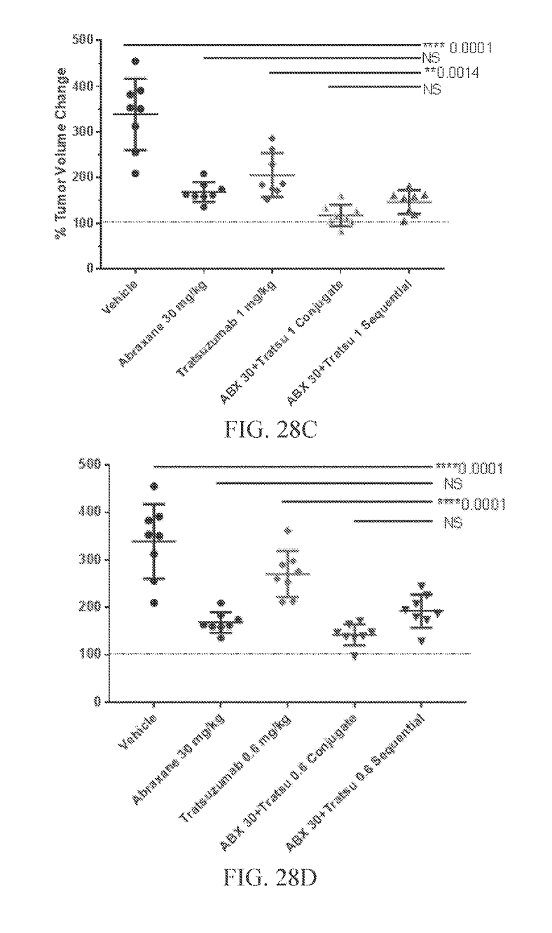

Tumor volumes were measured on day 7 after a single dose of above-identified treatments on day 0. Results of percentage tumor volume change on day 7 are shown in FIGS. 28A-28B.

Tumor volumes were measured on day 14 after a two doses of above-identified treatments on days 0 and 7. Results of percentage tumor volume change on day 14 are shown in FIGS. 28C-28D.

1. A composition comprising nanoparticles comprising (a) a hydrophobic drug, (b) an albumin, and (c) a bioactive polypeptide conjugated to the albumin.

2. The composition of claim 1, wherein the bioactive polypeptide is covalently crosslinked to the albumin.

3. The composition of claim 2, wherein the bioactive polypeptide is covalently crosslinked to the albumin through a chemical crosslinker.

4. The composition of claim 2, wherein the bioactive polypeptide is covalently crosslinked to the albumin through a disulfide bond.

5. The composition of claim 1, wherein the bioactive polypeptide is conjugated to the albumin through a non-covalent crosslinker.

6. The composition of claim 5, wherein the bioactive polypeptide comprises a first component of the non-covalent crosslinker and the albumin comprises a second component of the non-covalent crosslinker, and wherein the first component specifically binds to the second component.

7. The composition of claim 6, wherein the non-covalent crosslinker comprises nucleic acid molecules, wherein at least a portion of the nucleic acid molecules are complementary.

8. A composition comprising nanoparticles comprising (a) a solid core comprising a hydrophobic drug, (b) an albumin associated with a surface of the nanoparticle, and (c) a bioactive polypeptide embedded in the surface of the nanoparticle or the solid core.

9. The composition of claim 8, wherein the bioactive polypeptide is embedded in the surface of the nanoparticles.

10. The composition of claim 8, wherein the bioactive polypeptide is embedded in the solid core.

11-12. (canceled)

13. The composition of claim 1, wherein the bioactive polypeptide is an antibody or fragment thereof.

14. (canceled)

15. The composition of claim 1, wherein the weight ratio of the hydrophobic drug to the bioactive polypeptide in the nanoparticles in the composition is about 1:1 to about 100:1.

16. The composition of claim 1, wherein the weight ratio of the albumin to the bioactive polypeptide in the nanoparticles in the composition is about 1:1 to about 1000:1.

17. The composition of claim 1, wherein the weight ratio of the albumin to the hydrophobic drug in the nanoparticles in the composition is about 1:1 to about 20:1.

18. (canceled)

19. The composition of claim 1, wherein the composition further comprises bioactive polypeptide not associated with the nanoparticles.

20. (canceled)

21. The composition of claim 1, wherein the average diameter of the nanoparticles as measured by dynamic light scattering is no greater than about 200 nm.

22. The composition of claim 1, wherein the composition further comprises albumin not associated with the nanoparticles.

23. The composition of claim 1, wherein the hydrophobic drug is a taxane.

24. The composition of claim 1, wherein the hydrophobic drug is paclitaxel.

25. (canceled)

26. A method of making a composition comprising nanoparticles comprising a hydrophobic drug, an albumin and a bioactive polypeptide, the method comprising:
i) subjecting a mixture of an organic solution and an aqueous solution to high pressure homogenization, thereby forming an emulsion,
wherein the organic solution comprises the hydrophobic drug dissolved in one or more organic solvents, and
wherein the aqueous solution comprises the albumin and the bioactive polypeptide; and
ii) removing at least a portion of the one or more organic solvents from the emulsion, thereby forming the composition.

27-40. (canceled)

41. A method of treating a disease in an individual, comprising administering to the individual an effective amount of the composition of claim 1.

42-43. (canceled)


SEQUENCE LISTING (2 sequences)

SEQ ID NO: 1 - DNA, 20 bp, Artificial Sequence
  Synthetic Construct
cacacacacacacacacaca

SEQ ID NO: 2 - DNA, 15 bp, Artificial Sequence
  Synthetic Construct
gtgtgtgtgtgtgtg